(19) United States
(12) Patent Application Publication
(10) Pub. No.: US 2021/0155701 A1
(43) Pub. Date: May 27, 2021

(54) POLYPEPTIDE COMPRISING IL-1R1 BINDING DOMAIN AND CARRYING MOIETY

(71) Applicant: Chugai Seiyaku Kabushiki Kaisha, Kita-ku, Tokyo (JP)

(72) Inventors: Maiko HOSHINO, Singapore (SG); Tomoyuki IGAWA, Singapore (SG); Naoka HIRONIWA, Shizuoka (JP)

(21) Appl. No.: 17/058,889

(22) PCT Filed: May 30, 2019
(86) PCT No.: PCT/JP2019/021462

§ 371 Date: Nov. 25, 2020

(30) Foreign Application Priority Data
May 30, 2018 (JP) 2018-103232

(51) Int. Cl. C07K 16/28

(52) U.S. Cl. CPC C07K 16/2866; C07K 2317/94; C07K 2317/569

(57) ABSTRACT

The present invention relates to a polypeptide comprising an antigen binding domain and a carrying moiety having an inhibiting domain that inhibits the antigen binding activity of the antigen binding domain, and having a longer half-life than that of the antigen binding domain existing alone, methods for producing and screening for the polypeptide, a pharmaceutical composition comprising the polypeptide, methods for producing and screening for an antigen binding domain whose antigen binding activity is inhibited by associating with particular VL, VH or VHH, and a fusion polypeptide library including an antigen binding domain whose antigen binding activity is inhibited by associating with particular VL, VH or VHH.

### TECHNICAL FIELD

The present invention relates to a polypeptide comprising an antigen binding domain that binds to IL-1R1 (IL-1R1 binding domain) and a carrying moiety having an inhibiting domain that inhibits the antigen binding activity of the IL-1R1 binding domain, and having a longer half-life than the half-life of the antigen binding domain which exists alone, methods for producing and screening for the polypeptide, a pharmaceutical composition comprising the polypeptide, methods for producing and screening for an IL-1R1 binding domain whose antigen binding activity can be inhibited by associating with particular VL, VH or VHH, and a library of fusion polypeptides in which an IL-1R1 binding domain whose antigen binding activity can be inhibited by associating with particular VL, VH or VHH.

The present invention also relates to a polypeptide comprising an antigen binding domain VL and a carrying moiety having an inhibiting domain that inhibits the antigen binding activity of the antigen binding domain VL, and having a longer half-life than the half-life of the antigen binding domain VL which exists alone, methods for producing and screening for the polypeptide, a pharmaceutical composition comprising the polypeptide, methods for producing and screening for an antigen binding domain VL whose antigen binding activity can be inhibited by associating with particular VL, VH or VHH, and a library of fusion polypeptides in which an antigen binding domain VL whose antigen binding activity can be inhibited by associating with particular VL, VH or VHH.

### BACKGROUND ART

Antibodies have received attention as drugs because of being highly stable in plasma and causing few adverse reactions. Among them, many IgG-type antibody drugs have been launched, and a large number of antibody drugs are currently under development (NPL 1 and NPL 2).

Rituxan against CD20, cetuximab against EGFR, Herceptin against HER2, and the like have been approved so far as therapeutic drugs for cancer using antibody drugs (NPL 3). These antibody molecules bind to their antigens expressed on cancer cells and thereby exert cytotoxic activity against the cancer cells through ADCC activity, etc. Such cytotoxic activity based on ADCC activity, etc. is known to depend on the number of antigens expressed on target cells of therapeutic antibodies (NPL 4). Therefore, high expression levels of targeted antigens are preferred from the viewpoint of the effects of therapeutic antibodies. However, if an antigen, albeit having a high expression level, is expressed in normal tissues, the cytotoxic activity based on ADCC activity, etc. is exerted against the normal cells. Hence, adverse reactions become a serious problem. Therefore, it is preferred that antigens targeted by therapeutic antibodies as therapeutic drugs for cancer should be expressed specifically on cancer cells. For example, an antibody molecule against EpCAM known as a cancer antigen had been considered promising as a therapeutic drug for cancer. However, the EpCAM antigen is known to be also expressed in the pancreas. In actuality, it has been reported in clinical trials that the administration of an anti-EpCAM antibody causes pancreatitis as an adverse reaction due to cytotoxic activity against the pancreas (NPL 5).

In the wake of the success of antibody drugs exerting cytotoxic activity based on ADCC activity, second-generation improved antibody molecules exerting strong cytotoxic activity have been reported as a result of, for example, enhancing ADCC activity by the removal of fucose from the N-linked oligosaccharide of a natural human IgG1 Fc region (NPL 6) or enhancing ADCC activity by enhancing binding to Fc gamma RIIIa through the amino acid substitution of a natural human IgG1 Fc region (NPL 7). Improved antibody molecules exerting stronger cytotoxic activity, such as an antibody drug conjugate (ADC) containing an antibody conjugated with a drug having strong cytotoxic activity (NPL 8), and a low-molecular antibody exerting cytotoxic activity against cancer cells by recruiting T cells to the cancer cells (NPL 9) have also been reported as antibody drugs exerting cytotoxic activity against cancer cells under a mechanism other than NK cell-mediated ADCC activity as mentioned above.

Such antibody molecules exerting stronger cytotoxic activity can exert cytotoxic activity even against cancer cells expressing an antigen at a level that is not high, but also exert cytotoxic activity against normal tissues expressing the antigen at a low level, similarly to cancer cells. In actuality, EGFR-BiTE, a bispecific antibody against CD3 and EGFR, can exert strong cytotoxic activity against cancer cells and exert an antitumor effect, by recruiting T cells to the cancer cells, as compared with cetuximab, natural human IgG1 against the EGFR antigen. On the other hand, it has also been found that serious adverse reactions appear by the administration of EGFR-BiTE to cynomolgus monkeys, because EGFR is also expressed in normal tissues (NPL 10). Also, ADC bivatuzumab mertansine containing mertansine conjugated with an antibody against CD44v6 highly expressed on cancer cells has been clinically found to cause severe dermal toxicity and hepatoxicity, because CD44v6 is also expressed in normal tissues (NPL 11).

As mentioned above, use of an antibody that can exert strong cytotoxic activity even against cancer cells expressing an antigen at low levels requires the target antigen to be expressed in an exceedingly cancer-specific manner. However, considering that a target antigen HER2 of Herceptin or a target antigen EGFR of cetuximab is also expressed in normal tissues, only a limited number of cancer antigens may be expressed in an exceedingly cancer-specific manner. Therefore, adverse reactions ascribable to a cytotoxic effect on normal tissues may become a problem, though cytotoxic activity against cancer can be strengthened.

Recently, ipilimumab, which enhances tumor immunity by inhibiting CTLA4 contributing to immunosuppression in cancer, has been shown to extend overall survival in metastatic melanoma (NPL 12). However, ipilimumab systemically inhibits CTLA4 and therefore advantageously causes autoimmune disease-like severe adverse reactions due to the systemic activation of immunity, though enhancing the tumor immunity (NPL 13).

Meanwhile, antibody drugs exerting a therapeutic effect by inhibiting inflammatory cytokines in inflammatory or autoimmune diseases are known as antibody drugs against diseases other than cancer (NPL 14). It is known that, for example, Remicade or Humira targeting TNF, and Actemra targeting IL-6R exert a high therapeutic effect on rheumatoid arthritis, whereas infectious disease is seen as an adverse reaction due to the systemic neutralization of these cytokines (NPL 15). Drugs targeting IL-1 signaling are evaluated in clinical study for osteoarthritis treatment. Similar to cytokine blockers above, increasing of infection and neutropenia were seen in those clinical trials due to the systemic neutralization of IL-1 signaling (NPL 19 and NPL 20).

Osteoarthritis (OA) is the most common degenerative joint disease among elderly people. OA affects majority of individuals over the age of 65 and is a leading musculoskeletal cause of impaired mobility. OA patients are suffering from cartilage matrix degradation, osteophyte generation and chronic pain in affected joints. Because the precise molecular mechanisms which are involved in the degradation of cartilage matrix and development of OA are poorly understood, there are currently no disease modifying drug for OA (NPL 21 and NPL 22).

Various techniques have been developed as techniques applicable to second-generation antibody drugs. For example, techniques of improving effector functions, antigen binding capacity, pharmacokinetics, or stability or reducing a risk of immunogenicity have been reported (NPL 16). However, there are still a few reports on techniques that allow antibody drugs to act specifically on a target tissue in order to solve adverse reactions as described above. The reported techniques include a method which involves: connecting an antibody to a masking peptide via a linker that is cleaved by protease expressed at a lesion site such as a cancer tissue or an inflammatory tissue, thereby masking the antigen binding site of the antibody with the masking peptide and inhibiting the antigen binding activity of the antibody; and dissociating the masking peptide therefrom by the protease cleavage of this linker so that the antibody restores its antigen binding activity and becomes capable of binding to the antigen in a target pathological tissue (NPL 17 and NPL 18 and PTL 1).

### CITATION LIST

### Patent Literature

[PTL 1] International Publication No. WO2010/081173

### Non Patent Literature

[NPL 1] Monoclonal antibody successes in the clinic. Janice M Reichert, Clark J Rosensweig, Laura B Faden & Matthew C Dewitz, Nat. Biotechnol. (2005) 23, 1073-1078
[NPL 2] The therapeutic antibodies market to 2008. Pavlou A K, Belsey M J., Eur. J. Pharm. Biopharm. (2005) 59 (3), 389-396
[NPL 3] Monoclonal antibodies: versatile platforms for cancer immunotherapy. Weiner L M, Surana R, Wang S., Nat. Rev. Immunol. (2010) 10 (5), 317-327
[NPL 4] Differential responses of human tumor cell lines to anti-p185HER2 monoclonal antibodies. Lewis G D, Figari I, Fendly B, Wong W L, Carter P, Gorman C, Shepard H M, Cancer Immunol Immunotherapy (1993) 37, 255-263
[NPL 5] ING-1, a monoclonal antibody targeting Ep-CAM in patients with advanced adenocarcinomas. de Bono J S, Tolcher A W, Forero A, Vanhove G F, Takimoto C, Bauer R J, Hammond L A, Patnaik A, White M L, Shen S, Khazaeli M B, Rowinsky E K, LoBuglio A F, Clin. Cancer Res. (2004) 10 (22), 7555-7565
[NPL 6] Non-fucosylated therapeutic antibodies as next-generation therapeutic antibodies. Satoh M, Iida S, Shitara K., Expert Opin. Biol. Ther. (2006) 6 (11), 1161-1173
[NPL 7] Optimizing engagement of the immune system by anti-tumor antibodies: an engineer's perspective. Desjarlais J R, Lazar G A, Zhukovsky E A, Chu S Y., Drug Discov. Today (2007) 12 (21-22), 898-910
[NPL 8] Antibody-drug conjugates: targeted drug delivery for cancer. Alley S C, Okeley N M, Senter P D., Curr. Opin. Chem. Biol. (2010) 14 (4), 529-537
[NPL 9] BiTE: Teaching antibodies to engage T-cells for cancer therapy. Baeuerle P A, Kufer P, Bargou R., Curr. Opin. Mol. Ther. (2009) 11 (1), 22-30
[NPL 10] T cell-engaging BiTE antibodies specific for EGFR potently eliminate KRAS- and BRAF-mutated colorectal cancer cells. Lutterbuese R, Raum T, Kischel R, Hoffmann P, Mangold S, Rattel B, Friedrich M, Thomas O, Lorenczewski G, Rau D, Schaller E, Herrmann I, Wolf A, Urbig T, Baeuerle P A, Kufer P., Proc. Natl. Acad. Sci. U.S.A. (2010) 107 (28), 12605-12610
[NPL 11] Phase I trial with the CD44v6-targeting immunoconjugate bivatuzumab mertansine in head and neck squamous cell carcinoma. Riechelmann H, Sauter A, Golze W, Hanft G, Schroen C, Hoermann K, Erhardt T, Gronau S., Oral Oncol. (2008) 44 (9), 823-829
[NPL 12] Ipilimumab in the treatment of melanoma. Trinh V A, Hwu W J., Expert Opin. Biol. Ther., (2012) Apr. 14 (doi: 10.1517/14712598.2012.675325)
[NPL 13] IPILIMUMAB—A NOVEL IMMUNOMODULATING THERAPY CAUSING AUTOIMMUNE HYPOPHYSITIS: A CASE REPORT AND REVIEW. Juszczak A, Gupta A, Karavitaki N, Middleton M R, Grossman A., Eur. J. Endocrinol. (2012) Apr. 10 (doi: 10.1530/EJE-12-0167)
[NPL 14] The Japanese experience with biologic therapies for rheumatoid arthritis. Takeuchi T, Kameda H., Nat. Rev. Rheumatol. (2010) 6 (11), 644-652
[NPL 15] Current evidence for the management of rheumatoid arthritis with biological disease-modifying antirheumatic drugs: a systematic literature review informing the EULAR recommendations for the management of RA. Nam J L, Winthrop K L, van Vollenhoven R F, Pavelka K, Valesini G, Hensor E M, Worthy G, Landewe R, Smolen J S, Emery P, Buch M H., Ann. Rheum. Dis. (2010) 69 (6), 976-986
[NPL 16] Antibody engineering for the development of therapeutic antibodies. Kim S J, Park Y, Hong H J., Mol. Cells. (2005) 20 (1), 17-29
[NPL 17] Tumor-specific activation of an EGFR-targeting probody enhances therapeutic index. Desnoyers L R, Vasiljeva O, Richardson J H, Yang A, Menendez E E, Liang T W, Wong C, Bessette P H, Kamath K, Moore S J, Sagert J G, Hostetter D R, Han F, Gee J, Flandez J, Markham K, Nguyen M, Krimm M, Wong K R, Liu S, Daugherty P S, West J W, Lowman H B. Sci Transl Med. 2013 Oct. 16; 5(207): 207ra144.
[NPL 18] Probody therapeutics for targeting antibodies to diseased tissue. Polu K R, Lowman H B. Expert Opin Biol Ther. 2014 August; 14(8): 1049-53.
[NPL 19] Arthritis Res Ther. 2011 Jul. 29; 13(4):R125. doi: 10.1186/ar3430
[NPL 20] Abstract of ACR/ARHP Annual Meeting, https://plan.core-apps.com/tristar_acr17/abstract/7f9a3c05b0ca255af1fc655b034e5eaa, Date of Search: Apr. 23, 2018
[NPL 21] Nature Reviews Disease Primers volume 2, Article number: 16072 (2016)
[NPL 22] Calcif Tissue Int. 2014 December; 95(6): 495-505

### SUMMARY OF INVENTION

### Technical Problem

The present inventors have thought that the techniques of dissociating, by protease cleavage, a masking peptide inhibiting the antigen binding activity of an antibody so that the antibody restores its antigen binding activity, as described above might cause adverse reactions, because the antibody cleaved at a lesion site may distribute to normal tissues through blood flow, as the cleavage by protease is irreversible. Further, there has been a problem that it is difficult for conventional antibodies to target antigens present in a deep part of a cartilage tissue since such antibodies have a high molecular weight.

The present invention has been made on the basis of such an idea. An object of the present invention is to provide a pharmaceutical composition useful in disease treatment with a reduced adverse reaction, and an active ingredient thereof. Further, an object of the present invention is to provide a pharmaceutical composition that can reach a deep part in a target cartilage tissue well, and an active ingredient thereof. Another object of the present invention is to provide methods for screening for and producing the pharmaceutical composition and the active ingredient.

### Solution to Problem

The present inventors have conducted diligent studies and consequently developed a polypeptide comprising an antigen binding domain and a carrying moiety having an inhibiting domain that inhibits the binding activity of the antigen binding domain, and having a longer half-life than the half-life of the antigen binding domain which exists alone. It is considered that use of the polypeptide can allow the antigen binding domain to restore its antigen binding activity in a disease tissue and exert the antigen binding activity in the disease tissue. Furthermore, the systemic distribution of an activated form of the antigen binding domain can be suppressed owing to the difference in half-life between the polypeptide comprising the antigen binding domain whose antigen binding activity is inhibited and a polypeptide comprising the antigen binding domain whose antigen binding activity is restored. Moreover, the present inventors have found that the polypeptide or a pharmaceutical composition comprising the polypeptide is useful in disease treatment and also found that: the polypeptide or the pharmaceutical composition is useful in disease treatment which involves administering the polypeptide; and the polypeptide is useful in the production of a drug for disease treatment. The present inventors have further developed methods for screening for and producing the polypeptide, methods for producing and screening for an antigen binding domain whose antigen binding activity can be inhibited by associating with particular VL, VH or VHH, and a library including an antigen binding domain whose antigen binding activity can be inhibited by associating with particular VL, VH or VHH, completing the present invention.

The present invention is based on these findings and specifically encompasses exemplary embodiments described below.
(A0) A polypeptide comprising an antigen binding domain and a carrying moiety, wherein the carrying moiety has an inhibiting domain that inhibits the antigen binding activity of the antigen binding domain, wherein the antigen is one or more molecules involved in IL-1 mediated signal transduction.
(A1) A polypeptide comprising an antigen binding domain and a carrying moiety, wherein the antigen binding domain has a shorter half-life in blood than that of the carrying moiety that has an inhibiting domain that inhibits the antigen binding activity of the antigen binding domain, wherein the antigen is one or more molecules involved in IL-1 mediated signal transduction.
(A2) The polypeptide according to (A0) or (A1), wherein the molecular weight of the antigen binding domain is smaller than that of the carrying moiety.
(A3) The polypeptide according to any of (A0) to (A2), wherein the molecular weight of the antigen binding domain is 120 kDa, 100 kDa, 80 kDa, 60 kDa, 40 kDa, 20 kDa or smaller.
(A4) The polypeptide according to any of (A0) to (A3), wherein the carrying moiety has FcRn binding activity, and the antigen binding domain has no FcRn binding activity or has weaker FcRn binding activity than that of the carrying moiety.
(A5) The polypeptide according to any of (A0) to (A4), wherein the antigen binding domain is capable of being released from the polypeptide, and the antigen binding domain released from the polypeptide has higher antigen binding activity than that before the release.
(A6) The polypeptide according to any of (A0) to (A5), wherein the inhibiting domain of the carrying moiety associates with the antigen binding domain and thereby inhibits the antigen binding activity of the antigen binding domain.
(A7) The polypeptide according to (A5), wherein the polypeptide comprises a cleavage site, wherein the cleavage site is cleaved so that the antigen binding domain becomes capable of being released from the polypeptide.
(A8) The polypeptide according to (A6), wherein the polypeptide comprises a cleavage site, wherein the cleavage site is cleaved so that the association of the inhibiting domain of the carrying moiety with the antigen binding domain is canceled.
(A9) The polypeptide according to (A7) or (A8), wherein in the polypeptide, the N terminus of the carrying moiety and the C terminus of the antigen binding domain are fused via a linker or without a linker, or wherein in the polypeptide, the C terminus of the carrying moiety and the N terminus of the antigen binding domain are fused via a linker or without a linker.
(A10) The polypeptide according to (A9), wherein the polypeptide further has a protease cleavage sequence, wherein the cleavage sequence is located between the N terminus of the carrying moiety and the C terminus of the antigen binding domain, the C terminus of the carrying moiety and the N terminus of the antigen binding domain, within the sequence of the antigen binding domain, or within the sequence of the carrying moiety.
(A11) The polypeptide according to (A7) or (A8), wherein the cleavage site comprises a protease cleavage sequence.
(A12) The polypeptide according to (A10) or (A11), wherein the protease is a target tissue specific protease.
(A13) The polypeptide according to (A12), wherein the target tissue is an inflammatory tissue.
(A14) The polypeptide according to (A10) or (A11), wherein the protease is at least one protease selected from matriptase, urokinase (uPA), and metalloproteinase.
(A15) The polypeptide according to (A14), wherein the protease is at least one protease selected from MT-SP1, uPA, MMP1, MMP2, MMP3, MMP7, MMP9, MMP13, MMP14, ADAM17, ADAMTS4, and ADAMTS5.
(A16) The polypeptide according to (A10) or (A11), wherein the protease cleavage sequence comprises a sequence selected from SEQ ID NOs: 508, 509 and 510.
(A17) The polypeptide according to any of (A11) to (A16), wherein a first flexible linker is further attached to one end of the protease cleavage sequence.
(A18) The polypeptide according to (A17), wherein a second flexible linker is further attached to the other end of the protease cleavage sequence.
(A19) The polypeptide according to (A17), wherein the first flexible linker is a flexible linker consisting of a glycine-serine polymer.
(A20) The polypeptide according to (A18), wherein the second flexible linker is a flexible linker consisting of a glycine-serine polymer.
(A21) The polypeptide according to any of (A0) to (A20), wherein the antigen binding domain comprises a single-domain antibody or IL-1R antagonist (IL-1Ra) or is a single-domain antibody or IL-1R antagonist, wherein the inhibiting domain of the carrying moiety inhibits the antigen binding activity of the single-domain antibody or IL-1R antagonist.
(A22) The polypeptide according to (A21), wherein the single-domain antibody is VHH, VH having antigen binding activity by itself, or VL having antigen binding activity by itself.
(A23) The polypeptide according to any of (A0) to (A22), wherein the antigen binding domain comprises a single-domain antibody, and the inhibiting domain of the carrying moiety is VHH, antibody VH, or antibody VL, wherein the antigen binding activity of the single-domain antibody is inhibited by the VHH, the antibody VH, or the antibody VL.
(A24) The polypeptide according to any of (A0) to (A23), wherein the antigen binding domain comprises a single-domain antibody, and the inhibiting domain of the carrying moiety is VHH, antibody VH, or antibody VL, wherein the antigen binding activity of the single-domain antibody is inhibited by associating with the VHH, the antibody VH, or the antibody VL.
(A25) The polypeptide according to any of (A21) to (A24), wherein the single-domain antibody is VHH or VH having antigen binding activity by itself, and the inhibiting domain of the carrying moiety is antibody VL, wherein the antigen binding activity of the VHH or the VH having antigen binding activity by itself is inhibited by associating with the antibody VL.
(A26) The polypeptide according to any of (A21) to (A25), wherein the single-domain antibody is VHH, wherein the VHH has an amino acid substitution at least one position selected from amino acid positions 37, 44, 45, and 47 (all according to the Kabat numbering).
(A27) The polypeptide according to any of (A21) to (A25), wherein the single-domain antibody is VHH, wherein the VHH contains at least one amino acid selected from amino acids 37V, 44G, 45L, and 47W (all according to the Kabat numbering).
(A28) The polypeptide according to any of (A21) to (A25), wherein the single-domain antibody is VHH, wherein the VHH contains at least one amino acid substitution selected from amino acid substitutions F37V, Y37V, E44G, Q44G, R45L, H45L, G47W, F47W, L47W, T47W, and S47W (all according to the Kabat numbering).
(A29) The polypeptide according to any of (A21) to (A25), wherein the single-domain antibody is VHH, wherein the VHH has amino acid substitutions at least one set of positions selected from positions 37/44, positions 37/45, positions 37/47, positions 44/45, positions 44/47, positions 45/47, positions 37/44/45, positions 37/44/47, positions 37/45/47, positions 44/45/47, and positions 37/44/45/47 (all according to the Kabat numbering).
(A30) The polypeptide according to any of (A21) to (A25), wherein the single-domain antibody is VHH, wherein the VHH contains at least one set of amino acids selected from 37V/44G, 37V/45L, 37V/47W, 44G/45L, 44G/47W, 45L/47W, 37V/44G/45L, 37V/44G/47W, 37V/45L/47W, 44G/45L/47W, and 37V/44G/45L/47W (all according to the Kabat numbering).
(A31) The polypeptide according to any of (A21) to (A25), wherein the single-domain antibody is VHH, wherein the VHH contains at least one set of amino acid substitutions selected from F37V/R45L, F37V/G47W, R45L/G47W, and F37V/R45L/G47W (all according to the Kabat numbering).
(A32) The polypeptide according to any of (A21) to (A24), wherein the single-domain antibody is VL having antigen binding activity by itself, and the inhibiting domain of the carrying moiety is antibody VH, wherein the antigen binding activity of the VL having antigen binding activity by itself is inhibited by associating with the antibody VH.
(A33) The polypeptide according to any of (A21) to (A24), wherein the single-domain antibody is VL having antigen binding activity by itself, and the inhibiting domain of the carrying moiety is antibody VL, wherein the antigen binding activity of the VL having antigen binding activity by itself is inhibited by associating with the antibody VL.
(A34) The polypeptide according to (A33), wherein the VL having antigen binding activity by itself comprises the amino acid sequence of SEQ ID NO: 479 or 480.
(A35) A polypeptide comprising an antigen binding domain and a carrying moiety, wherein the antigen binding domain has a shorter half-life in blood than that of the carrying moiety that has an inhibiting domain that inhibits the antigen binding activity of the antigen binding domain, wherein the antigen binding domain binds to an epitope within IL-1R1, IL-1alpha, IL-1beta and/or IL-1RAcP, and/or wherein the antigen is IL1-R1 and the antigen binding domain competes for binding to the epitope with a single-domain antibody VL selected from the group consisting of 1) and 2) below:
   1) a single-domain antibody VL comprising the amino acid sequence of SEQ ID NO: 479, and
   2) a single-domain antibody VL comprising the amino acid sequence of SEQ ID NO: 480.
(A36) A polypeptide comprising an antigen binding domain that binds to IL-1R1 (IL-1R1 binding domain) and a carrying moiety, wherein the antigen binding domain has a shorter half-life in blood than that of the carrying moiety that has an inhibiting domain that inhibits the IL-1R1 binding activity of the antigen binding domain, wherein the IL-1R1 binding domain does not compete with IL-1Ra or competes with IL-1Ra.
(A37) The polypeptide according to any of (A0) to (A34), wherein the antigen binding domain is a soluble IL-1R or a single-domain antibody binding to IL-1R1, IL-1alpha, IL-1beta and/or IL-1RAcP.
(A38) The polypeptide according to (A37), wherein the antigen binding domain is a soluble IL-1R1, soluble IL-1R2, soluble IL-1RAcP or a single-domain antibody binding to IL-1alpha and/or IL-1beta.
(A39) The polypeptide according to any of (A23) to (A38), wherein the antibody VH comprises an amino acid sequence of SEQ ID NO: 486 and/or the antibody VL comprises an amino acid sequence of SEQ ID NO: 484 or 485.
(A40) The polypeptide according to any of (A0) to (A39), wherein the carrying moiety has an FcRn binding region.
(A41) The polypeptide according to any of (A0) to (A40), wherein the carrying moiety comprises an antibody constant region.
(A42) The polypeptide according to (A41), wherein the antibody constant region of the carrying moiety and the antigen binding domain are fused via a linker or without a linker.
(A43) The polypeptide according to (A41), wherein the carrying moiety comprises an antibody heavy chain constant region, wherein the antibody heavy chain constant region and the antigen binding domain are fused via a linker or without a linker.
(A44) The polypeptide according to (A41), wherein the carrying moiety comprises an antibody light chain constant region, wherein the antibody light chain constant region and the antigen binding domain are fused via a linker or without a linker.
(A45) The polypeptide according to (A43), wherein in the polypeptide, the N terminus of the antibody heavy chain constant region of the carrying moiety and the C terminus of the antigen binding domain are fused via a linker or without a linker, and the polypeptide further has a protease cleavage sequence, wherein the protease cleavage sequence is located within the sequence of the antigen binding domain, or on the antigen binding domain side compared with amino acid position 122 (EU numbering) of the antibody heavy chain constant region.
(A46) The polypeptide according to (A44), wherein in the polypeptide, the N terminus of the antibody light chain constant region of the carrying moiety and the C terminus of the antigen binding domain are fused via a linker or without a linker, and the polypeptide further has a protease cleavage sequence, wherein the protease cleavage sequence is located within the sequence of the antigen binding domain, or on the antigen binding domain side compared with amino acid position 113 (EU numbering) (Kabat numbering position 113) of the antibody light chain constant region.
(A47) The polypeptide according to any of (A42) to (A44), wherein in the polypeptide, the N terminus of the antibody constant region of the carrying moiety and the C terminus of the antigen binding domain are fused via a linker or without a linker, the antigen binding domain is a single-domain antibody prepared from VH, or VHH, and the polypeptide further has a protease cleavage sequence, wherein the protease cleavage sequence is located within the sequence of the antibody constant region, or on the antibody constant region side compared with amino acid position 109 (Kabat numbering) of the single-domain antibody of the antigen binding domain.
(A48) The polypeptide according to (A42), wherein in the polypeptide, the N terminus of the antibody constant region of the carrying moiety and the C terminus of the antigen binding domain are fused via a linker or without a linker, and the polypeptide further has a protease cleavage sequence, wherein the protease cleavage sequence is located near the boundary between the antigen binding domain and the antibody constant region.
(A49) The polypeptide according to (A43), wherein in the polypeptide, the N terminus of the antibody heavy chain constant region of the carrying moiety and the C terminus of the antigen binding domain are fused via a linker or without a linker, and the polypeptide further has a protease cleavage sequence, wherein the protease cleavage sequence is located near the boundary between the antigen binding domain and the antibody heavy chain constant region.
(A50) The polypeptide according to (A44), wherein in the polypeptide, the N terminus of the antibody light chain constant region of the carrying moiety and the C terminus of the antigen binding domain are fused via a linker or without a linker, and the polypeptide further has a protease cleavage sequence, wherein the protease cleavage sequence is located near the boundary between the antigen binding domain and the antibody light chain constant region.
(A51) The polypeptide according to (A49), wherein the antigen binding domain is a single-domain antibody prepared from VH, or VHH, and the protease cleavage sequence is located at any position between amino acid position 109 (Kabat numbering) of the single-domain antibody of the antigen binding domain and amino acid position 122 (EU numbering) of the antibody heavy chain constant region.
(A52) The polypeptide according to (A50), wherein the antigen binding domain is a single-domain antibody prepared from VH, or VHH, and the protease cleavage sequence is located at any position between amino acid position 109 (Kabat numbering) of the single-domain antibody of the antigen binding domain and amino acid position 113 (EU numbering) (Kabat numbering position 113) of the antibody light chain constant region.
(A53) The polypeptide according to (A49), wherein the antigen binding domain is a single-domain antibody prepared from VL, and the protease cleavage sequence is located at any position between amino acid position 104 (Kabat numbering) of the single-domain antibody of the antigen binding domain and amino acid position 122 (EU numbering) of the antibody heavy chain constant region.
(A54) The polypeptide according to (A50), wherein the antigen binding domain is a single-domain antibody prepared from VL, and the protease cleavage sequence is located at any position between amino acid position 109 (Kabat numbering) of the single-domain antibody of the antigen binding domain and amino acid position 113 (EU numbering) (Kabat numbering position 113) of the antibody light chain constant region.
(A55) The polypeptide according to any of (A41) to (A54), wherein the antibody constant region of the polypeptide is an IgG antibody constant region.
(A56) The polypeptide according to any of (A0) to (A55), wherein the polypeptide is an IgG antibody-like molecule.
(A57) The polypeptide according to any of (A0) to (A56), wherein when the antigen binding domain is assayed in an unreleased state by use of BLI (bio-layer interferometry) (Octet), the binding of the antigen binding domain to the antigen is not seen.
(A58) The polypeptide according to any of (A0) to (A57), wherein a second antigen binding domain is further linked to the antigen binding domain.
(A59) The polypeptide according to (A58), wherein the second antigen binding domain has antigen binding specificity different from that of the antigen binding domain.
(A60) The polypeptide according to (A58) or (A59), wherein the second antigen binding domain comprises a second single-domain antibody.
(A61) The polypeptide according to (A60), wherein the antigen binding domain is a single-domain antibody, the second antigen binding domain is a second single-domain antibody, and the antigen binding domain and the second antigen binding domain are capable of being released from the polypeptide, wherein the single-domain antibody and the second single-domain antibody form a bispecific antigen binding molecule in released states of the antigen binding domain and the second antigen binding domain.
(A62) The polypeptide according to any of (A58) to (A61), wherein the second antigen binding domain is directed to HER2 or GPC3 as a target antigen.
(A63) The polypeptide according to any of (A0) to (A62), wherein the polypeptide further has an additional antigen binding domain different from the antigen binding domain, wherein the antigen binding activity of the additional antigen binding domain is also inhibited by linking to the carrying moiety of the polypeptide.
(A64) The polypeptide according to (A63), wherein the additional antigen binding domain and the antigen binding domain differ in antigen binding specificity.
(A65) A pharmaceutical composition comprising the polypeptide of any of (A0) to (A64).

The present invention further encompasses exemplary embodiments described below.
(B1) A method for treating a subject having an IL-1R1 mediated disease or disorder comprising administering to the subject an effective amount of the polypeptide of any one of (A1) to (A64).
(B2) A method for treating a subject having osteoarthritis (OA) comprising administering to the subject an effective amount of the polypeptide of any one of (A1) to (A64).
(B3) A method for preventing cartilage degradation of a subject in osteoarthritis (OA) comprising administering to the subject an effective amount of the polypeptide of any one of (A1) to (A64).
(B4) A pharmaceutical composition for treating a subject having an IL-1R1 mediated disease or disorder, comprising an effective amount of the polypeptide of any one of (A1) to (A64).
(B5) A pharmaceutical composition for treating a subject having osteoarthritis (OA), comprising an effective amount of the polypeptide of any one of (A1) to (A64).
(B6) A pharmaceutical composition for preventing cartilage degradation of a subject in osteoarthritis (OA), comprising an effective amount of the polypeptide of any one of (A1) to (A64).
(B7) Use of the polypeptide of any one of (A1) to (A64) in a manufacture of a medicament for treating a subject having an IL-1R1 mediated disease or disorder.
(B8) Use of the polypeptide of any one of (A1) to (A64) in a manufacture of a medicament for treating a subject having osteoarthritis (OA).
(B9) Use of the polypeptide of any one of (A1) to (A64) in a manufacture of a medicament for preventing cartilage degradation of a subject in osteoarthritis (OA).
(B10) A polypeptide of any one of (A1) to (A64) for use in treating a subject having an IL-1R1 mediated disease or disorder.
(B11) A polypeptide of any one of (A1) to (A64) for use in treating a subject having osteoarthritis (OA).
(B12) A polypeptide of any one of (A1) to (A64) for use in preventing cartilage degradation of a subject in osteoarthritis (OA).

The present invention further encompasses exemplary embodiments described below.
(C1) A method for producing the polypeptide of any of (A1) to (A64).
(C2) The production method according to (C1), comprising the following steps:
   (a) obtaining an antigen binding domain binding to one or more antigens involved in IL-1 mediated signal transduction;
   (b) linking the antigen binding domain obtained in the step (a) to a carrying moiety such that the antigen binding activity of the antigen binding domain is inhibited by an inhibiting domain of the carrying moiety, to form a polypeptide precursor; and
   (c) introducing a protease cleavage sequence into the polypeptide precursor.
(C3) The production method according to (C1), comprising the following steps:
   (a) obtaining an antigen binding domain binding to one or more antigens involved in IL-1 mediated signal transduction;
   (b) linking the antigen binding domain obtained in the step (a) to a carrying moiety such that the antigen binding activity of the antigen binding domain is inhibited by an inhibiting domain of the carrying moiety, to form a polypeptide precursor; and
   (c) introducing a protease cleavage sequence to near the boundary between the antigen binding domain and the carrying moiety.
(C4) The production method according to (C1), comprising the following steps:
   (a) obtaining an antigen binding domain binding to one or more antigens involved in IL-1 mediated signal transduction; and
   (b) linking the antigen binding domain obtained in the step (a) to a carrying moiety via a protease cleavage sequence such that the antigen binding activity of the antigen binding domain is inhibited by an inhibiting domain of the carrying moiety, to form a polypeptide.
(C5) The production method according to any of (C2) to (C4), further comprising the following step:
   (d) confirming that the binding activity of the antigen binding domain incorporated in the polypeptide or the polypeptide precursor against the target antigen is weakened or lost.
(C6) The production method according to any of (C2) to (C5), further comprising the following step:
   (e) releasing the antigen binding domain by cleaving the protease cleavage sequence with a protease and confirming that the released antigen binding domain binds to the antigen.
(C7) The production method according to (C1), wherein the polypeptide is an IgG antibody-like molecule.
(C8) The production method according to (C7), comprising the following steps:
   (a) obtaining an antigen binding domain binding to one or more antigens involved in IL-1 mediated signal transduction;
   (b) associating the antigen binding domain obtained in the step (a) as a substitute for VH of an IgG antibody or a modified IgG antibody with VL or VH, or associating the antigen binding domain as a substitute for VL of an IgG antibody or a modified IgG antibody with VH or VL such that the antigen binding activity of the antigen binding domain is inhibited, to form an IgG antibody-like molecule precursor harboring the antigen binding domain; and
   (c) introducing a protease cleavage sequence into the IgG antibody-like molecule precursor harboring the antigen binding domain.
(C9) The production method according to (C7), comprising the following steps:
   (a) obtaining an antigen binding domain binding to one or more antigens involved in IL-1 mediated signal transduction;
   (b) associating the antigen binding domain obtained in the step (a) as a substitute for VH of an IgG antibody or a modified IgG antibody with VL or VH, or associating the antigen binding domain as a substitute for VL of an IgG antibody or a modified IgG antibody with VH or VL such that the antigen binding activity of the antigen binding domain is inhibited, to form an IgG antibody-like molecule precursor harboring the antigen binding domain; and
   (c) introducing a protease cleavage sequence to near the boundary between the antigen binding domain and an antibody constant region in the IgG antibody-like molecule precursor.
(C10) The production method according to (C7), comprising the following steps:
   (a) obtaining an antigen binding domain binding to one or more antigens involved in IL-1 mediated signal transduction; and
   (b) linking the antigen binding domain obtained in the step (a) as a substitute for IgG antibody VH or VL to an IgG antibody heavy chain constant region or light chain constant region via a protease cleavage sequence such that the antigen binding activity of the antigen binding domain is inhibited, to form an IgG antibody-like molecule harboring the antigen binding domain.
(C11) The production method according to any of (C8) to (C10), further comprising the following step:
   (d) confirming that the binding activity of the antigen binding domain introduced in the IgG antibody-like molecule or the IgG antibody-like molecule precursor against the target antigen is weakened or lost.
(C12) The production method according to any of (C8) to (C11), further comprising the following step:
   (e) releasing the antigen binding domain by cleaving the protease cleavage sequence with a protease and confirming that the released antigen binding domain binds to the target antigen.
(C13) The production method according to (C7), comprising the following steps:
   (a) substituting an amino acid residue in an antigen binding domain that involves in association of the antigen binding domain with antibody VH, or substituting an amino acid residue in an antigen binding domain that involves in association of the antigen binding domain with antibody VL, to prepare an antigen binding domain variant retaining the binding activity of the antigen binding domain against the target antigen;
   (b) associating the antigen binding domain variant prepared in the step (a) with the antibody VH, or associating the antigen binding domain variant with the antibody VL such that the antigen binding activity of the antigen binding domain variant is inhibited, to form an IgG antibody-like molecule precursor harboring the antigen binding domain variant; and
   (c) introducing a protease cleavage sequence into the IgG antibody-like molecule precursor harboring the antigen binding domain variant, wherein the antigen biding domain binds to one or more antigens involved in IL-1 mediated signal transduction.
(C14) The production method according to (C7), comprising the following steps:
   (a) substituting an amino acid residue in an antigen binding domain that involves in association of the antigen binding domain with antibody VH, or substituting an amino acid residue in an antigen binding domain that involves in association of the antigen binding domain with antibody VL, to prepare an antigen binding domain variant retaining the binding activity of the antigen binding domain against the target antigen;
   (b) associating the antigen binding domain variant prepared in the step (a) with the antibody VH, or associating the antigen binding domain variant with the antibody VL such that the antigen binding activity of the antigen binding domain variant is inhibited, to form an IgG antibody-like molecule precursor harboring the antigen binding domain variant; and
   (c) introducing a protease cleavage sequence to near the boundary between the antigen binding domain variant and a constant region in the IgG antibody-like molecule precursor, wherein the antigen biding domain binds to one or more antigens involved in IL-1 mediated signal transduction.
(C15) The production method according to (C7), comprising the following steps:
   (a) substituting an amino acid residue in an antigen binding domain that involves in association of the antigen binding domain with antibody VH, or substituting an amino acid residue in an antigen binding domain that involves in association of the antigen binding domain with antibody VL, to prepare an antigen binding domain variant retaining the binding activity of the antigen binding domain against the target antigen; and
   (b) linking the antigen binding domain variant prepared in the step (a) to an IgG antibody heavy chain constant region via a protease cleavage sequence, or linking the antigen binding domain variant to an IgG antibody light chain constant region via a protease cleavage sequence such that the antigen binding activity of the antigen binding domain variant is inhibited, to form an IgG antibody-like molecule harboring the antigen binding domain variant, wherein the antigen biding domain binds to one or more antigens involved in IL-1 mediated signal transduction.
(C16) The production method according to any of (C13) to (C15), further comprising the following step:
   (d) confirming that the binding activity of the antigen binding domain variant harbored in the IgG antibody-like molecule or the binding activity of the antigen binding domain variant harbored in the IgG antibody-like molecule precursor against the target antigen is weakened or lost.
(C17) The production method according to any of (C13) to (C16), further comprising the following step:
   (e) releasing the antigen binding domain variant by cleaving the protease cleavage sequence with a protease and confirming that the released antigen binding domain variant binds to the target antigen.
(C18) The production method according to any of (C2) to (C17), wherein the antigen binding domain binds to an epitope within one or more antigens involved in IL-1 mediated signal transduction.
(C19) The production method according to any of (C1) to (C18), wherein one or more antigens involved in IL-1 mediated signal transduction is IL-1R1, IL-1alpha, IL-1beta and/or IL-1RAcP.
(C20) The production method according to any of (C2) to (C18), wherein the antigen binding domain competes for binding the epitope with a single-domain antibody VL selected from the group consisting of 1) and 2) below:
   1) a single-domain antibody VL comprising the amino acid sequence of SEQ ID NO: 479, and
   2) a single-domain antibody VL comprising the amino acid sequence of SEQ ID NO: 480.
(C21) The production method according to any of (C2) to (C20), wherein the antigen binding domain comprises a single-domain antibody or an antagonist or is a single-domain antibody or an antagonist.
(C22) The production method according to (C21), wherein the single-domain antibody is a VL having antigen binding activity by itself.
(C23) The production method according to (C22), wherein the VL having antigen binding activity by itself comprises the amino acid sequence of SEQ ID NO: 479 or 480.
(C24) The production method according to any one of (C8) to (C23), wherein the VH of IgG in the step (b) and/or IgG antibody-like molecule precursor in in the step (b) comprises an amino acid sequence of SEQ ID NO: 486.
(C25) The production method according to any of (C8) to (C23), wherein the VL of IgG in the step (b) and/or IgG antibody-like molecule precursor in in the step (b) comprises an amino acid sequence of SEQ ID NO: 484 or 485.
(C26) A polynucleotide encoding the polypeptide according to any of (A1) to (A64).
(C27) A vector comprising the polynucleotide according to (C26).
(C28) A host cell comprising the polynucleotide according to (C26) or the vector according to (C26).
(C29) A method for producing the polypeptide of any of (A1) to (A64), comprising the step of culturing the host cell according to (C28).
(C30) The polypeptide produced by the method according to any of (C1) to (C25) and (C29).

The present invention further encompasses exemplary embodiments described below.
(D1) A method for screening for an antigen binding domain whose antigen binding activity can be inhibited by associating with particular VL, associating with particular VH, or associating with particular VHH, wherein the antigen is one or more molecules involved in IL-1 mediated signal transduction.
(D2) The screening method according to (D1), wherein the method is a method for screening for an antigen binding domain whose antigen binding activity can be inhibited by associating with particular VL.
(D3) The screening method according to (D2), comprising the following steps:
   (a) obtaining an antigen binding domain having target antigen binding activity;
   (b) associating the antigen binding domain obtained in the step (a) with a particular VL; and
   (c) confirming that the binding activity of the antigen binding domain associated with the particular VL in the step (b) against the antigen is weakened or lost as compared with that before the association.
(D4) The screening method according to (D2), comprising the following steps:
   (a) associating an antigen binding domain with a particular VL;
   (b) selecting an association of the VL and the antigen binding domain on the basis that the antigen binding domain associated with the particular VL in the step (a) has no binding activity or binding activity of a predetermined value or lower against the antigen; and
   (c) confirming that the antigen binding domain in the associate selected in the step (b) has stronger binding activity against the antigen in a state unassociated with the particular VL than that in a state associated therewith.
(D5) The screening method according to (D1), wherein the method is a method for screening for an antigen binding domain whose antigen binding activity can be inhibited by associating with particular VH.
(D6) The screening method according to (D5), comprising the following steps:
   (a) obtaining an antigen binding domain having target antigen binding activity;
   (b) associating the antigen binding domain obtained in the step (a) with a particular VH; and
   (c) confirming that the binding activity of the antigen binding domain associated with the particular VH in the step (b) against the antigen is weakened or lost as compared with that before the association.
(D7) The screening method according to (D5), comprising the following steps:
   (a) associating an antigen binding domain with a particular VH;
   (b) selecting an association of the VH and the antigen binding domain on the basis that the antigen binding domain associated with the particular VH in the step (a) has no binding activity or binding activity of a predetermined value or lower against the antigen; and
   (c) confirming that the antigen binding domain in the associate selected in the step (b) has stronger binding activity against the antigen in a state unassociated with the particular VH than that in a state associated therewith.
(D8) The screening method according to (D1), wherein the method is a method for screening for an antigen binding domain whose antigen binding activity can be inhibited by associating with particular VHH.
(D9) The screening method according to (D8), comprising the following steps:
   (a) obtaining an antigen binding domain having target antigen binding activity;
   (b) associating the antigen binding domain obtained in the step (a) with a particular VHH; and
   (c) confirming that the binding activity of the antigen binding domain associated with the particular VHH in the step (b) against the antigen is weakened or lost as compared with that before the association.
(D10) The screening method according to (D8), comprising the following steps:
   (a) associating an antigen binding domain with a particular VHH;
   (b) selecting an association of the VHH and the antigen binding domain on the basis that the antigen binding domain associated with the particular VHH in the step (a) has no binding activity or binding activity of a predetermined value or lower against the antigen; and
   (c) confirming that the antigen binding domain in the associate selected in the step (b) has stronger binding activity against the antigen in a state unassociated with the particular VHH than that in a state associated therewith.
(D11) The screening method according to any of (D1) to (D10), wherein the antigen binding domain comprises a single-domain antibody or an antagonist or is a single-domain antibody or an antagonist.
(D12) The screening method according to (D11), wherein the single-domain antibody is a VL having antigen binding activity by itself.
(D13) The screening method according to any of (D1) to (D12), wherein one or more molecules involved in IL-1 mediated signal transduction is selected from the group consisting of IL-1R1, IL-1alpha, IL-1beta and IL-1RAcP.
(D14) The screening method according to any of (D1) to (D13) for use in obtaining a candidate for
   (i) treating a subject having an IL-1R1 mediated disease or disorder,
   (ii) treating a subject having osteoarthritis (OA), and/or
   (iii) preventing cartilage degradation of a subject in osteoarthritis (OA).

The present invention further encompasses exemplary embodiments described below.
(E1) A method for producing an antigen binding domain whose antigen binding activity can be inhibited by associating with particular VL, associating with particular VH, or associating with particular VHH, wherein the antigen is one or more molecules involved in IL-1 mediated signal transduction.
(E2) The production method according to (E1), wherein the method is a method for producing an antigen binding domain whose antigen binding activity can be inhibited by associating with particular VL.
(E3) The production method according to (E2), comprising the following step:
   (a) substituting an amino acid residue in an antigen binding domain that involves in association of the antigen binding domain with antibody VL, to prepare an antigen binding domain variant retaining the binding activity of the antigen binding domain against the target antigen.
(E4) The production method according to (E3), further comprising the following steps:
   (b) associating the antigen binding domain variant prepared in the step (a) with the VL; and
   (c) confirming that the antigen binding activity of the antigen binding domain variant associated with the VL is weakened or lost as compared with that before the association.
(E5) The production method according to (E1), wherein the method is a method for producing an antigen binding domain whose antigen binding activity can be inhibited by associating with particular VH.
(E6) The production method according to (E5), comprising the following step:
   (a) substituting an amino acid residue in an antigen binding domain that involves in association of the antigen binding domain with antibody VH, to prepare an antigen binding domain variant retaining the binding activity of the antigen binding domain against the target antigen.
(E7) The production method according to (E6), further comprising the following steps:
   (b) associating the antigen binding domain variant prepared in the step (a) with the VH; and
   (c) confirming that the antigen binding activity of the antigen binding domain variant associated with the VH is weakened or lost as compared with that before the association.
(E8) The production method according to (E1), wherein the method is a method for producing an antigen binding domain whose antigen binding activity can be inhibited by associating with particular VHH.
(E9) The production method according to (E8), comprising the following step:
   (a) substituting an amino acid residue in an antigen binding domain that involves in association of the antigen binding domain with an VHH, to prepare an antigen binding domain variant retaining the binding activity of the antigen binding domain against the target antigen.
(E10) The production method according to (E9), further comprising the following steps:
   (b) associating the antigen binding domain variant prepared in the step (a) with the VHH; and
   (c) confirming that the antigen binding activity of the antigen binding domain variant associated with the VHH is weakened or lost as compared with that before the association.
(E11) The production method according to any of (E1) to (E10), wherein the antigen binding domain comprises a single-domain antibody or an antagonist or is a single-domain antibody or an antagonist.
(E12) The production method according to (E11), wherein the single-domain antibody is a VL having antigen binding activity by itself.
(E13) The production method according to any of (E1) to (E12), wherein one or more molecules involved in IL-1 mediated signal transduction is selected from the group consisting of IL-1R1, IL-1alpha, IL-1beta and IL-1RAcP.

The present invention further encompasses exemplary embodiments described below.
(F1) A library comprising a plurality of fusion polypeptides of antigen binding domains each linked to a first association sustaining domain, wherein the antigen binding domains include an antigen binding domain whose antigen binding activity can be inhibited or could be lost by associating with particular VL, an antigen binding domain whose antigen binding activity can be inhibited or could be lost by associating with particular VH, or an antigen binding domain whose antigen binding activity can be inhibited or could be lost by associating with particular VHH, wherein the antigen is one or more molecules involved in IL-1 mediated signal transduction.
(F2) The library according to (F1), wherein the antigen binding domain comprises a single-domain antibody or is a single-domain antibody, and the single-domain antibody moieties of the fusion polypeptides in the library include a single-domain antibody obtained from an animal of the family Camelidae or a transgenic animal harboring a gene capable of raising the single-domain antibody, or a humanized antibody thereof, a single-domain antibody obtained by the immunization of an animal of the family Camelidae or a transgenic animal harboring a gene capable of raising the single-domain antibody, or a humanized antibody thereof, or an artificially prepared single-domain antibody originating from human antibody VH or VL.
(F3) The library according to (F1) or (F2) which is a library comprising a plurality of fusion polypeptides of antigen binding domains each linked to a first association sustaining domain, wherein the antigen binding domains include an antigen binding domain whose antigen binding activity can be inhibited or could be lost by associating with particular VL.
(F4) The library according to (F1) or (F2) which is a library comprising a plurality of fusion polypeptides of antigen binding domains each linked to a first association sustaining domain, wherein the antigen binding domains include an antigen binding domain whose antigen binding activity can be inhibited or could be lost by associating with particular VH.
(F5) The library according to (F1) or (F2) which is a library comprising a plurality of fusion polypeptides of antigen binding domains each linked to a first association sustaining domain, wherein the antigen binding domains include an antigen binding domain whose antigen binding activity can be inhibited or could be lost by associating with particular VHH.
(F6) The library according to any of (F1) to (F5), wherein the first association sustaining domain comprises an IgG antibody CH1 domain or an antibody light chain constant region.
(F7) The library according to any of (F1) to (F6), wherein the antigen binding domain comprises a single-domain antibody or an antagonist or is a single-domain antibody or an antagonist.
(F8) The library according to (F7), wherein the single-domain antibody is a VL having antigen binding activity by itself.
(F9) The library according to any of (F1) to (F8), wherein one or more molecules involved in IL-1 mediated signal transduction is selected from the group consisting of IL-1R1, IL-1alpha, IL-1beta and IL-1RAcP.
(F10) The library according to any of (F1) to (F9), wherein the VL having antigen binding activity by itself comprises the amino acid sequence of SEQ ID NO: 479 or 480.

The present invention further encompasses exemplary embodiments described below.
(G1) A method for screening a library according to (F1) or (F2) for a fusion polypeptide comprising an antigen binding domain whose antigen binding activity can be inhibited or could be lost by associating with particular VL, an antigen binding domain whose antigen binding activity can be inhibited or could be lost by associating with particular VH, or an antigen binding domain whose antigen binding activity can be inhibited or could be lost by associating with particular VHH, wherein the antigen is one or more molecules involved in IL-1 mediated signal transduction.
(G2) A method for screening a library according to (F3) for a fusion polypeptide comprising an antigen binding domain whose antigen binding activity can be inhibited or could be lost by associating with particular VL, wherein the antigen is one or more molecules involved in IL-1 mediated signal transduction.
(G3) The screening method according to (G2), comprising the following steps:
   (a) in vitro displaying the fusion polypeptides of the library;
   (b) providing an association partner of a second association sustaining domain fused with a particular VL;
   (c) associating the fusion polypeptides displayed in the step (a) with the association partner provided in the step (b) and selecting a fusion polypeptide that does not bind to the antigen or has antigen binding activity of a predetermined value or lower in a state where the antigen binding domain associates with the VL; and
   (d) selecting, from the fusion polypeptides thus selected in the step (c), a fusion polypeptide that binds to the antigen or has antigen binding activity of a predetermined value or higher in a state where the antigen binding domain contained therein does not associate with the VL.
(G4) The screening method according to (G3), wherein the association partner provided in the step (b) further comprises a protease cleavage sequence, and the step (d) comprises cleaving the association partner by protease treatment so that the association of the antigen binding domain with the VL is canceled.
(G5) The screening method according to (G4), wherein the protease cleavage sequence of the association partner provided in the step (b) is located near the boundary between the particular VL and the second association sustaining domain.
(G6) The screening method according to (G3), wherein the fusion polypeptides of the library further comprise a protease cleavage sequence, and the step (d) comprises cleaving the fusion polypeptides by protease treatment so that the association of the antigen binding domain with the VL is canceled.
(G7) The screening method according to (G6), wherein the fusion polypeptide comprises a first association sustaining domain, and the protease cleavage sequence contained in each fusion polypeptide is located near the boundary between the antigen binding domain and the first association sustaining domain.
(G8) The screening method according to (G3), wherein the step (d) comprises in vitro displaying again the full lengths of the fusion polypeptides selected in the step (c) or their moieties comprising the antigen binding domains.
(G9) The screening method according to (G3), wherein the step (d) comprises in vitro displaying again the full lengths of the fusion polypeptides selected in the step (c) and selecting a fusion polypeptide that binds to the antigen or has antigen binding activity of a predetermined value or higher in a state associated only with the second association sustaining domain.
(G10) A method for screening a library according to (F4) for a fusion polypeptide comprising an antigen binding domain whose antigen binding activity can be inhibited or could be lost by associating with particular VH, wherein the antigen is one or more molecules involved in IL-1 mediated signal transduction.
(G11) The screening method according to (G10), comprising the following steps:
   (a) in vitro displaying the fusion polypeptides of the library;
   (b) providing an association partner of a second association sustaining domain fused with a particular VH;
   (c) associating the fusion polypeptides displayed in the step (a) with the association partner provided in the step (b) and selecting a fusion polypeptide that does not bind to the antigen or has antigen binding activity of a predetermined value or lower in a state where the antigen binding domain associates with the VH; and
   (d) selecting, from the fusion polypeptides thus selected in the step (c), a fusion polypeptide that binds to the antigen or has antigen binding activity of a predetermined value or higher in a state where the antigen binding domain contained therein does not associate with the VH.
(G12) The screening method according to (G11), wherein the association partner provided in the step (b) further comprises a protease cleavage sequence, and the step (d) comprises cleaving the association partner by protease treatment so that the association of the antigen binding domain with the VH is canceled.
(G13) The screening method according to (G12), wherein the protease cleavage sequence of the association partner provided in the step (b) is located near the boundary between the particular VH and the second association sustaining domain.
(G14) The screening method according to (G11), wherein the fusion polypeptides of the library further comprise a protease cleavage sequence, and the step (d) comprises cleaving the fusion polypeptides by protease treatment so that the association of the antigen binding domain with the VH is canceled.
(G15) The screening method according to (G14), wherein the fusion polypeptide comprises a first association sustaining domain, and the protease cleavage sequence contained in each fusion polypeptide is located near the boundary between the antigen binding domain and the first association sustaining domain.
(G16) The screening method according to (G11), wherein the step (d) comprises in vitro displaying again the full lengths of the fusion polypeptides selected in the step (c) or their moieties comprising the antigen binding domains.
(G17) The screening method according to (G11), wherein the step (d) comprises in vitro displaying again the full lengths of the fusion polypeptides selected in the step (c) and selecting a fusion polypeptide that binds to the antigen or has antigen binding activity of a predetermined value or higher in a state associated only with the second association sustaining domain.
(G18) A method for screening a library according to (F5) for a fusion polypeptide comprising an antigen binding domain whose antigen binding activity can be inhibited or could be lost by associating with particular VHH, wherein the antigen is one or more molecules involved in IL-1 mediated signal transduction.
(G19) The screening method according to (G18), comprising the following steps:
   (a) in vitro displaying the fusion polypeptides of the library;
   (b) providing an association partner of a second association sustaining domain fused with a particular VHH;
   (c) associating the fusion polypeptides displayed in the step (a) with the association partner provided in the step (b) and selecting a fusion polypeptide that does not bind to the antigen or has antigen binding activity of a predetermined value or lower in a state where the antigen binding domain associates with the particular VHH; and
   (d) selecting, from the fusion polypeptides thus selected in the step (c), a fusion polypeptide that binds to the antigen or has antigen binding activity of a predetermined value or higher in a state where the antigen binding domain contained therein does not associate with the VHH.
(G20) The screening method according to (G19), wherein the association partner provided in the step (b) further comprises a protease cleavage sequence, and the step (d) comprises cleaving the association partner by protease treatment so that the association of the antigen binding domain with the VHH is canceled.
(G21) The screening method according to (G20), wherein the protease cleavage sequence of the association partner provided in the step (b) is located near the boundary between the particular VHH and the second association sustaining domain.
(G22) The screening method according to (G19), wherein the fusion polypeptides of the library further comprise a protease cleavage sequence, and the step (d) comprises cleaving the fusion polypeptides by protease treatment so that the association of the antigen binding domain with the VHH is canceled.
(G23) The screening method according to (G22), wherein the fusion polypeptide comprises a first association sustaining domain, and the protease cleavage sequence contained in each fusion polypeptide is located near the boundary between the antigen binding domain and the first association sustaining domain.
(G24) The screening method according to (G19), wherein the step (d) comprises in vitro displaying again the full lengths of the fusion polypeptides selected in the step (c) or their moieties comprising the antigen binding domains.
(G25) The screening method according to (G19), wherein the step (d) comprises in vitro displaying again the full lengths of the fusion polypeptides selected in the step (c) and selecting a fusion polypeptide that binds to the antigen or has antigen binding activity of a predetermined value or higher in a state associated only with the second association sustaining domain.
(G26) The screening method according to any of (G3) to (G9), (G11) to (G17), and (G19) to (G25), wherein the step of providing an association partner in the step (b) is the step of displaying the association partner and the fusion polypeptides together.
(G27) The screening method according to any of (G7) to (G9), (G15) to (G17), and (G23) to (G25), wherein the first association sustaining domain comprises an IgG antibody CH1 domain or an antibody light chain constant region.
(G28) The screening method according to any of (G3) to (G9), (G11) to (G17), and (G19) to (G25), wherein the second association sustaining domain comprises an IgG antibody CH1 domain or an antibody light chain constant region.
(G29) The screening method according to (G27) or (G28), wherein the first association sustaining domain comprises an IgG antibody CH1 domain, and the second association sustaining domain comprises an antibody light chain constant region.
(G30) The screening method according to (G27) or (G28), wherein the first association sustaining domain comprises an antibody light chain constant region, and the second association sustaining domain comprises an IgG antibody CH1 domain.
(G31) The screening method according to (G2), comprising the following steps:
   (a) in vitro displaying the fusion polypeptides of the library;
   (b) providing an association partner of a second association sustaining domain fused with a particular VL;
   (c) selecting a fusion polypeptide comprising an antigen binding domain that binds to the antigen or has antigen binding activity of a predetermined value or higher; and
   (d) associating the fusion polypeptides thus selected in the step (c) with the association partner provided in the step (b) and selecting a fusion polypeptide that does not bind to the antigen or has antigen binding activity of a predetermined value or lower in a state where the antigen binding domain associates with the VL.
(G32) The screening method according to (G31), wherein the step (d) comprises in vitro displaying again the fusion polypeptides selected in the step (c).
(G33) The screening method according to (G31), wherein the step (c) comprises associating the fusion polypeptide only with the second association sustaining domain or confirming the antigen binding of the antigen binding domain contained in the fusion polypeptide associated only with the second association sustaining domain.
(G34) The screening method according to (G10), comprising the following steps:
   (a) in vitro displaying the fusion polypeptides of the library;
   (b) providing an association partner of a second association sustaining domain fused with a particular VH;
   (c) selecting a fusion polypeptide comprising an antigen binding domain that binds to the antigen or has antigen binding activity of a predetermined value or higher; and
   (d) associating the fusion polypeptides thus selected in the step (c) with the association partner provided in the step (b) and selecting a fusion polypeptide that does not bind to the antigen or has antigen binding activity of a predetermined value or lower in a state where the antigen binding domain associates with the VH.
(G35) The screening method according to (G34), wherein the step (d) comprises in vitro displaying again the fusion polypeptides selected in the step (c).
(G36) The screening method according to (G34), wherein the step (c) comprises associating the fusion polypeptide only with the second association sustaining domain or confirming the antigen binding of the antigen binding domain contained in the fusion polypeptide associated only with the second association sustaining domain.
(G37) The screening method according to (G18), comprising the following steps:
   (a) in vitro displaying the fusion polypeptides of the library;
   (b) providing an association partner of a second association sustaining domain fused with a particular VHH;
   (c) selecting a fusion polypeptide comprising an antigen binding domain that binds to the antigen or has antigen binding activity of a predetermined value or higher; and
   (d) associating the fusion polypeptides thus selected in the step (c) with the association partner provided in the step (b) and selecting a fusion polypeptide that does not bind to the antigen or has antigen binding activity of a predetermined value or lower in a state where the antigen binding domain associates with the VHH.
(G38) The screening method according to (G37), wherein the step (d) comprises in vitro displaying again the fusion polypeptides selected in the step (c).
(G39) The screening method according to (G37), wherein the step (c) comprises associating the fusion polypeptide only with the second association sustaining domain or confirming the antigen binding of the antigen binding domain contained in the fusion polypeptide associated only with the second association sustaining domain.
(G40) The screening method according to any of (G31) to (G39), wherein the fusion polypeptide comprises a first association sustaining domain, and the first association sustaining domain comprises an IgG antibody CH1 domain or an antibody light chain constant region.
(G41) The screening method according to any of (G31) to (G40), wherein the second association sustaining domain comprises an IgG antibody CH1 domain or an antibody light chain constant region.
(G42) The screening method according to any of (G31) to (G41), wherein the step of associating the fusion polypeptides with the association partner in the step (d) is the step of displaying the association partner and the fusion polypeptides together.
(G43) The screening method according to any of (G31) to (G42), wherein the fusion polypeptide comprises a first association sustaining domain, and the first association sustaining domain comprises an IgG antibody CH1 domain, and the second association sustaining domain comprises an antibody light chain constant region.
(G44) The screening method according to any of (G31) to (G42), wherein the fusion polypeptide comprises a first association sustaining domain, and the first association sustaining domain comprises an antibody light chain constant region, and the second association sustaining domain comprises an IgG antibody CH1 domain.
(G45) The screening method according to any of (G31) to (G44), wherein the antigen binding domain comprises a single-domain antibody or antagonist or is a single-domain antibody or antagonist.
(G46) The screening method according to (G45), wherein the single-domain antibody is a VL having antigen binding activity by itself.
(G47) The screening method according to any of (G1) to (G46), wherein one or more molecules involved in IL-1 mediated signal transduction is selected from the group consisting of IL-1R1, IL-1alpha, IL-1beta and IL-1RAcP.

The present invention further encompasses exemplary embodiments described below.
(H1) A polypeptide comprising an antigen binding domain VL and a carrying moiety, wherein the antigen binding domain VL has a shorter half-life in blood than that of the carrying moiety that has an inhibiting domain that inhibits the antigen binding activity of the antigen binding domain VL.
(H2) The polypeptide according to (H1), wherein the antigen binding domain VL comprises a VL having antigen binding activity by itself or is a VL having antigen binding activity by itself.
(H3) The polypeptide according to (H1) or (H2), wherein the inhibiting domain of the carrying moiety is VHH, antibody VH, or antibody VL.
(H4) A polypeptide comprising an antigen binding domain VL and a carrying moiety, wherein the antigen binding domain VL has a shorter half-life in blood than that of the carrying moiety that has an inhibiting domain VL that inhibits the antigen binding activity of the antigen binding domain VL.
(H5) The polypeptide according to (H4), wherein the antigen binding domain VL comprises a VL having antigen binding activity by itself or is a VL having antigen binding activity by itself.
(H6) The polypeptide according to (H4) or (H5), wherein the inhibiting domain VL is antibody VL.
(H7) The polypeptide according to any one of (H1) to (H6), wherein the molecular weight of the antigen binding domain VL is smaller than that of the carrying moiety.
(H8) The polypeptide according to any one of (H1) to (H7), wherein the molecular weight of the antigen binding domain VL is 120 kDa, 100 kDa, 80 kDa, 60 kDa, 40 kDa, 20 kDa or smaller.
(H9) The polypeptide according to any of (H1) to (H8), wherein the carrying moiety has FcRn binding activity, and the antigen binding domain VL has no FcRn binding activity or has weaker FcRn binding activity than that of the carrying moiety.
(H10) The polypeptide according to any of (H1) to (H9), wherein the antigen binding domain VL is capable of being released from the polypeptide, and the antigen binding domain VL released from the polypeptide has higher antigen binding activity than that before the release.
(H11) The polypeptide according to any of (H1) to (H10), wherein the inhibiting domain of the carrying moiety associates with the antigen binding domain VL and thereby inhibits the antigen binding activity of the antigen binding domain VL.
(H12) The polypeptide according to (H10), wherein the polypeptide comprises a cleavage site, wherein the cleavage site is cleaved so that the antigen binding domain VL becomes capable of being released from the polypeptide.
(H13) The polypeptide according to (H11), wherein the polypeptide comprises a cleavage site, wherein the cleavage site is cleaved so that the association of the inhibiting domain of the carrying moiety with the antigen binding domain VL is canceled.
(H14) The polypeptide according to (H12) or (H13), wherein the cleavage site comprises a protease cleavage sequence.
(H15) The polypeptide according to (H14), wherein the protease is a target tissue specific protease.
(H16) The polypeptide according to (H15), wherein the target tissue is a cancer tissue or an inflammatory tissue.
(H17) The polypeptide according to (H14), wherein the protease is at least one protease selected from matriptase, urokinase (uPA), and metalloproteinase.
(H18) The polypeptide according to (H17), wherein the protease is at least one protease selected from MT-SP1, uPA, MMP1, MMP2, MMP3, MMP7, MMP9, MMP13, MMP14, ADAM17 ADAMTS4, and ADAMTS5.
(H19) The polypeptide according to (H14), wherein the protease cleavage sequence comprises a sequence selected from SEQ ID NOs: 12, 25, 34, 35, 70 to 73, 75, 76, 91, 178, 193 to 195, and 508 to 510.
(H20) The polypeptide according to any of (H14) to (H19), wherein a first flexible linker is further attached to one end of the protease cleavage sequence.
(H21) The polypeptide according to (H20), wherein a second flexible linker is further attached to the other end of the protease cleavage sequence.
(H22) The polypeptide according to (H20), wherein the first flexible linker is a flexible linker consisting of a glycine-serine polymer.
(H23) The polypeptide according to (H21), wherein the second flexible linker is a flexible linker consisting of a glycine-serine polymer.
(H24) The polypeptide according to any of (H1) to (H23), wherein the antigen binding activity of the antigen binding domain VL is inhibited by associating with the VHH, the antibody VH, or the antibody VL.
(H25) The polypeptide according to any of (H1) to (H24), wherein the carrying moiety has an FcRn binding region.
(H26) The polypeptide according to any of (H1) to (H25), wherein the carrying moiety comprises an antibody constant region.
(H27) The polypeptide according to (H26), wherein the antibody constant region of the carrying moiety and the antigen binding domain VL are fused via a linker or without a linker.
(H28) The polypeptide according to (H26), wherein the carrying moiety comprises an antibody heavy chain constant region, wherein the antibody heavy chain constant region and the antigen binding domain VL are fused via a linker or without a linker.
(H29) The polypeptide according to (H26), wherein the carrying moiety comprises an antibody light chain constant region, wherein the antibody light chain constant region and the antigen binding domain VL are fused via a linker or without a linker.
(H30) The polypeptide according to (H28), wherein in the polypeptide, the N terminus of the antibody heavy chain constant region of the carrying moiety and the C terminus of the antigen binding domain VL are fused via a linker or without a linker, and the polypeptide further has a protease cleavage sequence, wherein the protease cleavage sequence is located within the sequence of the antigen binding domain VL, or on the antigen binding domain VL side compared with amino acid position 122 (EU numbering) of the antibody heavy chain constant region.
(H31) The polypeptide according to (H29), wherein in the polypeptide, the N terminus of the antibody light chain constant region of the carrying moiety and the C terminus of the antigen binding domain VL are fused via a linker or without a linker, and the polypeptide further has a protease cleavage sequence, wherein the protease cleavage sequence is located within the sequence of the antigen binding domain VL, or on the antigen binding domain VL side compared with amino acid position 113 (EU numbering) (Kabat numbering position 113) of the antibody light chain constant region.
(H32) The polypeptide according to (H27), wherein in the polypeptide, the N terminus of the antibody constant region of the carrying moiety and the C terminus of the antigen binding domain VL are fused via a linker or without a linker, and the polypeptide further has a protease cleavage sequence, wherein the protease cleavage sequence is located near the boundary between the antigen binding domain VL and the antibody constant region.
(H33) The polypeptide according to (H28), wherein in the polypeptide, the N terminus of the antibody heavy chain constant region of the carrying moiety and the C terminus of the antigen binding domain VL are fused via a linker or without a linker, and the polypeptide further has a protease cleavage sequence, wherein the protease cleavage sequence is located near the boundary between the antigen binding domain VL and the antibody heavy chain constant region.
(H34) The polypeptide according to (H29), wherein in the polypeptide, the N terminus of the antibody light chain constant region of the carrying moiety and the C terminus of the antigen binding domain VL are fused via a linker or without a linker, and the polypeptide further has a protease cleavage sequence, wherein the protease cleavage sequence is located near the boundary between the antigen binding domain VL and the antibody light chain constant region.
(H35) The polypeptide according to (H33), wherein the antigen binding domain VL is a single-domain antibody prepared from VL, and the protease cleavage sequence is located at any position between amino acid position 104 (Kabat numbering) of the single-domain antibody of the antigen binding domain VL and amino acid position 122 (EU numbering) of the antibody heavy chain constant region.
(H36) The polypeptide according to (H34), wherein the antigen binding domain VL is a single-domain antibody prepared from VL, and the protease cleavage sequence is located at any position between amino acid position 109 (Kabat numbering) of the single-domain antibody of the antigen binding domain VL and amino acid position 113 (EU numbering) (Kabat numbering position 113) of the antibody light chain constant region.
(H37) The polypeptide according to any of (H26) to (H36), wherein the antibody constant region of the polypeptide is an IgG antibody constant region.
(H38) The polypeptide according to any of (H1) to (H37), wherein the polypeptide is an IgG antibody-like molecule.
(H39) The polypeptide according to any of (H1) to (H38), wherein when the antigen binding domain VL is assayed in an unreleased state by use of BLI (bio-layer interferometry) (Octet), the binding of the antigen binding domain VL to the antigen is not seen.
(H40) The polypeptide according to any of (H1) to (H39), wherein a second antigen binding domain is further linked to the antigen binding domain VL.
(H41) The polypeptide according to (H40), wherein the second antigen binding domain has antigen binding specificity different from that of the antigen binding domain VL.
(H42) The polypeptide according to (H40) or (H41), wherein the second antigen binding domain comprises a second single-domain antibody.
(H43) The polypeptide according to (H42), wherein the second antigen binding domain is a second single-domain antibody, and the antigen binding domain VL and the second antigen binding domain are capable of being released from the polypeptide, wherein the antigen binding domain VL and the second single-domain antibody form a bispecific antigen binding molecule in released states of the antigen binding domain VL and the second antigen binding domain.
(H44) The polypeptide according to any of (H40) to (H43), wherein the second antigen binding domain is directed to HER2 or GPC3 as a target antigen.
(H45) The polypeptide according to any of (H1) to (H44), wherein the polypeptide further has an additional antigen binding domain different from the antigen binding domain VL, wherein the antigen binding activity of the additional antigen binding domain is also inhibited by linking to the carrying moiety of the polypeptide.
(H46) The polypeptide according to (H45), wherein the additional antigen binding domain and the antigen binding domain VL differ in antigen binding specificity.
(H47) The polypeptide according to any of (H1) to (H46), wherein the additional antigen binding domain is an antigen binding domain directed to plexin A1, IL6R or CD3 as a target antigen.
(H48) A pharmaceutical composition comprising the polypeptide of any of (H1) to (H47).

The present invention further encompasses exemplary embodiments described below.
(I1) A method for producing the polypeptide of any of (H1) to (H47).
(I2) The production method according to (I1), comprising the following steps:
   (a) obtaining a single-domain antibody binding to a target antigen;
   (b) linking the single-domain antibody obtained in the step (a) to a carrying moiety such that the antigen binding activity of the single-domain antibody is inhibited by an inhibiting domain of the carrying moiety, to form a polypeptide precursor; and
   (c) introducing a protease cleavage sequence into the polypeptide precursor,
   wherein the single-domain antibody is a VL having antigen binding activity by itself.
(I3) The production method according to (I1), comprising the following steps:
   (a) obtaining a single-domain antibody binding to a target antigen;
   (b) linking the single-domain antibody obtained in the step (a) to a carrying moiety such that the antigen binding activity of the single-domain antibody is inhibited by an inhibiting domain of the carrying moiety, to form a polypeptide precursor; and
   (c) introducing a protease cleavage sequence to near the boundary between the single-domain antibody and the carrying moiety,
   wherein the single-domain antibody is a VL having antigen binding activity by itself.
(I4) The production method according to (I1), comprising the following steps:
   (a) obtaining a single-domain antibody binding to a target antigen; and
   (b) linking the single-domain antibody obtained in the step (a) to the carrying moiety via a protease cleavage sequence such that the antigen binding activity of the single-domain antibody is inhibited by an inhibiting domain of the carrying moiety, to form a polypeptide,
   wherein the single-domain antibody is a VL having antigen binding activity by itself.
(I5) The production method according to any of (I2) to (I4), further comprising the following step:
   (d) confirming that the binding activity of the single-domain antibody incorporated in the polypeptide or the polypeptide precursor against the target antigen is weakened or lost.
(I6) The production method according to any of (I2) to (I5), further comprising the following step:
   (e) releasing the single-domain antibody by cleaving the protease cleavage sequence with a protease and confirming that the released single-domain antibody binds to the antigen.
(I7) The production method according to (I1), wherein the polypeptide is an IgG antibody-like molecule.
(I8) The production method according to (I7), comprising the following steps:
   (a) obtaining a single-domain antibody binding to a target antigen;
   (b) associating the single-domain antibody obtained in the step (a) as a substitute for VH of an IgG antibody or a modified IgG antibody with VL or VH, or associating the single-domain antibody as a substitute for VL of an IgG antibody or a modified IgG antibody with VH or VL such that the antigen binding activity of the single-domain antibody is inhibited, to form an IgG antibody-like molecule precursor harboring the single-domain antibody; and
   (c) introducing a protease cleavage sequence into the IgG antibody-like molecule precursor harboring the single-domain antibody,
   wherein the single-domain antibody is a VL having antigen binding activity by itself.
(I9) The production method according to (I7), comprising the following steps:
   (a) obtaining a single-domain antibody binding to a target antigen;
   (b) associating the single-domain antibody obtained in the step (a) as a substitute for VH of an IgG antibody or a modified IgG antibody with VL or VH, or associating the single-domain antibody as a substitute for VL of an IgG antibody or a modified IgG antibody with VH or VL such that the antigen binding activity of the single-domain antibody is inhibited, to form an IgG antibody-like molecule precursor harboring the single-domain antibody; and
   (c) introducing a protease cleavage sequence to near the boundary between the single-domain antibody and an antibody constant region in the IgG antibody-like molecule precursor,
   wherein the single-domain antibody is a VL having antigen binding activity by itself.
(I10) The production method according to (I7), comprising the following steps:
   (a) obtaining a single-domain antibody binding to a target antigen; and
   (b) linking the single-domain antibody obtained in the step (a) as a substitute for IgG antibody VH or VL to an IgG antibody heavy chain constant region or light chain constant region via a protease cleavage sequence such that the antigen binding activity of the single-domain antibody is inhibited, to form an IgG antibody-like molecule harboring the single-domain antibody,
   wherein the single-domain antibody is a VL having antigen binding activity by itself.
(I11) The production method according to any of (I8) to (I10), further comprising the following step:
   (d) confirming that the binding activity of the single-domain antibody introduced in the IgG antibody-like molecule or the IgG antibody-like molecule precursor against the target antigen is weakened or lost.
(I12) The production method according to any of (I8) to (I11), further comprising the following step:
   (e) releasing the single-domain antibody by cleaving the protease cleavage sequence with a protease and confirming that the released single-domain antibody binds to the target antigen.
(I13) The production method according to (I7), comprising the following steps:
   (a) substituting an amino acid residue in a single-domain antibody that involves in association of the single-domain antibody with antibody VH, or substituting an amino acid residue in a single-domain antibody that involves in association of the single-domain antibody with antibody VL, to prepare a single-domain antibody variant retaining the binding activity of the single-domain antibody against the target antigen;
   (b) associating the single-domain antibody variant prepared in the step (a) with the antibody VH, or associating the single-domain antibody variant with the antibody VL such that the antigen binding activity of the single-domain antibody variant is inhibited, to form an IgG antibody-like molecule precursor harboring the single-domain antibody variant; and
   (c) introducing a protease cleavage sequence into the IgG antibody-like molecule precursor harboring the single-domain antibody variant,
   wherein the single-domain antibody is a VL having antigen binding activity by itself.
(I14) The production method according to (I7), comprising the following steps:
   (a) substituting an amino acid residue in a single-domain antibody that involves in association of the single-domain antibody with antibody VH, or substituting an amino acid residue in a single-domain antibody that involves in association of the single-domain antibody with antibody VL, to prepare a single-domain antibody variant retaining the binding activity of the single-domain antibody against the target antigen;
   (b) associating the single-domain antibody variant prepared in the step (a) with the antibody VH, or associating the single-domain antibody variant with the antibody VL such that the antigen binding activity of the single-domain antibody variant is inhibited, to form an IgG antibody-like molecule precursor harboring the single-domain antibody variant; and
   (c) introducing a protease cleavage sequence to near the boundary between the single-domain antibody variant and a constant region in the IgG antibody-like molecule precursor,
   wherein the single-domain antibody is a VL having antigen binding activity by itself.
(I15) The production method according to (I7), comprising the following steps:
   (a) substituting an amino acid residue in a single-domain antibody that involves in association of the single-domain antibody with antibody VH, or substituting an amino acid residue in a single-domain antibody that involves in association of the single-domain antibody with antibody VL, to prepare a single-domain antibody variant retaining the binding activity of the single-domain antibody against the target antigen; and
   (b) linking the single-domain antibody variant prepared in the step (a) to an IgG antibody heavy chain constant region via a protease cleavage sequence, or linking the single-domain antibody variant to an IgG antibody light chain constant region via a protease cleavage sequence such that the antigen binding activity of the single-domain antibody variant is inhibited, to form an IgG antibody-like molecule harboring the single-domain antibody variant,
   wherein the single-domain antibody is a VL having antigen binding activity by itself.
(I16) The production method according to any of (I13) to (I15), further comprising the following step:
   (d) confirming that the binding activity of the single-domain antibody variant harbored in the IgG antibody-like molecule or the IgG antibody-like molecule precursor against the target antigen is weakened or lost.
(I17) The production method according to any of (I13) to (I16), further comprising the following step:
   (e) releasing the single-domain antibody variant by cleaving the protease cleavage sequence with a protease and confirming that the released single-domain antibody variant binds to the target antigen.
(I18) A polynucleotide encoding the polypeptide according to any of (H1) to (H47).
(I19) A vector comprising the polynucleotide according to (I18).
(I20) A host cell comprising the polynucleotide according to (I18) or the vector according to (I19).
(21) A method for producing the polypeptide of any of (H1) to (H47), comprising the step of culturing the host cell according to (I20).
(I22) The polypeptide produced by the method according to any of (I1) to (I17) and (I21).

The present invention further encompasses exemplary embodiments described below.
(J1) A method for screening for a single-domain antibody whose antigen binding activity can be inhibited by associating with particular VL, associating with particular VH, or associating with particular VHH, wherein the single-domain antibody is a VL having antigen binding activity by itself.
(J2) The screening method according to (J1), wherein the method is a method for screening for a single-domain antibody whose antigen binding activity can be inhibited by associating with particular VL.
(J3) The screening method according to (J2), comprising the following steps:
   (a) obtaining a single-domain antibody having target antigen binding activity;
   (b) associating the single-domain antibody obtained in the step (a) with a particular VL; and
   (c) confirming that the binding activity of the single-domain antibody associated with the particular VL in the step (b) against the antigen is weakened or lost as compared with that before the association.
(J4) The screening method according to (J2), comprising the following steps:
   (a) associating a single-domain antibody with a particular VL;
   (b) selecting an association of the VL and the single-domain antibody on the basis that the single-domain antibody associated with the particular VL in the step (a) has no binding activity or binding activity of a predetermined value or lower against the antigen; and
   (c) confirming that the single-domain antibody in the associate selected in the step (b) has stronger binding activity against the antigen in a state unassociated with the particular VL than that in a state associated therewith.
(J5) The screening method according to (J1), wherein the method is a method for screening for a single-domain antibody whose antigen binding activity can be inhibited by associating with particular VH.
(J6) The screening method according to (J5), comprising the following steps:
   (a) obtaining a single-domain antibody having target antigen binding activity;
   (b) associating the single-domain antibody obtained in the step (a) with a particular VH; and
   (c) confirming that the binding activity of the single-domain antibody associated with the particular VH in the step (b) against the antigen is weakened or lost as compared with that before the association.
(J7) The screening method according to (J5), comprising the following steps:
   (a) associating a single-domain antibody with a particular VH;
   (b) selecting an association of the VH and the single-domain antibody on the basis that the single-domain antibody associated with the particular VH in the step (a) has no binding activity or binding activity of a predetermined value or lower against the antigen; and
   (c) confirming that the single-domain antibody in the associate selected in the step (b) has stronger binding activity against the antigen in a state unassociated with the particular VH than that in a state associated therewith.
(J8) The screening method according to (J1), wherein the method is a method for screening for a single-domain antibody whose antigen binding activity can be inhibited by associating with particular VHH.
(J9) The screening method according to (J8), comprising the following steps:
   (a) obtaining a single-domain antibody having target antigen binding activity;
   (b) associating the single-domain antibody obtained in the step (a) with a particular VHH; and
   (c) confirming that the binding activity of the single-domain antibody associated with the particular VHH in the step (b) against the antigen is weakened or lost as compared with that before the association.
(J10) The screening method according to (J8), comprising the following steps:
   (a) associating a single-domain antibody with a particular VHH;
   (b) selecting an association of the VHH and the single-domain antibody on the basis that the single-domain antibody associated with the particular VHH in the step (a) has no binding activity or binding activity of a predetermined value or lower against the antigen; and
   (c) confirming that the single-domain antibody in the associate selected in the step (b) has stronger binding activity against the antigen in a state unassociated with the particular VHH than that in a state associated therewith.

The present invention further encompasses exemplary embodiments described below.
(K1) A method for producing a single-domain antibody whose antigen binding activity can be inhibited by associating with particular VL, associating with particular VH, or associating with particular VHH, wherein the single-domain antibody is a VL having antigen binding activity by itself.
(K2) The production method according to (K1), wherein the method is a method for producing a single-domain antibody whose antigen binding activity can be inhibited by associating with particular VL.
(K3) The production method according to (K2), comprising the following step:
   (a) substituting an amino acid residue in a single-domain antibody that involves in association of the single-domain antibody with antibody VL, to prepare a single-domain antibody variant retaining the binding activity of the single-domain antibody against the target antigen.
(K4) The production method according to (K3), further comprising the following steps:
   (b) associating the single-domain antibody variant prepared in the step (a) with the VL; and
   (c) confirming that the antigen binding activity of the single-domain antibody variant associated with the VL is weakened or lost as compared with that before the association.
(K5) The production method according to (K1), wherein the method is a method for producing a single-domain antibody whose antigen binding activity can be inhibited by associating with particular VH.
(K6) The production method according to (K5), comprising the following step:
   (a) substituting an amino acid residue in a single-domain antibody that involves in association of the single-domain antibody with antibody VH, to prepare a single-domain antibody variant retaining the binding activity of the single-domain antibody against the target antigen.
(K7) The production method according to (K6), further comprising the following steps:
   (b) associating the single-domain antibody variant prepared in the step (a) with the VH; and
   (c) confirming that the antigen binding activity of the single-domain antibody variant associated with the VH is weakened or lost as compared with that before the association.
(K8) The production method according to (K1), wherein the method is a method for producing a single-domain antibody whose antigen binding activity can be inhibited by associating with particular VHH.
(K9) The production method according to (K8), comprising the following step:
   (a) substituting an amino acid residue in a single-domain antibody that involves in association of the single-domain antibody with the VHH, to prepare a single-domain antibody variant retaining the binding activity of the single-domain antibody against the target antigen.
(K10) The production method according to (K9), further comprising the following steps:
   (b) associating the single-domain antibody variant prepared in the step (a) with the VHH; and
   (c) confirming that the antigen binding activity of the single-domain antibody variant associated with the VHH is weakened or lost as compared with that before the association.

The present invention further encompasses exemplary embodiments described below.
(L1) A library comprising a plurality of fusion polypeptides of single-domain antibodies each linked to a first association sustaining domain, wherein the single-domain antibodies include a single-domain antibody whose antigen binding activity can be inhibited or could be lost by associating with particular VL, a single-domain antibody whose antigen binding activity is inhibited or could be lost by associating with particular VH, or a single-domain antibody whose antigen binding activity can be inhibited or could be lost by associating with particular VHH, wherein the single-domain antibody is a VL having antigen binding activity by itself.
(L2) The library according to (L1), wherein the single-domain antibody moieties of the fusion polypeptides in the library include a single-domain antibody obtained from an animal of the family Camelidae or a transgenic animal harboring a gene capable of raising the single-domain antibody, or a humanized antibody thereof, a single-domain antibody obtained by the immunization of an animal of the family Camelidae or a transgenic animal harboring a gene capable of raising the single-domain antibody, or a humanized antibody thereof, or an artificially prepared single-domain antibody originating from human antibody VH or VL.
(L3) The library according to (L1) or (L2) which is a library comprising a plurality of fusion polypeptides of single-domain antibodies each linked to a first association sustaining domain, wherein the single-domain antibodies include a single-domain antibody whose antigen binding activity can be inhibited or could be lost by associating with particular VL.
(L4) The library according to (L1) or (L2) which is a library comprising a plurality of fusion polypeptides of single-domain antibodies each linked to a first association sustaining domain, wherein the single-domain antibodies include a single-domain antibody whose antigen binding activity can be inhibited or could be lost by associating with particular VH.
(L5) The library according to (L1) or (L2) which is a library comprising a plurality of fusion polypeptides of single-domain antibodies each linked to a first association sustaining domain, wherein the single-domain antibodies include a single-domain antibody whose antigen binding activity can be inhibited or could be lost by associating with particular VHH.
(L6) The library according to any of (L1) to (L5), wherein the first association sustaining domain comprises an IgG antibody CH1 domain or an antibody light chain constant region.

The present invention further encompasses exemplary embodiments described below.
(M1) A method for screening a library according to (L1) or (L2) for a fusion polypeptide comprising a single-domain antibody whose antigen binding activity can be inhibited or could be lost by associating with particular VL, a single-domain antibody whose antigen binding activity can be inhibited or could be lost by associating with particular VH, or a single-domain antibody whose antigen binding activity can be inhibited or could be lost by associating with particular VHH, wherein the single-domain antibody is a VL having antigen binding activity by itself.
(M2) A method for screening a library according to (L3) for a fusion polypeptide comprising a single-domain antibody whose antigen binding activity can be inhibited or could be lost by associating with particular VL, wherein the single-domain antibody is a VL having antigen binding activity by itself.
(M3) The screening method according to (M2), comprising the following steps:
   (a) in vitro displaying the fusion polypeptides of the library;
   (b) providing an association partner of a second association sustaining domain fused with a particular VL;
   (c) associating the fusion polypeptides displayed in the step (a) with the association partner provided in the step (b) and selecting a fusion polypeptide that does not bind to the antigen or has antigen binding activity of a predetermined value or lower in a state where the single-domain antibody associates with the VL; and
   (d) selecting, from the fusion polypeptides thus selected in the step (c), a fusion polypeptide that binds to the antigen or has antigen binding activity of a predetermined value or higher in a state where the single-domain antibody contained therein does not associate with the VL.
(M4) The screening method according to (M3), wherein the association partner provided in the step (b) further comprises a protease cleavage sequence, and the step (d) comprises cleaving the association partner by protease treatment so that the association of the single-domain antibody with the VL is canceled.
(M5) The screening method according to (M4), wherein the protease cleavage sequence of the association partner provided in the step (b) is located near the boundary between the particular VL and the second association sustaining domain.
(M6) The screening method according to (M3), wherein the fusion polypeptides of the library further comprise a protease cleavage sequence, and the step (d) comprises cleaving the fusion polypeptides by protease treatment so that the association of the single-domain antibody with the VL is canceled.
(M7) The screening method according to (M6), wherein the fusion polypeptide comprises a first association sustaining domain, and the protease cleavage sequence contained in each fusion polypeptide is located near the boundary between the single-domain antibody and the first association sustaining domain.
(M8) The screening method according to (M3), wherein the step (d) comprises in vitro displaying again the full lengths of the fusion polypeptides selected in the step (c) or their moieties comprising the single-domain antibodies.
(M9) The screening method according to (M3), wherein the step (d) comprises in vitro displaying again the full lengths of the fusion polypeptides selected in the step (c) and selecting a fusion polypeptide that binds to the antigen or has antigen binding activity of a predetermined value or higher in a state associated only with the second association sustaining domain.
(M10) A method for screening a library according to (L4) for a fusion polypeptide comprising a single-domain antibody whose antigen binding activity can be inhibited or could be lost by associating with particular VH, wherein the single-domain antibody is a VL having antigen binding activity by itself.
(M11) The screening method according to (M10), comprising the following steps:
   (a) in vitro displaying the fusion polypeptides of the library;
   (b) providing an association partner of a second association sustaining domain fused with a particular VH;
   (c) associating the fusion polypeptides displayed in the step (a) with the association partner provided in the step (b) and selecting a fusion polypeptide that does not bind to the antigen or has antigen binding activity of a predetermined value or lower in a state where the single-domain antibody associates with the VH; and
   (d) selecting, from the fusion polypeptides thus selected in the step (c), a fusion polypeptide that binds to the antigen or has antigen binding activity of a predetermined value or higher in a state where the single-domain antibody contained therein does not associate with the VH.
(M12) The screening method according to (M11), wherein the association partner provided in the step (b) further comprises a protease cleavage sequence, and the step (d) comprises cleaving the association partner by protease treatment so that the association of the single-domain antibody with the VH is canceled.
(M13) The screening method according to (M12), wherein the protease cleavage sequence of the association partner provided in the step (b) is located near the boundary between the particular VH and the second association sustaining domain.
(M14) The screening method according to (M11), wherein the fusion polypeptides of the library further comprise a protease cleavage sequence, and the step (d) comprises cleaving the fusion polypeptides by protease treatment so that the association of the single-domain antibody with the VH is canceled.
(M15) The screening method according to (M14), wherein the fusion polypeptide comprises a first association sustaining domain, and the protease cleavage sequence contained in each fusion polypeptide is located near the boundary between the single-domain antibody and the first association sustaining domain.
(M16) The screening method according to (M11), wherein the step (d) comprises in vitro displaying again the full lengths of the fusion polypeptides selected in the step (c) or their moieties comprising the single-domain antibodies.
(M17) The screening method according to (M11), wherein the step (d) comprises in vitro displaying again the full lengths of the fusion polypeptides selected in the step (c) and selecting a fusion polypeptide that binds to the antigen or has antigen binding activity of a predetermined value or higher in a state associated only with the second association sustaining domain.
(M18) A method for screening a library according to (L5) for a fusion polypeptide comprising a single-domain antibody whose antigen binding activity can be inhibited or could be lost by associating with particular VHH, wherein the single-domain antibody is a VL having antigen binding activity by itself.
(M19) The screening method according to (M18), comprising the following steps:
   (a) in vitro displaying the fusion polypeptides of the library;
   (b) providing an association partner of a second association sustaining domain fused with a particular VHH;
   (c) associating the fusion polypeptides displayed in the step (a) with the association partner provided in the step (b) and selecting a fusion polypeptide that does not bind to the antigen or has antigen binding activity of a predetermined value or lower in a state where the single-domain antibody associates with the particular VHH; and
   (d) selecting, from the fusion polypeptides thus selected in the step (c), a fusion polypeptide that binds to the antigen or has antigen binding activity of a predetermined value or higher in a state where the single-domain antibody contained therein does not associate with the VHH.
(M20) The screening method according to (M19), wherein the association partner provided in the step (b) further comprises a protease cleavage sequence, and the step (d) comprises cleaving the association partner by protease treatment so that the association of the single-domain antibody with the VHH is canceled.
(M21) The screening method according to (M20), wherein the protease cleavage sequence of the association partner provided in the step (b) is located near the boundary between the particular VHH and the second association sustaining domain.
(M22) The screening method according to (M19), wherein the fusion polypeptides of the library further comprise a protease cleavage sequence, and the step (d) comprises cleaving the fusion polypeptides by protease treatment so that the association of the single-domain antibody with the VHH is canceled.
(M23) The screening method according to (M22), wherein the fusion polypeptide comprises a first association sustaining domain, and the protease cleavage sequence contained in each fusion polypeptide is located near the boundary between the single-domain antibody and the first association sustaining domain.
(M24) The screening method according to (M19), wherein the step (d) comprises in vitro displaying again the full lengths of the fusion polypeptides selected in the step (c) or their moieties comprising the single-domain antibodies.
(M25) The screening method according to (M19), wherein the step (d) comprises in vitro displaying again the full lengths of the fusion polypeptides selected in the step (c) and selecting a fusion polypeptide that binds to the antigen or has antigen binding activity of a predetermined value or higher in a state associated only with the second association sustaining domain.
(M26) The screening method according to any of (M3) to (M9), (M11) to (M17), and (M19) to (M25), wherein the step of providing an association partner in the step (b) is the step of displaying the association partner and the fusion polypeptides together.
(M27) The screening method according to any of (M7) to (M9), (M15) to (M17), and (M23) to (M25), wherein the first association sustaining domain comprises an IgG antibody CH1 domain or an antibody light chain constant region.
(M28) The screening method according to any of (M3) to (M9), (M11) to (M17), and (M19) to (M25), wherein the second association sustaining domain comprises an IgG antibody CH1 domain or an antibody light chain constant region.
(M29) The screening method according to (M27) or (M28), wherein the first association sustaining domain comprises an IgG antibody CH1 domain, and the second association sustaining domain comprises an antibody light chain constant region.
(M30) The screening method according to (M27) or (M28), wherein the first association sustaining domain comprises an antibody light chain constant region, and the second association sustaining domain comprises an IgG antibody CH1 domain.
(M31) The screening method according to (M2), comprising the following steps:
   (a) in vitro displaying the fusion polypeptides of the library;
   (b) providing an association partner of a second association sustaining domain fused with a particular VL;
   (c) selecting a fusion polypeptide comprising a single-domain antibody that binds to the antigen or has antigen binding activity of a predetermined value or higher; and
   (d) associating the fusion polypeptides thus selected in the step (c) with the association partner provided in the step (b) and selecting a fusion polypeptide that does not bind to the antigen or has antigen binding activity of a predetermined value or lower in a state where the single-domain antibody associates with the VL.
(M32) The screening method according to (M26), wherein the step (d) comprises in vitro displaying again the fusion polypeptides selected in the step (c).
(M33) The screening method according to (M31), wherein the step (c) comprises associating the fusion polypeptide only with the second association sustaining domain or confirming the antigen binding of the single-domain antibody contained in the fusion polypeptide associated only with the second association sustaining domain.
(M34) The screening method according to (M10), comprising the following steps:
   (a) in vitro displaying the fusion polypeptides of the library;
   (b) providing an association partner of a second association sustaining domain fused with a particular VH;
   (c) selecting a fusion polypeptide comprising a single-domain antibody that binds to the antigen or has antigen binding activity of a predetermined value or higher; and
   (d) associating the fusion polypeptides thus selected in the step (c) with the association partner provided in the step (b) and selecting a fusion polypeptide that does not bind to the antigen or has antigen binding activity of a predetermined value or lower in a state where the single-domain antibody associates with the VH.
(M35) The screening method according to (M34), wherein the step (d) comprises in vitro displaying again the fusion polypeptides selected in the step (c).
(M36) The screening method according to (M34), wherein the step (c) comprises associating the fusion polypeptide only with the second association sustaining domain or confirming the antigen binding of the single-domain antibody contained in the fusion polypeptide associated only with the second association sustaining domain.
(M37) The screening method according to (M18), comprising the following steps:
   (a) in vitro displaying the fusion polypeptides of the library;
   (b) providing an association partner of a second association sustaining domain fused with a particular VHH;
   (c) selecting a fusion polypeptide comprising a single-domain antibody that binds to the antigen or has antigen binding activity of a predetermined value or higher; and
   (d) associating the fusion polypeptides thus selected in the step (c) with the association partner provided in the step (b) and selecting a fusion polypeptide that does not bind to the antigen or has antigen binding activity of a predetermined value or lower in a state where the single-domain antibody associates with the VHH.
(M38) The screening method according to (M37), wherein the step (d) comprises in vitro displaying again the fusion polypeptides selected in the step (c).
(M39) The screening method according to (M37), wherein the step (c) comprises associating the fusion polypeptide only with the second association sustaining domain or confirming the antigen binding of the single-domain antibody contained in the fusion polypeptide associated only with the second association sustaining domain.
(M40) The screening method according to any of (M31) to (M39), wherein the step of associating the fusion polypeptides with the association partner in the step (d) is the step of displaying the association partner and the fusion polypeptides together.
(M41) The screening method according to any of (M31) to (M40), wherein the fusion polypeptide comprises a first association sustaining domain, and the first association sustaining domain comprises an IgG antibody CH1 domain or an antibody light chain constant region.
(M42) The screening method according to any of (M31) to (G41), wherein the second association sustaining domain comprises an IgG antibody CH1 domain or an antibody light chain constant region.
(M43) The screening method according to (M41) or (M42), wherein the first association sustaining domain comprises an IgG antibody CH1 domain, and the second association sustaining domain comprises an antibody light chain constant region.
(M44) The screening method according to (M41) or (M42), wherein the first association sustaining domain comprises an antibody light chain constant region, and the second association sustaining domain comprises an IgG antibody CH1 domain.

The present invention relates to a polypeptide comprising an antigen biding domain and a carrying moiety. In the polypeptide of the present invention, the carrying moiety has an inhibiting domain that inhibits an antigen binding activity of the antigen biding domain. In one embodiment, the antigen biding domain has a shorter half-life in blood than the half-life of the carrying moiety.

In one embodiment, the antigen biding domain of the present invention may bind to or recognize one or more proteins present in cartilage. Alternatively, the antigen biding domain of the present invention may bind to or recognize one or more molecules involved in IL-1 mediated signal transduction. In the present application, IL-1 mediated signal transduction introduction includes, but are not limited to, the signal transduction involved in IL-1R1, IL-1alpha, IL-1beta and/or IL-1RAcP (interleukin-1 Receptor Accessory Protein). Accordingly, the antigen biding domain of the present invention may bind to or recognize one or more proteins selected from the group consisting of IL-1R1, IL-1alpha, IL-1beta and IL-1RAcP (interleukin-1 Receptor Accessory Protein).

The polypeptide comprising the above antigen binding domain is useful in treating and/or preventing a disease or disorder in which the above protein(s) is involved in. Specifically, the polypeptide of the present invention is useful in treating and/or preventing an IL-1R1 mediated disease or disorder. Examples of such a disease or disorder include, but are not limited to, osteoarthritis (OA) and cartilage degradation in osteoarthritis (OA). Further, following diseases or disorders are also exemplified as an IL-1R1 mediated disease or disorder: Acne, Acute coronary syndrome, Adult-onset Still's disease, Allergy, Alzheimer's disease, Anaemia, Aneurysm, Gauty arthritis, Juvenile arthritis, Osteo arthritis, Rheumatoid arthritis, Arthritis, Asthma, Atherosclerosis, Autoimmune disease, Behcet's disease, Cachexia, Leukaemia, Solid cancer, Lung cancer, Billiary cancer, Colorectal cancer, Lymphoma, Myeloma, Ceuroendocrine carcinoma, Ovarian cancer, Castleman's disease, Chronic obstructive pulmonary disease, Conjunctivitis, Dermatomyositis, “Type 1 diabetes, Type 1”, Type 2 diabetes, “Diabetes, undisclosed Type”, Familial cold autoinflammatory syndrome, Familial Mediterranean fever, Graft-versus-host disease, Cerebral haemorrhage, Heart failure, Hidradenitis suppurativa, Hypercholesterolaemia, Hyper-IgD syndrome, Hyperuricaemia, Immunological disease, Myocardial infarction, Burkholderia pseudomallei infection, Kawasaki disease, Vascular inflammation, Inflammatory bowel disease, Inflammatory disease, Intermittent claudication, Cerebral ischaemia, Age-related macular degeneration, Muckle-Wells syndrome, Mucositis, Multiple sclerosis, Myelodysplastic syndrome, Myelofibrosis, Neonatal onset multisystem inflammatory disease, “Nephropathy, diabetic”, Musculoskeletal pain, Neuropathic pain, Peripheral vascular disease, Polymyalgia rheumatic, Polymyositis, Psoriasis, Pyoderma gangrenosum, Reperfusion injury, Restenosis, Rosacea, Sarcoidosis, Schnitzler syndrome, Scleritis, Sepsis, Skin disorder, Spinal disc herniation, Thrombophlebitis, Thrombosis, TNF receptor associated periodic syndrome, Traumatic brain injury, Diabetic ulcer, Uveitis, Vestibular disorder and Xerophthalmia.

A polypeptide of the present invention is partially cleaved by a protease that is expressed in a disease tissue-specific manner. An antigen binding domain is released from the polypeptide by this cleavage. Such released antigen binding domain can penetrate to a deep part of a disease tissue due to the small molecular weight of the domain. Thus, the polypeptide of the present invention can target an antigen present in a deep part of a disease tissue.

In particular, chondrocytes are fully surrounded by cartilage matrix. In order to deliver sufficient amount of drug molecules to cartilage tissue and chondrocytes, the property of drug molecules needs to be carefully designed. It is known that molecular size affects its penetration ability into articular cartilage. As for the effect of molecular size, it is known that the scFv molecule (ESBA105) penetrates into deeper zone of cartilage than IgG (infliximab) even though they have the same variable region (Ann Rheum Dis. 2010 February; 69(2):443-9, Nat Rev Rheumatol. 2017 March; 13(3):183-193). It is considered that the antigen binding domain comprised in the polypeptide of the present invention can sufficiently penetrate into cartilage tissue and chondrocytes. Accordingly, the polypeptide of the present invention is useful in treating and/or preventing a disease or disorder in a bone tissue including osteoarthritis.

The polypeptide according to the present invention usually refers to a peptide having a length on the order of 4 amino acids or longer, and a protein. Also, the polypeptide according to the present invention is usually a polypeptide consisting of an artificially designed sequence, but is not limited thereto. For example, an organism-derived polypeptide may be used. Alternatively, the polypeptide according to the present invention may be any of a natural polypeptide, a synthetic polypeptide, a recombinant polypeptide, and the like. Furthermore, fragments of these polypeptides are also included in the polypeptide of the present invention.

In the present specification, each amino acid is indicated by one-letter code or three-letter code, or both, as represented by, for example, Ala/A, Leu/L, Arg/R, Lys/K, Asn/N, Met/M, Asp/D, Phe/F, Cys/C, Pro/P, Gln/Q, Ser/S, Glu/E, Thr/T, Gly/G, Trp/W, His/H, Tyr/Y, Ile/I, or Val/V. For expressing an amino acid located at a particular position, an expression using a number representing the particular position in combination with the one-letter code or the three-letter code of the amino acid can be appropriately used. For example, an amino acid 37V, which is an amino acid contained in a single-domain antibody, represents Val located at position 37 defined by the Kabat numbering.

For the alteration of an amino acid in the amino acid sequence of a polypeptide, a method known in the art such as site-directed mutagenesis (Kunkel et al. (Proc. Natl. Acad. Sci. USA (1985) 82, 488-492)) or overlap extension PCR can be appropriately adopted. A plurality of methods known in the art can also be adopted as alteration methods for substituting an amino acid by an amino acid other than a natural amino acid (Annu. Rev. Biophys. Biomol. Struct. (2006) 35, 225-249; and Proc. Natl. Acad. Sci. U.S.A. (2003) 100 (11), 6353-6357). For example, a tRNA-containing cell-free translation system (Clover Direct (Protein Express)) having a non-natural amino acid bound with amber suppressor tRNA complementary to UAG codon (amber codon), which is a stop codon, is also preferably used. In the present specification, examples of the alteration include, but are not limited to, substitution.

In the present specification, the term “and/or” used to represent amino acid alteration sites is meant to include every combination appropriately represented by “and” and “or”. Specifically, for example, the phrase “amino acids at positions 37, 45, and/or 47 are substituted” includes the following variations of amino acid alteration: (a) position 37, (b) position 45, (c) position 47, (d) positions 37 and 45, (e) positions 37 and 47, (f) positions 45 and 47, and (g) positions 37, 45 and 47.

In the present specification, expression in which the one-letter codes or three-letter-codes of amino acids before and after alteration are used previous and next to a number representing a particular position can be appropriately used for representing amino acid alteration. For example, an alteration F37V or Phe37Val used for substituting an amino acid contained in an antibody variable region or a single-domain antibody represents the substitution of Phe at position 37 defined by the Kabat numbering by Val. Specifically, the number represents an amino acid position defined by the Kabat numbering; the one-letter code or three-letter code of the amino acid previous to the number represents the amino acid before the substitution; and the one-letter code or three-letter code of the amino acid next to the number represents the amino acid after the substitution. Likewise, an alteration P238A or Pro238Ala used for substituting an amino acid in a Fc region contained in an antibody constant region represents the substitution of Pro at position 238 defined by the EU numbering by Ala. Specifically, the number represents an amino acid position defined by the EU numbering; the one-letter code or three-letter code of the amino acid previous to the number represents the amino acid before the substitution; and the one-letter code or three-letter code of the amino acid next to the number represents the amino acid after the substitution.

In the present specification, the term “antibody” is used in the broadest sense and encompasses various antibody structures including, but are not limited to, a monoclonal antibody, a polyclonal antibody, a multispecific antibody (e.g., a bispecific antibody), a single-domain antibody, and an antibody fragment as long as the antibody exhibits the desired antigen binding activity.

The “antibody fragment” refers to a molecule, other than a complete antibody, containing a portion of the complete antibody and binding to an antigen to which the complete antibody binds. Examples of the antibody fragment include, but are not limited to, Fv, Fab, Fab′, Fab′-SH, F(ab′)2, diabody, linear antibodies, single-chain antibody molecules (e.g., scFv), and multispecific antibodies formed from antibody fragments.

The terms “full-length antibody”, “complete antibody”, and “whole antibody” are used interchangeably with each other in the present specification and refer to an antibody having a structure substantially similar to a natural antibody structure, or having heavy chains containing a Fc region defined in the present specification.

The term “variable region” or “variable domain” refers to a region or a domain of an antibody heavy chain or light chain involved in the binding of the antibody to its antigen. Usually, antibody heavy chain and light chain variable domains (VH and VL, respectively) are structurally similar and each contain 4 conserved framework regions (FRs) and 3 complementarity determining regions (CDRs) (see e.g., Kindt et al., Kuby Immunology, 6th ed., W.H. Freeman and Co., page 91 (2007)). One VH or VL domain may suffice for conferring antigen binding specificity.

The term “complementarity determining region” or “CDR” used in the present specification is hypervariable in the sequence, and/or forms a structurally determined loop (“hypervariable loop”), and/or refers to antigen contact residues (“antigen contacts”) or each region of an antibody variable domain. Usually, an antibody contains 6 CDRs: three in VH (H1, H2, and H3), and three in VL (L1, L2, and L3). In the present specification, exemplary CDRs include the following:
(a) hypervariable loops formed at amino acid residues 26 to 32 (L1), 50 to 52 (L2), 91 to 96 (L3), 26 to 32 (H1), 53 to 55 (H2), and 96 to 101 (H3) (Chothia and Lesk, J. Mol. Biol. 196: 901-917 (1987));
(b) CDRs formed at amino acid residues 24 to 34 (L1), 50 to 56 (L2), 89 to 97 (L3), 31 to 35b (H1), 50 to 65 (H2), and 95 to 102 (H3) (Kabat et al., Sequences of Proteins of Immunological Interest, 5th Ed. Public Health Service, National Institutes of Health, Bethesda, Md. (1991));
(c) antigen contacts formed at amino acid residues 27c to 36 (L1), 46 to 55 (L2), 89 to 96 (L3), 30 to 35b (H1), 47 to 58 (H2), and 93 to 101 (H3) (MacCallum et al., J. Mol. Biol. 262: 732-745 (1996)); and
(d) a combination of (a), (b), and/or (c) containing HVR amino acid residues 46 to 56 (L2), 47 to 56 (L2), 48 to 56 (L2), 49 to 56 (L2), 26 to 35 (H1), 26 to 35b (H1), 49 to 65 (H2), 93 to 102 (H3), and 94 to 102 (H3).

In the present specification, CDR residues and other residues (e.g., FR residues) in a variable domain are numbered according to Kabat et al. (supra), unless otherwise specified.

The term “framework” or “FR” refers to variable domain residues other than complementarity determining region (CDR) residues. FRs in a variable domain consist of 4 FR domains: FR1, FR2, FR3, and FR4. Accordingly, the sequences of CDRs and FRs usually appear in VH (or VL) in the following order: FR1-H1 (L1)-FR2-H2 (L2)-FR3-H3 (L3)-FR4.

In the present specification, the term “constant region” or “constant domain” refers to a region or a domain other than variable regions in an antibody. For example, an IgG antibody is a heterotetrameric glycoprotein of approximately 150,000 Da constituted by two identical light chains and two identical heavy chains connected through disulfide bonds. Each heavy chain has a variable region (VH) also called variable heavy chain domain or heavy chain variable domain, followed by a heavy chain constant region (CH) containing a CH1 domain, a hinge region, a CH2 domain, and a CH3 domain, from the N terminus toward the C terminus. Likewise, each light chain has a variable region (VL) also called variable light chain domain or light chain variable domain, followed by a constant light chain (CL) domain, from the N terminus toward the C terminus. The light chains of natural antibodies may be attributed to one of two types called kappa and lambda on the basis of the amino acid sequences of their constant domains.

In the present specification, the term “Fc region” is used for defining the C-terminal region of immunoglobulin heavy chains, including at least a portion of constant regions. This term includes a Fc region having a natural sequence and a mutant Fc region. In one embodiment, the heavy chain Fc region of human IgG1 spans from Cys226 or Pro230 to the carboxyl terminus of the heavy chain. However, the C-terminal lysine (Lys447) or glycine-lysine (Gly446-Lys447) of the Fc region may be present or absent. In the present specification, amino acid residues in a Fc region or a constant region are numbered according to the EU numbering system (also called EU index) described in Kabat et al., Sequences of Proteins of Immunological Interest, 5th Ed. Public Health Service, National Institutes of Health, Bethesda, Md. 1991, unless otherwise specified.

The “class” of an antibody refers to the type of a constant domain or a constant region carried by the heavy chain of the antibody. Antibodies have 5 major classes: IgA, IgD, IgE, IgG, and IgM. Some of these classes may be further divided into subclasses (isotypes), for example, IgG1, IgG2, IgG3, IgG4, IgA1, and IgA2. Heavy chain constant domains corresponding to immunoglobulins of different classes are called alpha, delta, epsilon, gamma, and mu, respectively.

In the present specification, the “antigen binding domain” is limited only by binding to the antigen of interest. The antigen binding domain can be a domain having any structure as long as the domain used binds to the antigen of interest. Examples of such a domain include, but are not limited to, an antibody heavy chain variable region (VH), an antibody light chain variable region (VL), a single-domain antibody (sdAb), an antagonist, a module called A domain of approximately 35 amino acids contained in an in vivo cell membrane protein avimer (International Publication Nos. WO2004/044011 and WO2005/040229), adnectin containing a 10Fn3 domain serving as a protein binding domain derived from a glycoprotein fibronectin expressed on cell membranes (International Publication No. WO2002/032925), Affibody containing an IgG binding domain scaffold constituting a three-helix bundle composed of 58 amino acids of protein A (International Publication No. WO1995/001937), DARPins (designed ankyrin repeat proteins) which are molecular surface-exposed regions of ankyrin repeats (AR) each having a 33-amino acid residue structure folded into a subunit of a turn, two antiparallel helices, and a loop (International Publication No. WO2002/020565), anticalin having four loop regions connecting eight antiparallel strands bent toward the central axis in one end of a barrel structure highly conserved in lipocalin molecules such as neutrophil gelatinase-associated lipocalin (NGAL) (International Publication No. WO2003/029462), and a depressed region in the internal parallel sheet structure of a horseshoe-shaped fold composed of repeated leucine-rich-repeat (LRR) modules of an immunoglobulin structure-free variable lymphocyte receptor (VLR) as seen in the acquired immune systems of jawless vertebrates such as lamprey or hagfish (International Publication No. WO2008/016854).

Preferred examples of the antigen binding domain of the present invention include an antigen binding domain that can exert an antigen binding function by a molecule constituted only by the antigen binding domain, and an antigen binding domain that can exert an antigen binding function by itself after being released from an additional peptide linked thereto. Examples of such an antigen binding domain include, but are not limited to, a single-domain antibody, scFv, Fv, Fab, Fab′, F(ab′)2, and an antagonist.

One preferred example of the antigen binding domain of the present invention includes an antigen binding domain having a molecular weight of 120 kDa, 100 kDa, 80 kDa, 60 kDa, 40 kDa, 20 kDa or smaller. Examples of such an antigen binding domain include, but are not limited to, single-domain antibodies, scFv, Fab, Fab′, and an antagonist. The antigen binding domain having a molecular weight of 60 kDa or smaller is usually likely to cause clearance by the kidney when existing as a monomer in blood (see J Biol Chem. 1988 Oct. 15; 263 (29): 15064-70). In one embodiment, the antigen binding domain having a molecular weight of 120 kDa or smaller is likely to cause penetrate deeply in the cartilage tissue.

From another viewpoint, one preferred example of the antigen binding domain of the present invention includes an antigen binding domain having a half-life in blood of 12 hours or shorter. Examples of such an antigen binding domain include, but are not limited to, single-domain antibodies, scFv, Fab, Fab′, and an antagonist.

One preferred example of the antigen binding domain of the present invention includes a single-domain antibody (sdAb), and an antagonist.

In the present specification, the term “single-domain antibody” is not limited by its structure as long as the domain can exert antigen binding activity by itself. It is known that a general antibody, for example, an IgG antibody, exhibits antigen binding activity in a state where a variable region is formed by the pairing of VH and VL, whereas the own domain structure of the single-domain antibody can exert antigen binding activity by itself without pairing with another domain. Usually, the single-domain antibody has a relatively low molecular weight and exists in the form of a monomer.

Examples of the single-domain antibody include, but are not limited to, antigen binding molecules congenitally lacking a light chain, such as VHH of an animal of the family Camelidae and shark VNAR, and antibody fragments containing the whole or a portion of an antibody VH domain or the whole or a portion of an antibody VL domain. Examples of the single-domain antibody which is an antibody fragment containing the whole or a portion of an antibody VH or VL domain include, but are not limited to, artificially prepared single-domain antibodies originating from human antibody VH or human antibody VL as described in U.S. Pat. No. 6,248,516 B1, etc. In some embodiments of the present invention, one single-domain antibody has three CDRs (CDR1, CDR2 and CDR3).

The single-domain antibody can be obtained from an animal capable of producing the single-domain antibody or by the immunization of the animal capable of producing the single-domain antibody. Examples of the animal capable of producing the single-domain antibody include, but are not limited to, animals of the family Camelidae, and transgenic animals harboring a gene capable of raising the single-domain antibody. The animals of the family Camelidae include camels, lamas, alpacas, one-hump camels and guanacos, etc. Examples of the transgenic animals harboring a gene capable of raising the single-domain antibody include, but are not limited to, transgenic animals described in International Publication No. WO2015/143414 and U.S. Patent Publication No. US2011/0123527 A1. The framework sequences of the single-domain antibody obtained from the animal may be converted to human germline sequences or sequences similar thereto to obtain a humanized single-domain antibody. The humanized single-domain antibody (e.g., humanized VHH) is also one embodiment of the single-domain antibody of the present invention.

Alternatively, the single-domain antibody can be obtained by ELISA, panning, or the like from a polypeptide library containing single-domain antibodies. Examples of the polypeptide library containing single-domain antibodies include, but are not limited to, naive antibody libraries obtained from various animals or humans (e.g., Methods in Molecular Biology 2012 911 (65-78); and Biochimica et Biophysica Acta—Proteins and Proteomics 2006 1764: 8 (1307-1319)), antibody libraries obtained by the immunization of various animals (e.g., Journal of Applied Microbiology 2014 117:2 (528-536)), and synthetic antibody libraries prepared from antibody genes of various animals or humans (e.g., Journal of Biomolecular Screening 2016 21: 1 (35-43); Journal of Biological Chemistry 2016 291:24 (12641-12657); and AIDS 2016 30: 11 (1691-1701)).

In one embodiment, the sequences of CDRs and frameworks of a single domain antibody usually appear in the following order: FR1-H1 (L1)-FR2-H2 (L2)-FR3-H3 (L3)-FR4.

Generally, a single domain antibody of the present invention can be defined as a polypeptide comprising:
a) An amino acid sequence that is comprised of four framework regions/sequences interrupted by three complementarity determining regions/sequences (in which the amino acid residue at position 11 according to the Kabat numbering is chosen from the group consisting of L, M, S, V, and W, and is preferably L); and/or:
b) An amino acid sequence that is comprised of four framework regions/sequences interrupted by three complementarity determining regions/sequences (in which the amino acid residue at position 37 according to the Kabat numbering is chosen from the group consisting of F, Y, H, I, L, and V, and is preferably F or Y); and/or:
c) An amino acid sequence that is comprised of four framework regions/sequences interrupted by three complementarity determining regions/sequences (in which the amino acid residue at position 44 according to the Kabat numbering is chosen from the group consisting of G, E, A, D, Q, R, S, and L, and is preferably G, E, or Q); and/or:
d) An amino acid sequence that is comprised of four framework regions/sequences interrupted by three complementarity determining regions/sequences (in which the amino acid residue at position 45 according to the Kabat numbering is chosen from the group consisting of L, R, C, I, L, P, Q and V, and is preferably L or R); and/or:
e) An amino acid sequence that is comprised of four framework regions/sequences interrupted by three complementarity determining regions/sequences (in which the amino acid residue at position 47 according to the Kabat numbering is chosen from the group consisting of W, L, F, A, G, I, M, R, S, V, and Y, and is preferably W, L, F or R); and/or:
f) An amino acid sequence that is comprised of four framework regions/sequences interrupted by three complementarity determining regions/sequences (in which the amino acid residue at position 83 according to the Kabat numbering is chosen from the group consisting of R, K, N, E, G, I, M, Q and T, and is preferably K or R, and more preferably K); and/or:
g) An amino acid sequence that is comprised of four framework regions/sequences interrupted by three complementarity determining regions/sequences (in which the amino acid residue at position 84 according to the Kabat numbering is chosen from the group consisting of P, A, L, R, S, T, D and V, and is preferably P); and/or:
h) An amino acid sequence that is comprised of four framework regions/sequences interrupted by three complementarity determining regions/sequences (in which the amino acid residue at position 103 according to the Kabat numbering is chosen from the group consisting of W, P, R, and S, and is preferably and W); and/or:
i) An amino acid sequence that is comprised of four framework regions/sequences interrupted by three complementarity determining regions/sequences (in which the amino acid residue at position 104 according to the Kabat numbering is G or D, and preferably G); and/or:
j) An amino acid sequence that is comprised of four framework regions/sequences interrupted by three complementarity determining regions/sequences (in which the amino acid residue at position 108 according to the Kabat numbering is chosen from the group consisting of Q, L, and R, and is preferably Q or L).

More in particular, in one embodiment, a single domain antibody of the present invention can be defined as a polypeptide comprising an amino acid sequence that is comprised of four framework regions/sequences interrupted by three complementarity determining regions/sequences selected from the group consisting of 1) to 3) below:
k) Amino acid residues at position 43 to 46 according to the Kabat numbering are KERE (SEQ ID NO: 620) or KQRE (SEQ ID NO: 621),
l) Amino acid residues at position 44 to 47 according to the Kabat numbering are GLEW (SEQ ID NO: 622), and
m) Amino acid residues at position 83 to 84 according to the Kabat numbering are KP or EP.

In one embodiment, “Humanized single domain antibody” herein refers to a chimeric single domain antibody comprising amino acid residues from CDR of non-human and human Framework. In one embodiment, in humanized single domain antibody all or substantially all CDRs can be corresponded to those of non-human antibody, and all or substantially all Framework can be corresponded to those of human antibody. In case that in humanized antibody a part of amino acid residues in Framework cannot be corresponded to those of human antibody, it is considered that substantially all Framework can be corresponded to those of human antibody. For instance, in case that VHH as one embodiment of a single domain antibody is humanized, it is required that a part of amino acid residues in Framework is converted to amino acid residues which are not corresponded to those of human antibody (C Vincke, et al., The Journal of Biological Chemistry 284, 3273-3284.)

In one embodiment of the present invention, the single-domain antibody is preferably a VHH, a VH having antigen binding activity by itself or a VL having antigen binding activity by itself. In the present specification, from another viewpoint, “a VH having antigen binding activity by itself” and “a VL having antigen binding activity by itself” can also be defined as “a single-domain antibody prepared from VH,” and “a single-domain antibody prepared from VL”, respectively.

In one embodiment, the antigen binding domain of the present invention may bind to IL-1R1, IL-1alpha, IL-1beta and/or IL-1RAcP. Further, in one embodiment, the antigen binding domain of the present invention may be a single-domain antibody or an antagonist. Moreover, in one embodiment, the single-domain antibody may be a VHH, a VH having antigen binding activity by itself or a VL having antigen binding activity by itself. Accordingly, in one embodiment, the antigen binding domain of the present invention may be a VHH, a VH having antigen binding activity by itself or a VL having antigen binding activity by itself that may bind to or recognize IL-1R1, IL-1alpha, IL-1beta and/or IL-1RAcP.

In the present invention, a soluble IL-1R may be exemplified as an antigen binding domain binding to IL-1alpha and/or IL-1beta. Further, in an embodiment, the antigen-binding domain of the present invention may be a soluble IL-1R1, soluble IL-1R2, soluble IL-1RAcP or a single-domain antibody binding to IL-1alpha and/or IL-1beta. Moreover, the following substances are also exemplified as an antigen binding domain of the present application:
a VL having antigen binding activity by itself that binds to IL-1R1: DOM4-122-23 having an amino acid sequence of SEQ ID NO: 479 and DOM4-130-202 having an amino acid sequence of SEQ ID NO: 480,
a VH having antigen binding activity by itself that binds to IL-1R1:
a VHH having antigen binding activity by itself that binds to IL-1R1:
an antagonist to IL-1R (IL-1Ra): interleukin-1 receptor antagonist protein (Accession no. of protein: NP_776214 (SEQ ID NO:527), Accession no. of mRNA: NM_173842.2), AMG108 (Amgen, H chain of SEQ ID NO: 515, 516 or 517, L chain of SEQ ID NO: 522, 523, or 524), Anakinra (Amgen, SEQ ID NO: 526), ABT-981 (AbbVie, H chain of SEQ ID NO: 512, L chain of SEQ ID NO: 519), Canakinumab (Novartis, H chain of SEQ ID NO: 513, L chain of SEQ ID NO: 520), Gevokizumab (Xoma, H chain of SEQ ID NO: 514, L chain of SEQ ID NO: 521), MABp1 (XBiotech, H chain of SEQ ID NO: 518, L chain of SEQ ID NO: 525).

In the present invention, antibody fragments of the above antibodies can also be used as the antigen-binding domain so long as they exhibit the antagonistic activity to IL-1R. Examples of antibody fragments include but are not limited to Fv, Fab, Fab′, Fab′-SH, F(ab′)2; diabodies; linear antibodies; single-chain antibody molecules (e.g., scFv).
a VL having antigen binding activity by itself that binds to IL-1alpha:
a VH having antigen binding activity by itself that binds to IL-1alpha:
a VHH having antigen binding activity by itself that binds to IL-1alpha:
a VL having antigen binding activity by itself that binds to IL-1beta
a VH having antigen binding activity by itself that binds to IL-1beta:
a VHH having antigen binding activity by itself that binds to IL-1beta:
a VL having antigen binding activity by itself that binds to IL-1RAcP:
a VH having antigen binding activity by itself that binds to IL-1RAcP:
a VHH having antigen binding activity by itself that binds to IL-1RAcP:

Such an embodiment should be obvious to those skilled in the art with reference to the present specification and is included in the scope of the present invention.

In the present specification, the “antigen” is limited only by containing an epitope to which the antigen binding domain binds. Preferred examples of the antigen include, but are not limited to, animal- or human-derived peptides, polypeptides, and proteins. Preferred examples of the antigen for use in the treatment of a disease caused by a target tissue include, but are not limited to, molecules expressed on the surface of target cells (e.g., cancer cells and inflammatory cells), molecules expressed on the surface of other cells in tissues containing target cells, molecules expressed on the surface of cells having an immunological role against target cells and tissues containing target cells, and large molecules present in the stromata of tissues containing target cells.

In one embodiment, antigens may be derived from any animal species (for example, human; or nonhuman animals such as mouse, rat, hamster, guinea pig, rabbit, monkey, cynomolgus monkey, Rhesus monkey, hamadryas baboon, chimpanzee, goat, sheep, dog, horse, pig, bovine, or camel), or any birds; and the antigens are preferably derived from human, rabbit, monkey, rat, or mouse.

Examples of the antigen can include the following molecules: 17-IA, 4-1BB, 4Dc, 6-keto-PGF1a, 8-iso-PGF2a, 8-oxo-dG, A1 adenosine receptor, A33, ACE, ACE-2, activin, activin A, activin AB, activin B, activin C, activin RIA, activin RIA ALK-2, activin RIB ALK-4, activin RIIA, activin RIIB, ADAM, ADAM10, ADAM12, ADAM15, ADAM17/TACE, ADAM8, ADAM9, ADAMTS, ADAMTS4, ADAMTS5, addressin, aFGF, ALCAM, ALK, ALK-1, ALK-7, alpha-1-antitrypsin, alpha-V/beta-1 antagonist, ANG, Ang, APAF-1, APE, APJ, APP, APRIL, AR, ARC, ART, artemin, anti-Id, ASPARTIC, atrial natriuretic factor, av/b3 integrin, Ax1, b2M, B7-1, B7-2, B7-H, B-lymphocyte stimulator (BlyS), BACE, BACE-1, Bad, BAFF, BAFF-R, Bag-1, BAK, Bax, BCA-1, BCAM, Bcl, BCMA, BDNF, b-ECGF, bFGF, BID, Bik, BIM, BLC, BL-CAM, BLK, BMP, BMP-2 BMP-2a, BMP-3 osteogenin, BMP-4 BMP-2b, BMP-5, BMP-6 Vgr-1, BMP-7 (OP-1), BMP-8 (BMP-8a, OP-2), BMPR, BMPR-IA (ALK-3), BMPR-IB (ALK-6), BRK-2, RPK-1, BMPR-II (BRK-3), BMP, b-NGF, BOK, bombesin, bone-derived neurotrophic factor, BPDE, BPDE-DNA, BTC, complement factor 3 (C3), C3a, C4, C5, C5a, C10, CA125, CAD-8, calcitonin, cAMP, carcinoembryonic antigen (CEA), cancer-associated antigens, cathepsin A, cathepsin B, cathepsin C/DPPI, cathepsin D, cathepsin E, cathepsin H, cathepsin L, cathepsin O, cathepsin S, cathepsin V, cathepsin X/Z/P, CBL, CCI, CCK2, CCL, CCL1, CCL11, CCL12, CCL13, CCL14, CCL15, CCL16, CCL17, CCL18, CCL19, CCL2, CCL20, CCL21, CCL22, CCL23, CCL24, CCL25, CCL26, CCL27, CCL28, CCL3, CCL4, CCL5, CCL6, CCL7, CCL8, CCL9/10, CCR, CCR1, CCR10, CCR10, CCR2, CCR3, CCR4, CCR5, CCR6, CCR7, CCR8, CCR9, CD1, CD2, CD3, CD3E, CD4, CD5, CD6, CD7, CD8, CD10, CD11a, CD11b, CD11c, CD13, CD14, CD15, CD16, CD18, CD19, CD20, CD21, CD22, CD23, CD25, CD27L, CD28, CD29, CD30, CD30L, CD32, CD33 (p67 protein), CD34, CD38, CD40, CD40L, CD44, CD45, CD46, CD49a, CD52, CD54, CD55, CD56, CD61, CD64, CD66e, CD74, CD80 (B7-1), CD89, CD95, CD123, CD137, CD138, CD140a, CD146, CD147, CD148, CD152, CD164, CEACAM5, CFTR, cGMP, CINC, botulinum toxin, Clostridium perfringens toxin, CKb8-1, CLC, CMV, CMV UL, CNTF, CNTN-1, COX, C-Ret, CRG-2, CT-1, CTACK, CTGF, CTLA-4, PD1, PDL1, LAGS, TIM3, galectin-9, CX3CL1, CX3CR1, CXCL, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCR, CXCR1, CXCR2, CXCR3, CXCR4, CXCR5, CXCR6, cytokeratin tumor-associated antigens, DAN, DCC, DcR3, DC-SIGN, decay accelerating factor, des(1-3)-IGF-I (brain IGF-1), Dhh, digoxin, DNAM-1, Dnase, Dpp, DPPIV/CD26, Dtk, ECAD, EDA, EDA-A1, EDA-A2, EDAR, EGF, EGFR (ErbB-1), EMA, EMMPRIN, ENA, endothelin receptor, enkephalinase, eNOS, Eot, eotaxin 1, EpCAM, ephrin B2/EphB4, EPO, ERCC, E-selectin, ET-1, factor IIa, factor VII, factor VIIIc, factor IX, fibroblast-activating protein (FAP), Fas, FcR1, FEN-1, ferritin, FGF, FGF-19, FGF-2, FGF3, FGF-8, FGFR, FGFR-3, fibrin, FL, FLIP, Flt-3, Flt-4, follicle-stimulating hormone, fractalkine, FZD1, FZD2, FZD3, FZD4, FZD5, FZD6, FZD7, FZD8, FZD9, FZD10, G250, Gas6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (myostatin), GDF-9, GDF-15 (MIC-1), GDNF, GDNF, GFAP, GFRa-1, GFR-alpha 1, GFR-alpha 2, GFR-alpha 3, GITR, glucagon, Glut4, glycoprotein (GPIIb/IIIa), GM-CSF, gp130, gp72, GRO, growth hormone-releasing factor, hapten (NP-cap or NIP-cap), HB-EGF, HCC, HCMV gB envelope glycoprotein, HCMV gH envelope glycoprotein, HCMV UL, hematopoietic growth factor (HGF), Hep B gp120, heparanase, Her2, Her2/neu (ErbB-2), Her3 (ErbB-3), Her4 (ErbB-4), herpes simplex virus (HSV) gB glycoprotein, HSV gD glycoprotein, HGFA, high-molecular-weight melanoma-associated antigen (HMW-MAA), HIV gp120, HIV IIIB gp 120 V3 loop, HLA, HLA-DR, HM1.24, HMFG PEM, HRG, Hrk, human heart myosin, human cytomegalovirus (HCMV), human growth hormone (HGH), HVEM, 1-309, IAP, ICAM, ICAM-1, ICAM-3, ICE, ICOS, IFNg, Ig, IgA receptor, IgE, IGF, IGF binding protein, IGF-1R, IGFBP, IGF-I, IGF-II, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-18, IL-18R, IL-21, IL-23, IL-27, interferon (INF)-alpha, INF-beta, INF-gamma, inhibin, iNOS, insulin chain A, insulin chain B, insulin-like growth factor 1, integrin alpha 2, integrin alpha 3, integrin alpha 4, integrin alpha 4/beta 1, integrin alpha 4/beta 7, integrin alpha 5 (alpha V), integrin alpha 5/beta 1, integrin alpha 5/beta 3, integrin alpha 6, integrin beta 1, integrin beta 2, interferon gamma, IP-10, I-TAC, JE, kallikrein 2, kallikrein 5, kallikrein 6, kallikrein 11, kallikrein 12, kallikrein 14, kallikrein 15, kallikrein L1, kallikrein L2, kallikrein L3, kallikrein L4, KC, KDR, keratinocyte growth factor (KGF), laminin 5, LAMP, LAP, LAP (TGF-1), latent TGF-1, latent TGF-1 bp1, LBP, LDGF, LECT2, lefty, Lewis-Y antigen, Lewis-Y-related antigen, LFA-1, LFA-3, Lfo, LIF, LIGHT, lipoprotein, LIX, LKN, Lptn, L-selectin, LT-a, LT-b, LTB4, LTBP-1, lung surface, luteinizing hormone, lymphotoxin beta receptor, Mac-1, MAdCAM, MAG, MAP2, MARC, MCAM, MCAM, MCK-2, MCP, M-CSF, MDC, Mer, metalloproteinases, MGDF receptor, MGMT, MHC (HLA-DR), MIF, MIG, MIP, MIP-1-alpha, MK, MMAC1, MMP, MMP-1, MMP-10, MMP-11, MMP-12, MMP-13, MMP-14, MMP-15, MMP-2, MMP-24, MMP-3, MMP-7, MMP-8, MMP-9, MPIF, Mpo, MSK, MSP, mucin (Muc1), MUC18, mullerian-inhibiting substance, Mug, MuSK, NAIP, NAP, NCAD, N-C adherin, NCA 90, NCAM, NCAM, neprilysin, neurotrophin-3, -4, or -6, neurturin, nerve growth factor (NGF), NGFR, NGF-beta, nNOS, NO, NOS, Npn, NRG-3, NT, NTN, OB, OGG1, OPG, OPN, OSM, OX40L, OX40R, p150, p95, PADPr, parathyroid hormone, PARC, PARP, PBR, PBSF, PCAD, P-cadherin, PCNA, PDGF, PDGF, PDK-1, PECAM, PEM, PF4, PGE, PGF, PGI2, PGJ2, PIN, PLA2, placental alkaline phosphatase (PLAP), PlGF, PLP, PP14, proinsulin, prorelaxin, protein C, PS, PSA, PSCA, prostate-specific membrane antigen (PSMA), PTEN, PTHrp, Ptk, PTN, R51, RANK, RANKL, RANTES, RANTES, relaxin A chain, relaxin B chain, renin, respiratory syncytial virus (RSV) F, RSV Fgp, Ret, rheumatoid factor, RLIP76, RPA2, RSK, S100, SCF/KL, SDF-1, SERINE, serum albumin, sFRP-3, Shh, SIGIRR, SK-1, SLAM, SLPI, SMAC, SMDF, SMOH, SOD, SPARC, Stat, STEAP, STEAP-II, TACE, TACI, TAG-72 (tumor-associated glycoprotein-72), TARC, TCA-3, T cell receptor (e.g., T cell receptor alpha/beta), TdT, TECK, TEM1, TEMS, TEM7, TEM8, TERT, testicular PLAP-like alkaline phosphatase, TfR, TGF, TGF-alpha, TGF-beta, TGF-beta Pan Specific, TGF-beta RI (ALK-5), TGF-beta RII, TGF-beta RIIb, TGF-beta RIII, TGF-beta 1, TGF-beta 2, TGF-beta 3, TGF-beta 4, TGF-beta 5, thrombin, thymus Ck-1, thyroid stimulating hormone, Tie, TIMP, TIQ, tissue factor, TMEFF2, Tmpo, TMPRSS2, TNF, TNF-alpha, TNF-alpha/beta, TNF-beta 2, TNFc, TNF-RI, TNF-RII, TNFRSF10A (TRAIL R1 Apo-2, DR4), TNFRSF10B (TRAIL R2 DR5, KILLER, TRICK-2A, TRICK-B), TNFRSF10C (TRAIL R3 DcR1, LIT, TRID), TNFRSF10D (TRAIL R4 DcR2, TRUNDD), TNFRSF11A (RANK ODF R, TRANCE R), TNFRSF11B (OPG OCIF, TR1), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF18 (GITR AITR), TNFRSF19 (TROY TAJ, TRADE), TNFRSF19L (RELT), TNFRSF1A (TNF RI CD120a, p55-60), TNFRSF1B (TNF RII CD120b, p75-80), TNFRSF26 (TNFRH3), TNFRSF3 (LTbR TNF RIII, TNFC R), TNFRSF4 (OX40 ACT35, TXGP1 R), TNFRSF5 (CD40 p50), TNFRSF6 (Fas Apo-1, APT1, CD95), TNFRSF6B (DcR3 M68, TR6), TNFRSF7 (CD27), TNFRSF8 (CD30), TNFRSF9 (4-1BB CD137, ILA), TNFRSF21 (DR6), TNFRSF22 (DcTRAIL R2 TNFRH2), TNFRST23 (DcTRAIL R1 TNFRH1), TNFRSF25 (DR3 Apo-3, LARD, TR-3, TRAMP, WSL-1), TNFSF10 (TRAIL Apo-2 ligand, TL2), TNFSF11 (TRANCE/RANK ligand ODF, OPG ligand), TNFSF12 (TWEAK Apo-3 ligand, DR3 ligand), TNFSF13 (APRIL TALL2), TNFSF13B (BAFF BLYS, TALL1, THANK, TNFSF20), TNFSF14 (LIGHT HVEM ligand, LTg), TNFSF15 (TL1A/VEGI), TNFSF18 (GITR ligand AITR ligand, TL6), TNFSF1A (TNF-a Conectin, DIF, TNFSF2), TNFSF1B (TNF-b LTa, TNFSF1), TNFSF3 (LTb TNFC, p33), TNFSF4 (OX40 ligand gp34, TXGP1), TNFSF5 (CD40 ligand CD154, gp39, HIGM1, IMD3, TRAP), TNFSF6 (Fas ligand Apo-1 ligand, APT1 ligand), TNFSF7 (CD27 ligand CD70), TNFSF8 (CD30 ligand CD153), TNFSF9 (4-1BB ligand CD137 ligand), TP-1, t-PA, Tpo, TRAIL, TRAIL R, TRAIL-R1, TRAIL-R2, TRANCE, transferrin receptor, TRF, Trk, TROP-2, TLR (toll-like receptor) 1, TLR2, TLR3, TLR4, TLR5, TLR6, TLR7, TLR8, TLR9, TLR10, TSG, TSLP, tumor-associated antigen CA125, tumor-associated antigen-expressing Lewis-Y-related carbohydrate, TWEAK, TXB2, Ung, uPAR, uPAR-1, urokinase, VCAM, VCAM-1, VECAD, VE-cadherin, VE-cadherin-2, VEFGR-1 (flt-1), VEGF, VEGFR, VEGFR-3 (flt-4), VEGI, VIM, viral antigens, VLA, VLA-1, VLA-4, VNR integrin, von Willebrand factor, WIF-1, WNT1, WNT2, WNT2B/13, WNT3, WNT3A, WNT4, WNT5A, WNT5B, WNT6, WNT7A, WNT7B, WNT8A, WNT8B, WNT9A, WNT9A, WNT9B, WNT10A, WNT10B, WNT11, WNT16, XCL1, XCL2, XCR1, XCR1, XEDAR, XIAP, XPD, HMGB1, IgA, A beta, CD81, CD97, CD98, DDR1, DKK1, EREG, Hsp90, IL-17/IL-17R, IL-20/IL-20R, oxidized LDL, PCSK9, prekallikrein, RON, TMEM16F, SOD1, chromogranin A, chromogranin B, tau, VAP1, high-molecular-weight kininogen, IL-31, IL-31R, Nav1.1, Nav1.2, Nav1.3, Nav1.4, Nav1.5, Nav1.6, Nav1.7, Nav1.8, Nav1.9, EPCR, C1, C1q, C1r, C1s, C2, C2a, C2b, C3, C3a, C3b, C4, C4a, C4b, C5, C5a, C5b, C6, C7, C8, C9, factor B, factor D, factor H, properdin, sclerostin, fibrinogen, fibrin, prothrombin, thrombin, tissue factor, factor V, factor Va, factor VII, factor VIIa, factor VIII, factor VIIIa, factor IX, factor IXa, factor X, factor Xa, factor XI, factor XIa, factor XII, factor XIIa, factor XIII, factor XIIIa, TFPI, antithrombin III, EPCR, thrombomodulin, TAPI, tPA, plasminogen, plasmin, PAI-1, PAI-2, GPC3, syndecan-1, syndecan-2, syndecan-3, syndecan-4, LPA, SIP, receptors for hormones or growth factors, and a molecule present in a cartilage tissue.

The NCBI (National Center for Biotechnology Information, U.S. National Library of Medicine) database accession numbers and the SEQ ID NOs used herein are shown below for nucleic acid sequences and amino acid sequences for IL-1R1, IL-1R2, IL-1alpha, IL-1beta and IL-1RAcP:
IL-1R1: a nucleic acid sequence NM_001320982.1, an amino acid sequence NP_001307911 (SEQ ID NO: 528)
IL-1R2 transcript variant 1: a nucleic acid sequence NM_004633.3, an amino acid sequence NP_004624.1 (SEQ ID NO: 529)
IL-1alpha: a nucleic acid sequence NM_000575.4, an amino acid sequence NP_000566 (SEQ ID NO: 530)
IL-1beta: a nucleic acid sequence NM_000576.2, an amino acid sequence NP_000567 (SEQ ID NO: 531)
IL-1RAcP: a nucleic acid sequence NM_001167929.1, an amino acid sequence NP_001161401 (SEQ ID NO: 532)

In the present invention, IL1-R1, IL1-R2, IL-1alpha, IL-1beta and IL-1RAcP includes any isoforms, polymorphisms and variants (including allelic variants and splicing variants) of IL1-R1, IL1-R2, IL-1alpha, IL-1beta and IL-1RAcP.

Although the examples of the antigen listed above also include receptors, these receptors even existing in a soluble form in a body fluid can be used as the antigen to which the antigen binding domain of the present invention binds. One non-limiting example of the soluble form of such a receptor can include the protein represented by SEQ ID NO: 35 which is soluble IL-6R as described by Mullberg et al. (J. Immunol. (1994) 152 (10), 4958-4968).

The examples of the antigen listed above include membrane molecules expressed on cell membranes, and soluble molecules secreted from cells to the outside of the cells. When the antigen binding domain of the present invention binds to a soluble molecule secreted from cells, the antigen binding domain preferably has neutralizing activity.

The solution containing the soluble molecule is not limited, and this soluble molecule may exist in a body fluid, i.e., every vascular liquid or every liquid filling between tissues or cells in living bodies. In a non-limiting aspect, the soluble molecule to which the antigen binding domain of the present invention binds can exist in an extracellular fluid. The extracellular fluid refers to a generic name for plasma, intercellular fluid, lymph, tight connective tissues, cerebrospinal fluid, spinal fluid, aspirates, synovial fluid, or such components in the bone and cartilage, alveolar fluid (bronchioalveolar lavage fluid), ascitic fluid, pleural effusion, cardiac effusion, cyst fluid, aqueous humor (hydatoid), or such transcellular fluids (various fluids in glandular cavities resulting from the active transport or secretory activity of cells, and fluids in the lumen of the gut and other body cavities) in vertebrates.

The antigen binding domain of the present invention may recognize or bind to an epitope within an antigen. The epitope, which means an antigenic determinant, present in the antigen means a site on the antigen to which the antigen binding domain disclosed in the present specification binds. Accordingly, for example, the epitope can be defined by its structure. Alternatively, the epitope may be defined by the antigen-binding activity of the antigen binding domain recognizing the epitope. When the antigen is a peptide or a polypeptide, the epitope may be identified by amino acid residues constituting the epitope. When the epitope is a sugar chain, the epitope may be identified by a particular sugar chain structure.

A linear epitope refers to an epitope comprising an epitope that is recognized by its primary sequence of amino acids. The linear epitope contains typically at least 3 and most commonly at least 5, for example, approximately 8 to approximately 10 or 6 to 20 amino acids, in its unique sequence.

In contrast to the linear epitope, a conformational epitope refers to an epitope that is contained in a primary sequence of amino acids containing a component other than the single defined component of the epitope to be recognized (e.g., an epitope whose primary sequence of amino acids may not be recognized by an antibody that determines the epitope). The conformational epitope may contain an increased number of amino acids, as compared with the linear epitope. As for the recognition of the conformational epitope, the antigen binding domain recognizes the three-dimensional structure of the peptide or the protein. For example, when a protein molecule is folded to form a three-dimensional structure, certain amino acids and/or polypeptide backbone constituting the conformational epitope are arranged in parallel to allow the antibody to recognize the epitope. Examples of the method for determining the conformation of the epitope include, but are not limited to, X-ray crystallography, two-dimensional nuclear magnetic resonance spectroscopy, and site-specific spin labeling and electron paramagnetic resonance spectroscopy. See, for example, Epitope Mapping Protocols in Methods in Molecular Biology (1996), Vol. 66, Morris ed.

The structure of the antigen binding domain binding to the epitope is called paratope. The paratope stably binds to the epitope through a hydrogen bond, electrostatic force, van der Waals' forces, a hydrophobic bond, or the like acting between the epitope and the paratope. This binding force between the epitope and the paratope is called affinity. The total binding force when a plurality of antigen binding domains bind to a plurality of antigens is called avidity. The affinity works synergistically when, for example, an antibody comprising a plurality of antigen binding domains (i.e., a polyvalent antibody) bind to a plurality of epitopes. Therefore, the avidity is higher than the affinity.

In a particular embodiment, the antigen binding domain provided in the present specification has a dissociation constant (Kd) of 1 micro M or less, 100 nM or less, 10 nM or less, 1 nM or less, 0.1 nM or less, 0.01 nM or less, or 0.001 nM or less (e.g., 10⁻⁸ M or less, for example, 10⁻⁸ M to 10⁻¹³ M, for example, 10⁻⁹ M to 10⁻¹³ M).

Hereinafter, an exemplary method for confirming the binding of an antigen binding domain directed to IL-6R, or a polypeptide comprising the antigen binding domain to the epitope will be shown. However, a method for confirming the binding of an antigen binding domain directed to an antigen other than IL-6R (for example, but are not limited to, IL-1R1, IL-1alpha, IL-1beta and/or IL-1RAcP), or a polypeptide comprising the antigen binding domain to the epitope can also be appropriately carried out according to the example given below.

For example, whether the antigen binding domain directed to IL-6R recognizes a linear epitope present in the IL-6R molecule can be confirmed, for example, as follows: a linear peptide comprising an amino acid sequence constituting the extracellular domain of IL-6R is synthesized for the purpose described above. The peptide can be chemically synthesized. Alternatively, the peptide is obtained by a genetic engineering approach using a region encoding an amino acid sequence corresponding to the extracellular domain in IL-6R cDNA. Next, the antigen binding domain directed to IL-6R is evaluated for its binding activity against the linear peptide comprising an amino acid sequence constituting the extracellular domain. For example, the binding activity of the antigen binding domain against the peptide can be evaluated by ELISA using an immobilized linear peptide as an antigen. Alternatively, the binding activity against the linear peptide may be determined on the basis of a level at which the linear peptide inhibits the binding of the antigen binding domain to IL-6R-expressing cells. These tests can determine the binding activity of the antigen binding domain against the linear peptide.

In an embodiment, the antigen-binding domain of the present invention may bind to an epitope of IL-1R1, 1L-1alpha, 1L-1beta, and/or IL-1RAcP. An epitope sequence in an antigen can be obtained by epitope analysis techniques well-known to those skilled in the art. In an embodiment, the epitope of the present invention may be present within the D1 domain, D2 domain, or D3 domain of IL-1R1 (PLoS One. 2015 Feb. 23; 10(2): e0118671). Further, in an embodiment, the epitope of the present invention may be present within the D3 domain of IL-1R1. Further, in an embodiment, the epitope of the present invention may compete with IL-1beta but not compete with IL-1Ra. Further, in an embodiment, the epitope of the present invention may allow cross-reactivity between human IL-1R1 and rabbit IL-1R1.

In one embodiment, an IL-1R1 binding domain of the present invention may compete for binding the epitope with a single-domain antibody VL selected from the group consisting of 1) and 2) below:
1) a single-domain antibody VL comprising the amino acid sequence of SEQ ID NO: 479, and
2) a single-domain antibody VL comprising the amino acid sequence of SEQ ID NO: 480.

In one embodiment, an IL-1R1 binding domain of the present invention may compete for binding the epitope with interleukin-1 receptor antagonist protein or an antibody selected from the group consisting of 1) and 2) below:
1) interleukin-1 receptor antagonist protein (Accession no. of protein: NP_776214 (SEQ ID NO: 527), Anakinra (Amgen, SEQ ID NO: 526), and AMG108 (Amgen, H chain of SEQ ID NO: 515, 516 or 517, L chain of SEQ ID NO: 522, 523, or 524, WO2004/022718), and
2) SEQ ID NOs: 540 to 617.

In one embodiment, an IL-1alpha and/or an IL-1beta binding domain of the present invention may compete for binding the epitope with an antibody selected from the group consisting of 1) below:

ABT-981 (AbbVie, H chain of SEQ ID NO: 512, L chain of SEQ ID NO: 519), Canakinumab (Novartis, H chain of SEQ ID NO: 513, L chain of SEQ ID NO: 520, WO2013/082282), Gevokizumab (Xoma, H chain of SEQ ID NO: 514, L chain of SEQ ID NO: 521, U.S. Pat. No. 8,551,487), and MABp1 (XBiotech, H chain of SEQ ID NO: 518, L chain of SEQ ID NO: 525, U.S. Pat. No. 8,034,337).

In the present invention, the IL-1R1 binding domain may not compete with IL-1Ra or may compete with IL-1Ra.

Whether an antigen binding domain recognizes the same epitope recognized by other molecules including the single-domain antibody VL of any one of (1) and (2) above, IL-1Ra and IL-1beta can be confirmed by the competition between the antigen binding domain and the other molecules against the epitope. Competition can be evaluated by competitive binding assays using means such as ELISA, fluorescence energy transfer method (FRET), and fluorometric microvolume assay technology (FMAT™).

The amount of the single-domain antibody VL of any one of (1) and (2), IL-1Ra and IL-1beta that are bound to an antigen indirectly correlates with the binding ability of candidate competitor antigen binding domains (test antigen binding domains) that competitively bind to the same epitope. In other words, as the amount of or the affinity of test antigen binding domains against the same epitope increases, the amount of the single-domain antibody VL of any one of (a) and (b), IL-1Ra and IL-1beta that are bound to the antigen decreases, and the amount of test antigen binding domains bound to the antigen increases. Specifically, the single-domain antibody VL of any one of (a) and (b), IL-1Ra or IL-1beta that is appropriately labeled and an antigen binding domain to be evaluated are simultaneously added to an antigen, and thus the single-domain antibody VL of any one of (a) and (b), IL-1Ra or IL-1beta that is bound to the antigen is detected using the label. The amount of the single-domain antibody VL of any one of (a) and (b), IL-1Ra or IL-1beta that is bound to the antigen can be easily determined by labeling these molecules beforehand. This label is not particularly limited, and the labeling method is selected according to the assay technique used. The labeling method includes fluorescent labeling, radiolabeling, enzymatic labeling, and such.

Also, whether the antigen binding domain directed to IL-6R recognizes the conformational epitope can be confirmed as follows: IL-6R-expressing cells are prepared for the purpose described above. The recognition of the conformational epitope by the antigen binding domain directed to IL-6R is confirmed, for example, when the antigen binding domain directed to IL-6R strongly binds to the IL-6R-expressing cells upon contact with the cells, whereas the antigen binding domain does not substantially bind to an immobilized linear peptide comprising an amino acid sequence constituting the extracellular domain of IL-6R or a denatured (using a general denaturant such as guanidine) linear peptide comprising an amino acid sequence constituting the extracellular domain of IL-6R. In this context, the term “not substantially bind” means that the binding activity is 80% or less, usually 50% or less, preferably 30% or less, particularly preferably 15% or less of binding activity against cells expressing human IL-6R.

The method for confirming the antigen binding activity of the antigen binding domain also includes a method of measuring a Kd value by, for example, radiolabeled antigen binding assay (RIA). In one embodiment, RIA is carried out using the antigen binding domain of interest and its antigen. For example, the binding affinity in a solution of the antigen binding domain for the antigen is measured by equilibrating the antigen binding domain with the smallest concentration of a (125I)-labeled antigen in the presence of a titration series of an unlabeled antigen, and subsequently capturing the bound antigen by a plate coated with the antigen binding domain (see e.g., Chen et al., J. Mol. Biol. 293: 865-881 (1999)).

According to an alternative embodiment, Kd is measured by a surface plasmon resonance method using BIACORE®. For example, assay using BIACORE®-2000 or BIACORE®-3000 (BIAcore, Inc., Piscataway, N.J.) is carried out at 25 degrees C. using a CMS chip with approximately 10 response units (RU) of the antigen immobilized thereon. In one embodiment, a carboxymethylated dextran biosensor chip (CMS, BIAcore, Inc.) is activated using N-ethyl-N-(3-dimethylaminopropyl)-carbodiimide hydrochloride (EDC) and N-hydroxysuccinimide (NHS) according to the supplier's instruction. The antigen is diluted to 5 micro g/ml (approximately 0.2 micro M) with 10 mM sodium acetate (pH 4.8) and then injected thereto at a flow rate of 5 micro 1/min so as to attain protein binding at approximately 10 response units (RU). After the antigen injection, 1 M ethanolamine is injected thereto in order to block unreacted groups. For kinetic measurement, 2-fold dilutions (0.78 nM to 500 nM) of the antigen binding domain in PBS containing 0.05% Polysorbate 20 (TWEEN-20™) as a surfactant (PBST) are injected thereto at a flow rate of approximately 25 micro 1/min at 25 degrees C. An association rate (kon) and a dissociation rate (koff) are calculated by fitting sensorgrams of association and dissociation at the same time using a simple 1:1 Langmuir binding model (BIACORE® evaluation software version 3.2). An equilibrium dissociation constant (Kd) is calculated as a koff/kon ratio. Furthermore, an apparent dissociation constant (Kd) may be determined by use of equilibrium analysis. For these procedures, see the protocol attached to BIACORE®. See, for example, Chen et al., J. Mol. Biol. 293: 865-881 (1999) and Methods Enzymol. 2000; 323: 325-40. In the surface plasmon resonance assay, the amount of the protein immobilized, the amount of the protein used in reaction, temperature, and solution composition can be variously changed by those skilled in the art. When the on-rate in the surface plasmon resonance assay described above exceeds 106 M-1s-1, the on-rate can be determined by use of a fluorescence quenching technique of using a spectrometer (e.g. a stopped-flow spectrophotometer (Aviv Instruments, Inc.) or SLM-AMINCO™ spectrophotometer 8000 series (Thermo Spectronic/Thermo Fisher Scientific Inc.) using a stirring cuvette) to measure increase or decrease in fluorescence intensity (excitation=295 nm; emission=340 nm, band path: 16 nm) at 25 degrees C. for 20 nM antigen binding domain in PBS (pH 7.2) in the presence of gradually increased concentrations of the antigen.

Furthermore, the antigen binding activity of the antigen binding domain can also be measured by a known molecule-molecule interaction measurement method such as electrogenerated chemiluminescence.

Examples of the method for measuring the binding activity of the antigen binding domain directed to IL-6R against the IL-6R-expressing cells include methods described in Antibodies: A Laboratory Manual (Ed Harlow, David Lane, Cold Spring Harbor Laboratory (1988) 359-420). Specifically, the binding activity can be evaluated on the basis of the principle of ELISA or FACS (fluorescence activated cell sorting) using the IL-6R-expressing cells as an antigen.

In the ELISA format, the binding activity of the antigen binding domain directed to IL-6R against the IL-6R-expressing cells is quantitatively evaluated by comparing the levels of signals generated through enzymatic reaction. Specifically, a test antigen binding domain is added to an ELISA plate with the IL-6R-expressing cells immobilized thereon. Then, the test antigen binding domain bound with the cells is detected through the use of an enzyme-labeled antibody recognizing the test antigen binding domain. Alternatively, in the FACS, a dilution series of a test antigen binding domain is prepared, and the antibody binding titer for the IL-6R-expressing cells can be determined to compare the binding activity of the test antigen binding domain against the IL-6R-expressing cells.

The binding of the test antigen binding domain to the antigen expressed on the surface of cells suspended in a buffer solution or the like can be detected using a flow cytometer. For example, the following apparatuses are known as the flow cytometer:
FACSCanto™ II
FACSAria™
FACSArray™
FACSVantage™ SE
FACSCalibur™ (all are trade names of BD Biosciences)
EPICS ALTRA HyPerSort
Cytomics FC 500
EPICS XL-MCL ADC EPICS XL ADC
Cell Lab Quanta/Cell Lab Quanta SC (all are trade names of Beckman Coulter, Inc.)

One preferred example of the method for measuring the antigen binding activity of the antigen binding domain directed to IL-6R includes the following method: first, IL-6R-expressing cells reacted with a test antigen binding domain are stained with a FITC-labeled secondary antibody recognizing the test antigen binding domain. The test antigen binding domain is appropriately diluted with a suitable buffer solution to prepare the antigen binding domain at the desired concentration for use. The antigen binding domain can be used, for example, at any concentration from 10 micro g/ml to 10 ng/ml. Next, fluorescence intensity and the number of cells are measured using FACSCalibur (Becton, Dickinson and Company). The amount of the antigen binding domain bound to the cells is reflected in the fluorescence intensity obtained by analysis using CELL QUEST Software (Becton, Dickinson and Company), i.e., a geometric mean value. In short, the binding activity of the test antigen binding domain indicated by the amount of the test antigen binding domain bound can be determined by obtaining the geometric mean value.

Whether the antigen binding domain directed to IL-6R shares an epitope with a certain antigen binding domain can be confirmed by the competition between these antigen binding domains for the same epitope. The competition between the antigen binding domains is detected by cross-blocking assay or the like. The cross-blocking assay is preferably, for example, competitive ELISA assay.

Specifically, in the cross-blocking assay, IL-6R protein-coated wells of a microtiter plate are preincubated in the presence or absence of a candidate competitor antigen binding domain. Then, a test antigen binding domain is added thereto. The amount of the test antigen binding domain bound with the IL-6R protein in the wells indirectly correlates with the binding capacity of the candidate competitor antigen binding domain that competes for the binding to the same epitope. In short, larger affinity of the competitor antigen binding domain for the same epitope means lower binding activity of the test antigen binding domain against the IL-6R protein-coated wells.

The amount of the test antigen binding domain bound with the wells via the IL-6R protein can be easily measured by labeling the antigen binding domain in advance. For example, a biotin-labeled antigen binding domain is assayed by using an avidin-peroxidase conjugate and an appropriate substrate. In particular, cross-blocking assay that utilizes enzyme labels such as peroxidase is called competitive ELISA assay. The antigen binding domain can be labeled with an alternative detectable or measurable labeling material. Specifically, radiolabels, fluorescent labels, and the like are known in the art.

Provided that the competitor antigen binding domain can block the binding of the antigen binding domain directed to IL-6R by at least 20%, preferably at least 20 to 50%, more preferably at least 50% as compared with binding activity obtained in a control test carried out in the absence of the candidate competitor antigen binding domain, the test antigen binding domain is determined as an antigen binding domain substantially binding to the same epitope as that for the competitor antigen binding domain, or competing for the binding to the same epitope.

When the epitope to which the antigen binding domain directed to IL-6R binds has an identified structure, whether a test antigen binding domain and a control antigen binding domain share an epitope can be evaluated by comparing the binding activity of these antigen binding domains against a peptide or a polypeptide prepared by introducing an amino acid mutation to a peptide constituting the epitope.

In such a method for measuring binding activity, for example, the binding activity of a test antigen binding domain and a control antigen binding domain against a linear peptide containing an introduced mutation can be compared in the ELISA format described above. In a method other than ELISA, the binding activity against the mutated peptide bound with a column may be measured by flowing the test antigen binding domain and the control antigen binding domain in the column, and then quantifying the antigen binding domain eluted in the eluate. A method for adsorbing a mutated peptide, for example, as a fusion peptide with GST, to a column is known in the art.

When the identified epitope is a conformational epitope, whether a test antigen binding domain and a control antigen binding domain share an epitope can be evaluated by the following method: first, IL-6R-expressing cells and cells expressing IL-6R with a mutation introduced to the epitope are prepared. The test antigen binding domain and the control antigen binding domain are added to cell suspensions containing these cells suspended in an appropriate buffer solution such as PBS. Subsequently, the cell suspensions are appropriately washed with a buffer solution, and a FITC-labeled antibody capable of recognizing the test antigen binding domain and the control antigen binding domain is then added thereto. The fluorescence intensity and the number of cells stained with the labeled antibody are measured using FACSCalibur (Becton, Dickinson and Company). The test antigen binding domain and the control antigen binding domain are appropriately diluted with a suitable buffer solution and used at concentrations thereby adjusted to the desired ones. These antigen binding domains are used, for example, at any concentration from 10 micro g/ml to 10 ng/ml. The amount of the labeled antibody bound to the cells is reflected in the fluorescence intensity obtained by analysis using CELL QUEST Software (Becton, Dickinson and Company), i.e., a geometric mean value. In short, the binding activity of the test antigen binding domain and the control antigen binding domain indicated by the amount of the labeled antibody bound can be determined by obtaining the geometric mean value.

The competition of the antigen binding domain with another antigen binding domain for the same epitope can also be confirmed by use of radiolabeled antigen binding assay (RIA), BIACORE® surface plasmon resonance assay, electrogenerated chemiluminescence, or the like, in addition to ELISA or FACS described above.

In the present method, whether to “not substantially bind to cells expressing mutated IL-6R” can be determined, for example, by the following method: first, a test antigen binding domain and a control antigen binding domain bound with the cells expressing mutated IL-6R are stained with a labeled antibody. Subsequently, the fluorescence intensity of the cells is detected. In the case of using FACSCalibur in the fluorescence detection by flow cytometry, the obtained fluorescence intensity can be analyzed using the CELL QUEST Software. From geometric mean values obtained in the presence and absence of the polypeptide associate, their comparison value (delta Geo-Mean) can be calculated according to expression 1 given below to determine the rate of increase in fluorescence intensity caused by the binding of the antigen binding domain.

Delta Geo-Mean=Geo-Mean (in the presence of the polypeptide associate)/Geo-Mean (in the absence of the polypeptide associate) (Expression 1)

The geometric mean comparison value (delta Geo-Mean value for the mutated IL-6R molecule) thus obtained by analysis, which reflects the amount of the test antigen binding domain bound with the cells expressing mutated IL-6R, is compared with the delta Geo-Mean comparison value that reflects the amount of the test antigen binding domain bound to the IL-6R-expressing cells. In this case, the concentrations of the test antigen binding domain used for determining the delta Geo-Mean comparison values for the cells expressing mutated IL-6R and the IL-6R-expressing cells are particularly preferably adjusted to equal or substantially equal concentrations. An antigen binding domain already confirmed to recognize an epitope in IL-6R is used as the control antigen binding domain.

Provided that the delta Geo-Mean comparison value of the test antigen binding domain for the cells expressing mutated IL-6R is smaller than at least 80%, preferably 50%, more preferably 30%, particularly preferably 15% of the delta Geo-Mean comparison value of the test antigen binding domain for the IL-6R-expressing cells, the test antigen binding domain “does not substantially bind to cells expressing mutate IL-6R”. The calculation expression for determining the Geo-Mean (geometric mean) value is described in the CELL QUEST Software User's Guide (BD biosciences). The epitope for the test antigen binding domain and the control antigen binding domain can be assessed as being the same when their comparison values can be regarded as being substantially equivalent as a result of comparison.

In the present specification, the term “carrying moiety” refers to a moiety other than an antigen binding domain in a polypeptide. The carrying moiety of the present invention is usually a peptide or a polypeptide constituted by amino acids. In a specific embodiment, the carrying moiety in the polypeptide is linked to the antigen binding domain via a cleavage site. The carrying moiety of the present invention may be a series of peptides or polypeptides connected through an amide bond, or may be a complex formed from a plurality of peptides or polypeptides through a covalent bond such as a disulfide bond or a noncovalent bond such as a hydrogen bond or hydrophobic interaction.

The carrying moiety of the present invention has an inhibiting domain that inhibits the antigen binding activity of the antigen binding domain. In the present specification, the term “inhibiting domain” is limited only by the inhibition of the antigen binding activity of the antigen binding domain. The inhibiting domain can be a domain having any structure as long as the domain used can inhibit the antigen binding activity of the antigen binding domain. Examples of such an inhibiting domain include, but are not limited to, an antibody heavy chain variable region (VH), an antibody light chain variable region (VL), pre-B cell receptors, and single-domain antibodies. The inhibiting domain may constitute the whole of the carrying moiety or may constitute a portion of the carrying moiety.

In some embodiments of the present invention, the antigen binding domain released from the polypeptide has higher antigen binding activity than that before the release. In other words, the antigen binding activity of the antigen binding domain is inhibited by the inhibiting domain in a state where the antigen binding domain is unreleased from the polypeptide. Whether the antigen binding activity of the antigen binding domain is inhibited by the inhibiting domain is confirmed by a method such as FACS (fluorescence activated cell sorting), ELISA (enzyme-linked immunosorbent assay), ECL (electrogenerated chemiluminescence), a SPR (surface plasmon resonance) method (Biacore), BLI (biolayer interferometry) (Octet). In some embodiments of the present invention, the antigen binding activity of the antigen binding domain released from the polypeptide is a value equal to or larger than 2 times, 3 times, 4 times, 5 times, 6 times, 7 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times, 900 times, 1000 times, 2000 times, or 3000 times the binding activity of the antigen binding domain unreleased from the polypeptide. In some more specific embodiments of the present invention, the binding of the antigen binding domain before the release to the antigen is not seen when the antigen binding activity of the antigen binding domain is measured by one method selected from among the methods described above.

In some aspects of the present invention, the cleavage site is cleaved so that the antigen binding domain becomes capable of being released from the polypeptide. In such aspects, therefore, the antigen binding activity can be compared between before and after the cleavage of the polypeptide. Specifically, the antigen binding activity measured using the cleaved polypeptide is a value equal to or larger than 2 times, 3 times, 4 times, 5 times, 6 times, 7 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times, 900 times, 1000 times, 2000 times, or 3000 times the antigen binding activity measured using the uncleaved polypeptide. In some more specific embodiments, the binding of the antigen binding domain of the uncleaved polypeptide to the antigen is not seen when the antigen binding activity is measured by one method selected from among the methods described above.

In some aspects of the present invention, the cleavage site is cleaved by protease. In such aspects, therefore, the antigen binding activity can be compared between before and after the protease treatment of the polypeptide. Specifically, the antigen binding activity measured using the polypeptide after the protease treatment is a value equal to or larger than 2 times, 3 times, 4 times, 5 times, 6 times, 7 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times, 900 times, 1000 times, 2000 times, or 3000 times the antigen binding activity measured using the polypeptide without the protease treatment. In some more specific embodiments, the binding of the antigen binding domain of the protease-untreated polypeptide to the antigen is not seen when the antigen binding activity is measured by one method selected from among the methods described above.

In the present invention, the polypeptide comprising an antigen binding domain and a carrying moiety has a longer half-life in blood than the half-life of the antigen binding domain that exists alone. In some embodiments of the present invention, for the longer half-life of the polypeptide, the carrying moiety is designed so as to have a longer half-life in blood. In such embodiments, examples of the approach of extending the half-life in blood of the carrying moiety include, but are not limited to, a large molecular weight of the carrying moiety, FcRn binding activity possessed by the carrying moiety, albumin binding activity possessed by the carrying moiety, and the PEGylation of the carrying moiety. In some embodiments of the present invention, the carrying moiety has a longer half-life in blood than that of the antigen binding domain (in other words, the antigen binding domain has a shorter half-life in blood than the half-life of the carrying moiety).

In one embodiment, the half-life of the antigen binding domain present with the carrying moiety in the polypeptide of the present invention in blood is 10% or more longer (i.e., 1.1 times or more longer) than that of the antigen binding domain present separated from the carrying moiety in blood. In one embodiment, the half-life of the antigen binding domain present with the carrying moiety in the polypeptide of the present invention in blood is 20% or more, 30% or more, 40% or more, 50% or more, 60% or more, 70% or more, 80% or more, 90% or more, 100% or more, 200% or more, 300% or more, 500% or more, 1000% or more, 2000% or more, 3000% or more, 4000% or more, or 5000% or more longer than that of the antigen binding domain present separated from the carrying moiety in blood. In one embodiment, the half-life of the antigen binding domain present with the carrying moiety in the polypeptide of the present invention in blood is 2 times or more, 3 times or more, 4 times or more, 5 times or more, 6 times or more, 7 times or more, 8 times or more, 9 times or more, 10 times or more, 20 times or more, 30 times or more, or 50 times or more longer than that of the antigen binding domain present separated from the carrying moiety in blood.

In the present invention, the half-lives of the antigen binding domain alone and the polypeptide, or the half-lives in blood of the antigen binding domain and the carrying moiety are preferably compared in terms of their half-lives in blood in humans. If the half-lives in blood are difficult to measure in humans, the half-lives in blood in humans can be predicted on the basis of their half-lives in blood in mice (e.g., normal mice, transgenic mice expressing a human antigen, and transgenic mice expressing human FcRn) or monkeys (e.g., cynomolgus monkeys).

In one embodiment, the approach of extending the half-life in blood of the carrying moiety includes a large molecular weight of the carrying moiety. In one embodiment, the approach of rendering the half-life in blood of the carrying moiety longer than that of the antigen binding domain includes a larger molecular weight of the carrying moiety than that of the antigen binding domain.

In one embodiment, the approach of extending the half-life in blood of the carrying moiety includes FcRn binding activity possessed by the carrying moiety. The carrying moiety can usually possess FcRn binding activity by a method of establishing an FcRn binding region in the carrying moiety. The FcRn binding region refers to a region having binding activity against FcRn and may have any structure as long as the region used has binding activity against FcRn.

The carrying moiety containing a FcRn binding region is capable of being taken up into cells and then brought back into plasma through the salvage pathway of FcRn. For example, an IgG molecule has a relatively long circulation time in plasma (slow disappearance) because FcRn known as a salvage receptor of the IgG molecule functions. An IgG molecule taken up into the endosome through pinocytosis binds to FcRn expressed in the endosome under intraendosomal acidic conditions. An IgG molecule that has failed to bind to FcRn is moved to the lysosome and degraded therein, whereas the IgG molecule bound with FcRn is transferred to cell surface, then dissociated from the FcRn under neutral conditions in plasma, and thereby brought back into plasma.

The FcRn binding region is preferably a region binding directly to FcRn. Preferred examples of the FcRn binding region can include antibody Fc regions. However, a region capable of binding to a polypeptide, such as albumin or IgG, which has FcRn binding capacity is capable of binding indirectly to FcRn via albumin, IgG, or the like. Therefore, the FcRn binding region according to the present invention may be a region binding to such a polypeptide having FcRn binding capacity.

The binding activity of the FcRn binding region according to the present invention against FcRn, particularly, human FcRn may be measured by a method known to those skilled in the art, as mentioned in the above section about binding activity. The conditions therefor may be appropriately determined by those skilled in the art. The binding activity against human FcRn can be evaluated as KD (dissociation constant), apparent KD (apparent dissociation constant), kd (dissociation rate), or apparent kd (apparent dissociation rate), etc. These values can be measured by methods known to those skilled in the art. For example, Biacore (GE Healthcare Japan Corp.), Scatchard plot, a flow cytometer, and the like can be used.

The conditions for measuring the binding activity of the FcRn binding region against FcRn are not particularly limited and may be appropriately selected by those skilled in the art. The binding activity can be measured under conditions involving, for example, a MES buffer and 37 degrees C., as described in WO2009/125825. Also, the binding activity of the FcRn binding region of the present invention against FcRn may be measured by a method known to those skilled in the art and can be measured using, for example, Biacore (GE Healthcare Japan Corp.). In the measurement of the binding activity of the FcRn binding region against FcRn, FcRn and the FcRn binding region or the carrying moiety containing the FcRn binding region can be injected as analytes to chips on which the FcRn binding region or the carrying moiety containing the FcRn binding region and FcRn, respectively, are immobilized, followed by evaluation.

As for pH for use in the measurement conditions, the binding affinity of the FcRn binding region for FcRn may be evaluated at any pH of 4.0 to 6.5. Preferably, a pH of 5.8 to 6.0, which is close to pH in the early endosome in vivo, is used for determining the binding affinity of the FcRn binding region for human FcRn. As for temperature for use in the measurement conditions, the binding affinity of the FcRn binding region for FcRn may be evaluated at any temperature of 10 degrees C. to 50 degrees C. Preferably, a temperature of 15 degrees C. to 40 degrees C. is used for determining the binding affinity of the FcRn binding region for human FcRn. More preferably, any temperature from 20 degrees C. to 35 degrees C., for example, any one of 20, 21, 22, 23, 24, 25, 26, 27, 28, 29, 30, 31, 32, 33, 34, and 35 degrees C., is also used for determining the binding affinity of the FcRn binding region for FcRn. The temperature of 25 degrees C. is one non-limiting example of the temperature of the present invention.

One example of the FcRn binding region includes, but is not limited to, an IgG antibody Fc region. In the case of using an IgG antibody Fc region, its type is not limited, and for example, IgG1, IgG2, IgG3, or IgG4 Fc region may be used. For example, a Fc region containing one sequence selected from the amino acid sequences represented by SEQ ID NOs: 21, 22, 23, and 24 may be used.

A natural IgG antibody Fc region as well as an Fc region variant having one or more amino acid substitutions may be used as long as the Fc region has FcRn binding activity.

For example, an Fc region variant containing an amino acid sequence derived from an IgG antibody Fc region by the substitution of at least one amino acid selected from EU numbering positions 237, 238, 239, 248, 250, 252, 254, 255, 256, 257, 258, 265, 270, 286, 289, 297, 298, 303, 305, 307, 308, 309, 311, 312, 314, 315, 317, 325, 332, 334, 360, 376, 380, 382, 384, 385, 386, 387, 389, 424, 428, 433, 434 and 436 by another amino acid may be used.

More specifically, an Fc region variant containing at least one amino acid substitution selected from
an amino acid substitution to substitute Gly at position 237 by Met,
an amino acid substitution to substitute Pro at position 238 by Ala,
an amino acid substitution to substitute Ser at position 239 by Lys,
an amino acid substitution to substitute Lys at position 248 by Ile,
an amino acid substitution to substitute Thr at position 250 by Ala, Phe, Ile, Met, Gln, Ser, Val, Trp, or Tyr,
an amino acid substitution to substitute Met at position 252 by Phe, Trp, or Tyr,
an amino acid substitution to substitute Ser at position 254 by Thr,
an amino acid substitution to substitute Arg at position 255 by Glu,
an amino acid substitution to substitute Thr at position 256 by Asp, Glu, or Gln,
an amino acid substitution to substitute Pro at position 257 by Ala, Gly, Ile, Leu, Met, Asn, Ser, Thr, or Val,
an amino acid substitution to substitute Glu at position 258 by His,
an amino acid substitution to substitute Asp at position 265 by Ala,
an amino acid substitution to substitute Asp at position 270 by Phe,
an amino acid substitution to substitute Asn at position 286 by Ala or Glu,
an amino acid substitution to substitute Thr at position 289 by His,
an amino acid substitution to substitute Asn at position 297 by Ala,
an amino acid substitution to substitute Ser at position 298 by Gly,
an amino acid substitution to substitute Val at position 303 by Ala,
an amino acid substitution to substitute Val at position 305 by Ala,
an amino acid substitution to substitute Thr at position 307 by Ala, Asp, Phe, Gly, His, Ile, Lys, Leu, Met, Asn, Pro, Gln, Arg, Ser, Val, Trp, or Tyr,
an amino acid substitution to substitute Val at position 308 by Ala, Phe, Ile, Leu, Met, Pro, Gln, or Thr,
an amino acid substitution to substitute Leu or Val at position 309 by Ala, Asp, Glu, Pro, or Arg,
an amino acid substitution to substitute Gln at position 311 by Ala, His, or Ile,
an amino acid substitution to substitute Asp at position 312 by Ala or His,
an amino acid substitution to substitute Leu at position 314 by Lys or Arg,
an amino acid substitution to substitute Asn at position 315 by Ala or His,
an amino acid substitution to substitute Lys at position 317 by Ala,
an amino acid substitution to substitute Asn at position 325 by Gly,
an amino acid substitution to substitute Ile at position 332 by Val,
an amino acid substitution to substitute Lys at position 334 by Leu,
an amino acid substitution to substitute Lys at position 360 by His,
an amino acid substitution to substitute Asp at position 376 by Ala,
an amino acid substitution to substitute Glu at position 380 by Ala,
an amino acid substitution to substitute Glu at position 382 by Ala,
an amino acid substitution to substitute Asn or Ser at position 384 by Ala,
an amino acid substitution to substitute Gly at position 385 by Asp or His,
an amino acid substitution to substitute Gln at position 386 by Pro,
an amino acid substitution to substitute Pro at position 387 by Glu,
an amino acid substitution to substitute Asn at position 389 by Ala or Ser,
an amino acid substitution to substitute Ser at position 424 by Ala,
an amino acid substitution to substitute Met at position 428 by Ala, Asp, Phe, Gly, His, Ile, Lys, Leu, Asn, Pro, Gln, Ser, Thr, Val, Trp, or Tyr,
an amino acid substitution to substitute His at position 433 by Lys,
an amino acid substitution to substitute Asn at position 434 by Ala, Phe, His, Ser, Trp, or Tyr, and
an amino acid substitution to substitute Tyr or Phe at position 436 by His
(all according to the EU numbering)
in an IgG antibody Fc region may be used.

From another viewpoint, a Fc region containing at least one amino acid selected from
Met as the amino acid at position 237,
Ala as the amino acid at position 238,
Lys as the amino acid at position 239,
Ile as the amino acid at position 248,
Ala, Phe, Ile, Met, Gln, Ser, Val, Trp, or Tyr as the amino acid at position 250,
Phe, Trp, or Tyr as the amino acid at position 252,
Thr as the amino acid at position 254,
Glu as the amino acid at position 255,
Asp, Glu, or Gln as the amino acid at position 256,
Ala, Gly, Ile, Leu, Met, Asn, Ser, Thr, or Val as the amino acid at position 257,
His as the amino acid at position 258,
Ala as the amino acid at position 265,
Phe as the amino acid at position 270,
Ala or Glu as the amino acid at position 286,
His as the amino acid at position 289,
Ala as the amino acid at position 297,
Gly as the amino acid at position 298,
Ala as the amino acid at position 303,
Ala as the amino acid at position 305,
Ala, Asp, Phe, Gly, His, Ile, Lys, Leu, Met, Asn, Pro, Gln, Arg, Ser, Val, Trp, or Tyr as the amino acid at position 307,
Ala, Phe, Ile, Leu, Met, Pro, Gln, or Thr as the amino acid at position 308,
Ala, Asp, Glu, Pro, or Arg as the amino acid at position 309,
Ala, His, or Ile as the amino acid at position 311,
Ala or His as the amino acid at position 312,
Lys or Arg as the amino acid at position 314,
Ala or His as the amino acid at position 315,
Ala as the amino acid at position 317,
Gly as the amino acid at position 325,
Val as the amino acid at position 332,
Leu as the amino acid at position 334,
His as the amino acid at position 360,
Ala as the amino acid at position 376,
Ala as the amino acid at position 380,
Ala as the amino acid at position 382,
Ala as the amino acid at position 384,
Asp or His as the amino acid at position 385,
Pro as the amino acid at position 386,
Glu as the amino acid at position 387,
Ala or Ser as the amino acid at position 389,
Ala as the amino acid at position 424,
Ala, Asp, Phe, Gly, His, Ile, Lys, Leu, Asn, Pro, Gln, Ser, Thr, Val, Trp, or Tyr as the amino acid at position 428,
Lys as the amino acid at position 433,
Ala, Phe, His, Ser, Trp, or Tyr as the amino acid at position 434, and
His as the amino acid at position 436
(all according to the EU numbering)
in an IgG antibody Fc region may be used.

The FcRn binding activity possessed by the carrying moiety does not mean that the antigen binding domain has no FcRn binding activity. In the embodiments in which the carrying moiety has a longer half-life in blood than the half-life of the antigen binding domain, the antigen binding domain may have no FcRn binding activity, as a matter of course, or the antigen binding domain may have FcRn binding activity as long as the FcRn binding activity is weaker than that of the carrying moiety.

In one embodiment, the method for extending the half-life in blood of the carrying moiety involves binding the carrying moiety to albumin. Since albumin does not undergo renal excretion and has FcRn binding activity, its half-life in blood is as long as 17 to 19 days (J Clin Invest. 1953 August; 32 (8): 746-768). Hence, it has been reported that a protein bound with albumin becomes bulky and capable of binding indirectly to FcRn and therefore has an increased half-life in blood (Antibodies 2015, 4 (3), 141-156).

In one embodiment, the alternative method for extending the half-life in blood of the carrying moiety involves PEGylating the carrying moiety. The PEGylation of a protein is considered to render the protein bulky and also suppress its degradation by protease in blood, thereby extending the half-life in blood of the protein (J Pharm Sci. 2008 October; 97 (10): 4167-83).

In some embodiments of the present invention, the carrying moiety contains an antibody Fc region. In a specific embodiment, the carrying moiety contains a CH2 domain and a CH3 domain of a human IgG antibody. In a specific embodiment, the carrying moiety contains a moiety spanning from human IgG1 antibody heavy chain Cys226 or Pro230 to the carboxyl terminus of the heavy chain. However, the C-terminal lysine (Lys447) or glycine-lysine (Gly446-Lys447) of the Fc region may be present or absent.

In some embodiments of the present invention, the carrying moiety contains an antibody constant region. In a more preferred embodiment, the carrying moiety contains an IgG antibody constant region. In a further preferred embodiment, the carrying moiety contains a human IgG antibody constant region.

In some embodiments of the present invention, the carrying moiety contains: a region substantially similar in structure to an antibody heavy chain constant region; and a region substantially similar in structure to an antibody light chain, connected to the region via a covalent bond such as a disulfide bond or a noncovalent bond such as a hydrogen bond or hydrophobic interaction.

In the present specification, the “polypeptide comprising an antigen binding domain and a carrying moiety” is usually a series of polypeptides connected through an amide bond, or a protein containing a plurality of polypeptides connected through an amide bond.

In some embodiments of the present invention, the antigen binding domain is capable of being released from the polypeptide, and the antigen binding domain released from the polypeptide has higher antigen binding activity. In the present specification, the term “release” refers to the mutual separation of two moieties of the polypeptide. The release of the antigen binding domain from the polypeptide can be attributed to the cancelation of the interaction between the antigen binding domain and the carrying moiety. The antigen binding activity of the antigen binding domain incorporated in the polypeptide is inhibited. Hence, the antigen binding domain released from the polypeptide can be confirmed by measuring the antigen binding activity of a subject and comparing it with the antigen binding activity of the antigen binding domain incorporated in the polypeptide.

In some embodiments, the polypeptide comprises a cleavage site, and the cleavage site is cleaved so that the antigen binding domain is released from the polypeptide. The cleavage site can be cleaved by, for example, an enzyme, can be reduced with a reducing agent, or can be photodegraded. The cleavage site may be placed at any position in the polypeptide as long as the antigen binding domain can be released and does not lose its antigen binding activity after the release. The polypeptide may further contain an additional cleavage site other than the cleavage site for the release of the antigen binding domain. In one embodiment of the present invention, the cleavage site comprises a protease cleavage sequence and can be cleaved by protease.

In the present specification, the term “cleaved” refers to a state where the antigen binding domain and the carrying moiety are separated from each other after alteration of the cleavage site by protease, reduction of a cysteine-cysteine disulfide bond at the cleavage site, and/or photoactivation. In the present specification, the term “uncleaved” refers to a state where the antigen binding domain is linked to the carrying moiety in the absence of the protease cleavage of the cleavage site, in the absence of the reduction of a cysteine-cysteine disulfide bond at the cleavage site, and/or in the absence of light.

The cleavage of the cleavage site can be detected by subjecting a solution containing the cleavage site-containing polypeptide to SDS-PAGE (polyacrylamide gel electrophoresis) and measuring the molecular weights of the fragments or detecting change in molecular weight between before and after the cleavage.

The cleavage site can be specifically modified (cleaved, reduced or photodegraded) by an agent (i.e., protease, a reducing agent, or light) at a rate of approximately 0.001 to 1500×104 M-1S-1 or at least 0.001, 0.005, 0.01, 0.05, 0.1, 0.5, 1, 2.5, 5, 7.5, 10, 15, 20, 25, 50, 75, 100, 125, 150, 200, 250, 500, 750, 1000, 1250, or 1500×104 M-1S-1.

The specific cleavage by protease is performed by the contact between the protease and the cleavage site or a molecule containing the cleavage site. The cleavage site can be cleaved in the presence of sufficient enzyme activity. The sufficient enzyme activity can refer to the ability of the enzyme to bring about cleavage upon contact with the cleavage site.

In the present specification, the term “protease” refers to an enzyme such as endopeptidase or exopeptidase which hydrolyzes a peptide bond, typically, endopeptidase. The protease used in the present invention is limited only by being capable of cleaving the protease cleavage sequence and is not particularly limited by its type. In some embodiments, target tissue specific protease is used. The target tissue specific protease can refer to, for example, any of
(1) protease that is expressed at a higher level in the target tissue than in normal tissues,
(2) protease that has higher activity in the target tissue than in normal tissues,
(3) protease that is expressed at a higher level in the target cells than in normal cells, and
(4) protease that has higher activity in the target cells than in normal cells.

In a more specific embodiment, a cancer tissue specific protease or an inflammatory tissue specific protease is used.

In the present specification, the term “target tissue” means a tissue containing at least one target cell. In some embodiments of the present invention, the target tissue is a cancer tissue. In some embodiments of the present invention, the target tissue is an inflammatory tissue.

The term “cancer tissue” means a tissue containing at least one cancer cell. Thus, considering that, for example, the cancer tissue contains cancer cells and vascular vessels, every cell type that contributes to the formation of tumor mass containing cancer cells and endothelial cells is included in the scope of the present invention. In the present specification, the tumor mass refers to a foci of tumor tissue. The term “tumor” is generally used to mean benign neoplasm or malignant neoplasm.

In the present specification, examples of the “inflammatory tissue” include the following:
a joint tissue in rheumatoid arthritis or osteoarthritis,
a lung (alveolus) tissue in bronchial asthma or COPD,
a digestive organ tissue in inflammatory bowel disease, Crohn disease, or ulcerative colitis,
a fibrotic tissue in fibrosis in the liver, the kidney, or the lung,
a tissue under rejection of organ transplantation,
a vascular vessel or heart (cardiac muscle) tissue in arteriosclerosis or heart failure,
a visceral fat tissue in metabolic syndrome,
a skin tissue in atopic dermatitis and other dermatitides, and
a spinal nerve tissue in disk herniation or chronic lumbago.

Specifically expressed or specifically activated protease, or protease considered to be related to the disease condition of a target tissue (target tissue specific protease) is known for some types of target tissues. For example, International Publication Nos. WO2013/128194, WO2010/081173, and WO2009/025846 disclose protease specifically expressed in a cancer tissue. Also, J Inflamm (Lond). 2010; 7: 45, Nat Rev Immunol. 2006 July; 6 (7): 541-50, Nat Rev Drug Discov. 2014 December; 13 (12): 904-27, Respir Res. 2016 Mar. 4; 17: 23, Dis Model Mech. 2014 February; 7 (2): 193-203, and Biochim Biophys Acta. 2012 January; 1824 (1): 133-45 disclose protease considered to be related to inflammation.

In addition to the protease specifically expressed in a target tissue, there also exists protease specifically activated in a target tissue. For example, protease may be expressed in an inactive form and then converted to an active form. Many tissues contain a substance inhibiting active protease and control the activity by the process of activation and the presence of the inhibitor (Nat Rev Cancer. 2003 July; 3 (7): 489-501). In a target tissue, the active protease may be specifically activated by escaping inhibition.

The active protease can be measured by use of a method using an antibody recognizing the active protease (PNAS 2013 Jan. 2; 110 (1): 93-98) or a method of fluorescently labeling a peptide recognizable by protease so that the fluorescence is quenched before cleavage, but emitted after cleavage (Nat Rev Drug Discov. 2010 September; 9 (9): 690-701. doi: 10.1038/nrd3053).

From one viewpoint, the term “target tissue specific protease” can refer to any of
(i) protease that is expressed at a higher level in the target tissue than in normal tissues,
(ii) protease that has higher activity in the target tissue than in normal tissues,
(iii) protease that is expressed at a higher level in the target cells than in normal cells, and
(iv) protease that has higher activity in the target cells than in normal cells.

Specific examples of the protease include, but are not limited to, cysteine protease (including cathepsin families B, L, S, etc.), aspartyl protease (cathepsins D, E, K, O, etc.), serine protease (including matriptase (including MT-SP1), cathepsins A and G, thrombin, plasmin, urokinase (uPA), tissue plasminogen activator (tPA), elastase, proteinase 3, thrombin, kallikrein, tryptase, and chymase), metalloproteinase (metalloproteinase (MMP1-28) including both membrane-bound forms (MMP14-17 and MMP24-25) and secreted forms (MMP1-13, MMP18-23 and MMP26-28), A disintegrin and metalloproteinase (ADAM), specifically ADAM17, A disintegrin and metalloproteinase with thrombospondin motifs (ADAMTSs (ADAMTS1, ADAMTS2, ADAMTS3, ADAMTS4, ADAMTS5, ADAMTS6, ADAMTS7, ADAMTS8, ADAMTS9, ADAMTS10, ADAMTS12, ADAMTS13, ADAMTS14, ADAMTS15, ADAMTS16, ADAMTS17, ADAMTS18, ADAMTS19 and ADAMTS20)), meprin (meprin alpha and meprin beta), CD10 (CALLA), prostate-specific antigen (PSA), legumain, TMPRSS3, TMPRSS4, human neutrophil elastase (HNE), beta secretase (BACE), fibroblast activation protein alpha (FAP), granzyme B, guanidinobenzoatase (GB), hepsin, neprilysin, NS3/4A, HCV-NS3/4, calpain, ADAMDEC1, renin, cathepsin C, cathepsin V/L2, cathepsin X/Z/P, cruzipain, otubain 2, kallikrein-related peptidases (KLKs (KLK3, KLK4, KLK5, KLK6, KLK7, KLK8, KLK10, KLK11, KLK13, and KLK14)), bone morphogenetic protein 1 (BMP-1), activated protein C, blood coagulation-related protease (Factor VIIa, Factor IXa, Factor Xa, Factor XIa, and Factor XIIa), HtrA1, lactoferrin, marapsin, PACE4, DESC1, dipeptidyl peptidase 4 (DPP-4), TMPRSS2, cathepsin F, cathepsin H, cathepsin L2, cathepsin O, cathepsin S, granzyme A, Gepsin calpain 2, glutamate carboxypeptidase 2, AMSH-like proteases, AMSH, gamma secretase, antiplasmin cleaving enzyme (APCE), decysin 1, N-acetylated alpha-linked acidic dipeptidase-like 1 (NAALADL1), and furin.

From another viewpoint, the target tissue specific protease can refer to a cancer tissue specific protease or an inflammatory tissue specific protease.

Examples of cancer tissue specific protease include protease specifically expressed in a cancer tissue disclosed in International Publication Nos. WO2013/128194, WO2010/081173, and WO2009/025846.

As for the type of cancer tissue specific protease, the protease having higher expression specificity in the cancer tissue to be treated is more effective for reducing adverse reactions. Preferable cancer tissue specific protease has a concentration in the cancer tissue at least 5 times, more preferably at least 10 times, further preferably at least 100 times, particularly preferably at least 500 times, most preferably at least 1000 times higher than its concentration in normal tissues. Also, preferable cancer tissue specific protease has activity in the cancer tissue at least 2 times, more preferably at least 3 times, at least 4 times, at least 5 times, or at least 10 times, further preferably at least 100 times, particularly preferably at least 500 times, most preferably at least 1000 times higher than its activity in normal tissues.

The cancer tissue specific protease may be in a form bound with a cancer cell membrane or may be in a form secreted extracellularly without being bound with a cell membrane. When the cancer tissue specific protease is not bound with a cancer cell membrane, it is preferred for immunocyte-mediated cytotoxicity specific for cancer cells that the cancer tissue specific protease should exist within or in the vicinity of the cancer tissue. In the present specification, the “vicinity of the cancer tissue” means to fall within the scope of location where the protease cleavage sequence specific for the cancer tissue is cleaved so that the antigen binding domain exerts antigen binding activity. However, it is preferred that damage on normal cells should be minimized in this scope of location.

From an alternative viewpoint, cancer tissue specific protease is any of
(i) protease that is expressed at a higher level in the cancer tissue than in normal tissues,
(ii) protease that has higher activity in the cancer tissue than in normal tissues,
(iii) protease that is expressed at a higher level in the cancer cells than in normal cells, and
(iv) protease that has higher activity in the cancer cells than in normal cells.

One type of cancer tissue specific protease may be used alone, or two or more types of cancer tissue specific proteases may be combined. The number of types of cancer tissue specific protease can be appropriately set by those skilled in the art in consideration of the cancer type to be treated.

From these viewpoints, cancer tissue specific protease is preferably serine protease or metalloproteinase, more preferably matriptase (including MT-SP1), urokinase (uPA), or metalloproteinase, further preferably MT-SP1, uPA, MMP2, or MMP9, among the proteases listed above.

As for the type of inflammatory tissue specific protease, the protease having higher expression specificity in the inflammatory tissue to be treated is more effective for reducing adverse reactions. Preferable inflammatory tissue specific protease has a concentration in the inflammatory tissue at least 5 times, more preferably at least 10 times, further preferably at least 100 times, particularly preferably at least 500 times, most preferably at least 1000 times higher than its concentration in normal tissues. Also, preferable inflammatory tissue specific protease has activity in the inflammatory tissues at least 2 times, more preferably at least 3 times, at least 4 times, at least 5 times, or at least 10 times, further preferably at least 100 times, particularly preferably at least 500 times, most preferably at least 1000 times higher than its activity in normal tissues.

The inflammatory tissue specific protease may be in a form bound with an inflammatory cell membrane or may be in a form secreted extracellularly without being bound with a cell membrane. When the inflammatory tissue specific protease is not bound with an inflammatory cell membrane, it is preferred for immunocyte-mediated cytotoxicity specific for inflammatory cells that the inflammatory tissue specific protease should exist within or in the vicinity of the inflammatory tissue. In the present specification, the “vicinity of the inflammatory tissue” means to fall within the scope of location where the protease cleavage sequence specific for the inflammatory tissue is cleaved so that the antigen binding domain exerts antigen binding activity. However, it is preferred that damage on normal cells should be minimized in this scope of location.

From an alternative viewpoint, inflammatory tissue specific protease is any of
(i) protease that is expressed at a higher level in the inflammatory tissue than in normal tissues,
(ii) protease that has higher activity in the inflammatory tissue than in normal tissues,
(iii) protease that is expressed at a higher level in the inflammatory cells than in normal cells, and
(iv) protease that has higher activity in the inflammatory cells than in normal cells.

One type of inflammatory tissue specific protease may be used alone, or two or more types of inflammatory tissue specific proteases may be combined. The number of types of inflammatory tissue specific protease can be appropriately set by those skilled in the art in consideration of the pathological condition to be treated.

From these viewpoints, t inflammatory tissue specific protease is preferably metalloproteinase among the proteases listed above. The metalloproteinase is more preferably ADAMTS4, ADAMTS5, ADAM17, MMP1, MMP2, MMP3, MMP7, MMP9, MMP13, MMP14, or MMP17.

The protease cleavage sequence is a particular amino acid sequence that is specifically recognized by target tissue specific protease when the polypeptide is hydrolyzed by the target tissue specific protease in an aqueous solution.

The protease cleavage sequence is preferably an amino acid sequence that is hydrolyzed with high specificity by target tissue specific protease more specifically expressed in the target tissue or cells to be treated or more specifically activated in the target tissue/cells to be treated, from the viewpoint of reduction in adverse reactions.

Specific examples of the protease cleavage sequence include target sequences that are specifically hydrolyzed by the above-listed protease specifically expressed in a cancer tissue disclosed in International Publication Nos. WO2013/128194, WO2010/081173, and WO2009/025846, the protease specific for an inflammatory tissue, and the like. A sequence artificially altered by, for example, introducing an appropriate amino acid mutation to a target sequence that is specifically hydrolyzed by known protease can also be used. Alternatively, a protease cleavage sequence identified by a method known to those skilled in the art as described in Nature Biotechnology 19, 661-667 (2001) may be used.

Furthermore, a naturally occurring protease cleavage sequence may be used. For example, TGFbeta is converted to a latent form by protease cleavage. Likewise, a protease cleavage sequence in a protein that changes its molecular form by protease cleavage can also be used.

Examples of the protease cleavage sequence that can be used include, but are not limited to, sequences disclosed in International Publication No. WO2015/116933, International Publication No. WO2015/048329, International Publication No. WO2016/118629, International Publication No. WO2016/179257, International Publication No. WO2016/179285, International Publication No. WO2016/179335, International Publication No. WO2016/179003, International Publication No. WO2016/046778, International Publication No. WO2016/014974, U.S. Patent Publication No. US2016/0289324, U.S. Patent Publication No. US2016/0311903, PNAS (2000) 97: 7754-7759, Biochemical Journal (2010) 426: 219-228, and Beilstein J Nanotechnol. (2016) 7: 364-373.

The protease cleavage sequence is more preferably an amino acid sequence that is specifically hydrolyzed by suitable target tissue specific protease as mentioned above. The amino acid sequence that is specifically hydrolyzed by target tissue specific protease is preferably a sequence comprising any of the following amino acid sequences:

(SEQ ID NO: 12, cleavable by MT-SP1 or uPA)
LSGRSDNH
(SEQ ID NO: 25, cleavable by MMP2 or MMP9)
PLALAG,
and
(SEQ ID NO: 26, cleavable by MMP7)
VPLSLTMG.

Any of the following sequences can also be used as the protease cleavage sequence:

(SEQ ID NO: 74, cleavable by MT-SP1 or uPA)
TSTSGRSANPRG,
(SEQ ID NO: 75, cleavable by MT-SP1 or uPA)
ISSGLLSGRSDNH,
(SEQ ID NO: 76, cleavable by MT-SP1 or uPA)
AVGLLAPPGGLSGRSDNH,
(SEQ ID NO: 77, cleavable by MMP1)
GAGVPMSMRGGAG,
(SEQ ID NO: 78, cleavable by MMP2)
GAGIPVSLRSGAG,
(SEQ ID NO: 79, cleavable by MMP2)
GPLGIAGQ,
(SEQ ID NO: 80, cleavable by MMP2)
GGPLGMLSQS,
(SEQ ID NO: 81, cleavable by MMP2)
PLGLWA,
(SEQ ID NO: 82, cleavable by MMP3)
GAGRPFSMIMGAG,
(SEQ ID NO: 83, cleavable by MMP7)
GAGVPLSLTMGAG,
(SEQ ID NO: 84, cleavable by MMP9)
GAGVPLSLYSGAG,
(SEQ ID NO: 85, cleavable by MMP11)
AANLRN,
(SEQ ID NO: 86, cleavable by MMP11)
AQAYVK,
(SEQ ID NO: 87, cleavable by MMP11)
AANYMR,
(SEQ ID NO: 88, cleavable by MMP11)
AAALTR,
(SEQ ID NO: 89, cleavable by MMP11)
AQNLMR,
(SEQ ID NO: 90, cleavable by MMP11)
AANYTK,
(SEQ ID NO: 91, cleavable by MMP13)
GAGPQGLAGQRGIVAG,
(SEQ ID NO: 92, cleavable by pro-urokinase)
PRFKIIGG
(SEQ ID NO: 93, cleavable by pro-urokinase)
PRFRIIGG
(SEQ ID NO: 94, cleavable by uPA)
GAGSGRSAG,
(SEQ ID NO: 95, cleavable by uPA)
SGRSA
(SEQ ID NO: 96, cleavable by uPA)
GSGRSA,
(SEQ ID NO: 97, cleavable by uPA)
SGKSA
(SEQ ID NO: 98, cleavable by uPA)
SGRSS,
(SEQ ID NO: 99, cleavable by uPA)
SGRRA,
(SEQ ID NO: 100, cleavable by uPA)
SGRNA,
(SEQ ID NO: 101, cleavable by uPA)
SGRKA,
(SEQ ID NO: 102, cleavable by tPA)
QRGRSA,
(SEQ ID NO: 103, cleavable by cathepsin B)
GAGSLLKSRMVPNFNAG,
(SEQ ID NO: 104, cleavable by cathepsin B)
TQGAAA,
(SEQ ID NO: 105, cleavable by cathepsin B)
GAAAAA,
(SEQ ID NO: 106, cleavable by cathepsin B)
GAGAAG,
(SEQ ID NO: 107, cleavable by cathepsin B)
AAAAAG,
(SEQ ID NO: 108, cleavable by cathepsin B)
LCGAAI,
(SEQ ID NO: 109, cleavable by cathepsin B)
FAQALG,
(SEQ ID NO: 110, cleavable by cathepsin B)
LLQANP,
(SEQ ID NO: 111, cleavable by cathepsin B)
LAAANP,
(SEQ ID NO: 112, cleavable by cathepsin B)
LYGAQF,
(SEQ ID NO: 113, cleavable by cathepsin B)
LSQAQG,
(SEQ ID NO: 114, cleavable by cathepsin B)
ASAASG,
(SEQ ID NO: 115, cleavable by cathepsin B)
FLGASL,
(SEQ ID NO: 116, cleavable by cathepsin B)
AYGATG,
(SEQ ID NO: 117, cleavable by cathepsin B)
LAQATG,
(SEQ ID NO: 118, cleavable by cathepsin L)
GAGSGVVIATVIVITAG,
(SEQ ID NO: 119, cleavable by meprin alpha or meprin beta)
APMAEGGG,
(SEQ ID NO: 120, cleavable by meprin alpha or meprin beta)
EAQGDKII,
(SEQ ID NO: 121, cleavable by meprin alpha or meprin beta)
LAFSDAGP,
(SEQ ID NO: 122, cleavable by meprin alpha or meprin beta)
YVADAPK,
(SEQ ID NO: 123, cleavable by furin)
RRRRR,
(SEQ ID NO: 124, cleavable by furin)
RRRRRR,
(SEQ ID NO: 125, cleavable by furin)
GQSSRHRRAL,
(SEQ ID NO: 126)
SSRHRRALD,
(SEQ ID NO: 127, cleavable by plasminogen)
RKSSIIIRMRDVVL,
(SEQ ID NO: 128, cleavable by staphylokinase)
SSSFDKGKYKKGDDA,
(SEQ ID NO: 129, cleavable by staphylokinase)
SSSFDKGKYKRGDDA,
(SEQ ID NO: 130, cleavable by Factor Xa)
IEGR,
(SEQ ID NO: 131, cleavable by Factor Xa)
IDGR,
(SEQ ID NO: 132, cleavable by Factor Xa)
GGSIDGR,
(SEQ ID NO: 133, cleavable by collagenase)
GPQGIAGQ,
(SEQ ID NO: 134, cleavable by collagenase)
GPQGLLGA,
(SEQ ID NO: 135, cleavable by collagenase)
GIAGQ,
(SEQ ID NO: 136, cleavable by collagenase)
GPLGIAG,
(SEQ ID NO: 137, cleavable by collagenase)
GPEGLRVG,
(SEQ ID NO: 138, cleavable by collagenase)
YGAGLGVV,
(SEQ ID NO: 139, cleavable by collagenase)
AGLGVVER,
(SEQ ID NO: 140, cleavable by collagenase)
AGLGISST,
(SEQ ID NO: 141, cleavable by collagenase)
EPQALAMS,
(SEQ ID NO: 142, cleavable by collagenase)
QALAMSAI,
(SEQ ID NO: 143, cleavable by collagenase)
AAYHLVSQ,
(SEQ ID NO: 144, cleavable by collagenase)
MDAFLESS,
(SEQ ID NO: 145, cleavable by collagenase)
ESLPVVAV,
(SEQ ID NO: 146, cleavable by collagenase)
SAPAVESE,
(SEQ ID NO: 147, cleavable by collagenase)
DVAQFVLT,
(SEQ ID NO: 148, cleavable by collagenase)
VAQFVLTE,
(SEQ ID NO: 149, cleavable by collagenase)
AQFVLTEG,
(SEQ ID NO: 150, cleavable by collagenase)
PVQPIGPQ,
(SEQ ID NO: 151, cleavable by thrombin)
LVPRGS,
(SEQ ID NO: 178, cleavable by uPA or MT-SP1)
TSTSGRSANPRG,
and
(SEQ ID NO: 509, cleavable by MMP13)
GPPGPQGLAGQRGIVGL.

In one embodiment of the present invention, a flexible linker is further attached to either one end or both ends of the protease cleavage sequence. The flexible linker at one end of the protease cleavage sequence can be referred to as a first flexible linker, and the flexible linker at the other end can be referred to as a second flexible linker. In a particular embodiment, the protease cleavage sequence and the flexible linker have any of the following formulas:

(protease cleavage sequence),

(first flexible linker)−(protease cleavage sequence),

(protease cleavage sequence)−(second flexible linker), and

(first flexible linker)−(protease cleavage sequence)−(second flexible linker).

The flexible linker according to the present embodiment is preferably a peptide linker. The first flexible linker and the second flexible linker each independently and arbitrarily exist and are identical or different flexible linkers each containing at least one flexible amino acid (Gly, etc.). The flexible linker contains, for example, a sufficient number of residues (amino acids arbitrarily selected from Arg, Ile, Gln, Glu, Cys, Tyr, Trp, Thr, Val, His, Phe, Pro, Met, Lys, Gly, Ser, Asp, Asn, Ala, etc., particularly Gly, Ser, Asp, Asn, and Ala, in particular, Gly and Ser, especially Gly, etc.) for the protease cleavage sequence to obtain the desired protease accessibility.

The flexible linker suable for use at both ends of the protease cleavage sequence is usually a flexible linker that improves the access of protease to the protease cleavage sequence and elevates the cleavage efficiency of the protease. A suitable flexible linker may be readily selected and can be preferably selected from among different lengths such as 1 amino acid (Gly, etc.) to 20 amino acids, 2 amino acids to 15 amino acids, or 3 amino acids to 12 amino acids including 4 amino acids to 10 amino acids, 5 amino acids to 9 amino acids, 6 amino acids to 8 amino acids, or 7 amino acids to 8 amino acids. In some embodiments of the present invention, the flexible linker is a peptide linker of 1 to 7 amino acids.

Examples of the flexible linker include, but are not limited to, glycine polymers (G)n, glycine-serine polymers (including e.g., (GS)n, (GSGGS: SEQ ID NO: 27)n and (GGGS: SEQ ID NO: 28)n, wherein n is an integer of at least 1), glycine-alanine polymers, alanine-serine polymers, and other flexible linkers well known in conventional techniques.

Among them, glycine and glycine-serine polymers are receiving attention because these amino acids are relatively unstructured and easily function as neutral tethers between components.

Examples of the flexible linker consisting of the glycine-serine polymer include, but are not limited to,

Ser (S)
Gly Ser (GS)
Ser Gly (SG)
Gly Gly Ser (GGS)
Gly Ser Gly (GSG)
Ser Gly Gly (SGG)
Gly Ser Ser (GSS)
Ser Ser Gly (SSG)
Ser Gly Ser (SGS)
Gly Gly Gly Ser (GGGS, SEQ ID NO: 28)
Gly Gly Ser Gly (GGSG, SEQ ID NO: 29)
Gly Ser Gly Gly (GSGG, SEQ ID NO: 46)
Ser Gly Gly Gly (SGGG, SEQ ID NO: 47)
Gly Ser Ser Gly (GSSG, SEQ ID NO: 48)
Gly Gly Gly Gly Ser (GGGGS, SEQ ID NO: 49)
Gly Gly Gly Ser Gly (GGGSG, SEQ ID NO: 33)
Gly Gly Ser Gly Gly (GGSGG, SEQ ID NO: 30)
Gly Ser Gly Gly Gly (GSGGG, SEQ ID NO: 32)
Gly Ser Gly Gly Ser (GSGGS, SEQ ID NO: 27)
Ser Gly Gly Gly Gly (SGGGG, SEQ ID NO: 51)
Gly Ser Ser Gly Gly (GSSGG, SEQ ID NO: 52)
Gly Ser Gly Ser Gly (GSGSG, SEQ ID NO: 31)
Ser Gly Gly Ser Gly (SGGSG, SEQ ID NO: 53)
Gly Ser Ser Ser Gly (GSSSG, SEQ ID NO: 34)
Gly Gly Gly Gly Gly Ser (GGGGGS, SEQ ID NO: 50)
Ser Gly Gly Gly Gly Gly (SGGGGG, SEQ ID NO: 54)
Gly Gly Gly Gly Gly Gly Ser
(GGGGGGS, SEQ ID NO: 55)
Ser Gly Gly Gly Gly Gly Gly
(SGGGGGG, SEQ ID NO: 56)
(Gly Gly Gly Gly Ser (GGGGS, SEQ ID NO: 49))n
(Ser Gly Gly Gly Gly (SGGGG, SEQ ID NO: 51))n

In the present specification, the “association” can refer to, for example, a state where two or more polypeptide regions interact with each other. In general, a hydrophobic bond, a hydrogen bond, an ionic bond, or the like is formed between the intended polypeptide regions to form an associate. As one example of common association, an antibody typified by a natural antibody is known to retain a paired structure of a heavy chain variable region (VH) and a light chain variable region (VL) through a noncovalent bond or the like therebetween.

In some embodiments of the present invention, the inhibiting domain of the carrying moiety associates with the antigen binding domain. The inhibiting domain may constitute a portion of the carrying moiety or may constitute the whole of the carrying moiety. From another viewpoint, the inhibiting domain can also be defined as a moiety associating with the antigen binding domain, in the carrying moiety.

In a more specific embodiment, the antigen binding domain and the inhibiting domain which is VL, VH or VHH form association as found between antibody VH and antibody VL, or association between antibody VH or antibody VH, or between antibody VL and antibody VL. In a further specific embodiment, the antigen binding domain and the inhibiting domain which is VL, VH or VHH form association as found between antibody VH and antibody VL, and in a state of the association thus formed, the inhibiting domain conformationally inhibits the binding of the antigen binding domain to the antigen or conformationally changes the antigen binding site of the antigen binding domain so that the antigen binding activity of the antigen binding domain is inhibited by the VL, the VH or the VHH. In an embodiment using VHH as the antigen binding domain, it is considered that the binding of the VHH to the antigen is conformationally inhibited by the inhibiting domain when CDR3, a main antigen binding site of the VHH, or its neighboring site exists at the interface of association with the inhibiting domain.

The association of the antigen binding domain with the inhibiting domain may be canceled, for example, by cleaving the cleavage site. The cancelation of the association can be used interchangeably with, for example, the cancelation of the state where two or more polypeptide regions interact with each other. The interaction between the two or more polypeptide regions may be wholly canceled, or the interaction between the two or more polypeptide regions may be partially canceled.

In the present specification, the “interface” usually refers to a face at which two regions associate or interact with each other Amino acid residues forming the interface are usually one or more amino acid residues contained in each polypeptide region subjected to the association and more preferably refer to amino acid residues that approach each other upon association and participate in interaction. Specifically, the interaction includes a noncovalent bond such as a hydrogen bond, electrostatic interaction, or salt bridge formation between the amino acid residues approaching each other upon association.

In the present specification, the “amino acid residues forming the interface” specifically refers to amino acid residues contained in polypeptide regions constituting the interface. As one example, the polypeptide regions constituting the interface refer to polypeptide regions responsible for intramolecular or intermolecular selective binding in antibodies, ligands, antagonists, receptors, substrates, etc. Specific examples of such polypeptide regions in antibodies can include a heavy chain variable region and a light chain variable region. In some embodiments of the present invention, examples of such polypeptide regions can include an antigen binding domain and an inhibiting domain.

Examples of the amino acid residues forming the interface include, but are not limited to, amino acid residues approaching each other upon association. The amino acid residues approaching each other upon association can be found, for example, by analyzing the conformations of polypeptides and examining the amino acid sequences of polypeptide regions forming the interface upon association of the polypeptides.

In some embodiments of the present invention, an amino acid residue involved in association in the antigen binding domain, or an amino acid residue involved in association in the inhibiting domain can be altered in order to promote the association of the antigen binding domain with the inhibiting domain. In a further specific embodiment, an amino acid residue forming the interface with the inhibiting domain, in the antigen binding domain, or an amino acid residue forming the interface with the antigen binding domain, in the inhibiting domain can be altered. In a preferred embodiment, the amino acid residue forming the interface can be altered by a method of introducing a mutation to the interface amino acid residue such that two or more amino acid residues forming the interface have different charges. The alteration of the amino acid residue to result in different charges includes the alteration of a positively charged amino acid residue to a negatively charged amino acid residue or an uncharged amino acid residue, the alteration of a negatively charged amino acid residue to a positively charged amino acid residue or an uncharged amino acid residue, and the alteration of an uncharged amino acid residue to a positively or negatively charged amino acid residue. Such an amino acid alteration is performed for the purpose of promoting the association and is not limited by the position of the amino acid alteration or the type of the amino acid as long as the purpose of promoting the association can be achieved. Examples of the alteration include, but are not limited to, substitution.

In some embodiments of the present invention, VHH serving as the antigen binding domain associates with VL serving as the inhibiting domain. The amino acid residue involved in association with VL, in VHH can refer to, for example, an amino acid residue forming the interface between the VHH and the VL. Examples of the amino acid residue involved in association with VL, in VHH include, but are not limited to, amino acid residues at positions 37, 44, 45, and 47 (J. Mol. Biol. (2005) 350, 112-125). The activity of the VHH is inhibited by promoting the association between the VHH and the VL. Likewise, the amino acid residue involved in association with VHH, in VL can refer to, for example, an amino acid residue forming the interface between the VHH and the VL.

An amino acid residue involved in association with VL, in VHH can be altered in order to promote the association between the VHH and the VL. Examples of such an amino acid substitution include, but are not limited to, F37V, Y37V, E44G, Q44G, R45L, H45L, G47W, F47W, L47W, T47W, or/and S47W. Instead of altering each residue in VHH, VHH originally having an amino acid residue 37V, 44G, 45L, or/and 47W may be used.

Instead of the VHH amino acid, an amino acid residue involved in association with VHH, in VL may be altered, and amino acid alterations may also be introduced to both VHH and VL, as long as the purpose of promoting the association between the VHH and the VL can be achieved.

In some embodiments of the present invention, VHH or VH serving as the antigen binding domain associates with VL serving as the inhibiting domain. The amino acid residue involved in association with VL, in VHH or VH can refer to, for example, an amino acid residue forming the interface between the VHH or VH and the VL, and an amino acid residue forming the interface between the CH1 and CL. Examples of the amino acid residue involved in association with VL, in VHH or VH and VL and CH1 and CL include, but are not limited to, amino acid residues at the following combinations position:

| VH | CH1 | VL | CL |
|---|---|---|---|
| 39K, 62E | H172A, F174G | 1R, 38D, (36F) | L135Y, S176W |
| 39Y | — | 38R | — |
| | T192E | | N137K, S114A |
| | L143Q, S188V | | V133T, S176V |
| | F126C | | S121C |
| 39E | — | 38K | — |
| 39K | — | 38E | — |
| V37F, L45W | — | Y87A, F98M | — |

As for enhancement of association between VH and VL several mutations have been reported in Nature Biotechnology volume 32, pages 191-198 (2014), Biophys. J. 75, 1473-1482 (1998), Protein Eng. Des. Sel. 23, 667-677 (2010), MAbs. 2017 February-March; 9(2): 182-212 and WO2013065708.

The activity of the VHH or VL is inhibited by promoting the association between the VHH or VH and the VL.

In some alternative embodiments of the present invention, the antigen binding domain and the inhibiting domain can be associated with each other by using VHH as the antigen binding domain and using VH or VHH as the inhibiting domain. An amino acid residue involved in association with VH or VHH serving as the inhibiting domain, in VHH serving as the antigen binding domain can be identified and altered in order to promote the association of the antigen binding domain VHH with the inhibiting domain VH or VHH. Also, an amino acid residue involved in association with VHH serving as the antigen binding domain, in VH or VHH serving as the inhibiting domain, can be identified and altered.

In the case of using an antigen binding domain other than VHH, an amino acid residue involved in association, in the antigen binding domain or the inhibiting domain can also be identified and altered similarly to above.

In some embodiments of the present invention, the carrying moiety and the antigen binding domain are fused via a linker. In a more specific embodiment, the carrying moiety and the antigen binding domain are fused via a linker containing a cleavage site. In an alternative specific embodiment, the carrying moiety and the antigen binding domain are fused via a linker, and the fusion protein thus formed contains a cleavage site.

In another embodiment of the present invention, the carrying moiety and the antigen binding domain are fused without a linker. In a more specific embodiment, an amino bond is formed between the N-terminal amino acid of the carrying moiety and the C-terminal amino acid of the antigen binding domain to form a fusion protein. The formed fusion protein contains a cleavage site. In a particular embodiment, one to several N-terminal amino acids of the carrying moiety or/and one to several C-terminal amino acids of the antigen binding domain are altered, and the N terminus of the carrying moiety and the C terminus of the antigen binding domain are fused to form a cleavage site near the fusion position. More specifically, the cleavage site can be formed, for example, by converting four C-terminal amino acids of the antigen binding domain to a LSGR sequence and converting four N-terminal amino acids of the carrying moiety to a SDNH sequence.

In some embodiments of the present invention, the cleavage site of the polypeptide comprising a carrying moiety and an antigen binding domain comprises a protease cleavage sequence. The protease cleavage sequence may be placed at any position in the polypeptide as long as the antigen binding domain is released by protease cleavage and does not lose its antigen binding activity after the release.

In some embodiments of the present invention, the carrying moiety comprises an antibody constant region, and the N terminus of the antibody constant region and the C terminus of the antigen binding domain are fused via a linker or without a linker.

In a particular embodiment, the protease cleavage sequence is located within the antibody constant region contained in the carrying moiety. In this case, the protease cleavage sequence can be located within the antibody constant region such that the antigen binding domain is released by protease cleavage. In a specific embodiment, the protease cleavage sequence is located within an antibody heavy chain constant region contained in the carrying moiety, and more specifically located on the antigen binding domain side with respect to amino acid position 140 (EU numbering) in the antibody heavy chain constant region, preferably on the antigen binding domain side with respect to amino acid position 122 (EU numbering) in the antibody heavy chain constant region. In an alternative specific embodiment, the protease cleavage sequence is located within an antibody light chain constant region contained in the carrying moiety, and more specifically located on the antigen binding domain side with respect to amino acid position 130 (EU numbering) (Kabat numbering position 130) in the antibody light chain constant region, preferably on the antigen binding domain side with respect to amino acid position 113 (EU numbering) (Kabat numbering position 113) in the antibody light chain constant region.

In some embodiments of the present invention, the antigen binding domain is a single-domain antibody, and the C terminus of the single-domain antibody and the N terminus of the carrying moiety are fused via a linker or without a linker.

In a particular embodiment, the protease cleavage sequence is located within the antigen binding domain. In a more specific embodiment, the antigen binding domain is a single-domain antibody, and the single-domain antibody is a single-domain antibody prepared from VH, or VHH, and the protease cleavage sequence is located on the carrying moiety side with respect to amino acid position 35b (Kabat numbering) of the single-domain antibody, preferably on the carrying moiety side with respect to amino acid position 95 (Kabat numbering) of the single-domain antibody, more preferably on the carrying moiety side with respect to amino acid position 109 (Kabat numbering) of the single-domain antibody. In an alternative specific embodiment, the antigen binding domain is a single-domain antibody, and the single-domain antibody is a single-domain antibody prepared from VL, and the protease cleavage sequence is located on the carrying moiety side with respect to amino acid position 32 (Kabat numbering) of the single-domain antibody, preferably on the carrying moiety side with respect to amino acid position 91 (Kabat numbering) of the single-domain antibody, more preferably on the carrying moiety side with respect to amino acid position 104 (Kabat numbering) of the single-domain antibody.

In some embodiments of the present invention, the carrying moiety comprises an antibody constant region, the antigen binding domain is a single-domain antibody, and the antibody constant region and the single-domain antibody are fused via a linker or without a linker. In a more specific embodiment, the N terminus of the antibody constant region and the C terminus of the single-domain antibody are fused via a linker or without a linker. In an alternative specific embodiment, the C terminus of the antibody constant region and the N terminus of the single-domain antibody are fused via a linker or without a linker.

In a particular embodiment, the protease cleavage sequence is located within the antibody constant region contained in the carrying moiety. In a more specific embodiment, the protease cleavage sequence is located on the antigen binding domain side with respect to amino acid position 140 (EU numbering) in an antibody heavy chain constant region, preferably on the antigen binding domain side with respect to amino acid position 122 (EU numbering) in an antibody heavy chain constant region. In an alternative specific embodiment, the protease cleavage sequence is located on the antigen binding domain side with respect to amino acid position 130 (EU numbering) (Kabat numbering position 130) in an antibody light chain constant region, preferably on the antigen binding domain side with respect to amino acid position 113 (EU numbering) (Kabat numbering position 113) in an antibody light chain constant region.

In a particular embodiment, the protease cleavage sequence is located within the antigen binding domain. In a more specific embodiment, the antigen binding domain is a single-domain antibody, and the single-domain antibody is a single-domain antibody prepared from VH, or VHH, and the protease cleavage sequence is located on the antibody constant region side with respect to amino acid position 35b (Kabat numbering) of the single-domain antibody, preferably on the antibody constant region side with respect to amino acid position 95 (Kabat numbering) of the single-domain antibody, more preferably on the antibody constant region side with respect to amino acid position 109 (Kabat numbering) of the single-domain antibody. In an alternative specific embodiment, the antigen binding domain is a single-domain antibody, and the single-domain antibody is a single-domain antibody prepared from VL, and the protease cleavage sequence is located on the antibody constant region side with respect to amino acid position 32 (Kabat numbering) of the single-domain antibody, preferably on the antibody constant region side with respect to amino acid position 91 (Kabat numbering) of the single-domain antibody, more preferably on the antibody constant region side with respect to amino acid position 104 (Kabat numbering) of the single-domain antibody.

In a particular embodiment, the protease cleavage sequence is located near the boundary between the antigen binding domain and the carrying moiety. The phrase “near the boundary between the antigen binding domain and the carrying moiety” refers to a moiety that resides upstream or downstream of the linking site between the antigen binding domain and the carrying moiety and does not largely influence the secondary structure of the antigen binding domain.

In a more specific embodiment, the antigen binding domain is linked to the antibody constant region contained in the carrying moiety, and the protease cleavage sequence is located near the boundary between the antigen binding domain and the antibody constant region. The phrase “near the boundary between the antigen binding domain and the antibody constant region” can refer to near the boundary between the antigen binding domain and an antibody heavy chain constant region, or near the boundary between the antigen binding domain and an antibody light chain constant region. When the antigen binding domain is a single-domain antibody, and the single-domain antibody is a single-domain antibody prepared from VH, or VHH and is connected to an antibody heavy chain constant region, the phrase “near the boundary between the antigen binding domain and the antibody constant region” can refer to between amino acid position 101 (Kabat numbering) of the single-domain antibody and amino acid position 140 (EU numbering) of the antibody heavy chain constant region and can preferably refer to between amino acid position 109 (Kabat numbering) of the single-domain antibody and amino acid position 122 (EU numbering) of the antibody heavy chain constant region. When the antigen binding domain is a single-domain antibody, and the single-domain antibody is a single-domain antibody prepared from VH, or VHH and is connected to an antibody light chain constant region, the phrase “near the boundary between the antigen binding domain and the antibody light chain constant region” can refer to between amino acid position 101 (Kabat numbering) of the single-domain antibody and amino acid position 130 (EU numbering) (Kabat numbering position 130) of the antibody light chain constant region and can preferably refer to between amino acid position 109 (Kabat numbering) of the single-domain antibody and amino acid position 113 (EU numbering) (Kabat numbering position 113) of the antibody light chain constant region. When the antigen binding domain is a single-domain antibody, and the single-domain antibody is a single-domain antibody prepared from VL, the phrase “near the boundary between the antigen binding domain and the antibody constant region” refers to between amino acid position 96 (Kabat numbering) of the single-domain antibody and the prescribed position of the antibody constant region, preferably between amino acid position 104 (Kabat numbering) of the single-domain antibody and the prescribed position of the antibody constant region.

In some embodiments of the present invention, the polypeptide is an IgG antibody-like molecule. Examples of such embodiments include, but are not limited to: an embodiment in which the carrying moiety comprises an IgG antibody constant region, an antigen binding domain takes the place of VH of an IgG antibody or a modified IgG antibody, and the antigen binding activity is inhibited by VL or VH; an embodiment in which the carrying moiety comprises an IgG antibody constant region, an antigen binding domain takes the place of VL of an IgG antibody or a modified IgG antibody, and the antigen binding activity is inhibited by VH or VL; and an embodiment in which the carrying moiety comprises an IgG antibody constant region, an antigen binding domain takes the place of one of VH and VL of an IgG antibody or a modified IgG antibody, and an additional antigen binding domain that inhibits the antigen binding activity of the antigen binding domain takes the place of the other domain of the IgG antibody or a modified IgG antibody.

The term “IgG antibody-like molecule” and “modified IgG antibody” used in the present specification is used to define a molecule having moieties substantially similar in structure to constant domains or constant regions as in an IgG antibody, and moieties substantially similar in structure to variable domains or variable regions as in the IgG antibody, and having conformation substantially similar to that of the IgG antibody. However, in the present specification, the “IgG antibody-like molecule” and “modified IgG antibody” may or may not exert antigen binding activity while retaining the structures similar to those of the IgG antibody. The terms “IgG antibody-like molecule”, “IgG-antibody like molecule” and “IgG-antibody-like molecule” are used interchangeably with each other herein.

In some embodiments of the present invention, the following types of antibodies are exemplified as the “modified IgG antibody”;
(i) a “modified IgG antibody” in which a H chain variable domain or a H chain variable region as in the IgG antibody is substituted with a L chain variable domain or a L chain variable region,
(ii) a “modified IgG antibody” in which a L chain variable domain or a L chain variable region as in the IgG antibody is substituted with a H chain variable domain or a H chain variable region,
(iii) a “modified IgG antibody” in which a H chain constant domain or a H chain constant region as in the IgG antibody is substituted with a L chain constant domain or a L chain constant region,
(iv) a “modified IgG antibody” in which a L chain constant domain or a L chain constant region as in the IgG antibody is substituted with a H chain constant domain or a H chain constant region,
(v) a “modified IgG antibody” having the features defined in the above (i) and (ii),
(vi) a “modified IgG antibody” having the features defined in the above (i) and (iii),
(vii) a “modified IgG antibody” having the features defined in the above (i) and (iv),
(viii) a “modified IgG antibody” having the features defined in the above (ii) and (iii), and
(ix) a “modified IgG antibody” having the features defined in the above (ii) and (iv),
(x) a “modified IgG antibody” having the features defined in the above (ii) and (iv)
(xi) a “modified IgG antibody” having the features defined in the above (i), (ii) and (iii),
(xii) a “modified IgG antibody” having the features defined in the above (i), (ii) and (iv),
(xiii) a “modified IgG antibody” having the features defined in the above (i), (iii) and (iv), and
(xiv) a “modified IgG antibody” having the features defined in the above (ii), (iii) and (iv).

The polypeptide may comprise one or more antigen binding domains. One or more inhibiting domains may inhibit the antigen binding activity of a plurality of antigen binding domains. A plurality of antigen binding domains may each be associated with the inhibiting domain. A plurality of antigen binding domains may each be fused with the carrying moiety. A plurality of antigen binding domains may each be capable of released from the polypeptide. The cleavage site(s) for releasing a plurality of antigen binding domains may be a plurality of cleavage sites corresponding to the number of antigen binding domains.

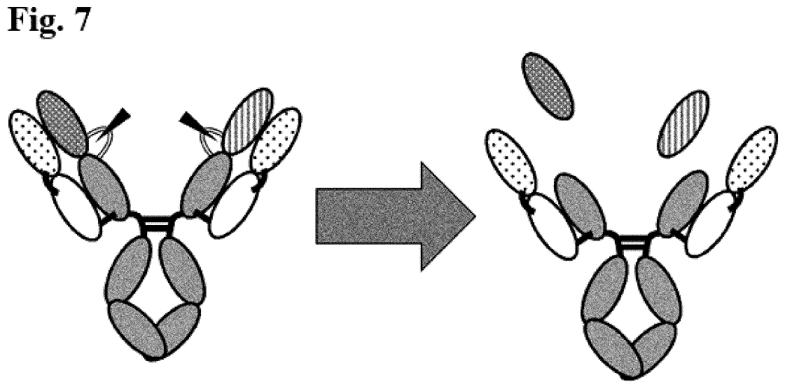

When the polypeptide is an IgG antibody-like molecule, antigen binding domains may be respectively established at moieties corresponding to two variable regions of the IgG antibody, as shown in FIG. 7. Such an embodiment should be understandable by those skilled in the art with reference to the present invention. The antigen binding domains incorporated in both arms may have the same antigen binding specificity or may differ in antigen binding specificity. Such an embodiment should be understandable by those skilled in the art with reference to the present invention. It is obvious that these embodiments are included in the scope of the present invention.

In some embodiments of the present invention, the antigen binding domain is further linked to a second antigen binding domain. Examples of the second antigen binding domain include, but are not limited to, single-domain antibodies, antibody fragments, antagonists, a module called A domain of approximately 35 amino acids contained in an in vivo cell membrane protein avimer (International Publication Nos. WO2004/044011 and WO2005/040229), adnectin containing a 10Fn3 domain serving as a protein binding domain derived from a glycoprotein fibronectin expressed on cell membranes (International Publication No. WO2002/032925), Affibody containing an IgG binding domain scaffold constituting a three-helix bundle composed of 58 amino acids of protein A (International Publication No. WO1995/001937), DARPins (designed ankyrin repeat proteins) which are molecular surface-exposed regions of ankyrin repeats (AR) each having a 33-amino acid residue structure folded into a subunit of a turn, two antiparallel helices, and a loop (International Publication No. WO2002/020565), anticalin having four loop regions connecting eight antiparallel strands bent toward the central axis in one end of a barrel structure highly conserved in lipocalin molecules such as neutrophil gelatinase-associated lipocalin (NGAL) (International Publication No. WO2003/029462), and a depressed region in the internal parallel sheet structure of a horseshoe-shaped fold composed of repeated leucine-rich-repeat (LRR) modules of an immunoglobulin structure-free variable lymphocyte receptor (VLR) as seen in the acquired immune systems of jawless vertebrates such as lamprey or hagfish (International Publication No. WO2008/016854). In a preferred embodiment, the second antigen binding domain has antigen binding specificity different from that of the antigen binding domain. In a preferred embodiment, the molecular weight of the antigen binding domain and the second antigen binding domain linked is 120 kDa, 100 kDa, 80 kDa, 60 kDa, 40 kDa, 20 kDa or smaller.

In some more specific embodiments, the antigen binding domain and the second antigen binding domain are single-domain antibodies differing in antigen binding specificity, the antigen binding domain and the second antigen binding domain linked are capable of being released from the polypeptide, and the antigen binding domain and the second antigen binding domain form a bispecific antigen binding molecule after release. Examples of such a bispecific antigen binding molecule include, but are not limited to, a bispecific antigen binding molecule having an antigen binding domain specifically binding to the target cell surface antigen and a second antigen binding domain specifically binding to an immunocyte surface antigen, a bispecific antigen binding molecule having an antigen binding domain and a second antigen binding domain binding to different subunits of the same antigen, and a bispecific antigen binding molecule having an antigen binding domain and a second antigen binding domain binding to different epitopes in the same antigen. Such a bispecific antigen binding molecule can recruit immunocytes to the vicinity of target cells and is thus considered useful in the treatment of a disease caused by the target cells.

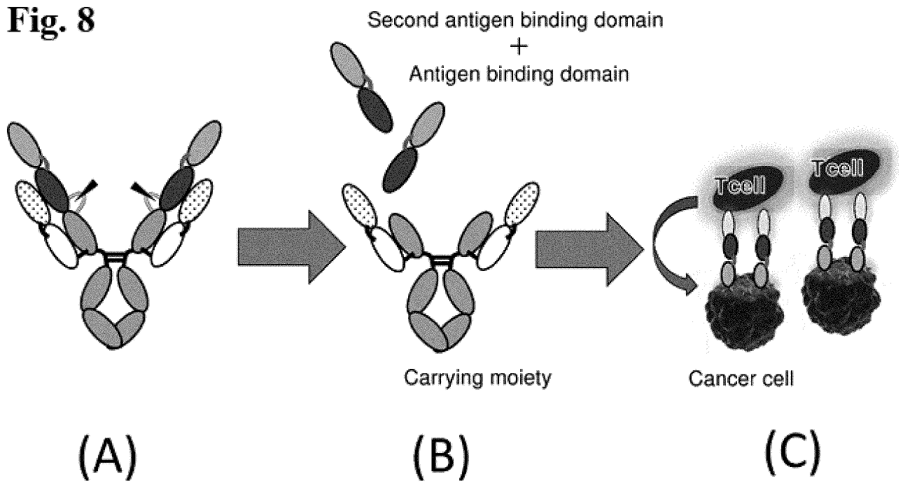

The antigen binding activity of the second antigen binding domain may or may not be inhibited by the carrying moiety. The second antigen binding domain may or may not be associated with a partial structure of the carrying moiety. Particularly, when the antigen binding domain and the second antigen binding domain differ in antigen binding specificity, the antigen binding domain in an unreleased state cannot exert antigen binding activity, as shown in, for example, FIG. 8, even if the antigen binding activity of the second antigen binding domain is not inhibited and even if the second antigen binding domain is not associated with a partial structure of the carrying moiety. This bispecific antigen binding molecule comprising the antigen binding domain linked to the second antigen binding domain cannot exert a function of bispecifically binding to two types of antigens.

FIG. 8 shows one exemplary form in which the antigen binding domain is further linked to the second antigen binding domain.

In the present specification, the term “specificity” refers to a property by which one of specifically binding molecules does not substantially bind to a molecule other than its one or more binding partner molecules. This term is also used when the antigen binding domain has specificity for an epitope contained in a particular antigen. The term is also used when the antigen binding domain has specificity for a particular epitope among a plurality of epitopes contained in an antigen. In this context, the term “not substantially bind” is determined according to the method described in the section about binding activity and means that the binding activity of a specific binding molecule for a molecule other than the binding partner(s) is 80% or less, usually 50% or less, preferably 30% or less, particularly preferably 15% or less, of its binding activity for the binding partner molecule(s).

The present invention also relates to a pharmaceutical composition (drug) comprising the polypeptide of the present invention and a pharmaceutically acceptable carrier.

The “treatment” (and its grammatically derived words, for example, “treat” and “treating”) used in the present specification means clinical intervention that intends to alter the natural course of an individual to be treated and can be carried out both for prevention and during the course of a clinical pathological condition. The desirable effect of the treatment includes, but is not limited to, the prevention of the development or recurrence of a disease, the alleviation of symptoms, the attenuation of any direct or indirect pathological influence of the disease, the prevention of metastasis, reduction in the rate of progression of the disease, recovery from or alleviation of a disease condition, and ameliorated or improved prognosis. In some embodiments, the polypeptide of the present invention is used for delaying the onset of a disease or delaying the progression of the disease.

In the present invention, the pharmaceutical composition usually refers to a drug for the treatment or prevention of a disease or for examination or diagnosis. In the present invention, the term “pharmaceutical composition comprising the polypeptide” may be used interchangeably with a “method for treating a disease, comprising administering the polypeptide to a subject to be treated” and may be used interchangeably with “use of the polypeptide for the production of a drug for the treatment of a disease”. Also, the term “pharmaceutical composition comprising the polypeptide” may be used interchangeably with “use of the polypeptide for treating a disease”.

The pharmaceutical composition of the present invention can be formulated by use of a method known to those skilled in the art. For example, the pharmaceutical composition can be parenterally used in an injection form of a sterile solution or suspension with water or any of other pharmaceutically acceptable liquids. The pharmaceutical composition can be formulated, for example, by appropriately combining the polypeptide with a pharmacologically acceptable carrier or medium, specifically, sterile water or physiological saline, a plant oil, an emulsifier, a suspending agent, a surfactant, a stabilizer, a flavoring agent, an excipient, a vehicle, an antiseptic, a binder, etc. and mixing them into a unit dosage form required for generally accepted pharmaceutical practice. The amount of the active ingredient in these formulations is set so as to give an appropriate volume in a prescribed range.

A sterile composition for injection can be formulated according to usual pharmaceutical practice using a vehicle such as injectable distilled water. Examples of the injectable aqueous solution include isotonic solutions containing physiological saline, glucose, or other adjuvants (e.g., D-sorbitol, D-mannose, D-mannitol, and sodium chloride). The aqueous solution can be used in combination with an appropriate solubilizer, for example, an alcohol (ethanol, etc.), a polyalcohol (propylene glycol, polyethylene glycol, etc.), or a nonionic surfactant (Polysorbate 80™, HCO-50, etc.).

Examples of the oil solution include sesame oil and soybean oil. The oil solution can also be used in combination with benzyl benzoate and/or benzyl alcohol as a solubilizer. The oil solution can be supplemented with a buffer (e.g., a phosphate buffer solution and a sodium acetate buffer solution), a soothing agent (e.g., procaine hydrochloride), a stabilizer (e.g., benzyl alcohol and phenol), and an antioxidant. The prepared injection solution is usually filled into an appropriate ampule.

The pharmaceutical composition of the present invention is preferably administered through a parenteral route. For example, a composition having an injection, intraarticular, transnasal, transpulmonary, or percutaneous dosage form is administered. The pharmaceutical composition can be administered systemically or locally by, for example, intravenous injection, intramuscular injection, intraperitoneal injection, or subcutaneous injection.

The administration method can be appropriately selected according to the age and symptoms of a patient. The dose of the pharmaceutical composition containing the polypeptide can be set to the range of, for example, 0.0001 mg to 1000 mg per kg body weight per dose. Alternatively, the dose of the pharmaceutical composition containing the polypeptide can be set to a dose of, for example, 0.001 to 100000 mg per patient. However, the present invention is not necessarily limited by these numerical values. Although the dose and the administration method vary depending on the body weight, age, symptoms, etc. of a patient, those skilled in the art can set an appropriate dose and administration method in consideration of these conditions. Subjects to be administered (applied) with the polypeptide of the present invention include, which can be virtually any animal, for example, a human, monkey, mouse, rabbit, etc.

The present invention also relates to a method for producing a polypeptide comprising a carrying moiety having an inhibiting domain, and an antigen binding domain.

One method for producing the polypeptide of the present invention is a method comprising: obtaining an antigen binding domain having antigen binding activity; linking the antigen binding domain to a carrying moiety such that the antigen binding activity of the antigen binding domain is inhibited by an inhibiting domain, to form a polypeptide precursor; and further inserting a cleavage site into the polypeptide precursor or altering a portion of the polypeptide precursor to a cleavage site. The method for introducing the cleavage site can be any of the insertion of the cleavage site and the alteration of a portion of the polypeptide precursor as long as the cleavage site can be introduced into the polypeptide precursor. Alternatively, an alteration site may be introduced into the polypeptide precursor by the combination of both the approaches. Such an embodiment should be obvious to those skilled in the art with reference to the present specification and is included in the scope of the present invention.

Another method for producing the polypeptide of the present invention is a method comprising: obtaining an antigen binding domain having antigen binding activity; and linking the antigen binding domain to a carrying moiety via a cleavage site such that the antigen binding activity of the antigen binding domain is inhibited by an inhibiting domain, to form a polypeptide. When the antigen binding domain is linked to the carrying moiety via a cleavage site, the cleavage site may be sandwiched between the antigen binding domain and the carrying moiety, or a portion of the antigen binding domain or/and a portion of the carrying moiety may be altered and used as a portion of the cleavage site.

The method for producing the polypeptide will be described below. In one embodiment of the present invention, the antigen binding domain is preferably a VL having antigen binding activity by itself. Further, in one embodiment of the present invention, an antigen is preferably IL-1R1, IL-1alpha, IL-1beta or IL-1RAcP.

In one embodiment of the present invention, the method for producing a polypeptide comprising a carrying moiety having an inhibiting domain, and an antigen binding domain is a production method comprising the following steps:
(a) obtaining an antigen binding domain binding to a target antigen;
(b) linking the antigen binding domain obtained in the step (a) to a carrying moiety such that the antigen binding activity of the antigen binding domain is inhibited by an inhibiting domain of the carrying moiety, to form a polypeptide precursor; and
(c) introducing a protease cleavage sequence into the polypeptide precursor.

In one embodiment of the present invention, the method for producing a polypeptide comprising a carrying moiety having an inhibiting domain, and an antigen binding domain is a production method comprising the following steps:
(a) obtaining an antigen binding domain binding to a target antigen;
(b) linking the antigen binding domain obtained in the step (a) to a carrying moiety such that the antigen binding activity of the antigen binding domain is inhibited by an inhibiting domain of the carrying moiety, to form a polypeptide precursor; and
(c) introducing a protease cleavage sequence to near the boundary between the antigen binding domain and the carrying moiety.

In one embodiment of the present invention, the method for producing a polypeptide comprising a carrying moiety having an inhibiting domain, and an antigen binding domain is a production method comprising the following steps:
(a) obtaining an antigen binding domain binding to a target antigen; and
(b) linking the antigen binding domain obtained in the step (a) to the carrying moiety via a protease cleavage sequence such that the antigen binding activity of the antigen binding domain is inhibited by an inhibiting domain of the carrying moiety, to form a polypeptide.

In a particular embodiment, the method for producing a polypeptide comprising a carrying moiety having an inhibiting domain, and an antigen binding domain is the production method further comprising the following step:
(d) confirming that the binding activity of the antigen binding domain incorporated in the polypeptide or the polypeptide precursor against the target antigen is weakened or lost.

In the present invention, the phrase “binding activity is weakened” means that the binding activity against the target antigen is decreased as compared with that before the linking, and the degree of this decrease is not limited.

In a particular embodiment, the method for producing a polypeptide comprising a carrying moiety having an inhibiting domain, and an antigen binding domain is the production method further comprising the following step:
(e) releasing the antigen binding domain by the protease cleavage of the protease cleavage sequence and confirming that the released antigen binding domain binds to the antigen.

In one embodiment of the present invention, the method for producing a polypeptide which is an IgG antibody-like molecule comprising a carrying moiety having an inhibiting domain, and an antigen binding domain is a production method comprising the following steps:
(a) obtaining an antigen binding domain binding to a target antigen;
(b) associating the antigen binding domain obtained in the step (a) as a substitute for VH of an IgG antibody or a modified IgG antibody with VL or VH, or associating the antigen binding domain as a substitute for VL of an IgG antibody or a modified IgG antibody with VH or VL such that the antigen binding activity of the antigen binding domain is inhibited, to form an IgG antibody-like molecule precursor harboring the antigen binding domain; and
(c) introducing a protease cleavage sequence into the IgG antibody-like molecule precursor harboring the antigen binding domain.

In one embodiment of the present invention, the method for producing a polypeptide which is an IgG antibody-like molecule comprising a carrying moiety having an inhibiting domain, and an antigen binding domain is a production method comprising the following steps:
(a) obtaining an antigen binding domain binding to a target antigen;
(b) associating the antigen binding domain obtained in the step (a) as a substitute for VH of an IgG antibody or a modified IgG antibody with VL or VH, or associating the antigen binding domain as a substitute for VL of an IgG antibody or a modified IgG antibody with VH or VL such that the antigen binding activity of the antigen binding domain is inhibited, to form an IgG antibody-like molecule precursor harboring the antigen binding domain; and
(c) introducing a protease cleavage sequence to near the boundary between the antigen binding domain and an antibody constant region in the IgG antibody-like molecule precursor.

In one embodiment of the present invention, the method for producing a polypeptide which is an IgG antibody-like molecule comprising a carrying moiety having an inhibiting domain, and an antigen binding domain is a production method comprising the following steps:
(a) obtaining an antigen binding domain binding to a target antigen; and
(b) linking the antigen binding domain obtained in the step (a) as a substitute for IgG antibody VH or VL to an IgG antibody heavy chain constant region or light chain constant region via a protease cleavage sequence such that the antigen binding activity of the antigen binding domain is inhibited, to form an IgG antibody-like molecule harboring the antigen binding domain.

In a particular embodiment, the method for producing a polypeptide which is an IgG antibody-like molecule comprising a carrying moiety having an inhibiting domain, and an antigen binding domain is the production method further comprising the following step:
(d) confirming that the binding activity of the antigen binding domain harbored in the IgG antibody-like molecule or the IgG antibody-like molecule precursor against the target antigen is weakened or lost.

In the present invention, the phrase “binding activity is weakened” means that the binding activity against the target antigen is decreased as compared with that before the association or the linking, and the degree of this decrease is not limited.

In a particular embodiment, the method for producing a polypeptide which is an IgG antibody-like molecule comprising a carrying moiety having an inhibiting domain, and an antigen binding domain is the production method further comprising the following step:
(e) releasing the antigen binding domain by the protease cleavage of the protease cleavage sequence and confirming that the released antigen binding domain binds to the target antigen.

In the case of using VH, VL or VHH as the inhibiting domain, the method for inhibiting the antigen binding activity of the antigen binding domain by the inhibiting domain of the carrying moiety includes a method of associating the antigen binding domain with VH, VL or VHH. The VH, the VL or the VHH that inhibits the antigen binding activity of the provided antigen binding domain can be screened for by associating known VH, VL or VHH with the antigen binding domain and comparing the antigen binding activity of the antigen binding domain between before and after the association.

In another method for inhibiting the antigen binding activity of the antigen binding domain by particular VH, VL or VHH, an amino acid residue involved in association with VH, VL or VHH, in the antigen binding domain can be substituted to promote the association, or an antigen binding domain/inhibiting domain pair having the desired level of difference in antigen binding activity between before and after the association can also be provided by using an antigen binding domain originally having, as such an amino acid residue, an amino acid that can promote the association.

In one embodiment of the present invention, the method for producing a polypeptide which is an IgG antibody-like molecule comprising a carrying moiety having an inhibiting domain, and an antigen binding domain is a production method comprising the following steps:
(a) substituting an amino acid residue in an antigen binding domain that involves in association of the antigen binding domain with antibody VH, or substituting an amino acid residue in an antigen binding domain that involves in association of the antigen binding domain with antibody VL to prepare an antigen binding domain variant retaining the binding activity of the antigen binding domain against the target antigen;
(b) associating the antigen binding domain variant prepared in the step (a) with antibody VH, or associating the antigen binding domain variant with antibody VL such that the antigen binding activity of the antigen binding domain variant is inhibited, to form an IgG antibody-like molecule precursor harboring the antigen binding domain variant; and
(c) introducing a protease cleavage sequence into the IgG antibody-like molecule precursor harboring the antigen binding domain variant.

In one embodiment of the present invention, the method for producing a polypeptide which is an IgG antibody-like molecule comprising a carrying moiety having an inhibiting domain, and an antigen binding domain is a production method comprising the following steps:
(a) substituting an amino acid residue in an antigen binding domain that involves in association of the antigen binding domain with antibody VH, or substituting an amino acid residue in an antigen binding domain that involves in association of the antigen binding domain with antibody VL, to prepare an antigen binding domain variant retaining the binding activity of the antigen binding domain against the target antigen;
(b) associating the antigen binding domain variant prepared in the step (a) with antibody VH, or associating the antigen binding domain variant with antibody VL such that the antigen binding activity of the antigen binding domain is inhibited, to form an IgG antibody-like molecule precursor harboring the antigen binding domain variant; and
(c) introducing a protease cleavage sequence to near the boundary between the antigen binding domain variant and a constant region in the IgG antibody-like molecule precursor.

In one embodiment of the present invention, the method for producing a polypeptide which is an IgG antibody-like molecule comprising a carrying moiety having an inhibiting domain, and an antigen binding domain is a production method comprising the following steps:
(a) substituting an amino acid residue in an antigen binding domain that involves in association of the antigen binding domain with antibody VH, or substituting an amino acid residue in an antigen binding domain that involves in association of the antigen binding domain with antibody VL, to prepare an antigen binding domain variant retaining the binding activity of the antigen binding domain against the target antigen; and
(b) linking the antigen binding domain variant prepared in the step (a) to an IgG antibody heavy chain constant region via a protease cleavage sequence, or linking the antigen binding domain variant to an IgG antibody light chain constant region via a protease cleavage sequence such that the antigen binding activity of the antigen binding domain variant is inhibited, to form an IgG antibody-like molecule harboring the antigen binding domain variant.

In a particular embodiment, the method for producing a polypeptide which is an IgG antibody-like molecule comprising a carrying moiety having an inhibiting domain, and an antigen binding domain is the production method further comprising the following step:
(d) confirming that the binding activity of the antigen binding domain variant harbored in the IgG antibody-like molecule or the IgG antibody-like molecule precursor against the target antigen is weakened or lost.

In the present invention, the phrase “binding activity is weakened” means that the binding activity against the target antigen is decreased as compared with that before the association or the linking, and the degree of this decrease is not limited.

In a particular embodiment, the method for producing a polypeptide which is an IgG antibody-like molecule comprising a carrying moiety having an inhibiting domain, and an antigen binding domain is the production method further comprising the following step:
(e) releasing the antigen binding domain variant by the protease cleavage of the protease cleavage sequence and confirming that the released antigen binding domain variant binds to the target antigen.

The present invention also relates to a polynucleotide encoding the polypeptide comprising a carrying moiety having an inhibiting domain, and an antigen binding domain.

The polynucleotide according to the present invention is usually carried by (or inserted in) an appropriate vector and transferred to host cells. The vector is not particularly limited as long as the vector can stably retain an inserted nucleic acid. For example, when E. coli is used as the host, a pBluescript vector (manufactured by Stratagene Corp.) or the like is preferred as a vector for cloning. Various commercially available vectors can be used. In the case of using the vector for the purpose of producing the polypeptide of the present invention, an expression vector is particularly useful. The expression vector is not particularly limited as long as the vector permits expression of the polypeptide in vitro, in E. coli, in cultured cells, or in organism individuals. The expression vector is preferably, for example, a pBEST vector (manufactured by Promega Corp.) for in vitro expression, a pET vector (manufactured by Invitrogen Corp.) for E. coli, a pME18S-FL3 vector (GenBank Accession No. AB009864) for cultured cells, and a pME18S vector (Mol Cell Biol. 8: 466-472 (1988)) for organism individuals. The insertion of the DNA of the present invention into the vector can be performed by a routine method, for example, ligase reaction using restriction sites (Current protocols in Molecular Biology edit. Ausubel et al. (1987) Publish. John Wiley & Sons. Section 11.4-11.11).

The host cells are not particularly limited, and various host cells are used according to the purpose. Examples of the cells for expressing the polypeptide can include bacterial cells (e.g., Streptococcus, Staphylococcus, E. coli, Streptomyces, and Bacillus subtilis), fungal cells (e.g., yeasts and Aspergillus), insect cells (e.g., Drosophila S2 and Spodoptera SF9), animal cells (e.g., CHO, COS, HeLa, C127, 3T3, BHK, HEK293, and Bowes melanoma cells) and plant cells. The transfer of the vector to the host cells may be performed by a method known in the art, for example, a calcium phosphate precipitation method, an electroporation method (Current protocols in Molecular Biology edit. Ausubel et al., (1987) Publish. John Wiley & Sons. Section 9.1-9.9), a Lipofectamine method (manufactured by GIBCO-BRL/Thermo Fisher Scientific Inc.), or a microinjection method.

An appropriate secretory signal can be incorporated into the polypeptide of interest in order to secrete the polypeptide expressed in the host cells to the lumen of the endoplasmic reticulum, periplasmic space, or an extracellular environment. The signal may be endogenous to the polypeptide of interest or may be a foreign signal.

When the polypeptide of the present invention is secreted into a medium, the recovery of the polypeptide in the production method is performed by the recovery of the medium. When the polypeptide of the present invention is produced into cells, the cells are first lysed, followed by the recovery of the polypeptide.

A method known in the art including ammonium sulfate or ethanol precipitation, acid extraction, anion- or cation-exchange chromatography, phosphocellulose chromatography, hydrophobic interaction chromatography, affinity chromatography, hydroxyapatite chromatography, and lectin chromatography can be used for recovering and purifying the polypeptide of the present invention from the recombinant cell cultures.

In some embodiments of the present invention, the antigen binding activity of the antigen binding domain can be inhibited by associating with particular VL, associating with particular VH, or associating with particular VHH. The present invention also relates to a method for screening for such an antigen binding domain. In one embodiment of the present invention, the antigen binding domain is preferably a VL having antigen binding activity by itself. Further, in one embodiment of the present invention, an antigen is preferably IL-1R1, IL-1alpha, IL-1beta or IL-1RAcP.

The screening methods of the present invention can be used for screening for a candidate substance of a pharmaceutical. When a screening method of the present invention is conducted on, for example, but not limited to, IL-1R1, IL-1alpha, IL-1beta, or IL-1RAcP as a target antigen, an obtained antigen-binding domain can be a candidate for treatment and/or prevention of a disease or disorder mediated by IL-1R1, IL-1alpha, IL-1beta, or IL-1RAcP. Thus, the present invention provides methods of screening for a candidate substance for treatment and/or prevention of a disease or disorder mediated by a target antigen.

VL, VH or VHH having a known sequence, for example, VL, VH or VHH having a sequence registered in the IMGT or Kabat database, can be used as the VL, the VH or the VHH that inhibits the antigen binding activity of the antigen binding domain. Also, a VL, VH or VHH sequence newly identified from a human antibody library or the like can be used. The VL, the VH or the VHH that inhibits the binding activity of the antigen binding domain can be selected by preparing a protein by the combination of these sequences and measuring the binding activity by use of the method described above.

In some embodiments of the present invention, VL, VH or VHH having a human antibody germline sequence can be used as the VL, the VH or the VHH that inhibits the antigen binding activity of the antigen binding domain. In the case of using, for example, VL as the inhibiting domain, VL having kappa chain framework sequences or VL having lambda chain framework sequences can be used. Also, VL having modified framework sequences such as combined framework sequences of kappa chain and lambda chain framework sequences can be used. In one embodiment of the present invention, the VH that inhibits the antibody binding activity of an antibody binding domain may be a VH having an amino acid sequence of SEQ ID NO: 486. In one embodiment of the present invention, the VL that inhibits the antibody binding activity of an antibody binding domain may be a VL having an amino acid sequence of SEQ ID NO: 484 or 485.

In one embodiment, the present invention provides a method for screening for an antigen binding domain whose antigen binding activity can be inhibited by associating with particular VL, comprising the following steps:
(a) obtaining an antigen binding domain having target antigen binding activity;
(b) associating the antigen binding domain obtained in the step (a) with a particular VL; and
(c) confirming that the binding activity of the antigen binding domain associated with the particular VL in the step (b) against the antigen is weakened or lost.

In the present invention, the phrase “binding activity is weakened” means that the binding activity against the target antigen is decreased as compared with that before the association, and the degree of this decrease is not limited.

In one embodiment, the present invention provides a method for screening for an antigen binding domain whose antigen binding activity can be inhibited by associating with particular VH, comprising the following steps:
(a) obtaining an antigen binding domain having target antigen binding activity;
(b) associating the antigen binding domain obtained in the step (a) with a particular VH; and
(c) confirming that the binding activity of the antigen binding domain associated with the particular VH in the step (b) against the antigen is weakened or lost.

In the present invention, the phrase “binding activity is weakened” means that the binding activity against the target antigen is decreased as compared with that before the association, and the degree of this decrease is not limited.

In one embodiment, the present invention provides a method for screening for an antigen binding domain whose antigen binding activity can be inhibited by associating with particular VHH, comprising the following steps:
(a) obtaining an antigen binding domain having target antigen binding activity;
(b) associating the antigen binding domain obtained in the step (a) with a particular VHH; and
(c) confirming that the binding activity of the antigen binding domain associated with the particular VHH in the step (b) against the antigen is weakened or lost.

In the present invention, the phrase “binding activity is weakened” means that the binding activity against the target antigen is decreased as compared with that before the association, and the degree of this decrease is not limited.

Examples of the method for associating the antigen binding domain with the particular VL, VH or VHH include a method of designing a molecule having the sequence of the antigen binding domain as a substitute for the sequence of one of VH and VL in an antibody or an antibody fragment comprising both VH and VL, such as a complete antibody, Fab, Fab′, or (Fab)2, and expressing a polypeptide having the sequence.

The present invention also relates to a method for producing an antigen binding domain whose antigen binding activity is inhibited by promoting the association of the antigen binding domain with particular VL, promoting the association of the antigen binding domain with particular VH, or promoting the association of the antigen binding domain with particular VHH, in addition to screening for an antigen binding domain whose antigen binding activity is inhibited by associating with particular VL, associating with particular VH, or associating with particular VHH.

In one embodiment, the present invention provides a method for producing an antigen binding domain whose antigen binding activity is inhibited by associating with particular VL, comprising the following step:
(a) substituting an amino acid residue in an antigen binding domain that involves in association of the antigen binding domain with antibody VL, to prepare an antigen binding domain variant retaining the binding activity of the antigen binding domain against the target antigen.

In a particular embodiment, the present invention provides the method for producing an antigen binding domain whose antigen binding activity is inhibited by associating with particular VL, further comprising the following steps:
(b) associating the antigen binding domain variant prepared in the step (a) with the particular VL; and
(c) confirming that the antigen binding activity of the antigen binding domain variant associated with the VL is weakened or lost.

In the present invention, the phrase “binding activity is weakened” means that the binding activity against the target antigen is decreased as compared with that before the association, and the degree of this decrease is not limited.

In one embodiment, the present invention provides a method for producing an antigen binding domain whose antigen binding activity is inhibited by associating with particular VH, comprising the following step:
(a) substituting an amino acid residue in an antigen binding domain that involves in association of the antigen binding domain with antibody VH, to prepare an antigen binding domain variant retaining the binding activity of the antigen binding domain against the target antigen.

In a particular embodiment, the present invention provides the method for producing an antigen binding domain whose antigen binding activity is inhibited by associating with particular VH, further comprising the following steps:
(b) associating the antigen binding domain variant prepared in the step (a) with the particular VH; and
(c) confirming that the antigen binding activity of the antigen binding domain variant associated with the VH is weakened or lost.

In the present invention, the phrase “binding activity is weakened” means that the binding activity against the target antigen is decreased as compared with that before the association, and the degree of this decrease is not limited.

In one embodiment, the present invention provides a method for producing an antigen binding domain whose antigen binding activity is inhibited by associating with particular VHH, comprising the following step:
(a) substituting an amino acid residue in an antigen binding domain that involves in association of the antigen binding domain with VHH, to prepare an antigen binding domain variant retaining the binding activity of the antigen binding domain against the target antigen.

In a particular embodiment, the present invention provides the method for producing an antigen binding domain whose antigen binding activity is inhibited by associating with particular VHH, further comprising the following steps:
(b) associating the antigen binding domain variant prepared in the step (a) with the particular VHH; and
(c) confirming that the antigen binding activity of the antigen binding domain variant associated with the VHH is weakened or lost.

In the present invention, the phrase “binding activity is weakened” means that the binding activity against the target antigen is decreased as compared with that before the association, and the degree of this decrease is not limited.

The step of associating the antigen binding domain with the particular VL, VH or VHH is performed by a method of designing a molecule having the sequence of the antigen binding domain as a substitute for the sequence of one of VH and VL in an antibody or an antibody fragment comprising both VH and VL, such as a complete antibody, Fab, Fab′, or (Fab)2, and expressing a polypeptide having the sequence.

According to a certain embodiment of the present invention, the antigen binding domain of the present invention whose antigen binding activity can be inhibited or could be lost by associating with particular VL, VH or VHH can be obtained from a library comprising a plurality of fusion polypeptides of antigen binding domains each linked to a first association sustaining domain.

In the present specification, an embodiment of the “library” can provide a library that permits efficient obtainment of an antigen binding domain whose antigen binding activity can be inhibited or could be lost by associating with particular VL, VH or VHH.

In the present specification, the “library” refers to a set of a plurality of fusion polypeptides having different sequences, or nucleic acids or polynucleotides encoding these fusion polypeptides. A plurality of fusion polypeptides contained in the library are fusion polypeptides differing in sequence from each other, not having a single sequence.

In the present specification, the term “differing in sequence from each other” in a plurality of fusion polypeptides differing in sequence from each other means that the individual fusion polypeptides in the library have distinct sequences. More preferably, the term means that the antigen binding domain moieties of the individual fusion polypeptides in the library have distinct sequences. Specifically, the number of the distinct sequences in the library reflects the number of independent clones differing in sequences in the library and is also referred to as a “library size”. The library size of a usual phage display library is 106 to 1012 and may be expanded to 1014 by the application of a technique known in the art such as a ribosome display method. However, the actual number of phage particles for use in panning selection for the phage library is usually 10 to 10,000 times larger than the library size. This excessive multiple, also called the “number of equivalents of the library”, represents that 10 to 10,000 individual clones may have the same amino acid sequence. Accordingly, the term “differing in sequence from each other” according to the present invention means that the individual polypeptides in the library excluding the number of equivalents of the library have distinct sequences and more specifically means that the library has 106 to 1014 molecules, preferably 107 to 1012 molecules, of polypeptides differing in sequence from each other.

The term “plurality or in the library consisting essentially of” a plurality of fusion polypeptides according to the present invention usually refers to a set of two or more types of substances as to, for example, the polypeptide, polynucleotide molecule, vector, or virus of the present invention. Provided that, for example, two or more substances differ in particular trait from each other, this means that the substances are of two or more types. Examples thereof can include a mutant amino acid observed at a particular amino acid position in an amino acid sequence. For example, two or more polypeptides of the present invention having substantially the same, preferably identical sequences, except for particular mutant amino acids at surface-exposed, highly diverse amino acid positions are regarded as a plurality of polypeptides of the present invention. In another example, two or more polynucleotide molecules of the present invention having substantially the same, preferably identical sequences except for bases encoding particular mutant amino acids at surface-exposed, highly diverse amino acid positions are regarded as a plurality of polynucleotide molecules of the present invention.

A panning method that utilizes phage vectors is also preferably used as a method for screening the fusion polypeptides with binding activity as an index. A gene encoding each antigen binding domain and a gene encoding an IgG antibody CH1 domain or a light chain constant region can be linked in an appropriate form to form a fusion polypeptide. Genes encoding the fusion polypeptides thus formed can be inserted into phage vectors to obtain phages expressing the fusion polypeptides on the surface. After contact of the phages with the desired antigen, phages bound with the antigen can be recovered to recover DNAs encoding fusion polypeptides having the binding activity of interest. This operation can be repeated, if necessary, to enrich fusion polypeptides having the desired binding activity.

In addition to the phage display method, a technique using a cell-free translation system, a technique of presenting or displaying fusion polypeptides on cell or virus surface, a technique of using an emulsion, and the like are known as techniques of obtaining fusion polypeptides by panning using a library. For example, a ribosome display method of forming a complex of mRNA and a translated protein via ribosome by the removal of a stop codon, etc., a cDNA or mRNA display method of covalently binding a gene sequence to a translated protein using a compound such as puromycin, or a CIS display method of forming a complex of a gene and a translated protein using a nucleic acid binding protein can be used as the technique using a cell-free translation system. For example, the phage display method as well as an E. coli display method, a gram-positive bacterium display method, a yeast display method, a mammalian cell display method, or a virus display method can be used as the technique of presenting or displaying fusion polypeptides on cell or virus surface. For example, an in vitro virus display method using an emulsion containing a gene and a translation-related molecule can be used as the technique using an emulsion. These methods are already known in the art (Nat Biotechnol. 2000 December; 18(12): 1287-92, Nucleic Acids Res. 2006; 34(19): e127, Proc Natl Acad Sci USA. 2004 Mar. 2; 101(9): 2806-10, Proc Natl Acad Sci USA. 2004 Jun. 22; 101(25): 9193-8, Protein Eng Des Sel. 2008 April; 21(4): 247-55, Proc Natl Acad Sci USA. 2000 Sep. 26; 97(20): 10701-5, MAbs. 2010 September-October; 2(5): 508-18, Methods Mol Biol. 2012; 911: 183-98).

An association partner of an inhibiting domain linked to a second association sustaining domain can be used in a method for obtaining the antigen binding domain of interest from the library comprising a plurality of fusion polypeptides of antigen binding domains each linked to a first association sustaining domain.

In the present specification, the “first association sustaining domain” and the “second association sustaining domain” refer to domains that can interact with each other through a bond such as a hydrophobic bond, a hydrogen bond, or an ionic bond to form an associate. Preferred examples of the first association sustaining domain and the second association sustaining domain include, but are not limited to, an antibody light chain constant region (CL) and a CH1 domain of a heavy chain constant region.

The first association sustaining domain and the second association sustaining domain can interact with each other and form the association of the fusion polypeptide with the association partner, regardless of the degree of associativity between the antigen binding domain and the inhibiting domain.

In an alternative embodiment, the present invention provides a library comprising a plurality of fusion polypeptides of antigen binding domains linked to an IgG antibody light chain constant region, wherein the antigen binding domains include an antigen binding domain whose antigen binding activity can be inhibited or could be lost by associating with particular VL, VH or VHH, and a method for screening the library for an antigen binding domain whose antigen binding activity can be inhibited or could be lost by associating with particular VL, VH or VHH.

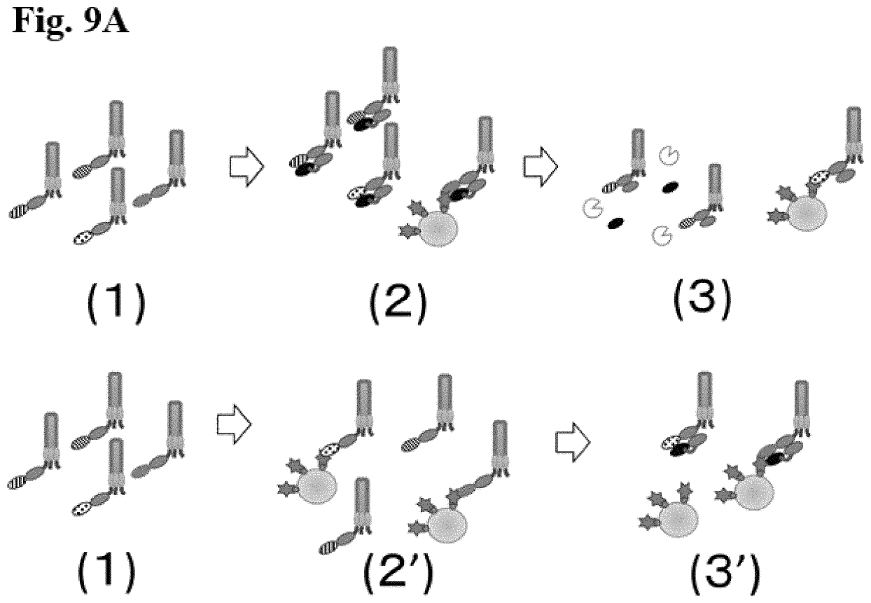

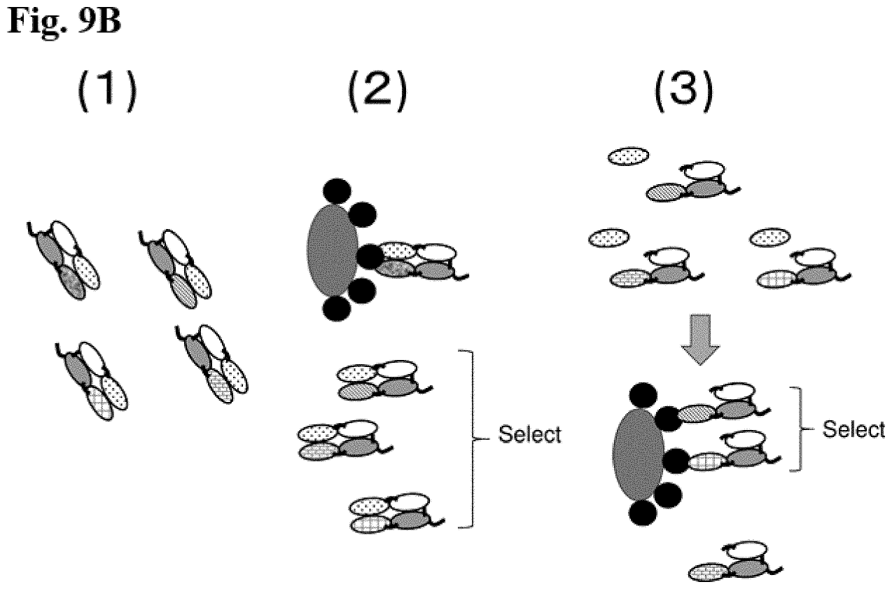

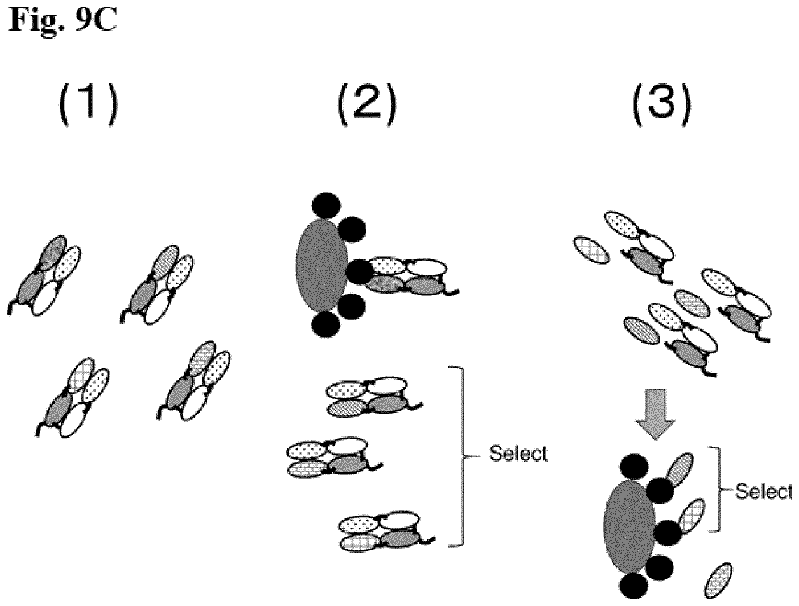

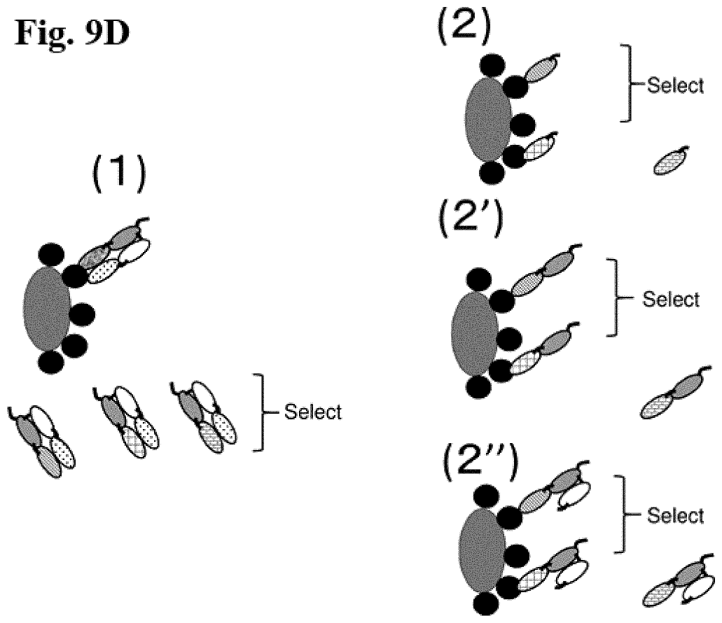

In a specific embodiment, as shown in FIGS. 9A(1), 9A(2), 9A(3), 9B, and 9C,
(1) fusion polypeptides of antigen binding domains each linked to a first association sustaining domain are displayed on the surface of phages or the like by a display method such as phage display.
(2) An association partner of an inhibiting domain linked to a second association sustaining domain is provided, and the fusion polypeptides are associated with the association partner. A fusion polypeptide that does not bind to the target antigen or has antigen binding activity of a predetermined value or lower in this state of the fusion polypeptide associated with the association partner is selected.
(3) The association of the antigen binding domain in the fusion polypeptide selected in (2) with the inhibiting domain in the association partner is canceled. A fusion polypeptide that binds to the target antigen or has antigen binding activity of a predetermined value or higher in a state where the antigen binding domain does not associate with the inhibiting domain is selected.

In this context, for example, a method of cleaving the association partner near the boundary between the inhibiting domain and the second association sustaining domain as shown in FIG. 9B, or a method of cleaving the fusion polypeptide near the boundary between the antigen binding domain and the first association sustaining domain as shown in FIG. 9C can be used as a method for canceling the association of the antigen binding domain with the inhibiting domain.

In a further embodiment, the present invention provides a method comprising, as shown in FIG. 9D, comparing the difference in the binding activity of the antigen binding domain between when the antigen binding domain and the inhibiting domain are expressed together and when the antigen binding domain is expressed so as not to express the inhibiting domain together therewith, instead of comparing the difference in the binding activity of the antigen binding domain between the canceled association and non-canceled association of the antigen binding domain with the inhibiting domain as shown in FIGS. 9A to 9C.

As shown in FIG. 9D(1), the antigen binding domain and the inhibiting domain are expressed together to form association. A fusion polypeptide comprising an antigen binding domain that does not bind to the antigen or has antigen binding activity of a predetermined value or lower in this state is selected. As shown in FIGS. 9D(2), 9D(2′), and 9D(2″), the antigen binding domain is expressed so as not to express the inhibiting domain together therewith. A fusion polypeptide comprising an antigen binding domain that binds to the antigen or has antigen binding activity of a predetermined value or higher in this state is selected. As a result, the antigen binding domain whose antigen binding activity can be inhibited or could be lost by associating with a particular inhibiting domain, for example, VH, VL or VHH may be screened for from the library comprising a plurality of fusion polypeptides of antigen binding domains each linked to a first association sustaining domain. Alternatively, the antigen binding domain is expressed so as not to express the inhibiting domain together therewith. A polypeptide comprising an antigen binding domain that binds to the antigen or has antigen binding activity of a predetermined value or higher in this state is selected. Then, the antigen binding domain and the inhibiting domain are expressed together to form association. A polypeptide comprising an antigen binding domain that does not bind to the antigen or has antigen binding activity of a predetermined value or lower in this state is selected. By this method as well, the antigen binding domain whose antigen binding activity can be inhibited or could be lost by associating with a particular inhibiting domain, for example, VH, VL or VHH may be screened for from the library comprising a plurality of fusion polypeptides of antigen binding domains each linked to a first association sustaining domain. Alternatively, as shown in FIGS. 9D(2), 9D(2′), and 9D(2″), the antigen binding domain is expressed so as not to express the inhibiting domain together therewith (only the antigen binding domain is expressed; only the fusion polypeptide comprising an antigen binding domain and a first association sustaining domain is expressed; or the fusion polypeptide comprising an antigen binding domain and a first association sustaining domain is associated only with the second association sustaining domain), and a fusion polypeptide comprising an antigen binding domain that binds to the antigen or has antigen binding activity of a predetermined value or higher in this state is selected. Then, as shown in FIG. 9D(1), the antigen binding domain in the selected fusion polypeptide and the inhibiting domain are expressed together to form association. A fusion polypeptide comprising an antigen binding domain that does not bind to the antigen or has antigen binding activity of a predetermined value or lower in this state is selected. As a result, the antigen binding domain whose antigen binding activity can be inhibited or could be lost by associating with a particular inhibiting domain, for example, VH, VL or VHH may also be screened for from the library comprising a plurality of fusion polypeptides of antigen binding domains each linked to a first association sustaining domain.

The “antigen binding activity of a predetermined value or lower” can refer to, for example, antigen binding activity that falls below a predetermined reference when the antigen binding activity is measured by the method listed in the present specification. Likewise, the “antigen binding activity of a predetermined value or higher” can refer to, for example, antigen binding activity that exceeds a predetermined reference when the antigen binding activity is measured by the method listed in the present specification. A fusion polypeptide having the antigen binding activity of a predetermined value or higher binds more strongly to the antigen than a fusion polypeptide having the antigen binding activity of a predetermined value or lower.

The fusion polypeptide selected in (3) described above comprises an antigen binding domain that has no or weak antigen binding activity in a state of association with the inhibiting domain and has (strong) antigen binding activity in a state of non-association with the inhibiting domain. The sequence of the fusion polypeptide selected by such a method can be analyzed to also elucidate the sequence of the antigen binding domain contained therein. Thus, the antigen binding domain can be produced.

For the method for screening for a fusion polypeptide comprising the antigen binding domain of interest by using fusion polypeptides and an association partner, it is important to compare the antigen binding activity of the antigen binding domain between states of association and non-association with the inhibiting domain. As shown in FIGS. 9A(2′) and 9A(3′), the antigen binding activity of the displayed fusion polypeptides is first confirmed, and a fusion polypeptide that binds to the antigen or has antigen binding activity of a predetermined value or higher is selected. Then, the fusion polypeptides thus selected are associated with the association partner. A fusion polypeptide that does not binds to the antigen or has antigen binding activity of a predetermined value or lower in this state of association is selected. By this method as well, the fusion polypeptide comprising the antigen binding domain of interest can be obtained.

Hereinafter, some embodiments using an IgG antibody CH1 domain as the first association sustaining domain and using IgG antibody CL as the second association sustaining domain will be described.

A fusion polypeptide comprising the antigen binding domain of interest can be screened for from a library comprising a plurality of fusion polypeptides of antigen binding domains each linked to an IgG antibody CH1 domain.

In some embodiments, the present invention provides a library comprising a plurality of fusion polypeptides of antigen binding domains each linked to an IgG antibody CH1 domain, wherein the antigen binding domains include an antigen binding domain whose antigen binding activity is can be inhibited or could be lost by associating with particular VL, VH or VHH, and a method for screening the library for a fusion polypeptide comprising an antigen binding domain whose antigen binding activity can be inhibited or could be lost by associating with particular VL, VH or VHH.

In a particular embodiment, the present invention provides a method for screening for a fusion polypeptide comprising an antigen binding domain whose antigen binding activity can be inhibited or could be lost by associating with particular VL, from a library comprising a plurality of fusion polypeptides of antigen binding domains each linked to an IgG antibody CH1 domain. Specifically, the present invention provides a method for screening for an antigen binding domain, comprising the following steps:
(a) in vitro displaying the fusion polypeptides of the library according to the present invention;
(b) providing an association partner of an IgG antibody light chain constant region fused with the particular VL;
(c) associating the fusion polypeptides displayed in the step (a) with the association partner provided in the step (b) and selecting a fusion polypeptide that does not bind to the antigen or has antigen binding activity of a predetermined value or lower in a state where the antigen binding domain associates with the VL; and
(d) selecting, from the fusion polypeptides thus selected in the step (c), a fusion polypeptide that binds to the antigen or has antigen binding activity of a predetermined value or higher in a state where the antigen binding domain contained therein does not associate with the VL.

The association partner provided in the step (b) further comprises a protease cleavage sequence. In this case, in the step (d), the association of the antigen binding domain with the VL is canceled by protease treatment, and the antigen binding activity of the antigen binding domain may be confirmed in a state where the antigen binding domain does not associate with the VL. The protease cleavage sequence in the association partner is not limited by its position as long as the association of the antigen binding domain with the VL is canceled by cleavage. As an example of the position, the protease cleavage sequence may be located, for example, near the boundary between the VL and the IgG antibody light chain constant region in the association partner, preferably at any position between amino acid position 96 (Kabat numbering) of the VL and amino acid position 130 (EU numbering) (Kabat numbering position 130) of the antibody light chain constant region, more preferably at any position between amino acid position 104 (Kabat numbering) of the VL and amino acid position 113 (EU numbering) (Kabat numbering position 113) of the antibody light chain constant region.

Instead of using the association partner comprising a protease cleavage sequence, the protease cleavage sequence may be introduced into the fusion polypeptides in the library, and the fusion polypeptides can be cleaved by protease so that the association of the antigen binding domain with the VL is canceled. The protease cleavage sequence in each fusion polypeptide is not limited by its position as long as the association of the antigen binding domain with the VL is canceled by cleavage and the antigen binding domain retains its antigen binding activity even after the cleavage. As an example of the position, the protease cleavage sequence may be located, for example, near the boundary between the antigen binding domain and the IgG antibody CH1 domain in the fusion polypeptide.

In the step (d), the full lengths of the fusion polypeptides selected in the step (c) or their moieties comprising the antigen binding domains may be displayed again, and the antigen binding activity of the antigen binding domain can be confirmed in a state where the antigen binding domain does not associate with the VL.

In a particular embodiment, the present invention provides a method for screening for a fusion polypeptide comprising an antigen binding domain whose antigen binding activity can be inhibited or could be lost by associating with particular VH, from a library comprising a plurality of fusion polypeptides of antigen binding domains each linked to an IgG antibody light chain constant region. Specifically, the present invention provides a method for screening for a fusion polypeptide comprising an antigen binding domain, comprising the following steps:
(a) in vitro displaying the fusion polypeptides of the library according to the present invention;
(b) providing an association partner of an IgG antibody CH1 domain fused with the particular VH;
(c) associating the fusion polypeptides displayed in the step (a) with the association partner provided in the step (b) and selecting a fusion polypeptide that does not bind to the antigen or has antigen binding activity of a predetermined value or lower in a state where the antigen binding domain associates with the VH; and
(d) selecting, from the fusion polypeptides thus selected in the step (c), a fusion polypeptide that binds to the antigen or has antigen binding activity of a predetermined value or higher in a state where the antigen binding domain contained therein does not associate with the VH.

The association partner provided in the step (b) further comprises a protease cleavage sequence. In this case, in the step (d), the association of the antigen binding domain with the VH is canceled by protease treatment, and the antigen binding activity of the antigen binding domain may be confirmed in a state where the antigen binding domain does not associate with the VH. The protease cleavage sequence in the association partner is not limited by its position as long as the association of the antigen binding domain with the VH is canceled by cleavage. As an example of the position, the protease cleavage sequence may be located, for example, near the boundary between the VH and the IgG antibody CH1 domain in the association partner, preferably at any position between amino acid position 101 (Kabat numbering) of the VH and amino acid position 140 (EU numbering) of the antibody heavy chain constant region, more preferably at any position between amino acid position 109 (Kabat numbering) of the VH and amino acid position 122 (EU numbering) of the antibody heavy chain constant region.

Instead of using the association partner comprising a protease cleavage sequence, the protease cleavage sequence may be introduced into the fusion polypeptides in the library, and the fusion polypeptides can be cleaved by protease so that the association of the antigen binding domain with the VH is canceled. The protease cleavage sequence in each fusion polypeptide is not limited by its position as long as the association of the antigen binding domain with the VH is canceled by cleavage and the antigen binding domain retains its antigen binding activity even after the cleavage. As an example of the position, the protease cleavage sequence may be located, for example, near the boundary between the antigen binding domain and the IgG antibody light chain constant region in the fusion polypeptide.

In the step (d), the full lengths of the fusion polypeptides selected in the step (c) or their moieties comprising the antigen binding domains may be displayed again, and the antigen binding activity of the antigen binding domain can be confirmed in a state where the antigen binding domain does not associate with the VH.

An amino acid contained in each amino acid sequence described in the present invention may be posttranslationally modified (e.g., the modification of N-terminal glutamine to pyroglutamic acid by pyroglutamylation is a modification well known to those skilled in the art). Such an amino acid sequence containing the posttranslationally modified amino acid is also included in the amino acid sequence described in the present invention, as a matter of course.

It should be understood by those skilled in the art that arbitrary combinations of one or more embodiments described in the present specification are also included in the present invention unless there is technical contradiction on the basis of the technical common sense of those skilled in the art.

### EXAMPLES

Hereinafter, Examples of the method and the composition of the present invention will be described. It shall be understood that various other embodiments can be carried out in light of the general description mentioned above.

### Example 1: Problem of Existing Protease-Activated Antibody

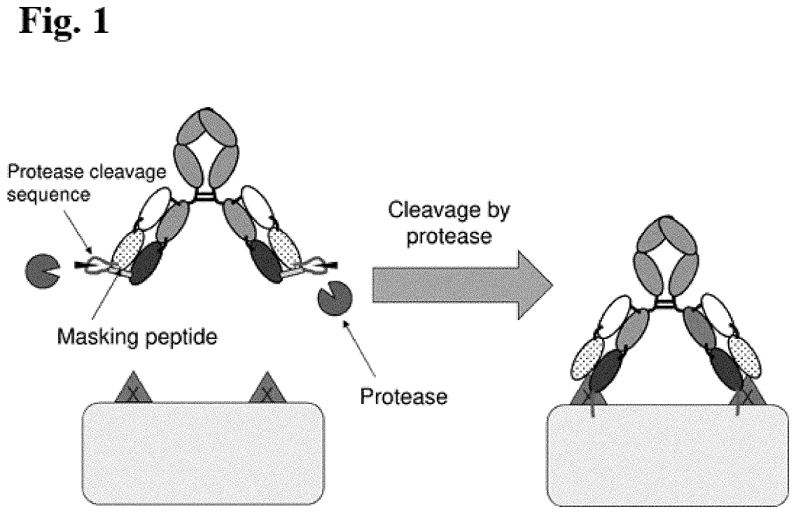

A method for preparing an antibody that exerts antigen binding activity only through cleavage by protease expressed at a lesion site such as a cancer tissue or an inflammatory tissue has been reported. This antibody, called Probody, is an antibody molecule, as shown in FIG. 1, whose antigen binding activity is inhibited by connecting an antibody to a peptide masking the antigen binding site of the antibody via a linker that is cleaved by protease expressed at a lesion site (Non Patent Literature 18). The masking peptide is dissociated from the Probody by the cleavage of the constituent linker by the protease expressed at the target pathological site so that the resulting antibody molecule restores its antigen binding activity and becomes capable of binding to the antigen in the target pathological tissue.

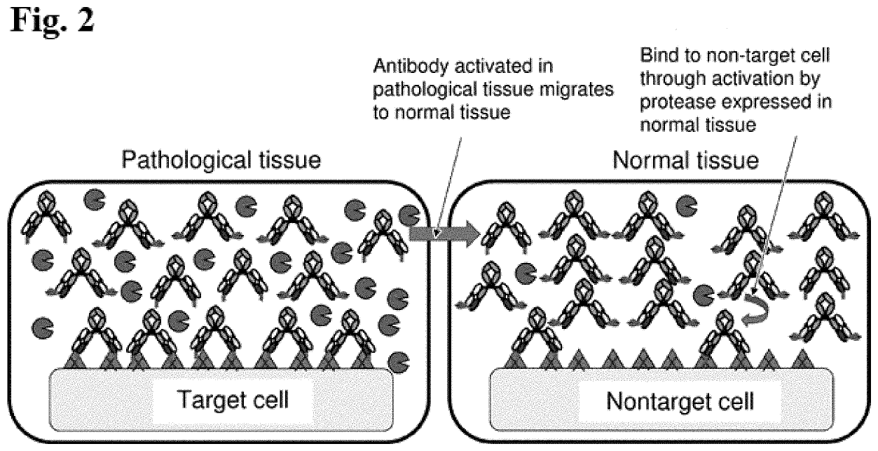

It is believed that the Probody can bind to the antigen selectively at the target pathological site under the mechanism as mentioned above and thereby expand the therapeutic window. However, for the Probody, there may be the possibility that the antibody cleaved at the pathological site is capable of being brought back into blood from the pathological site and binds to the antigen expressed in normal tissue as a result of distributing the antibody to the normal tissues through blood flow, because the cleavage of the antibody by protease is irreversible. The Probody activated by protease retains a Fc region as in the Probody before the activation and therefore possesses a long circulation time in blood. Therefore, the antibody activated by protease expressed at a pathological site might circulate long in blood. Even protease expressed at an elevated level at a pathological site is also expressed at a low level in normal tissues, and free protease produced at a pathological site may be leaked into blood (The Chinese-German Journal of Clinical Oncology June 2004, Vol. 3, No. 2 P78-P80). Therefore, the Probody may be activated by such free protease. Hence, there may be a possibility that the Probody is activated at a site other than a pathological site. The Probody thus activated also circulates long in blood. Thus, there is a possibility that the Probody is continuously activated at a pathological site, in normal tissues, and in blood, and the activated Probody, if having a long circulation time in blood, accumulates in blood. The activated Probody accumulated in blood might exhibit adverse reactions by binding to the antigen expressed in normal tissues (FIG. 2).

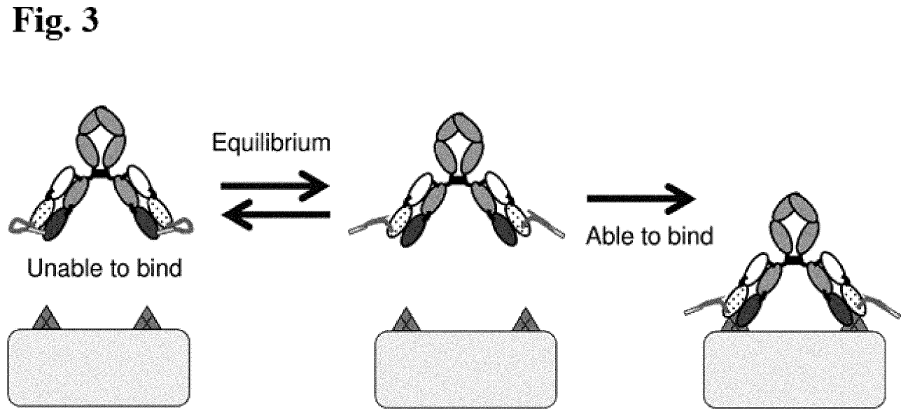

The antigen binding activity of the Probody is inhibited by a masking peptide linked to an antibody via a linker, but is not completely inhibited. The Probody is in equilibrium between a state where the masking peptide linked via the linker is bound with the antigen binding site and a state where the masking peptide is dissociated. A molecule in the dissociated state can bind to the antigen (FIG. 3). In actuality, anti-EGFR Probody described in Non Patent Literature 17 has binding activity against EGFR even before protease cleavage of the linker. Although 30 to 100-fold rise in binding activity is seen by the protease cleavage of the linker, the Probody present at a high concentration before activation might exhibit adverse reactions by binding to the antigen expressed in normal tissues, because the Probody before activation has 1/30 to 1/100 of the binding activity of the activated Probody.

The Probody employs an artificial peptide for masking the antigen binding site of the antibody. The artificial peptide has a sequence absent in natural human proteins and might therefore has immunogenicity in humans Such immunogenicity is known to decrease the effects of antibody drugs by inducing anti-drug antibodies (Blood. 2016 Mar. 31; 127 (13): 1633-41).

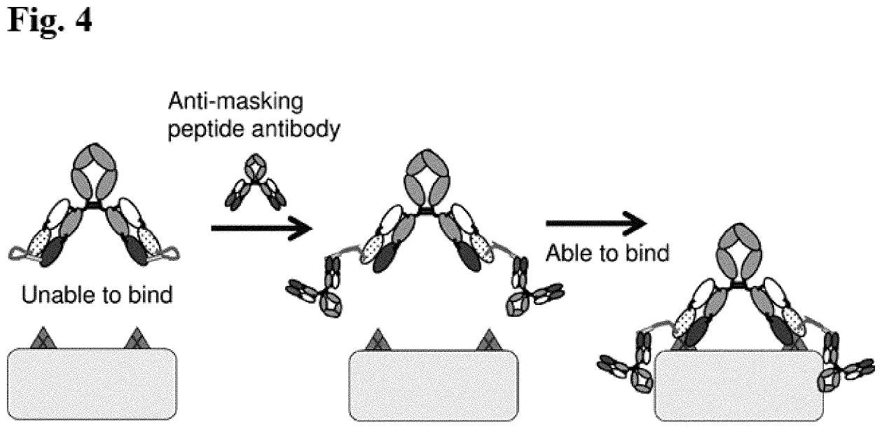

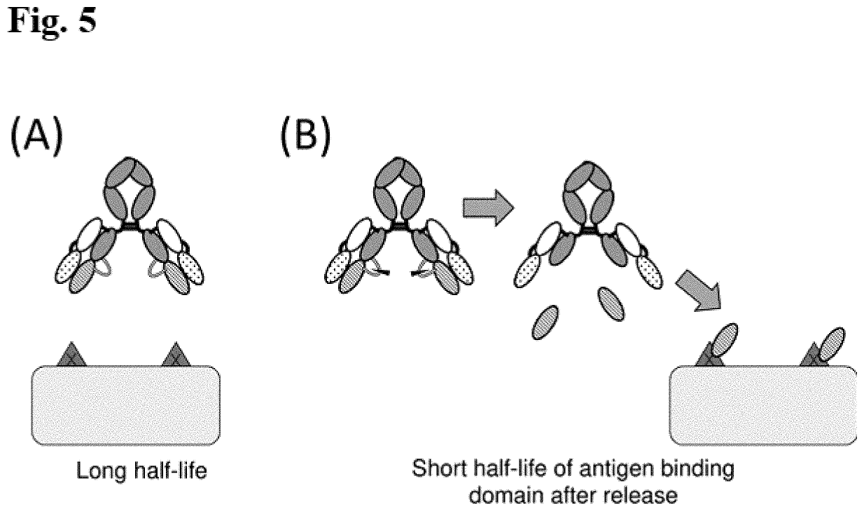

Possible anti-drug antibodies against Probody are an anti-drug antibody against a complex of the antibody and the masking peptide (Probody before activation), an anti-drug antibody against the antibody dissociated from the masking peptide (activated Probody), an anti-drug antibody against the masking peptide (masking peptide dissociated from the activated Probody), and the like. Among them, the anti-drug antibody against the masking peptide (anti-masking peptide antibody) might bind to the masking peptide of Probody before activation and thereby activate the Probody without protease cleavage (FIG. 4). The Probody activated by the anti-masking peptide antibody might exhibit adverse reactions by binding to the antigen expressed in normal tissues.

### Example: 2 Concept of Protease-Activated Polypeptide Comprising Single-Domain Antibody

As shown in Example 1, the Probody technology presents the following problems:
1. Probody activated by protease cleavage has a long circulation time in blood.
2. Even Probody before protease cleavage has binding activity against the antigen.
3. The masking peptide is an artificial non-human sequence and may induce an anti-masking peptide antibody.

The present inventors thought that a useful way for solving these problems and providing an antibody drug exerting activity at a pathological site is to satisfy the following conditions:
1. An antigen binding domain activated by protease cleavage has a short half-life in blood.
2. The antigen binding activity of a molecule before protease cleavage is minimized
3. The masking peptide having an artificial non-human sequence is not used.

The present inventors devised a molecule shown in FIG. 5 as one example of a polypeptide that satisfied the conditions described above. The polypeptide with an antigen binding domain linked to a carrying moiety has a long half-life and does not bind to the antigen because the antigen binding activity of the antigen binding domain is inhibited (A). The antigen binding domain is released, and the antigen binding domain thus released restores its antigen binding activity and also has a short half-life (B).

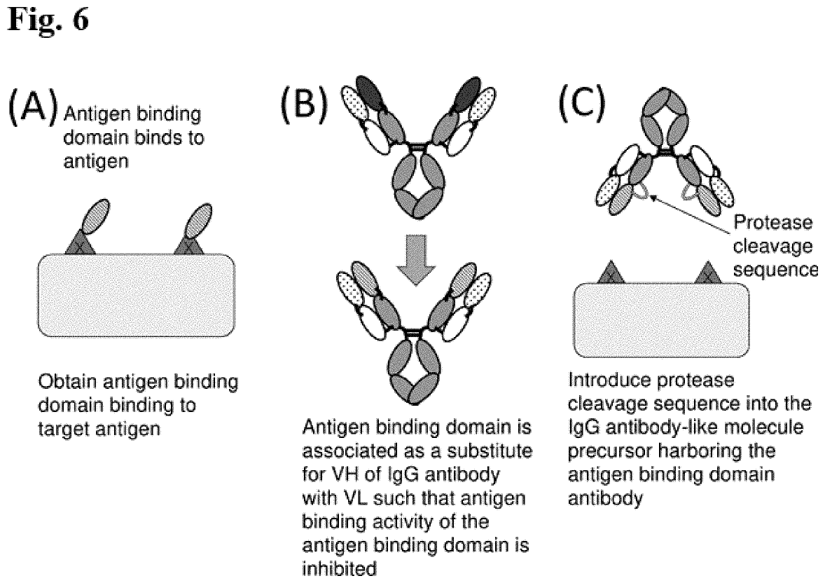

The polypeptide shown in FIG. 5 has various variations. In the case of using an IgG antibody-like molecule, the polypeptide may be produced by a production method as illustrated in FIG. 6. First, a single-domain antibody (e.g., VH or VHH) binding to the target antigen is obtained (A). The obtained single-domain antibody is associated, as a substitute for one of VH and VL of an IgG antibody having a germline sequence, with the other one (VL or VH) to form an IgG antibody-like molecule (B). A protease cleavage sequence is introduced into the IgG antibody-like molecule (C). Examples of the introduction position include a position near the boundary between the harbored single-domain antibody (VH or VHH) and the constant region (CH1 or CL).

The single-domain antibody has antigen binding activity when existing alone, but loses its antigen binding activity upon formation of a variable region with VL, VH, VHH, or the like. VL or VH is a natural human antibody sequence having a germline sequence and therefore has a low risk of immunogenicity and is unlikely to induce an anti-drug antibody recognizing this VL or VH. In the case of forming a variable region of the single-domain antibody with VHH, the humanization of the VHH reduces the risk of immunogenicity and reduces the likelihood of inducing an anti-drug antibody recognizing this humanized VHH. The protease cleavage sequence inserted into the IgG antibody-like molecule is cleaved by protease so that the single-domain antibody is released. The released single-domain antibody has antigen binding activity. The IgG antibody-like molecule before protease cleavage is structurally similar to general IgG molecules and therefore has a long circulation time in blood, whereas the single-domain antibody released by protease cleavage has a molecular weight of approximately 13 kDa without retaining a Fc region and therefore disappears rapidly by renal excretion. In actuality, the half-life of full-length IgG is on the order of 2 to 3 weeks (Blood. 2016 Mar. 31; 127 (13): 1633-41), whereas the half-life of the single-domain antibody is approximately 2 hours (Antibodies 2015, 4 (3), 141-156). Hence, the antigen binding molecule activated by protease has a short half-life in blood and becomes unlikely to bind to the antigen in normal tissues.

When the single-domain antibody is VL, the same concept as above may be achieved, for example, by introducing the protease cleavage sequence to near the boundary between VL and CL.

### Example 3: Preparation of Protease-Activated Polypeptide Using VHH Binding to IL6R

3-1 Preparation of Polypeptide with Incorporated VHH Binding to IL6R

An expression vector encoding IL6R90-G1m (SEQ ID NO: 2) containing IL6R90 (SEQ ID NO: 1), VHH having binding and neutralizing activities against human IL6R as described in International Publication No. WO2010/115998, fused with a human IgG1 constant region (CH1-hinge-CH2-CH3) was prepared by a method known to those skilled in the art.

Expression vectors encoding VK1-39-k0MT (SEQ ID NO: 3), VK2-28-k0MT (SEQ ID NO: 4), VK3-20-k0MT (SEQ ID NO: 5), VL1-40-lamL (SEQ ID NO: 6), VL1-44-lamL (SEQ ID NO: 7), VL2-14-lamL (SEQ ID NO: 8), VL3-21-lamL (SEQ ID NO: 9), k0 (SEQ ID NO: 10), and lamL (SEQ ID NO: 11) as light chains (variable region-constant region) of various subclasses having a human germline sequence were prepared by a method known to those skilled in the art.

IgG antibody-like molecules IL6R90-G1m/VK1-39-k0MT (heavy chain: SEQ ID NO: 2, light chain: SEQ ID NO: 3), IL6R90-G1m/VK2-28-k0MT (heavy chain: SEQ ID NO: 2, light chain: SEQ ID NO: 4), IL6R90-G1m/VK3-20-k0MT (heavy chain: SEQ ID NO: 2, light chain: SEQ ID NO: 5), IL6R90-G1m/VL1-40-lamL (heavy chain: SEQ ID NO: 2, light chain: SEQ ID NO: 6), IL6R90-G1m/VL1-44-lamL (heavy chain: SEQ ID NO: 2, light chain: SEQ ID NO: 7), IL6R90-G1m/VL2-14-lamL (heavy chain: SEQ ID NO: 2, light chain: SEQ ID NO: 8), IL6R90-G1m/VL3-21-lamL (heavy chain: SEQ ID NO: 2, light chain: SEQ ID NO: 9), IL6R90-G1m/k0 (heavy chain: SEQ ID NO: 2, light chain: SEQ ID NO: 10), and IL6R90-G1m/lamL (heavy chain: SEQ ID NO: 2, light chain: SEQ ID NO: 11) were expressed by transient expression using FreeStyle 293 cells (Invitrogen Corp.) by a method known to those skilled in the art, and purified by a method known to those skilled in the art using protein A.

3-2 IL6R Binding Evaluation of Polypeptide with Incorporated VHH Binding to Human IL6R

IL6R90-G1m/VK1-39-k0MT, IL6R90-G1m/VK2-28-k0MT, IL6R90-G1m/VK3-20-k0MT, IL6R90-G1m/VL1-40-lamL, IL6R90-G1m/VL1-44-lamL, IL6R90-G1m/VL2-14-lamL, IL6R90-G1m/VL3-21-lamL, IL6R90-G1m/k0, and IL6R90-G1m/lamL were evaluated for their binding activity against human IL6R by the following method.

Recombinant human IL6R used as an antigen was prepared as follows: a CHO line stably expressing soluble human IL-6R (hereinafter, also referred to as hsIL-6R, IL6R or IL-6R) consisting of an amino acid sequence from positions 1 to 357 counted from the N terminus as reported in J. Immunol. 152, 4958-4968 (1994) was constructed by a method known to those skilled in the art, cultured, and caused to express hsIL-6R. From the obtained culture supernatant, hsIL-6R was purified by 2 steps of Blue Sepharose 6 FF column chromatography and gel filtration column chromatography. A fraction eluted as a main peak in the final step was used as a final purified product.

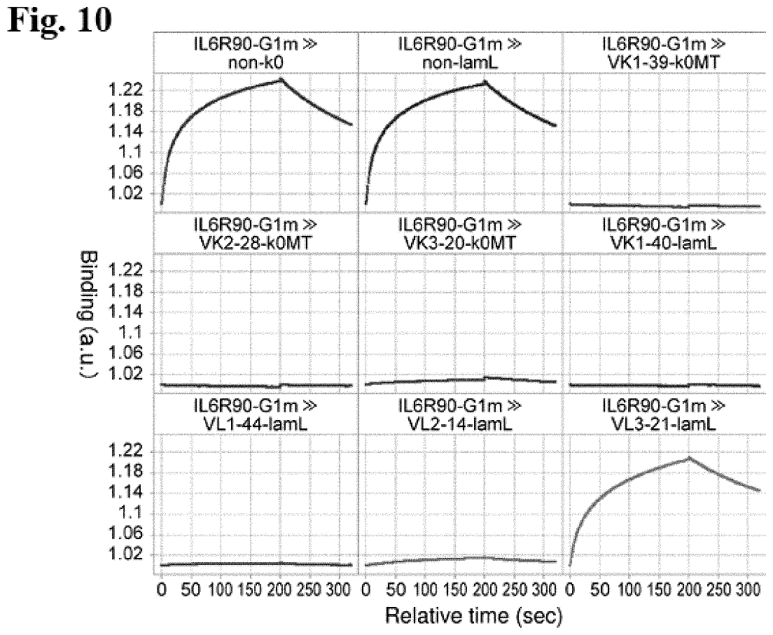

The hsIL-6R binding evaluation of each molecule was conducted using Octet HTX (Pall ForteBio Corp.). Specifically, each molecule was bound to Biosensor/Protein A (ProA) (Pall ForteBio Corp., 18-5013), and hsIL-6R was allowed to act thereon, followed by binding evaluation at 30 degrees C. Sensorgrams showing continuous responses measured using Octet HTX are shown in FIG. 10. IL6R90-G1m/k0 and IL6R90-G1m/lamL lacking VL bound to hsIL-6R, whereas IL6R90-G1m/VK1-39-k0MT, IL6R90-G1m/VK2-28-k0MT, IL6R90-G1m/VK3-20-k0MT, IL6R90-G1m/VL1-40-lamL, IL6R90-G1m/VL1-44-lamL, and IL6R90-G1m/VL2-14-lamL containing a variable region formed with VL were shown to be unable to bind to hsIL-6R. From this, it was found that VHH having binding activity against human IL6R can lose its IL6R binding activity by forming a variable region through association with VL.

3-3 Introduction of Protease Cleavage Sequence to Polypeptide with Incorporated VHH Binding to IL6R

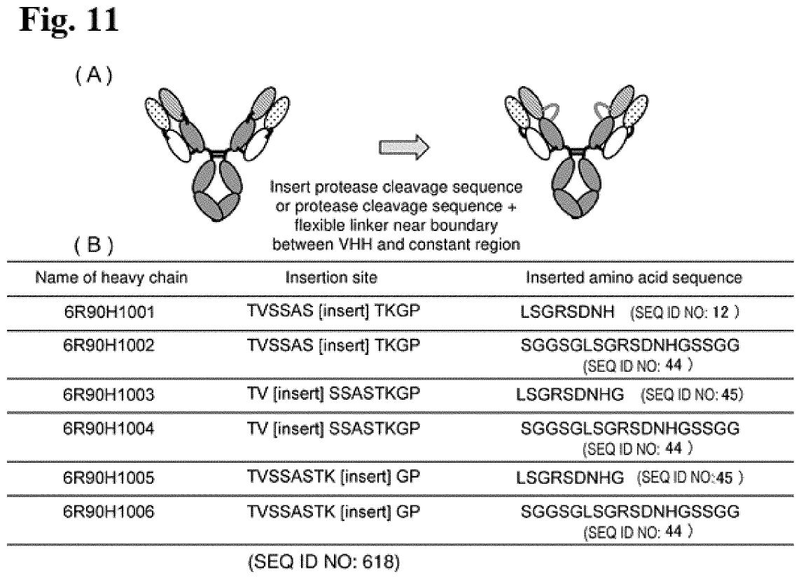

A protease cleavage sequence was inserted to near the boundary between the anti-human IL6R VHH IL6R90 and CH1. Six types of heavy chains shown in FIG. 11 were designed such that peptide sequence A (SEQ ID NO: 12), a reported sequence cleavable by cancer-specifically expressed urokinase (uPA) and MT-SP1, was inserted at 3 sites near the boundary between IL6R90 and CH1 with or without a glycine-serine linker. Expression vectors encoding IL6R90H1001 (SEQ ID NO: 13), IL6R90H1002 (SEQ ID NO: 14), IL6R90H1003 (SEQ ID NO: 15), IL6R90H1004 (SEQ ID NO: 16), IL6R90H1005 (SEQ ID NO: 17), and IL6R90H1006 (SEQ ID NO: 18) were prepared by a method known to those skilled in the art.

IgG antibody-like molecules IL6R90H1001/VK1-39-k0MT (heavy chain: SEQ ID NO: 13, light chain: SEQ ID NO: 3), IL6R90H1002/VK1-39-k0MT (heavy chain: SEQ ID NO: 14, light chain: SEQ ID NO: 3), IL6R90H1003/VK1-39-k0MT (heavy chain: SEQ ID NO: 15, light chain: SEQ ID NO: 3), IL6R90H1004/VK1-39-k0MT (heavy chain: SEQ ID NO: 16, light chain: SEQ ID NO: 3), IL6R90H1005/VK1-39-k0MT (heavy chain: SEQ ID NO: 17, light chain: SEQ ID NO: 3), and IL6R90H1006/VK1-39-k0MT (heavy chain: SEQ ID NO: 18, light chain: SEQ ID NO: 3) were expressed by transient expression using these heavy chains and VK1-39-k0MT (SEQ ID NO: 3) as light chain and using FreeStyle 293 cells (Invitrogen Corp.) by a method known to those skilled in the art, and purified by a method known to those skilled in the art using protein A.

### 3-4 Activation of Polypeptide Harboring Protease Cleavage Sequence by Protease Cleavage

Whether IL6R90H1001/VK1-39-k0MT, IL6R90H1002/VK1-39-k0MT, IL6R90H1003/VK1-39-k0MT, IL6R90H1004/VK1-39-k0MT, IL6R90H1005/VK1-39-k0MT, and IL6R90H1006/VK1-39-k0MT would release VHH having binding activity against IL6R by protease cleavage was verified.

Soluble human IL6R was prepared by a method known to those skilled in the art. The prepared soluble human IL6R was biotinylated by a method known to those skilled in the art.

For the purpose of attaching biotin to the C terminus of soluble human IL-6R (also referred to as hsIL-6R or soluble human IL6R; SEQ ID NO: 35), a gene fragment encoding a specific sequence (AviTag sequence; SEQ ID NO: 36) to be biotinylated by biotin ligase was linked via a gene fragment encoding a linker to downstream of a gene fragment encoding hsIL-6R. A gene fragment encoding a protein containing hsIL-6R linked to the AviTag sequence (hsIL-6R-Avitag; SEQ ID NO: 37) was integrated to a vector for expression in animal cells. The constructed plasmid vector was transfected to FreeStyle 293 cells (Invitrogen Corp.) using 293Fectin (Invitrogen Corp.). In this operation, the cells were cotransfected with a gene for EBNA1 (SEQ ID NO: 57) expression and a gene for biotin ligase (BirA; SEQ ID NO: 58) expression, and biotin was further added thereto for the purpose of biotin-labeling hsIL-6R-Avitag. The cells transfected according to the procedures mentioned above were cultured at 37 degrees C. under 8% CO₂ and the protein of interest (hsIL-6R-BAP1) was secreted into the culture supernatant. This cell culture solution was filtered through a 0.22 micro m bottle-top filter to obtain a culture supernatant.

An anti-human IL-6R antibody was immobilized on HiTrap NHS-activated HP (GE Healthcare Japan Corp.) according to the protocol of the manufacturer to prepare a column (anti-human IL-6R antibody column). The culture supernatant was applied to the anti-human IL-6R antibody column equilibrated with TBS, followed by the elution of the bound hsIL-6R with 2 M arginine (pH 4.0). Next, the eluate from the anti-human IL-6R antibody column was diluted with TBS and then applied to SoftLink Avidin column (Promega Corp.) equilibrated with TBS, followed by the elution of hsIL-6R-BAP1 with 5 mM biotin, 50 mM Tris-HCl (pH 8.0) and 2 M arginine (pH 4.0). From this eluate, aggregates of hsIL-6R-BAP1 were removed by gel filtration chromatography using Superdex 200 (GE Healthcare Japan Corp.) to obtain purified hsIL-6R-BAP1 with the buffer exchanged with D-PBS and 0.05% CHAPS.

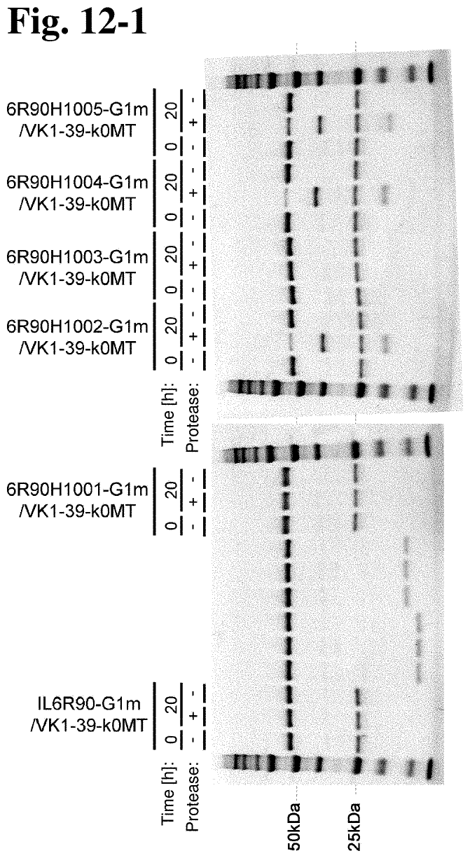

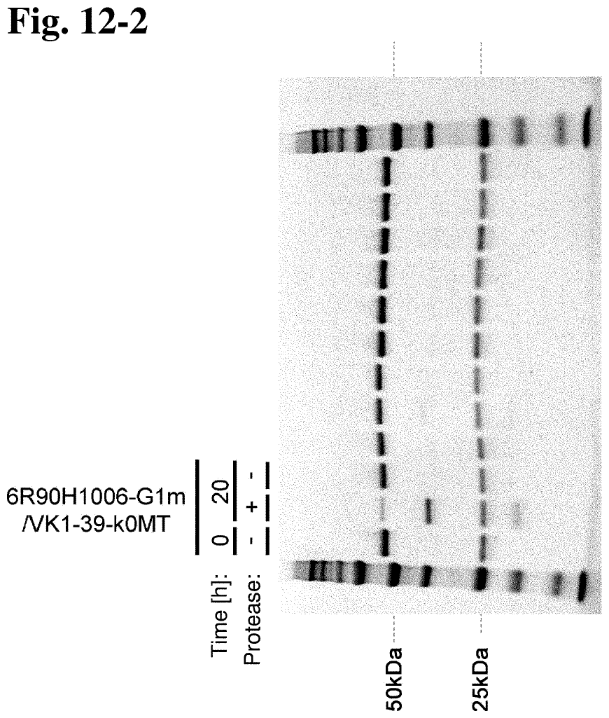

Recombinant Human Matriptase/ST14 Catalytic Domain (R&D Systems, Inc., 3946-SE-010) was used as the protease. 12.5 nM protease and 100 micro g/mL of each IgG antibody-like molecule were incubated in PBS under a condition of 37 degrees C. for 20 hours. Then, cleavage by the protease was evaluated by reducing SDS-PAGE. The results are shown in FIG. 12. As a result, the protease cleavage of the protease cleavage sequence near the boundary between the VHH and the heavy chain constant region was confirmed in IL6R90H1002/VK1-39-k0MT, IL6R90H1004/VK1-39-k0MT, IL6R90H1005/VK1-39-k0MT, and IL6R90H1006/VK1-39-k0MT.

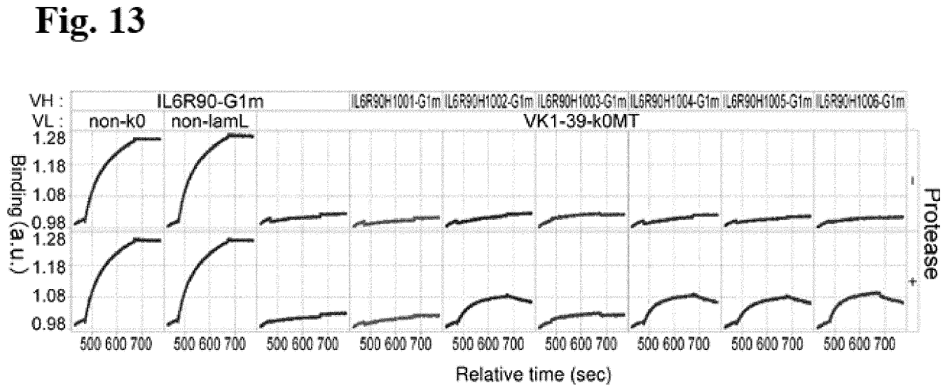

Next, the IL6R binding evaluation of VHH released by protease treatment was conducted using Octet HTX (Pall ForteBio Corp.). Specifically, hsIL-6R-BAP1 was bound to a streptavidin sensor (Pall ForteBio Corp., 18-5021), and each cleaved IgG antibody-like molecule was allowed to act thereon, followed by binding evaluation at 30 degrees C. Sensorgrams showing continuous responses measured using Octet HTX are shown in FIG. 13. As a result, the binding was confirmed in IL6R90H1002/VK1-39-k0MT, IL6R90H1004/VK1-39-k0MT, IL6R90H1005/VK1-39-k0MT, and IL6R90H1006/VK1-39-k0MT. IL6R90-G1m/k0 and IL6R90-G1m/lamL divalently bound with avidity, whereas the released VHH bound with affinity. Therefore, the protease-treated IL6R90H1002/VK1-39-k0MT, IL6R90H1004/VK1-39-k0MT, IL6R90H1005/VK1-39-k0MT, and IL6R90H1006/VK1-39-k0MT exhibited a faster dissociation rate from IL6R than that of IL6R90-G1m/k0 and IL6R90-G1m/lamL Also, the VHH had a smaller molecular weight than that of IL6R90-G1m/k0 and IL6R90-G1m/lamL Therefore, its response was lower.

These results demonstrated that IL6R90H1002/VK1-39-k0MT, IL6R90H1004/VK1-39-k0MT, IL6R90H1005/VK1-39-k0MT, or IL6R90H1006/VK1-39-k0MT does not exhibit binding activity against IL6R as is, whereas the peptide sequence A inserted near the boundary between the VHH and the heavy chain constant region is cleaved by protease treatment so that the VHH domain is released, and the released VHH can bind to IL6R. From this, it was concluded that the molecule conforming to the concept described in Example 2 was actually able to be prepared.

### Example 4: Preparation of Protease-Activated Polypeptide by Alteration Using VHH Binding to IL6R

4-1. IL6R Binding Evaluation of Polypeptide with Incorporated VHH Binding to IL6R

An expression vector encoding 20A11-G1m (SEQ ID NO: 38) containing 20A11 (SEQ ID NO: 19), VHH having binding and neutralizing activities against IL6R as described in International Publication No. WO2010/115998, fused with a human IgG1 constant region (CH1-hinge-CH2-CH3) in the same way as in Example 3 was prepared by a method known to those skilled in the art.

Polypeptides 20A11-G1m/VK1-39-k0MT, 20A11-G1m/VK2-28-k0MT, 20A11-G1m/VK3-20-k0MT, 20A11-G1m/VL1-40-lamL, 20A11-G1m/VL1-44-lamL, 20A11-G1m/VL2-14-lamL, and 20A11-G1m/VL3-21-lamL were expressed and purified in the same way as in Example 3 using this heavy chain and VK1-39-k0MT (SEQ ID NO: 3), VK2-28-k0MT (SEQ ID NO: 4), VK3-20-k0MT (SEQ ID NO: 5), VL1-40-lamL (SEQ ID NO: 6), VL1-44-lamL (SEQ ID NO: 7), VL2-14-lamL (SEQ ID NO: 8), and VL3-21-lamL (SEQ ID NO: 9) as light chains.

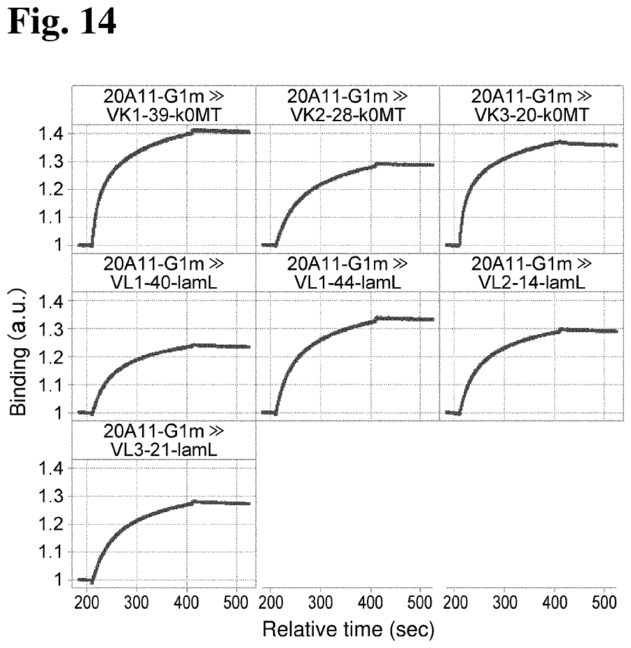

The obtained 20A11-G1m/VK1-39-k0MT (heavy chain: SEQ ID NO: 38, light chain: SEQ ID NO: 3), 20A11-G1m/VK2-28-k0MT (heavy chain: SEQ ID NO: 38, light chain: SEQ ID NO: 4), 20A11-G1m/VK3-20-k0MT (heavy chain: SEQ ID NO: 38, light chain: SEQ ID NO: 5), 20A11-G1m/VL1-40-lamL (heavy chain: SEQ ID NO: 38, light chain: SEQ ID NO: 6), 20A11-G1m/VL1-44-lamL (heavy chain: SEQ ID NO: 38, light chain: SEQ ID NO: 7), 20A11-G1m/VL2-14-lamL (heavy chain: SEQ ID NO: 38, light chain: SEQ ID NO: 8), and 20A11-G1m/VL3-21-lamL (heavy chain: SEQ ID NO: 38, light chain: SEQ ID NO: 9) were evaluated for their binding to IL6R in the same way as in Example 3. The results are shown in FIG. 14. As a result, none of the light chains used in this Example inhibited the IL6R binding activity of 20A11 by associating with the heavy chain containing the 20A11 fused with the human germline IgG1 constant region (CH1-hinge-CH2-CH3).

This is probably because 20A11 did not form a stable variable region with VL used in this Example.

4-2. Introduction of Amino Acid Alteration to Interface Site Between VHH and VL in Polypeptide with Incorporated VHH not Losing Antigen Binding

In order to form a stable variable region between 20A11 and VL, mutations were introduced to amino acids present at the interface between the 20A11 and the VL. An expression vector encoding 20A11hu-G1m (SEQ ID NO: 39) containing 20A11hu (derived from 20A11 by the introduction of mutations to substitute F at position 37 by V (F37V), R at position 45 by L, and G at position 47 by W (all according to the Kabat numbering)) (SEQ ID NO: 20) fused with a human IgG1 constant region (CH1-hinge-CH2-CH3) in the same way as in Example 3 was prepared by a method known to those skilled in the art.

Polypeptides 20A11hu-G1m/VK1-39-k0MT (heavy chain: SEQ ID NO: 39, light chain: SEQ ID NO: 3), 20A11hu-G1m/VK2-28-k0MT (heavy chain: SEQ ID NO: 39, light chain: SEQ ID NO: 4), 20A11hu-G1m/VK3-20-k0MT (heavy chain: SEQ ID NO: 39, light chain: SEQ ID NO: 5), 20A11hu-G1m/VL1-40-lamL (heavy chain: SEQ ID NO: 39, light chain: SEQ ID NO: 6), 20A11hu-G1m/VL1-44-lamL (heavy chain: SEQ ID NO: 39, light chain: SEQ ID NO: 7), 20A11hu-G1m/VL2-14-lamL (heavy chain: SEQ ID NO: 39, light chain: SEQ ID NO: 8), and 20A11hu-G1m/VL3-21-lamL (heavy chain: SEQ ID NO: 39, light chain: SEQ ID NO: 9) were expressed and purified in the same way as in Example 3 using this heavy chain and VK1-39-k0MT (SEQ ID NO: 3), VK2-28-k0MT (SEQ ID NO: 4), VK3-20-k0MT (SEQ ID NO: 5), VL1-40-lamL (SEQ ID NO: 6), VL1-44-lamL (SEQ ID NO: 7), VL2-14-lamL (SEQ ID NO: 8), and VL3-21-lamL (SEQ ID NO: 9) as light chains.

4-3. IL6R Binding Evaluation of Polypeptide with Incorporated VHH Containing Amino Acid Alteration at Interface Site Between the VHH and VL

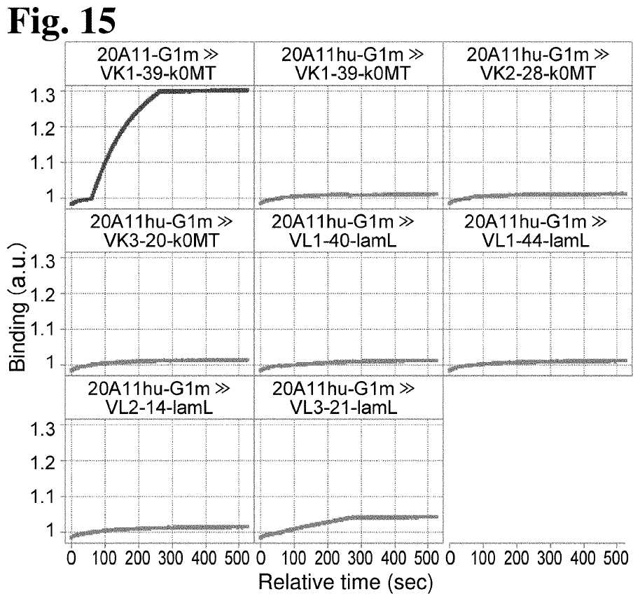

The obtained 20A11hu-G1m/VK1-39-k0MT, 20A11hu-G1m/VK2-28-k0MT, 20A11hu-G1m/VK3-20-k0MT, 20A11hu-G1m/VL1-40-lamL, 20A11hu-G1m/VL1-44-lamL, 20A11hu-G1m/VL2-14-lamL, and 20A11hu-G1m/VL3-21-lamL were evaluated for their binding to IL6R at 30 degrees C. or 25 degrees C. in the same way as in Example 3. The results are shown in FIG. 15.

As a result, 20A11hu-G1m/VK1-39-k0MT, 20A11hu-G1m/VK2-28-k0MT, 20A11hu-G1m/VK3-20-k0MT, 20A11hu-G1m/VL1-40-lamL, 20A11hu-G1m/VL1-44-lamL, and 20A11hu-G1m/VL2-14-lamL were shown to be unable to bind to IL6R.

These results demonstrated that the VHH 20A11, which did not lose its IL6R binding activity by associating with VL, used in Example 3, can form a stable variable region with VL and can lose its IL6R binding activity, by converting amino acids present at the interface site between the VHH and the VL to 37V, 45L, and 47W (Kabat numbering) and thereby altering the 20A11 to 20A11hu.

4-4. Introduction of Protease Cleavage Sequence to Polypeptide with Incorporated VHH Containing Amino Acid Alteration at Interface Site Between the VHH and VL

Heavy chains 20A11huH1001 (SEQ ID NO: 40), 20A11huH1002 (SEQ ID NO: 41), 20A11huH1004 (SEQ ID NO: 42), and 20A11huH1006 (SEQ ID NO: 43) were prepared in the same way as in Example 3 such that a protease cleavage sequence (SEQ ID NO: 12) or a protease cleavage sequence linked to a flexible linker (SEQ ID NO: 44) was inserted near the boundary between 20A11hu and CH1.

Polypeptides 20A11huH1001/VK1-39-k0MT (heavy chain: SEQ ID NO: 40, light chain: SEQ ID NO: 3), 20A11huH1002/VK1-39-k0MT (heavy chain: SEQ ID NO: 41, light chain: SEQ ID NO: 3), 20A11huH1004/VK1-39-k0MT (heavy chain: SEQ ID NO: 42, light chain: SEQ ID NO: 3), and 20A11huH1006/VK1-39-k0MT (heavy chain: SEQ ID NO: 43, light chain: SEQ ID NO: 3) were expressed and purified in the same way as in Example 3 using these heavy chains and VK1-39-k0MT (SEQ ID NO: 3) as a light chain.

### 4-5. Activation of Polypeptide Harboring Protease Cleavage Sequence by Protease Cleavage

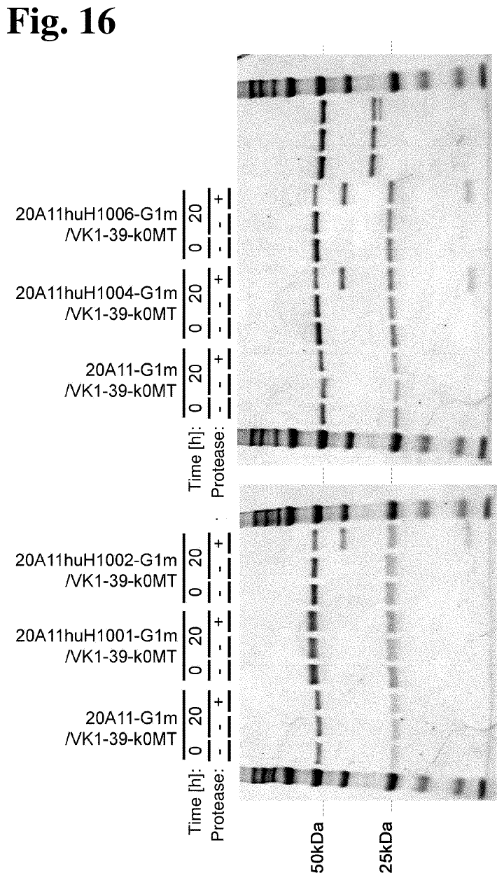

20A11huH1001/VK1-39-k0MT, 20A11huH1002/VK1-39-k0MT, 20A11huH1004/VK1-39-k0MT, and 20A11huH1006/VK1-39-k0MT were cleaved by protease in the same way as in Example 3, and the degree of the cleavage was evaluated by reducing SDS-PAGE. The results are shown in FIG. 16.

As a result, 20A11huH1002/VK1-39-k0MT, 20A11huH1004/VK1-39-k0MT, and 20A11huH1006/VK1-39-k0MT were confirmed to undergo protease cleavage near the boundary between VHH and CH1.

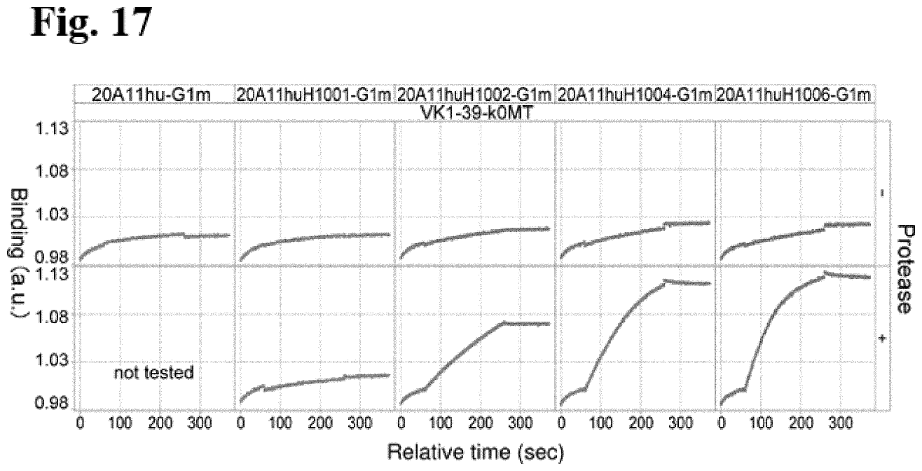

Next, the IL6R binding evaluation of VHH released by protease treatment was conducted at 30 degrees C. or 25 degrees C. in the same way as in Example 3. Octet sensorgrams are shown in FIG. 17.

As a result, the IL6R binding was confirmed in 20A11huH1002/VK1-39-k0MT, 20A11huH1004/VK1-39-k0MT, and 20A11huH1006/VK1-39-k0MT confirmed to undergo cleavage near the boundary between VHH and CH1 by protease treatment.

These results demonstrated that even if VHH incorporated in a polypeptide does not lose its antigen binding activity immediately after association with particular VL, the antigen binding activity can be lost by introducing an association promoting mutation to an amino acid present at the interface between the VHH and the VL.

From these results, it was concluded that the molecule conforming to the concept described in Example 2 can also be prepared by a method of combining a light chain with VHH containing a substituted amino acid involved in association with the light chain, in addition to the method of combining a light chain with VHH obtained in advance as in Example 3.

### Example 5: Preparation of Protease-Activated Polypeptide Using VHH Derived from Immunized Alpaca

5-1. Obtainment of VHH Derived from Immunized Alpaca

Alpacas were immunized with IL6R, CD3 or plexin A1 by a method known to those skilled in the art. 4 and 8 weeks later, PBMC was corrected. From the corrected PBMC, VHH gene was amplified with reference to a method described in J. Immunol. Methods (2007) 324, 13. The amplified VHH gene fragment was connected with gene 3 gene and inserted into a phagemid vector. The phagemid vector having the insert of the VHH fragment was transferred to E. coli by the electroporation method, and phages displaying g VHH were obtained by a method already known to those skilled in the art. The obtained phages were evaluated for their binding to IL6R, CD3 or plexin A1 by ELISA. The sequence of a bound clone was analyzed by a method known to those skilled in the art to identify VHH binding to the antigen.

### 5-2. Enrichment of VHH Binding to CD3

VHH binding to human CD3 was identified from the VHH library constructed in Example 5-1. VHH clones having binding capacity against human CD3 were enriched using a biotin-labeled protein containing human CD3 epsilon and human CD3 delta linked to a human antibody constant region (human CD3ed-Fc) as an antigen. The human CD3ed-Fc was prepared as follows: an expression vector for animal cells having a gene encoding the amino acid sequence represented by SEQ ID NO: 59, a gene encoding the amino acid sequence represented by SEQ ID NO: 60 and a gene encoding BirA (SEQ ID NO: 58) was transferred to FreeStyle 293 cells (Invitrogen Corp.). After the transfer, L-biotin was added thereto, and biotinylation was carried out in a culture solution. Cell culture was performed by shake culture at 37 degrees C. according to the protocol. 4 to 5 days later, the supernatant was recovered. From the supernatant, a protein fused with the antibody constant region was obtained using a protein A column (Eshmuno A (Merck KGaA)). For the purpose of further obtaining only a CD3 epsilon delta heterodimer, a fraction of the CD3 epsilon delta heterodimer fused with the antibody constant region (referred to as human CD3ed-Fc) was separated using Anti-FLAG M2 column. Subsequently, gel filtration chromatography (Superdex 200, GE Healthcare Japan Corp.) was carried out to obtain the fraction of the CD3 epsilon delta heterodimer of interest (referred to as human CD3ed-Fc).

Phage production was performed from E. coli retaining the constructed phagemids for phage display. A phage population was precipitated by the addition of 2.5 M NaCl/10% PEG to the culture solution of the E. coli after the phage production, and then diluted with TB S to obtain a phage library solution. Next, BSA was added to the phage library solution so as to attain a final BSA concentration of 4% Panning was performed with reference to a general panning method using an antigen immobilized on magnetic beads (J. Immunol. Methods. (2008) 332 (1-2), 2-9; J. Immunol. Methods. (2001) 247 (1-2), 191-203; Biotechnol. Frog. (2002) 18 (2) 212-20; and Mol. Cell Proteomics (2003) 2 (2), 61-9). The magnetic beads used were NeutrAvidin coated beads (FG beads NeutrAvidin) or Streptavidin coated beads (Dynabeads MyOne Streptavidin T1).

Specifically, 100 pmol of the biotin-labeled antigen was added to the prepared phage library solution, and the phage library solution was contacted with the antigen at room temperature for 60 minutes. The magnetic beads blocked with BSA were added thereto, and the complexes of the antigen and the phages were bound to the magnetic beads at room temperature for 15 minutes. The beads were washed twice with 0.5 mL of TBST (TBS containing 0.1% Tween 20; TBS was manufactured by Takara Bio Inc.) and then further washed once with 0.5 mL of TBS. Then, 0.5 mL of 1 mg/mL trypsin was added thereto, and the beads were suspended at room temperature for 15 minutes and immediately thereafter, separated using a magnetic stand to recover a phage solution. The recovered phage solution was added to 20 mL of an E. coli line ER2738 in an exponential stage of growth (OD600: 0.4-0.5). The E. coli was cultured with mild stirring at 37 degrees C. for 1 hour and thereby infected by the phages. The infected E. coli was inoculated to a 225 mm×225 mm plate. Next, the phages were recovered from the culture supernatant of the inoculated E. coli to prepare a phage library solution. This cycle, called panning, was repeated a total of twice. In the second cycle of panning, the beads were washed three times with TBST and subsequently twice with TBS. Also, 4 nmol of human Fc was added when the human CD3ed-Fc contacted with phages.

5-3. Preparation of Protease-Activated IgG Antibody-Like Molecule with Incorporated VHH Binding to CD3

A nucleotide sequence encoding the VHH sequence (Table 1) of each binding clone for human CD3 obtained in Example 5-1 or 5-2 was connected to a nucleotide sequence encoding a protease cleavage site and a constant region by the method described in Example 3 and inserted into an expression vector for animal cells. The resultant was used as the heavy chain of an IgG antibody-like molecule.

TABLE 1
VHH binding to human CD3

| VHH | SEQ ID NO |
|---|---|
| bC3edL1R1N160H01 | 61 |
| bC3edL1R1N161H01 | 62 |
| bC3edL1R1N164H01 | 63 |

Protease-activated IgG antibody-like molecules shown in Table 2 below were expressed by transient expression using FreeStyle 293 cells (Invitrogen Corp.) by a method known to those skilled in the art, and purified by a method known to those skilled in the art using protein A.

TABLE 2
Protease-activated IgG antibody-like molecules
with incorporated VHH binding to CD3

| | SEQ ID NO of | SEQ ID NO of |
|---|---|---|
| IgG antibody-like molecule | heavy chain | light chain |
| bC3edL1R1N160H01-G1mISHI01/ | 64 | 3 |
| VK1-39-k0MT | | |
| bC3edL1R1N161H01-G1mISHI01/ | 65 | |
| VK1-39-k0MT | | |
| bC3edL1R1N164H01-G1mISHI01/ | 66 | |
| VK1-39-k0MT | | |

### 5-4. Activation of Protease-Activated IgG Antibody-Like Molecule by Protease Cleavage

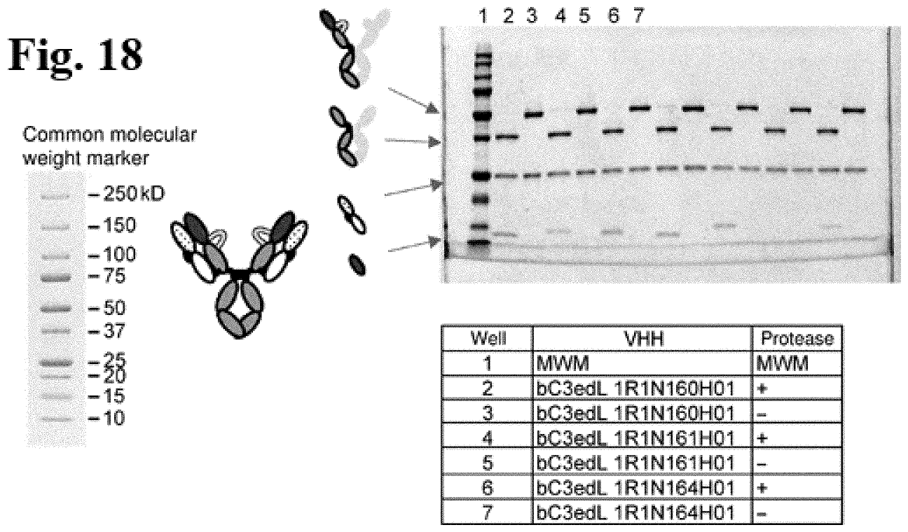

The IgG antibody-like molecules prepared in Example 5-3 were cleaved by protease in the same way as in Example 3, and the degree of the cleavage was evaluated by reducing SDS-PAGE. The results are shown in FIG. 18. The protease concentration was set to 25 nM, and Octet RED (Pall ForteBio Corp.) was used in the assay.

As a result, the IgG antibody-like molecules were confirmed to undergo protease cleavage at the protease cleavage sequence.

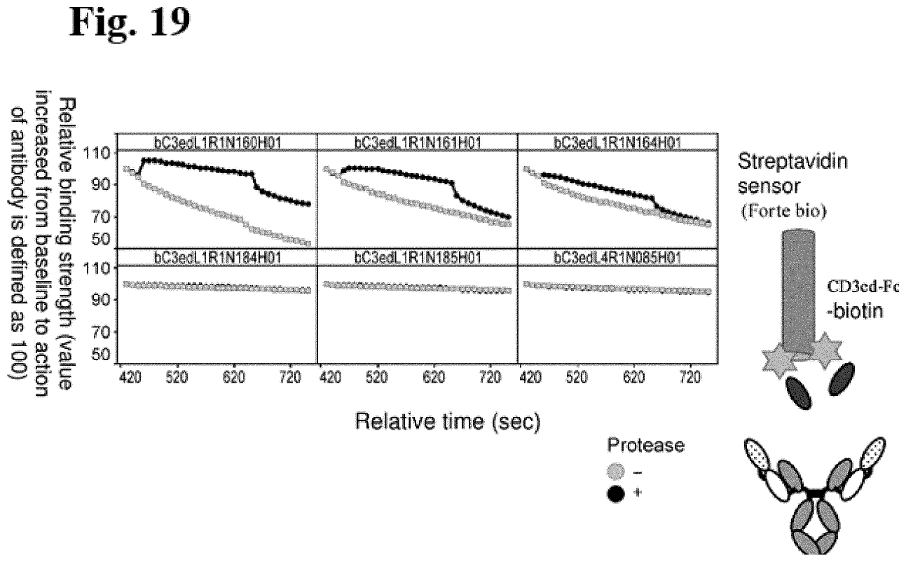

Next, the CD3 binding evaluation of VHH released by protease treatment was conducted in the same way as in Example 3. Octet sensorgrams are shown in FIG. 19.

As a result, the IgG antibody-like molecules bC3edL1R1N160H01-G1mISHI01/VK1-39-k0MT, bC3edL1R1N161H01-G1mISHI01/VK1-39-k0MT, and bC3edL1R1N164H01-G1mISHI01/VK1-39-k0MT did not exhibit antigen binding before the protease treatment, whereas the antigen binding was confirmed after the protease treatment. The plurality of VHH molecules binding to CD3 molecules, obtained in the same way as in the VHH described in Table 1, was also used to prepare an IgG-like molecule containing the same protease cleavage site as in the IgG antibody-like molecules described in Table 2. As a result, the antigen binding was confirmed by protease treatment. These results demonstrated that in addition to the polypeptides shown in Examples 3 and 4, an IgG antibody-like molecule harboring a protease cleavage sequence can undergo cleavage at the protease cleavage sequence by protease treatment and thereby release the antigen binding domain, and the released antigen binding domain can bind to the antigen.

### Example 6: Polypeptide Harboring Protease Cleavage Sequence in its Light Chain

Light chains VK1-39P-2-Pk0MT (SEQ ID NO: 67), VK1-39P-1-Pk0MT (SEQ ID NO: 68), VK1-39P-Pk0MT (SEQ ID NO: 69), VK1-39P+2-Pk0MT (SEQ ID NO: 70), VK1-39P+3-Pk0MT (SEQ ID NO: 71), VK1-39P+4-Pk0MT (SEQ ID NO: 72), and VK1-39P+5-Pk0MT (SEQ ID NO: 73) harboring a protease cleavage sequence at each position were prepared in the same way as in Example 3.

IgG antibody-like molecules were expressed and purified in the same way as in Example 3 using these light chains and IL6R90-G1m (SEQ ID NO: 2) as a heavy chain. The protease concentration was set to 25 nM. IL6R90-G1m/VK1-39-k0MT (heavy chain: SEQ ID NO: 2, light chain: SEQ ID NO: 3) was used as an IgG antibody-like molecule harboring no cleavage sequence.

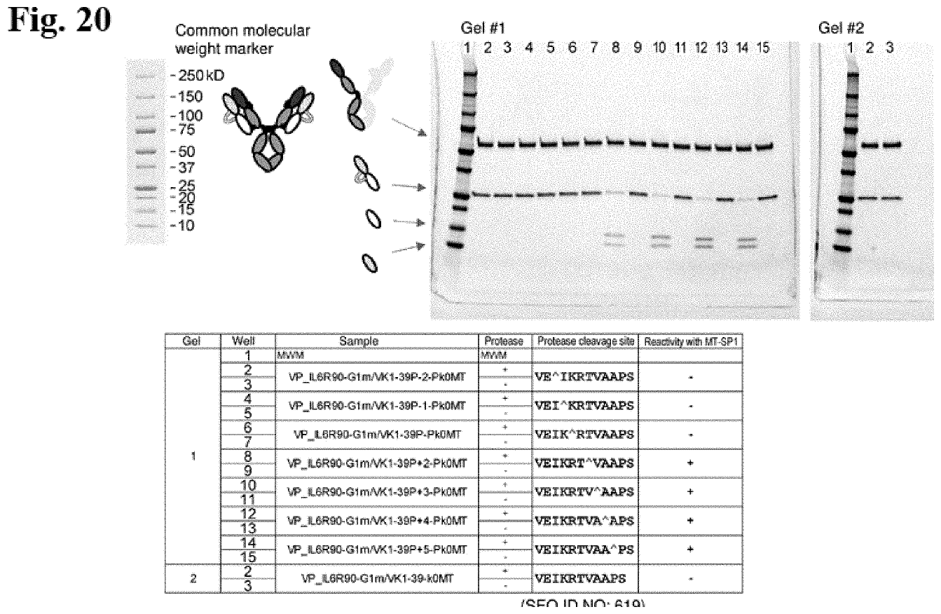

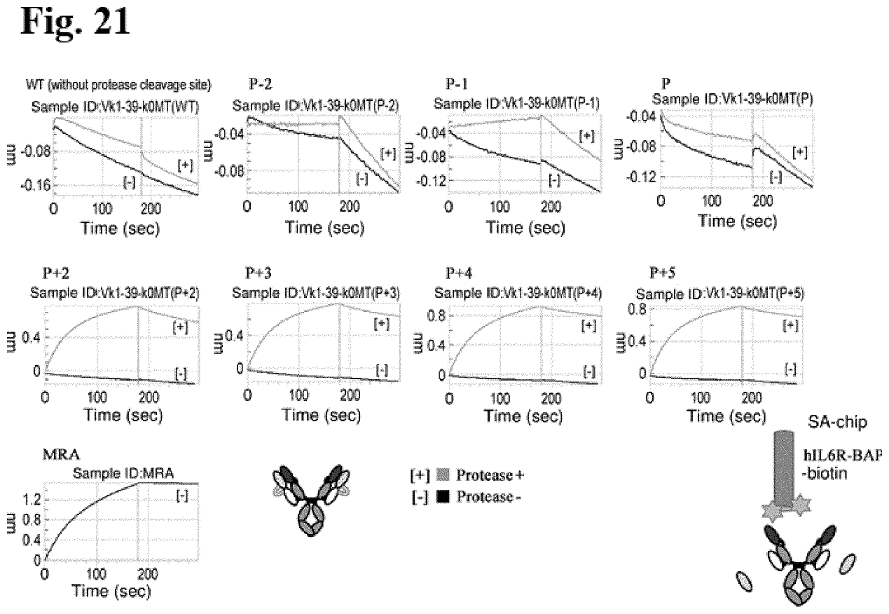

Subsequently, the prepared IgG antibody-like molecules were cleaved by protease in the same way as in Example 3, and the degree of the cleavage was evaluated by reducing SDS-PAGE. The results are shown in FIG. 20. As a result, VK1-39P+2-Pk0MT (SEQ ID NO: 70), VK1-39P+3-Pk0MT (SEQ ID NO: 71), VK1-39P+4-Pk0MT (SEQ ID NO: 72), and VK1-39P+5-Pk0MT (SEQ ID NO: 73) were confirmed to undergo protease cleavage at the protease cleavage sequence. The IL6R binding evaluation of VHH exposed by protease treatment was further conducted in the same way as in Example 3. Octet sensorgrams are shown in FIG. 21. As a result, the binding was also confirmed by the protease treatment of the cleavage sequence introduced into the light chain, demonstrating that a protease-activated polypeptide harboring a protease cleavage sequence in its light chain can be obtained such that the antigen binding domain is exposed to exhibit antigen binding capacity by the protease cleavage of the light chain.

### Example 7: Library Containing Heavy Chain Having Antigen Binding Domain and Light Chain Harboring Protease Cleavage Sequence, and Obtainment of Protease-Activated Polypeptide by Phage Display Method from the Library

As confirmed in Example 6, even when a protease cleavage sequence is introduced into the light chain of a protease-activated polypeptide, the antigen binding domain is exposed after cleavage of the light chain to bind to the antigen.

Accordingly, a heavy chain containing an antigen binding domain such as a single-domain antibody and a light chain harboring a protease cleavage sequence are incorporated in a phagemid and presented by a phage. A plurality of phagemids for phage display containing different types of antigen binding domains are constructed, followed by phage production from E. coli retaining these phagemids. A phage population is precipitated by the addition of 2.5 M NaCl/10% PEG to the culture solution of the E. coli after the phage production, and then diluted with TBS to obtain a phage library solution. BSA is added to the phage library solution so as to attain a final BSA concentration of 4%.

The protease-activated polypeptide is obtained by panning from the phage library thus prepared. The panning is performed with reference to a general panning method using an antigen immobilized on magnetic beads (J. Immunol. Methods. (2008) 332 (1-2), 2-9; J. Immunol. Methods. (2001) 247 (1-2), 191-203; Biotechnol. Frog. (2002) 18 (2) 212-20; and Mol. Cell Proteomics (2003) 2 (2), 61-9). Phages unbound with the antigen-immobilized magnetic beads are recovered before addition of protease, and phages bound with the antigen-immobilized magnetic beads are recovered after addition of protease. The magnetic beads used are NeutrAvidin coated beads (Sera-Mag SpeedBeads NeutrAvidin-coated, FG beads NeutrAvidin) or Streptavidin coated beads (Dynabeads M-280 Streptavidin). An antigen binding clone may be selected from the recovered phages by phage ELISA described in the preceding section, or the antibody gene is subcloned into a vector for expression in animals and expressed using animal cells, and the binding activity is compared between before and after protease treatment to select a binding clone.

### Example 8: Library Containing Heavy Chain Having Antigen Binding Domain and Light Chain, and Obtainment of Heavy Chain Whose Antigen Binding Capacity is Controlled by Light Chain by Phage Display Method from the Library

As confirmed in Example 3, the antigen binding capacity of a heavy chain containing an antigen binding domain is controlled by the association of a light chain. Accordingly, a heavy chain that loses its antigen binding capacity when associated with a light chain and exhibits antigen binding capacity when presented alone or in combination with a light chain constant region is obtained by the phage display method.

A heavy chain containing an antigen binding domain such as a single-domain antibody is incorporated in a phagemid and presented by a phage. A plurality of phagemids for phage display containing different types of antigen binding domains are constructed, followed by phage production from E. coli retaining these phagemids. A phage population is precipitated by the addition of 2.5 M NaCl/10% PEG to the culture solution of the E. coli after the phage production, and then diluted with TBS to obtain a phage library solution. BSA is added to the phage library solution so as to attain a final BSA concentration of 4%.

The heavy chain that exhibits antigen binding capacity when presented alone or in combination with a light chain constant region and loses its antigen binding capacity when associated with the light chain variable region is obtained by panning from the phage library thus prepared. The panning is performed with reference to the panning method using an antigen immobilized on magnetic beads described in Example 5. Phages bound with the antigen-immobilized magnetic beads are recovered from the phage library displaying heavy chains or heavy chains with light chain constant regions. The recovered phages are allowed to infect E. coli, and phages displaying heavy and light chains are produced using a helper phage expressing a light chain. Phages displaying a heavy chain containing an antigen binding domain and a light chain are obtained by the method mentioned above from the culture solution of the E. coli after the phage production. Phages unbound with the antigen-immobilized magnetic beads are recovered from the population of phages displaying heavy and light chains.

As shown in FIG. 9D, the panning may be carried out by changing the order of the recovery of a phage population displaying a heavy chain, either alone or in combination with a light chain constant region, binding to antigen-immobilized magnetic beads, and the recovery of a phage population displaying heavy and light chains without binding to antigen-immobilized magnetic beads. In addition to the method of expressing a light chain using a helper phage, a region encoding a light chain and a region encoding a heavy chain may be incorporated to the same phagemid as usual, and a gene encoding only a light chain constant region or a full-length light chain may be incorporated in each cycle of panning and used.

An antigen binding clone may be selected from the recovered phages by phage ELISA described in the preceding section, or the antibody gene is subcloned into a vector for expression in animals and expressed using animal cells, and the binding activity is compared between before and after protease treatment to select a binding clone.

### Example 9: Obtainment of VHH Whose Antigen Binding Capacity is Controlled by Light Chain by Use of Phage Display Method, and Preparation of IgG Antibody-Like Molecule Containing the VHH

In Example 3, it was confirmed that the antigen binding capacity of VHH contained as a substitute for VH in a heavy chain is controlled by association with a light chain. Accordingly, VHH that lost its antigen binding capacity when associated with a particular light chain and exhibited antigen binding capacity when the heavy chain was presented alone or in combination with a light chain constant region, i.e., when not associated with a light chain variable region, was obtained from a phage library displaying CH1 linked to VHH derived from immunized alpaca PBMC. An IgG antibody-like molecule containing the VHH was prepared.

9-1. Construction of Light Chain-Expressing Helper Phage with Integrated Light Chain Expression Unit

On the basis of a method described in International Publication No. WO2015/046554, a promoter, a signal sequence, antibody light chain variable region and light chain constant region genes or a light chain constant region gene, etc. were integrated into the genome of a helper phage to construct a light chain-expressing helper phage. E. coli infected with this helper phage is capable of expressing the antibody light chain variable region and the light chain constant region, or only the light chain constant region.

Specifically, the genome was extracted from a helper phage M13KO7TC constructed by the method described in International Publication No. WO2015/046554, and a light chain expression unit was introduced to the genome. A gene encoding a light chain variable region and a light chain constant region (VK1-39-k0MTdC; SEQ ID NO: 152), or a gene encoding a light chain constant region (k0MTdC; SEQ ID NO: 153) was used as the light chain gene to be introduced. lac promoter-pelB signal sequence-light chain gene was inserted into M13KO7TC/SacI by the method described above and transferred to an E. coli line ER2738 by the electroporation method.

The obtained E. coli was cultured, and 2.5 M NaCl/10% PEG was added to the culture supernatant to purify helper phages by the PEG precipitation method. The titers of the obtained helper phages M13KO7TC-Vk1-39-k0MTdC and M13KO7TC-k0MTdC were confirmed by the general plaque formation method.

### 9-2. Preparation of Library Containing a Plurality of VHH-CH1 Molecules

Alpacas were immunized by a method known to those skilled in the art using 4 types of immunogens: a human IL6R extracellular domain, a human CD3epsilongamma heterodimer, a monkey CD3epsilongamma heterodimer and a cell domain of human plexin A1. 4 weeks later, PBMC was recovered. The CD3epsilongamma heterodimers were prepared with reference to Journal of Molecular Biology (2000) 302: 899-916. From the recovered PBMC, VHH gene was amplified with reference to a method described in J. Immunol. Methods (2007) 324, 13. The amplified VHH gene fragment was connected with CH1-gene 3 gene and inserted into phagemid vectors to prepare a library containing a plurality of VHH-CH1 molecules containing VHH linked to CH1.

### 9-3. Method for Preparing Phage Population Displaying VHH-CH1/Full-Length Light Chain or VHH-CH1/Light Chain Constant Region

A phagemid vector having an insert of a gene encoding VHH-CH1 is transferred to E. coli by the electroporation method. The obtained E. coli can be cultured and infected by the helper phage M13KO7TC-Vk1-39-k0MTdC prepared in Example 9-1 so that VHH-CH1 expressed from the phagemid vector and the full-length light chain expressed from the helper phage form a Fab structure to prepare a phage population displaying VHH-CH1/full-length light chain (VHH-CH1/Vk1-39-k0MTdC) on the surface of phagemids containing the gene encoding VHH-CH1. Also, the E. coli harboring the phagemid vector having an insert of a gene encoding VHH-CH1 can be cultured and infected by the helper phage M13KO7TC-k0MTdC prepared in Example 9-1 so that VHH-CH1 expressed from the phagemid vector and the light chain constant region expressed from the helper phage form a structure of VHH-CH1 and CL associated to prepare a phage population displaying VHH-CH1/light chain constant region (VHH-CH1/k0MTdC). 2.5 M NaCl/10% PEG can be added to the culture supernatant to purify phages by the PEG precipitation method. The titers of the obtained phages can be confirmed by the general plaque formation method.

9-4. Obtainment of VHH-CH1 Containing Plexin A1 VHH Whose Antigen Binding is Inhibited by Association with Light Chain Variable Region and that Exhibits Antigen Binding Capacity in Absence of Light Chain Variable Region, from VHH-CH1 Phage Library

VHH-CH1 containing VHH whose antigen binding was inhibited by association with a light chain variable region and that exhibited antigen binding capacity in absence of the light chain variable region was obtained by panning from the VHH-CH1 library prepared in Example 9-2.

The antigen used was biotin-labeled human plexin A1 prepared in Reference Example.

The panning method was performed according to the following steps:
(1) A phage population displaying VHH-CH1/light chain constant region (VHH-CH1/k0MTdC) is produced by the method of Example 9-3 from the VHH-CH1 phage library prepared in Example 9-2, and phages bound with antigen-immobilized magnetic beads are recovered from the population.
(2) A phage population displaying VHH-CH1/full-length light chain (VHH-CH1/Vk1-39-k0MTdC) is produced by the method of Example 9-3 from the recovered phages, and phages unbound with the antigen-immobilized magnetic beads are recovered from the population.
(3) The recovered phages are repetitively subjected to the steps (1) and (2) to recover the desired phage.

As a result of the panning, a plurality of VHH-CH1 molecules were able to be selected whose plexin A1 binding was inhibited by association with the light chain Vk1-39-k0MTdC and that exhibited binding capacity against plexin A1 in the absence of the light chain variable region.

Another panning method was performed according to the following steps:
(1) A phage population displaying VHH-CH1/light chain constant region (VHH-CH1/k0MTdC) is produced by the method of Example 9-3 from the VHH-CH1 phage library prepared in Example 9-2, and phages bound with antigen-immobilized magnetic beads are recovered from the population.
(2) A phage population displaying VHH-CH1/full-length light chain (VHH-CH1/Vk1-39-k0MTdC) is produced by the method of Example 9-3 from the recovered phages, and phages unbound with the antigen-immobilized magnetic beads are recovered from the population. Phages binding to anti-light chain antibody (EY Laboratories, Inc., Cat. BAT-2107-2)-immobilized magnetic beads are further recovered from the recovered phages.
(3) The recovered phages are repetitively subjected to the steps (1) and (2) to recover the desired phage.

As a result of the panning, a plurality of VHH-CH1 molecules were able to be selected whose plexin A1 binding was inhibited by association with the light chain Vk1-39-k0MTdC and that exhibited binding capacity against plexin A1 in the absence of the light chain variable region.

The VHH in the VHH-CH1 thus selected by panning can be used in the preparation of IgG antibody-like molecules.

9-5. Preparation of Protease-Activated IgG Antibody-Like Molecule with Incorporated VHH Binding to Plexin A1

A nucleotide sequence encoding the VHH contained in each VHH-CH1 molecule selected in Example 9-4 was connected to a nucleotide sequence encoding a protease cleavage site and a heavy chain constant region by the method described in Example 3. The resultant was used as the heavy chain of an IgG antibody-like molecule and combined with a full-length light chain VK1-39-k0MT (SEQ ID NO: 3). IgG antibody-like molecules were expressed by transient expression using FreeStyle 293 cells (Invitrogen Corp.) by a method known to those skilled in the art, and purified by a method known to those skilled in the art using protein A.

The prepared IgG antibody-like molecules are shown in Table 3.

TABLE 3
IgG antibody-like molecules containing VHH binding to human plexin A1

| | Heavy chain | | Light chain | |
|---|---|---|---|---|
| IgG antibody-like molecule | Name | SEQ ID NO | Name | SEQ ID NO |
| PX02-R2_001-G1mISHI01/ | PX02-R2_001- | 154 | VK1-39-k0MT | 3 |
| VK1-39-k0MT | G1mISHI01 | | | |
| PX02-R4_004-G1mISHI01/ | PX02-R4_004- | 155 | | |
| VK1-39-k0MT | G1mISHI01 | | | |
| PX02-R4_017-G1mISHI01/ | PX02-R4_017- | 156 | | |
| VK1-39-k0MT | G1mISHI01 | | | |
| PX03-R2_006-G1mISHI01/ | PX03-R2_006- | 157 | | |
| VK1-39-k0MT | G1mISHI01 | | | |
| PX03-R4_009-G1mISHI01/ | PX03-R4_009- | 158 | | |
| VK1-39-k0MT | G1mISHI01 | | | |

### 9-6 Activation of Protease-Activated IgG Antibody-Like Molecule by Protease Cleavage

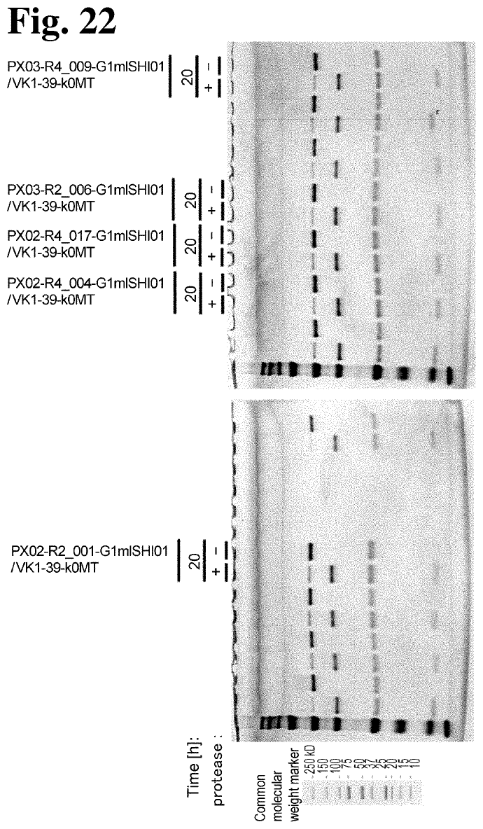

The IgG antibody-like molecules prepared in Example 9-4 were cleaved by protease in the same way as in Example 3, and the degree of the cleavage was evaluated by reducing SDS-PAGE. The results are shown in FIG. 22. The protease concentration was set to 25 nM.

As a result, the prepared IgG antibody-like molecules were each confirmed to undergo protease cleavage at the protease cleavage sequence.

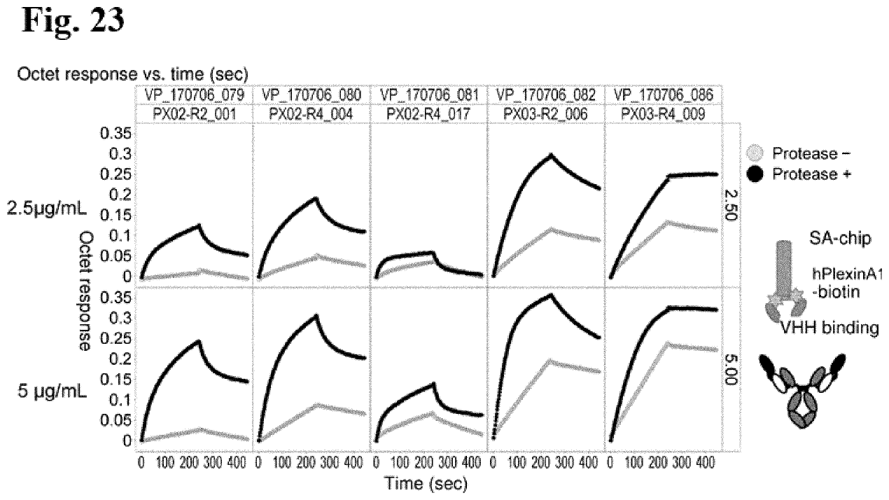

Next, the human plexin A1 binding evaluation of VHH released by protease treatment was conducted in the same way as in Example 3. Octet sensorgrams are shown in FIG. 23.

As a result, each of the prepared IgG antibody-like molecule did not exhibit antigen binding before the protease treatment, whereas the antigen binding of the released VHH was confirmed after the protease treatment.

### Example 10: Polypeptide Containing Bispecific VHH-VHH

### 10-1. Bispecific VHH-VHH Binding to Cancer Antigen and CD3, and Preparation of Polypeptide Containing the Bispecific VHH-VHH

As shown in FIG. 8, a protease-activated antigen binding domain may form a bispecific antigen binding molecule with a second antigen binding domain.

VHH HN3 (SEQ ID NO: 159) recognizing human glypican 3 and VHH G03 (SEQ ID NO: 160) recognizing CD3 were connected via a linker constituted by glycine and serine to prepare bispecific VHH-VHH HN3G03. An antibody heavy chain constant region shown in SEQ ID NO: 161 was further connected thereto via a protease cleavage sequence, and the resulting heavy chain HN3G03-cF760mnHIF (SEQ ID NO: 162) containing the bispecific VHH-VHH was inserted into a vector for expression in animals.

VHH HerF07 (SEQ ID NO: 163) recognizing Her2 and VHH G03 (SEQ ID NO: 160) recognizing CD3 were connected via a linker constituted by glycine and serine to prepare bispecific VHH-VHH HerF07G03. An antibody heavy chain constant region shown in SEQ ID NO: 161 was further connected thereto via a protease cleavage sequence, and the resulting heavy chain HerF07G03-cF760mnHIF (SEQ ID NO: 164) containing the bispecific VHH-VHH was inserted into a vector for expression in animals.

Expi293 cells (Life Technologies Corp.) were cotransfected with each heavy chain containing the bispecific VHH-VHH and vectors for expression in animals respectively having inserts of a light chain VK1.39-k0MT (SEQ ID NO: 3) and a human constant region sequence VHn-Kn010dGK (SEQ ID NO: 166) from the hinge region to the C terminus, to express a polypeptide containing the bispecific VHH-VHH. Then, the polypeptide containing the bispecific VHH-VHH was purified by a method known to those skilled in the art using a MonoSpin ProA 96-well plate type (GL Sciences Inc., Cat No.: 7510-11312). The polypeptide containing the bispecific VHH-VHH HN3G03 is HN3G03-cF760mnHIF/VHn-Kn010dGK/VK1.39-k0MT, and the polypeptide containing the bispecific VHH-VHH HerF07G03 is HerF07G03-cF760mnHIF/VHn-Kn010dGK/VK1.39-k0MT.

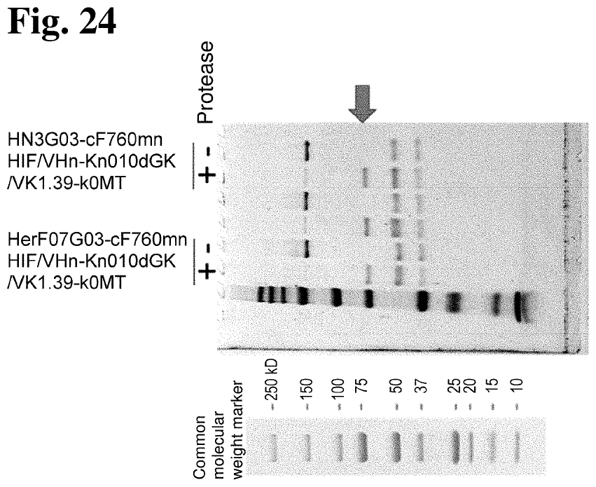

For protease treatment, uPA (Recombinant Human u-Plasminogen Activator, R&D Systems, Inc.) (final concentration: 25 nM) was added to 40 micro g of each purified polypeptide containing the bispecific VHH-VHH and incubated at 37 degrees C. for 20 hours or longer. Protease-untreated samples were incubated after addition of PBS instead of protease in the same amount as in the protease. Whether the protease-cleaved polypeptide containing the bispecific VHH-VHH underwent the cleavage as intended was confirmed by reducing SDS-PAGE. The results are shown in FIG. 24. As shown in FIG. 24, it was suggested that the bispecific VHH-VHH was separated from the whole molecule by the protease cleavage.

### 10-2. CD3 Activation Evaluation of Polypeptide Containing Bispecific VHH-VHH Against GPC3 and CD3 by Protease Cleavage

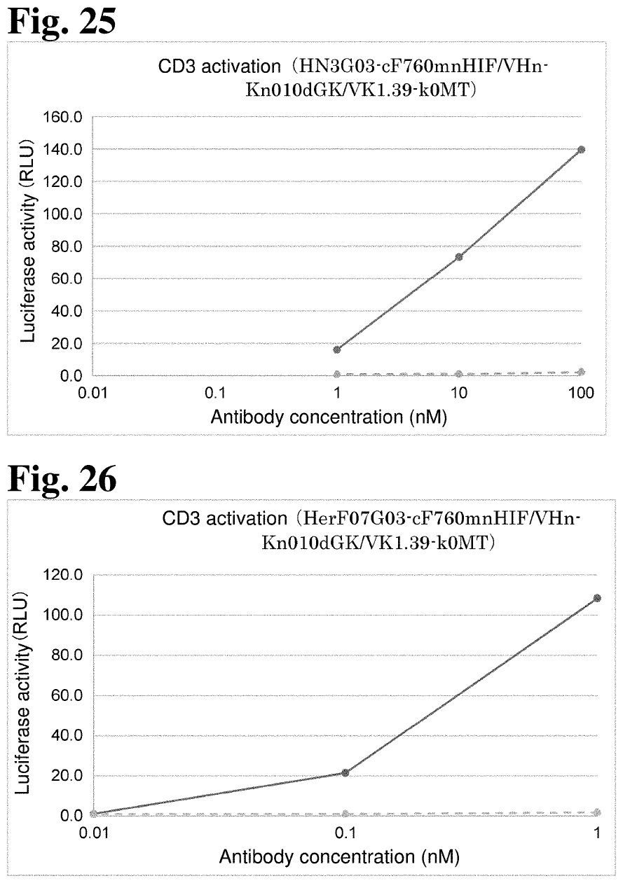

Agonist activity against CD3 was evaluated using Jurkat-NFAT reporter cells (NFAT luc2_jurkat cell). The Jurkat-NFAT reporter cells are a cell line of CD3-expressing human acute T-cell leukemia-derived cells fused with a NFAT response element and luciferase (luc2P) and express luciferase by the activation of a signal downstream of CD3. The target cells used for antibodies based on GPC3 were a SK-pca60 cell line established by forcing a human liver cancer-derived cell line SK-HEP-1 to express human GPC3. The target cells and the effector cells were added at 1.25E+04 cells/well and 7.50E+04 cells/well, respectively, to each well of White-bottomed, 96-well assay plate (Costar, 3917). HN3G03-cF760mnHIF/VHn-Kn010dGK/VK1.39-k0MT with or without protease treatment was added at a final concentration of 1, 10, or 100 nM to the well. After 24-hour incubation at 37 degrees C. in the presence of 5% CO₂, the luciferase enzyme activity was measured as luminescence intensity using Bio-Glo luciferase assay system (Promega Corp., G7940) according to the attached protocol. 2104 EnVision was used in detection. The results are shown in FIG. 25. No elevation in luciferase activity was seen in the sample without protease treatment, whereas elevation in luciferase activity was shown in HN3G03-cF760mnHIF/VHn-Kn010dGK/VK1.39-k0MT treated with protease. Specifically, HN3G03-cF760mnHIF/VHn-Kn010dGK/VK1.39-k0MT treated with protease was able to be confirmed to have agonist activity against CD3, while the bispecific VHH-VHH against GPC3 and CD3 was released from HN3G03-cF760mnHIF/VHn-Kn010dGK/VK1.39-k0MT by the protease cleavage and exerted the CD3 binding activity inhibited without cleavage.

### 10-3. CD3 Activation Evaluation of Polypeptide Containing Bispecific VHH-VHH Against Her2 and CD3 by Protease Cleavage

Agonist activity against CD3 was evaluated using Jurkat-NFAT reporter cells (NFAT luc2_jurkat cell). The Jurkat-NFAT reporter cells (effector cells) are a cell line of CD3-expressing human acute T-cell leukemia-derived cells fused with a NFAT response element and luciferase (luc2P) and express luciferase by the activation of a signal downstream of CD3. The target cells used were a LS1034 cell line. The target cells and the effector cells were added at 2.50E+04 cells/well and 7.50E+04 cells/well, respectively, to each well of White-bottomed, 96-well assay plate (Costar, 3917). HerF07G03-cF760mnHIF/VHn-Kn010dGK/VK1.39-k0MT with or without protease treatment was added at a final concentration of 0.01, 0.1, and 1 nM to the well. After 24-hour incubation at 37 degrees C. in the presence of 5% CO₂, the luciferase enzyme activity was measured as luminescence intensity using Bio-Glo luciferase assay system (Promega Corp., G7940) according to the attached protocol. 2104 EnVision was used in detection. The results are shown in FIG. 26. No elevation in luciferase activity was seen in the sample without protease treatment, whereas elevation in luciferase activity was shown in HerF07G03-cF760mnHIF/VHn-Kn010dGK/VK1.39-k0MT treated with protease. Specifically, HerF07G03-cF760mnHIF/VHn-Kn010dGK/VK1.39-k0MT treated with protease was able to be confirmed to have agonist activity against CD3, while the bispecific VHH-VHH against Her2 and CD3 was released from HerF07G03-cF760mnHIF/VHn-Kn010dGK/VK1.39-k0MT by the protease cleavage and exerted the CD3 binding activity inhibited without cleavage.

### Example 11: Introduction of Protease Cleavage Site to Polypeptide with Incorporated VHH

11-1. Introduction of Protease Cleavage Sequence to Polypeptide with Incorporated VHH Binding to IL6R

An expression vector encoding IL6R90-G1T4 (SEQ ID NO: 167) containing IL6R90 (SEQ ID NO: 1), VHH having binding and neutralizing activities against human IL6R as described in International Publication No. WO2010/115998, fused with a human IgG1 constant region (CH1-hinge-CH2-CH3) was prepared by a method known to those skilled in the art. An IgG antibody-like molecule IL6R90-G1T4/VK1-39-k0MT (heavy chain: SEQ ID NO: 167, light chain: SEQ ID NO: 3) was expressed by transient expression using FreeStyle 293 cells (Invitrogen Corp.) or Expi293 cells (Life Technologies Corp.) by a method known to those skilled in the art, and purified by a method known to those skilled in the art using protein A.

A protease cleavage sequence shown in SEQ ID NO: 178 was inserted near the boundary between VHH and CH1 in the heavy chain of IL6R90-G1T4/VK1-39-k0MT to prepare a VHH-containing heavy chain IL6R90.12aa-G1T4 (SEQ ID NO: 189) harboring the protease cleavage sequence. An IL6R90.12aa-G1T4 expression vector was prepared by a method known to those skilled in the art.

IL6R90.12aa-G1T4 was combined with a light chain shown in SEQ ID NO: 3. An IgG1 antibody-like molecule IL6R90.12aa-G1T4/VK1-39-k0MT harboring the protease cleavage sequence near the boundary between VHH and CH1 was expressed by transient expression using FreeStyle 293 cells (Invitrogen Corp.) or Expi293 cells (Life Technologies Corp.) by a method known to those skilled in the art, and purified by a method known to those skilled in the art using protein A.

### 11-2. Protease Cleavage Evaluation of IgG Antibody-Like Molecule Containing Anti-Human IL6R VHH and Harboring Protease Cleavage Sequence in its Heavy Chain Region

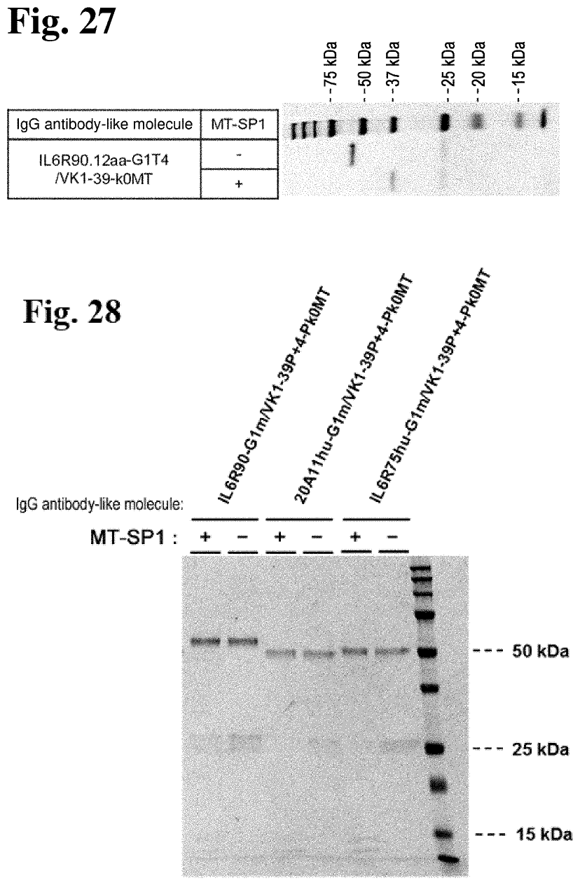

Whether the IgG antibody-like molecule prepared in Example 11-1 would be cleaved by protease was verified. Recombinant Human Matriptase/ST14 Catalytic Domain (MT-SP1) (R&D Systems, Inc., 3946-SE-010) was used as the protease. 10 nM protease and 50 micro g/mL of the antibody were reacted in PBS under a condition of 37 degrees C. for 20 hours. Then, cleavage by the protease was evaluated by reducing SDS-PAGE. The results are shown in FIG. 27. As a result, the protease treatment of the IgG antibody-like molecule IL6R90.12aa generated a new band around 37 kDa. Thus, the IgG antibody-like molecule was confirmed to undergo protease cleavage at the protease cleavage sequence (SEQ ID NO: 178) inserted near the boundary between VHH and CH1. Also, a protease cleavage sequence represented by SEQ ID NO: 178 was also confirmed to be cleaved by human uPA and mouse uPA when incorporated in an IgG antibody by a similar method.

### Example 12: Evaluation of Degree of Activation by Protease Cleavage of IgG Antibody-Like Molecule Harboring Protease Cleavage Sequence in its Light Chain

An expression vector encoding IL6R75-G1m (SEQ ID NO: 191) containing IL6R75 (SEQ ID NO: 190), VHH having binding and neutralizing activities against human IL6R as described in International Publication No. WO2010/115998, fused with a human IgG1 constant region (CH1-hinge-CH2-CH3) was prepared by a method known to those skilled in the art. IL6R75hu-G1m (SEQ ID NO: 192) was prepared by introducing amino acid alterations to the interface site between VHH and VL in the same way as in Example 4-2. IgG antibody-like molecules IL6R90-G1m/VK1-39P+4-Pk0MT (heavy chain: SEQ ID NO: 2, light chain: SEQ ID NO: 72), 20A11hu-G1m/K1-39P+4-Pk0MT (heavy chain: SEQ ID NO: 39, light chain: SEQ ID NO: 72), and IL6R75hu-G1m/VK1-39P+4-Pk0MT (heavy chain: SEQ ID NO: 192, light chain: SEQ ID NO: 72) were expressed and purified in the same way as in Example 3 using the protease cleavage sequence-incorporated light chain VK1-39P+4-Pk0MT (SEQ ID NO: 72) and IL6R90-G1m (SEQ ID NO: 2), 20A11hu-G1m (SEQ ID NO: 39), and IL6R75hu-G1m (SEQ ID NO: 192) as heavy chains.

IL6R90-G1m/VK1-39P+4-Pk0MT, 20A11hu-G1m/VK1-39P+4-Pk0MT, and IL6R75hu-G1m/VK1-39P+4-Pk0MT were cleaved by protease in the same way as in Example 3, and the degree of the cleavage was evaluated. The results are shown in FIG. 28. Specifically, recombinant Human Matriptase/ST14 Catalytic Domain (R&D Systems, Inc., 3946-SE-010) was used as the protease. 50 nM protease and 50 micro g/mL of each IgG antibody-like molecule were reacted in PBS under a condition of 37 degrees C. for 20 hours. Then, cleavage by the protease was evaluated by reducing SDS-PAGE. As a result, IL6R90-G1m/VK1-39P+4-Pk0MT, 20A11hu-G1m/VK1-39P+4-Pk0MT, and IL6R75hu-G1m/VK1-39P+4-Pk0MT were confirmed to undergo protease cleavage near the boundary between VL and CL.

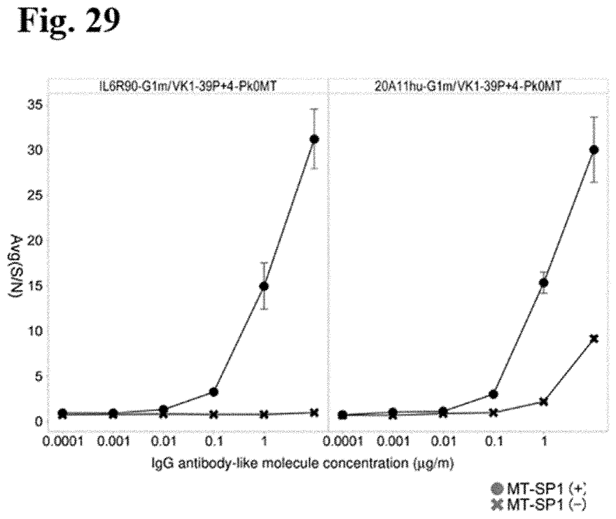

Next, the IL6R binding of VHH exposed by protease treatment was evaluated by ELISA. Specifically, the hsIL-6R-BAP1 used in Example 3 was immobilized onto a streptavidin-coated 384-well plate (Greiner Bio-One GmbH, 781990), and each cleaved IgG antibody-like molecule was bound thereto at room temperature. After reaction for 30 minutes, a HRP-labeled anti-human IgG antibody (Sigma-Aldrich Co. LLC, SAB3701362-2MG) was allowed to act thereon at room temperature for 10 minutes, and TMB Chromogen Solution (Life Technologies Corp., 002023) was reacted therewith. After reaction at room temperature for 30 minutes, the reaction was terminated with sulfuric acid, followed by the measurement of absorbance at 450 nm using Synergy HTX multi-mode reader (BioTek Instruments, Inc.). The absorbance ratio of the antigen-immobilized wells to unimmobilized wells was calculated and used as a S/N ratio. The S/N ratio (mean) of ELISA was plotted on the ordinate against the concentration of each IgG antibody-like molecule on the abscissa. The results are shown in FIG. 29. These results showed that the protease-treated IgG antibody-like molecule 20A11hu-G1m/VK1-39P+4-Pk0MT harboring the cleavage sequence in its light chain had 10 or more times the IL6R binding activity of the protease-untreated IgG antibody-like molecule, and the protease-treated IgG antibody-like molecule IL6R90-G1m/VK1-39P+4-Pk0MT had 1000 or more times the IL6R binding activity of the protease-untreated one.

### Example 13: Preparation and Evaluation of IgG Antibody-Like Molecules Harboring Diverse Protease Cleavage Sequences

### 13-1. Preparation of Polypeptides Harboring Diverse Protease Cleavage Sequences

IgG antibody-like molecules were prepared in the same way as in Example 3 using recognition sequences for proteases other than urokinase or matriptase. Various peptide sequences known to be cleaved by MMP-2, MMP-7, MMP-9, or MMP-13 were each inserted near the boundary between the variable and constant regions of IL6R90-G1m, and a peptide sequence containing a flexible linker consisting of a glycine-serine polymer was inserted in the vicinity of these cleavage sequences. The inserted sequences are shown in Table 4.

TABLE 4
Various inserted sequences

| Protease | Inserted sequence | SEQ ID NO |
|---|---|---|
| MMP-2 | PLGLAG | 34 |
| MMP-9 | | |
| MMP-2 | GAGIPVSLRSGAG | 70 |
| MMP-2 | GPLGIAGQ | 71 |
| MMP-2 | GGPLGMLSQS | 72 |
| MMP-2 | PLGLWA | 73 |
| MMP-7 | VPLSLTMG | 35 |
| MMP-7 | GAGVPLSLTMGAG | 75 |
| MMP-9 | GAGVPLSLYSGAG | 76 |
| MMP-13 | GAGPQGLAGQRGIVAG | 91 |
| MMP-2 | GGGGSPLGLAGGGGGS | 193 |
| MMP-9 | | |
| MMP-2 | GGGGSGPLGIAGQGGGGS | 194 |
| MMP-9 | GGGGSGAGVPLSLYSGAGGGGGS | 195 |

Heavy chains were designed such that these sequences were inserted near the boundary between the variable and constant regions of IL6R90-G1m. Expression vectors encoding the heavy chain variants 6R90EIVHEMP2.1-6R90EICHEMP2.1G1m (SEQ ID NO: 165), 6R90EIVHEMP2.2-6R90EICHEMP2.2G1m (SEQ ID NO: 202), 6R90EIVHEMP2.3-6R90EICHEMP2.3G1m (SEQ ID NO: 203), 6R90EIVHEMP2.4-6R90EICHEMP2.4G1m (SEQ ID NO: 204), 6R90EIVHEMP7.1-6R90EICHEMP7.1G1m (SEQ ID NO: 205), 6R90EIVHEMP7.2-6R90EICHEMP7.2G1m (SEQ ID NO: 206), 6R90EIVHEMP13-6R90EICHEMP13G1m (SEQ ID NO: 207), 6R90EIVHEG4SMP2MP9G4S-6R90EICHEG4SMP2MP9G4SG1m (SEQ ID NO: 196), 6R90EIVHEG4SMP2.2G4S-6R90EIVHEG4SMP2.2G4SG1m (SEQ ID NO: 197), and 6R90EIVHEG4SMP9G4S-6R90EIVHEG4SMP9G4SG1m (SEQ ID NO: 198) were prepared by a method known to those skilled in the art.

Table 5 shows the IgG antibody-like molecules combining these heavy chain variants with a light chain and harboring the protease cleavage sequence near the boundary between the variable and constant regions of the heavy chain. These IgG antibody-like molecules were expressed by transient expression using FreeStyle 293 cells (Invitrogen Corp.) or Expi293 cells (Life Technologies Corp.) by a method known to those skilled in the art, and purified by a method known to those skilled in the art using protein A.

TABLE 5
IgG antibody-like molecules

| | | SEQ ID NO of | SEQ ID NO of |
|---|---|---|---|
| Protease | IgG antibody-like molecule | heavy chain | light chain |
| MMP-2 | 6R90EIVHEMP2.1-6R90EICHEMP2.1G1m/ | 165 | 3 |
| | VK1-39-k0MT | | |
| MMP-2 | 6R90EIVHEMP2.2-6R90EICHEMP2.2G1m/ | 202 | 3 |
| | VK1-39-k0MT, | | |
| MMP-2 | 6R90EIVHEMP2.3-6R90EICHEMP2.3G1m/ | 203 | 3 |
| | VK1-39-k0MT, | | |
| MMP-2 | 6R90EIVHEMP2.4-6R90EICHEMP2.4G1m/ | 204 | 3 |
| | VK1-39-k0MT, | | |
| MMP-7 | 6R90EIVHEMP7.1-6R90EICHEMP7.1G1m/ | 205 | 3 |
| | VK1-39-k0MT, | | |
| MMP-7 | 6R90EIVHEMP7.2-6R90EICHEMP7.2G1m/ | 206 | 3 |
| | VK1-39-k0MT | | |
| MMP-13 | 6R90EIVHEMP13-6R90EICHEMP13G1m/ | 207 | 3 |
| | VK1-39-k0MT | | |
| MMP-2 | 6R90EIVHEG4SMP2MP9G4S-6R90E1CHEG4SMP2MP9G4SG1m/ | 196 | 3 |
| MMP-9 | VK1-39-k0MT | | |
| MMP-2 | 6R90EIVHEG4SMP2.2G4S-6R90EICHEG4SMP2.2G4SG1m/ | 197 | 3 |
| | VK1-39-k0MT | | |
| MMP-9 | 6R90EIVHEG4SMP9G4S-6R90EICHEG4SMP9G4SG1m/ | 198 | 3 |
| | VK1-39-k0MT | | |

### 13-2. Protease Cleavage Evaluation of IgG Antibody-Like Molecules Harboring Diverse Protease Cleavage Sequences

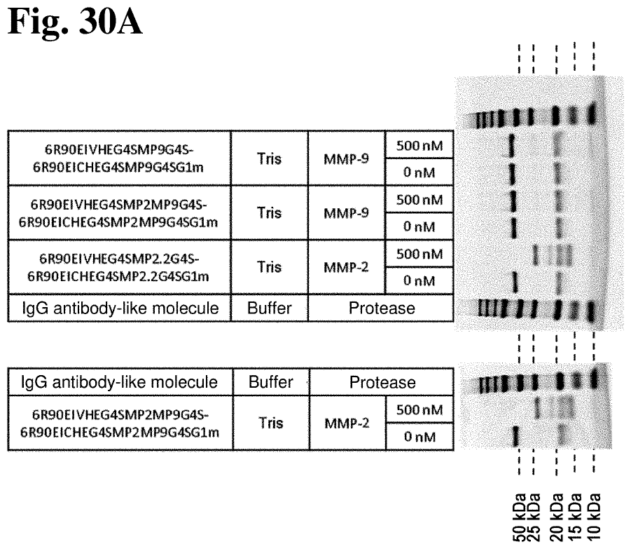

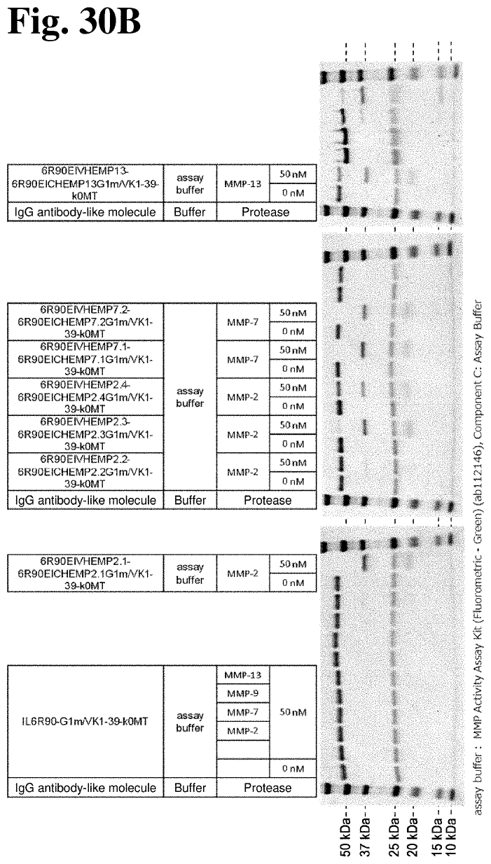

Whether the IgG antibody-like molecules prepared in Example 13-1 would be cleaved by protease was verified. Recombinant human MMP-2 (R&D Systems, Inc., 902-MP-010), recombinant human MMP-7 (R&D Systems, Inc., 907-MP-010), recombinant human MMP-9 (R&D Systems, Inc., 911-MP-010), or recombinant human MMP-13 (R&D Systems, Inc., 511-MM-010) was used as the protease. MMP-2, MMP-7, MMP-9, and MMP-13 were used after being each mixed with 1 MMP-aminophenylmercuric acetate (APMA; Abcam PLC, ab112146) and activated at 37 degrees C. for 1 or 24 hours. 50 nM, 100 nM, or 500 nM protease and 50 micro g/mL or 100 micro g/mL of each IgG-antibody like molecule were reacted in PBS or 20 mM Tris-HCl, 150 mM NaCl, and 5 mM CaCl₂) (pH 7.2) (hereinafter, referred to as Tris) under a condition of 37 degrees C. for 20 hours. Then, cleavage by the protease was evaluated by reducing SDS-PAGE. The results are shown in FIGS. 30A and 30B. In FIG. 30B, the protease cleavage was carried out using an assay buffer (MMP Activity Assay Kit (Fluorometric—Green) (ab112146), Component C: Assay Buffer).

As a result, 6R90EIVHEMP2.1-6R90EICHEMP2.1G1m/VK1-39-k0MT, 6R90EIVHEMP2.2-6R90EICHEMP2.2G1m/VK1-39-k0MT, 6R90EIVHEMP2.3-6R90EICHEMP2.3G1m/VK1-39-k0MT, 6R90EIVHEMP2.4-6R90EICHEMP2.4G1m/VK1-39-k0MT, 6R90EIVHEG4SMP2MP9G4S-6R90EICHEG4SMP2MP9G4SG1m/VK1-39-k0MT, and 6R90EIVHEG4SMP2.2G4S-6R90EICHEG4SMP2.2G4SG1m/VK1-39-k0MT were confirmed to be cleaved by MMP-2. 6R90EIVHEMP7.1-6R90EICHEMP7.1G1m/VK1-39-k0MT and 6R90EIVHEMP7.2-6R90EICHEMP7.2G1m/VK1-39-k0MT were confirmed to be cleaved by MMP-7. 6R90EIVHEG4SMP2MP9G4S-6R90EICHEG4SMP2MP9G4SG1m/VK1-39-k0MT and 6R90EIVHEG4SMP9G4S-6R90EICHEG4SMP9G4SG1m/VK1-39-k0MT were confirmed to be cleaved by MMP-9. 6R90EIVHEMP13-6R90EICHEMP13G1m/VK1-39-k0MT was confirmed to be cleaved by MMP-13.

### Example 14: Evaluation of Antibodies Harboring Protease Cleavage Sequence at Diverse Positions of Heavy Chain

### 14-1. Preparation of Antibodies Harboring Protease Cleavage Sequence at Diverse Positions of Heavy Chain

Peptide sequence B (SEQ ID NO: 210) reportedly cleavable by urokinase (uPA) and matriptase (MT-SP1) was inserted at each of different positions within a MRA heavy chain variable region (MRAH; SEQ ID NO: 211) to prepare engineered MRA heavy chain variable regions shown in Table 6. These engineered MRA heavy chain variable regions were each linked to a MRA heavy chain constant region (G1T4; SEQ ID NO: 212) to prepare engineered MRA heavy chains. The corresponding gene expression vectors were prepared by a method known to those skilled in the art. Also, peptide sequence B (SEQ ID NO: 210) was inserted at each of different positions within a MRA heavy chain constant region (G1T4; SEQ ID NO: 212) to prepare engineered MRA heavy chain constant regions shown in Table 7. These engineered MRA heavy chain constant regions were each linked to a MRA heavy chain variable region (MRAH; SEQ ID NO: 211) to prepare engineered MRA heavy chains. The corresponding gene expression vectors were prepared by a method known to those skilled in the art. Tables 6 and 7 also show the protease cleavage sequence insertion positions in the prepared engineered MRA heavy chain variable regions and engineered MRA heavy chain constant regions. In Table 6, the inserted sequence was located adjacent on the constant region side to the described position (Kabat numbering) in the antibody heavy chain variable region. In Table 7, the inserted sequence was located adjacent on the variable region side to the described position (EU numbering) in the antibody heavy chain constant region.

TABLE 6
Engineered MRA heavy chain variable regions and
protease cleavage sequence insertion positions

| | Protease cleavage sequence | |
|---|---|---|
| Engineered MRA heavy | insertion position | SEQ |
| chain variable region | (Kabat numbering) | ID NO |
| MRAVH007.12aa | 7 | 213 |
| MRAVH008.12aa | 8 | 214 |
| MRAVH009.12aa | 9 | 215 |
| MRAVH010.12aa | 10 | 216 |
| MRAVH011.12aa | 11 | 217 |
| MRAVH012.12aa | 12 | 218 |
| MRAVH013.12aa | 13 | 219 |
| MRAVH014.12aa | 14 | 220 |
| MRAVH015.12aa | 15 | 221 |
| MRAVH041.12aa | 40 | 222 |
| MRAVH042.12aa | 41 | 223 |
| MRAVH043.12aa | 42 | 224 |
| MRAVH044.12aa | 43 | 225 |
| MRAVH045.12aa | 44 | 226 |
| MRAVH046.12aa | 45 | 227 |
| MRAVH056.12aa | 55 | 228 |
| MRAVH057.12aa | 56 | 229 |
| MRAVH058.12aa | 57 | 230 |
| MRAVH059.12aa | 58 | 231 |
| MRAVH060.12aa | 59 | 232 |
| MRAVH061.12aa | 60 | 233 |
| MRAVH062.12aa | 61 | 234 |
| MRAVH063.12aa | 62 | 235 |
| MRAVH064.12aa | 63 | 236 |
| MRAVH065.12aa | 64 | 237 |
| MRAVH066.12aa | 65 | 238 |
| MRAVH067.12aa | 66 | 239 |
| MRAVH068.12aa | 67 | 240 |
| MRAVH069.12aa | 68 | 241 |
| MRAVH074.12aa | 73 | 242 |
| MRAVH075.12aa | 74 | 243 |
| MRAVH076.12aa | 75 | 244 |
| MRAVH077.12aa | 76 | 245 |
| MRAVH078.12aa | 77 | 246 |
| MRAVH087.12aa | 83 | 247 |
| MRAVH088.12aa | 84 | 248 |
| MRAVH089.12aa | 85 | 249 |
| MRAVH099.12aa | 95 | 250 |
| MRAVH100.12aa | 96 | 251 |
| MRAVH101.12aa | 97 | 252 |
| MRAVH102.12aa | 98 | 253 |
| MRAVH109.12aa | 103 | 254 |
| MRAVH110.12aa | 104 | 255 |
| MRAVH111.12aa | 105 | 256 |
| MRAVH112.12aa | 106 | 257 |
| MRAVH113.12aa | 107 | 258 |
| MRAVH114.12aa | 108 | 259 |
| MRAVH115.12aa | 109 | 260 |
| MRAVH116.12aa | 110 | 261 |
| MRAVH117.12aa | 111 | 262 |
| MRAVH118.12aa | 112 | 263 |
| MRAVH119.12aa | 113 | 264 |

TABLE 7
Engineered MRA heavy chain constant regions and
protease cleavage sequence insertion positions

| | Protease cleavage sequence | |
|---|---|---|
| Engineered MRA heavy | insertion position | SEQ |
| chain constant region | (EU numbering) | ID NO |
| G1T4.118.12aa | 119 | 265 |
| G1T4.119.12aa | 120 | 266 |
| G1T4.120.12aa | 121 | 267 |
| G1T4.121.12aa | 122 | 268 |
| G1T4.122.12aa | 123 | 269 |
| G1T4.123.12aa | 124 | 270 |
| G1T4.124.12aa | 125 | 271 |
| G1T4.129.12aa | 130 | 272 |
| G1T4.130.12aa | 131 | 273 |
| G1T4.131.12aa | 132 | 274 |
| G1T4.132.12aa | 133 | 275 |
| G1T4.134.12aa | 135 | 276 |
| G1T4.135.12aa | 136 | 277 |
| G1T4.137.12aa | 138 | 278 |
| G1T4.139.12aa | 140 | 279 |

Engineered MRA antibodies shown in Table 8 were expressed by transient expression using the engineered MRA heavy chains thus prepared in combination with the MRA light chain and using FreeStyle 293 cells (Invitrogen Corp.) or Expi293 cells (Life Technologies Corp.) by a method known to those skilled in the art, and purified by a method known to those skilled in the art using protein A.

TABLE 8
Engineered MRA antibodies

| | SEQ ID NO of | SEQ ID NO of |
|---|---|---|
| Antibody name | heavy chain | light chain |
| MRAVH007.12aa-G1T4/MRAL-k0 | 347 | 209 |
| MRAVH008.12aa-G1T4/MRAL-k0 | 348 | 209 |
| MRAVH009.12aa-G1T4/MRAL-k0 | 349 | 209 |
| MRAVH010.12aa-G1T4/MRAL-k0 | 350 | 209 |
| MRAVH011.12aa-G1T4/MRAL-k0 | 351 | 209 |
| MRAVH012.12aa-G1T4/MRAL-k0 | 352 | 209 |
| MRAVH013.12aa-G1T4/MRAL-k0 | 353 | 209 |
| MRAVH014.12aa-G1T4/MRAL-k0 | 354 | 209 |
| MRAVH015.12aa-G1T4/MRAL-k0 | 355 | 209 |
| MRAVH041.12aa-G1T4/MRAL-k0 | 356 | 209 |
| MRAVH042.12aa-G1T4/MRAL-k0 | 357 | 209 |
| MRAVH043.12aa-G1T4/MRAL-k0 | 358 | 209 |
| MRAVH044.12aa-G1T4/MRAL-k0 | 359 | 209 |
| MRAVH045.12aa-G1T4/MRAL-k0 | 360 | 209 |
| MRAVH046.12aa-G1T4/MRAL-k0 | 361 | 209 |
| MRAVH056.12aa-G1T4/MRAL-k0 | 362 | 209 |
| MRAVH057.12aa-G1T4/MRAL-k0 | 363 | 209 |
| MRAVH058.12aa-G1T4/MRAL-k0 | 364 | 209 |
| MRAVH059.12aa-G1T4/MRAL-k0 | 365 | 209 |
| MRAVH060.12aa-G1T4/MRAL-k0 | 366 | 209 |
| MRAVH061.12aa-G1T4/MRAL-k0 | 367 | 209 |
| MRAVH062.12aa-G1T4/MRAL-k0 | 368 | 209 |
| MRAVH063.12aa-G1T4/MRAL-k0 | 369 | 209 |
| MRAVH064.12aa-G1T4/MRAL-k0 | 370 | 209 |
| MRAVH065.12aa-G1T4/MRAL-k0 | 371 | 209 |
| MRAVH066.12aa-G1T4/MRAL-k0 | 372 | 209 |
| MRAVH067.12aa-G1T4/MRAL-k0 | 373 | 209 |
| MRAVH068.12aa-G1T4/MRAL-k0 | 374 | 209 |
| MRAVH069.12aa-G1T4/MRAL-k0 | 375 | 209 |
| MRAVH074.12aa-G1T4/MRAL-k0 | 376 | 209 |
| MRAVH075.12aa-G1T4/MRAL-k0 | 377 | 209 |
| MRAVH076.12aa-G1T4/MRAL-k0 | 378 | 209 |
| MRAVH077.12aa-G1T4/MRAL-k0 | 379 | 209 |
| MRAVH078.12aa-G1T4/MRAL-k0 | 380 | 209 |
| MRAVH087.12aa-G1T4/MRAL-k0 | 381 | 209 |
| MRAVH088.12aa-G1T4/MRAL-k0 | 382 | 209 |
| MRAVH089.12aa-G1T4/MRAL-k0 | 383 | 209 |
| MRAVH099.12aa-G1T4/MRAL-k0 | 384 | 209 |
| MRAVH100.12aa-G1T4/MRAL-k0 | 385 | 209 |
| MRAVH101.12aa-G1T4/MRAL-k0 | 386 | 209 |
| MRAVH102.12aa-G1T4/MRAL-k0 | 387 | 209 |
| MRAVH109.12aa-G1T4/MRAL-k0 | 388 | 209 |
| MRAVH110.12aa-G1T4/MRAL-k0 | 389 | 209 |
| MRAVH111.12aa-G1T4/MRAL-k0 | 390 | 209 |
| MRAVH112.12aa-G1T4/MRAL-k0 | 391 | 209 |
| MRAVH113.12aa-G1T4/MRAL-k0 | 392 | 209 |
| MRAVH114.12aa-G1T4/MRAL-k0 | 393 | 209 |
| MRAVH115.12aa-G1T4/MRAL-k0 | 394 | 209 |
| MRAVH116.12aa-G1T4/MRAL-k0 | 395 | 209 |
| MRAVH117.12aa-G1T4/MRAL-k0 | 396 | 209 |
| MRAVH118.12aa-G1T4/MRAL-k0 | 397 | 209 |
| MRAVH119.12aa-G1T4/MRAL-k0 | 398 | 209 |
| MRAH-G1T4.118.12aa/MRAL-k0 | 399 | 209 |
| MRAH-G1T4.119.12aa/MRAL-k0 | 400 | 209 |
| MRAH-G1T4.120.12aa/MRAL-k0 | 401 | 209 |
| MRAH-G1T4.121.12aa/MRAL-k0 | 402 | 209 |
| MRAH-G1T4.122.12aa/MRAL-k0 | 403 | 209 |
| MRAH-G1T4.123.12aa/MRAL-k0 | 404 | 209 |
| MRAH-G1T4.124.12aa/MRAL-k0 | 405 | 209 |
| MRAH-G1T4.129.12aa/MRAL-k0 | 406 | 209 |
| MRAH-G1T4.130.12aa/MRAL-k0 | 407 | 209 |
| MRAH-G1T4.131.12aa/MRAL-k0 | 408 | 209 |
| MRAH-G1T4.132.12aa/MRAL-k0 | 409 | 209 |
| MRAH-G1T4.134.12aa/MRAL-k0 | 410 | 209 |
| MRAH-G1T4.135.12aa/MRAL-k0 | 411 | 209 |
| MRAH-G1T4.137.12aa/MRAL-k0 | 412 | 209 |
| MRAH-G1T4.139.12aa/MRAL-k0 | 413 | 209 |

### 14-2. Protease Cleavage Evaluation of Anti-Human IL6R Neutralizing Antibody Harboring Protease Cleavage Sequence in its Antibody Heavy Chain

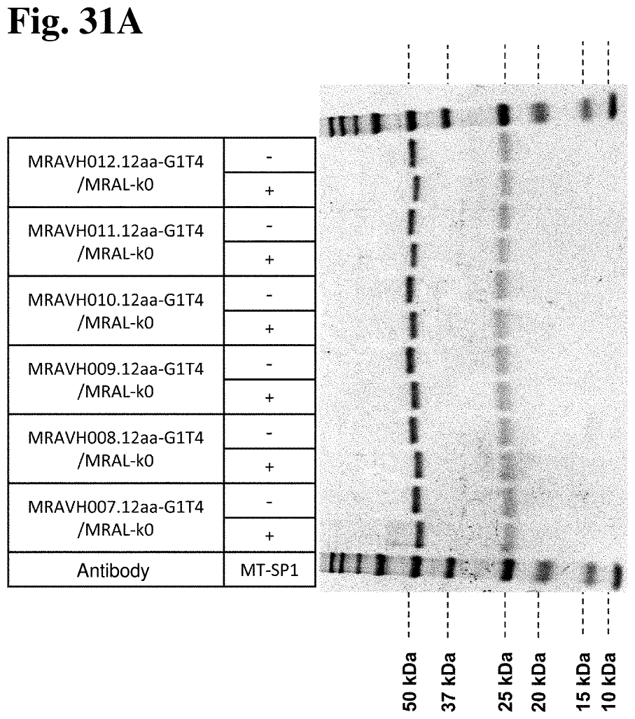

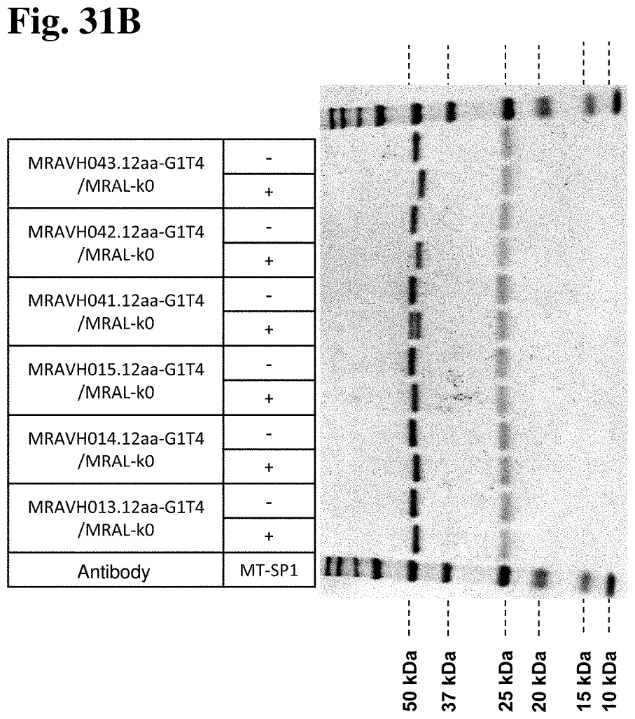

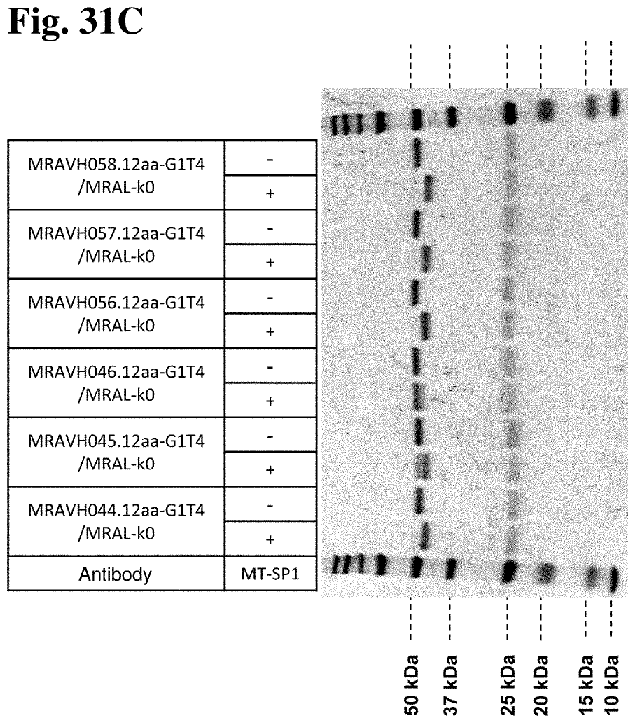

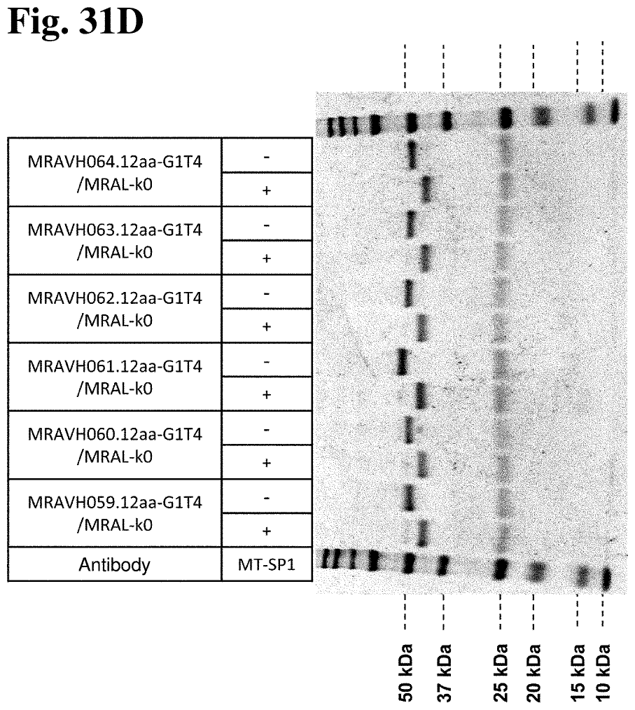

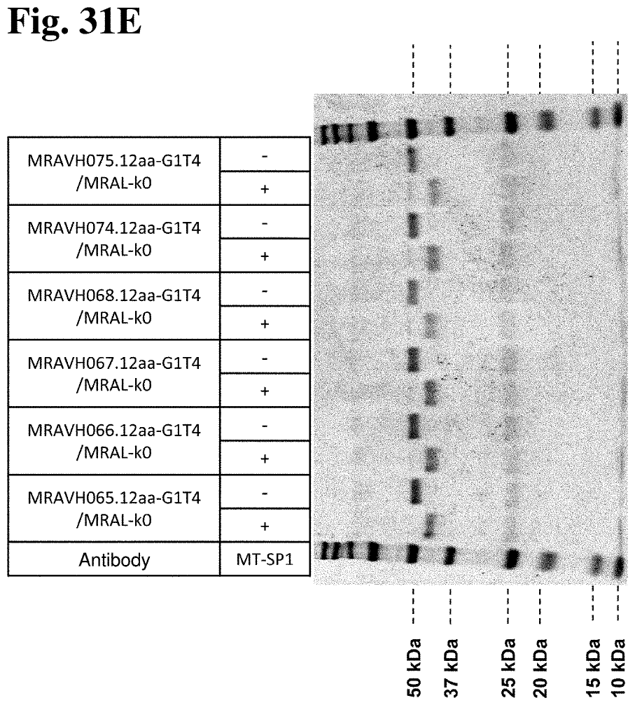

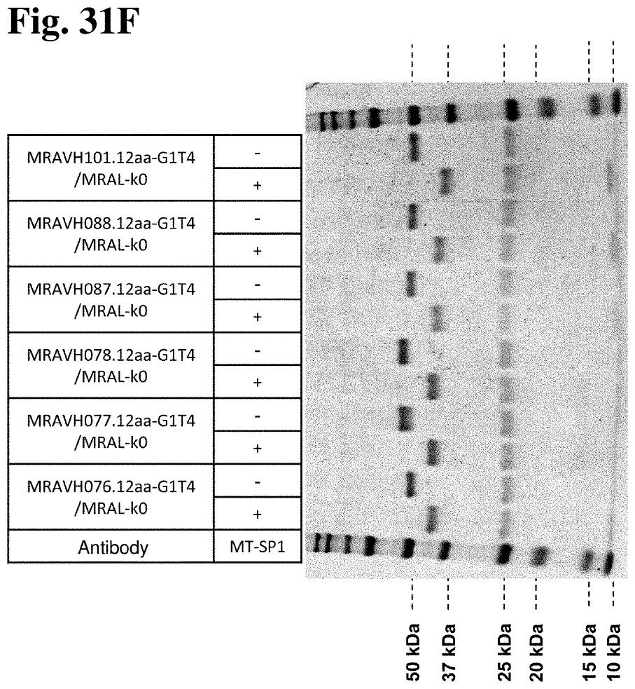

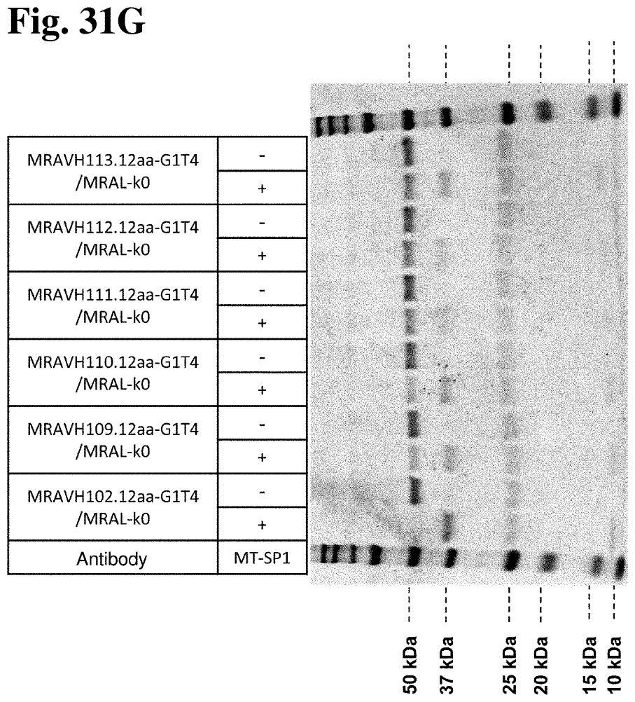

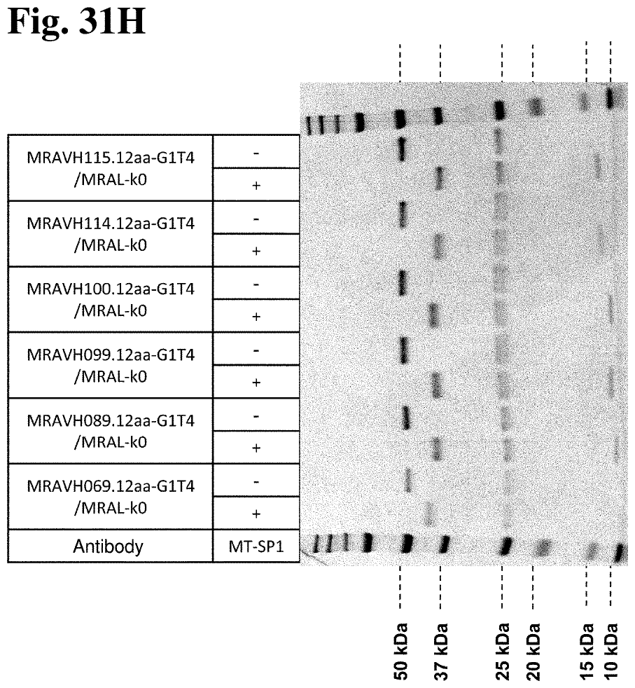

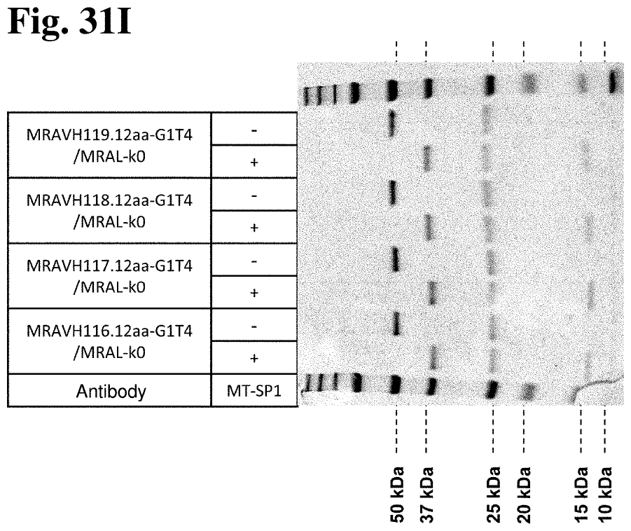

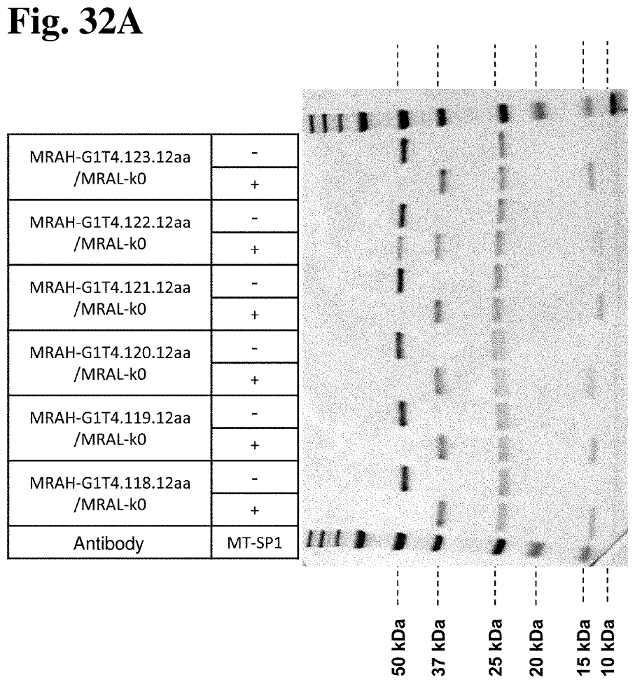

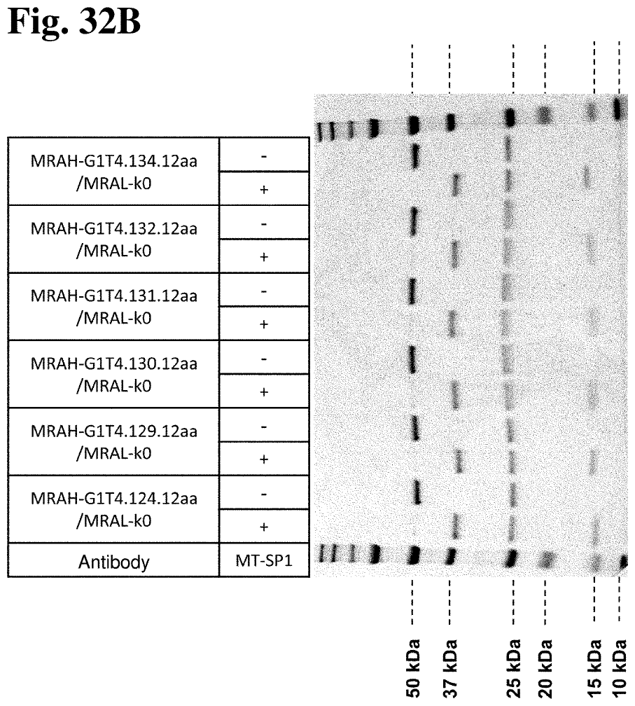

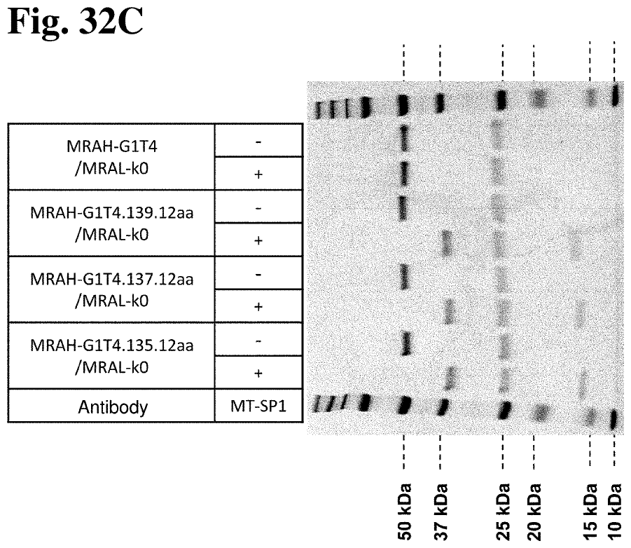

Whether the engineered MRA antibodies prepared in Example 14-1 would be cleaved by protease was verified. Recombinant Human Matriptase/ST14 Catalytic Domain (human MT-SP1, hMT-SP1) (R&D Systems, Inc., 3946-SE-010) was used as the protease. 10 nM protease and 50 micro g/mL of each antibody were reacted in PBS under a condition of 37 degrees C. for 20 hours, followed by reducing SDS-PAGE. The results are shown in FIGS. 31A, 31B, 31C, 31D, 31E, 31F, 31G, 31H, 31I, 32A, 32B, and 32C. The protease-treated engineered MRA antibodies underwent cleavage at their heavy chains and generated a heavy chain band at a position with a smaller molecular weight than that of the heavy chains of protease-untreated engineered MRA antibodies (in the drawings, a band appearing around 50 kDa in the MT-SP1(−) lane). From this result, the engineered MRA antibodies prepared in Example 14-1 were confirmed to be cleaved by hMT-SP1.

### Example 15: Evaluation of Antibodies Harboring Protease Cleavage Sequence at Diverse Positions of Light Chain

### 15-1. Preparation of Antibodies Harboring Protease Cleavage Sequence at Diverse Positions of Light Chain

Peptide sequence B (SEQ ID NO: 210) reportedly cleavable by urokinase (uPA) and matriptase (MT-SP1) was inserted at each of different positions within a MRA light chain variable region (MRAL; SEQ ID NO: 280) to prepare engineered MRA light chain variable regions shown in Table 9. These engineered MRA light chain variable regions were each linked to a MRA light chain constant region (k0; SEQ ID NO: 281) to prepare engineered MRA light chains. The corresponding gene expression vectors were prepared by a method known to those skilled in the art. Also, peptide sequence B (SEQ ID NO: 210) was inserted at each of different positions within a MRA light chain constant region (k0; SEQ ID NO: 281) to prepare engineered MRA light chain constant regions shown in Table 10. These engineered MRA light chain constant regions were each linked to a MRA light chain variable region (MRAL; SEQ ID NO: 280) to prepare engineered MRA light chains. The corresponding gene expression vectors were prepared by a method known to those skilled in the art. Tables 9 and 10 also show the protease cleavage sequence insertion positions in the prepared engineered MRA light chain variable regions and engineered MRA light chain constant regions. In Table 9, the inserted sequence was located adjacent on the constant region side to the described amino acid position (Kabat numbering) in the antibody light chain variable region. In Table 10, the inserted sequence was located adjacent on the variable region side to the described amino acid position (EU numbering) in the antibody light chain constant region.

TABLE 9
Engineered MRA light chain variable regions and
protease cleavage sequence insertion positions

| | Protease cleavage sequence | |
|---|---|---|
| Engineered MRA light | insertion position | SEQ |
| chain variable region | (Kabat numbering) | ID NO |
| MRAVL007.12aa | 7 | 282 |
| MRAVL008.12aa | 8 | 283 |
| MRAVL009.12aa | 9 | 284 |
| MRAVL010.12aa | 10 | 285 |
| MRAVL011.12aa | 11 | 286 |
| MRAVL012.12aa | 12 | 287 |
| MRAVL013.12aa | 13 | 288 |
| MRAVL014.12aa | 14 | 289 |
| MRAVL015.12aa | 15 | 290 |
| MRAVL016.12aa | 16 | 291 |
| MRAVL017.12aa | 17 | 292 |
| MRAVL018.12aa | 18 | 293 |
| MRAVL039.12aa | 39 | 294 |
| MRAVL040.12aa | 40 | 295 |
| MRAVL041.12aa | 41 | 296 |
| MRAVL042.12aa | 42 | 297 |
| MRAVL043.12aa | 43 | 298 |
| MRAVL044.12aa | 44 | 299 |
| MRAVL045.12aa | 45 | 300 |
| MRAVL049.12aa | 49 | 301 |
| MRAVL050.12aa | 50 | 302 |
| MRAVL051.12aa | 51 | 303 |
| MRAVL052.12aa | 52 | 304 |
| MRAVL053.12aa | 53 | 305 |
| MRAVL054.12aa | 54 | 306 |
| MRAVL055.12aa | 55 | 307 |
| MRAVL056.12aa | 56 | 308 |
| MRAVL057.12aa | 57 | 309 |
| MRAVL058.12aa | 58 | 310 |
| MRAVL059.12aa | 59 | 311 |
| MRAVL060.12aa | 60 | 312 |
| MRAVL096.12aa | 96 | 313 |
| MRAVL097.12aa | 97 | 314 |
| MRAVL098.12aa | 98 | 315 |
| MRAVL099.12aa | 99 | 316 |
| MRAVL100.12aa | 100 | 317 |
| MRAVL101.12aa | 101 | 318 |
| MRAVL102.12aa | 102 | 319 |
| MRAVL103.12aa | 103 | 320 |
| MRAVL104.12aa | 104 | 321 |
| MRAVL105.12aa | 105 | 322 |
| MRAVL106.12aa | 106 | 323 |
| MRAVL107.12aa | 107 | 324 |

TABLE 10
Engineered MRA light chain constant regions and
protease cleavage sequence insertion positions

| | Protease cleavage sequence | |
|---|---|---|
| Engineered MRA light | insertion position | SEQ |
| chain constant region | (EU numbering) | ID NO |
| k0.108.12aa | 109 (Kabat numbering 109) | 325 |
| k0.109.12aa | 110 (Kabat numbering 110) | 326 |
| k0.110.12aa | 111 (Kabat numbering 111) | 327 |
| k0.111.12aa | 112 (Kabat numbering 112) | 328 |
| k0.112.12aa | 113 (Kabat numbering 113) | 329 |
| k0.113.12aa | 114 (Kabat numbering 114) | 330 |
| k0.115.12aa | 116 (Kabat numbering 116) | 331 |
| k0.116.12aa | 117 (Kabat numbering 117) | 332 |
| k0.117.12aa | 118 (Kabat numbering 118) | 333 |
| k0.118.12aa | 119 (Kabat numbering 119) | 334 |
| k0.119.12aa | 120 (Kabat numbering 120) | 335 |
| k0.120.12aa | 121 (Kabat numbering 121) | 336 |
| k0.121.12aa | 122 (Kabat numbering 122) | 337 |
| k0.122.12aa | 123 (Kabat numbering 123) | 338 |
| k0.123.12aa | 124 (Kabat numbering 124) | 339 |
| k0.124.12aa | 125 (Kabat numbering 125) | 340 |
| k0.125.12aa | 126 (Kabat numbering 126) | 341 |
| k0.126.12aa | 127 (Kabat numbering 127) | 342 |
| k0.127.12aa | 128 (Kabat numbering 128) | 343 |
| k0.128.12aa | 129 (Kabat numbering 129) | 344 |
| k0.129.12aa | 130 (Kabat numbering 130) | 345 |
| k0.130.12aa | 131 (Kabat numbering 131) | 346 |

Engineered MRA antibodies shown in Table 11 were expressed by transient expression using the engineered MRA light chains thus prepared in combination with the MRA heavy chain and using FreeStyle 293 cells (Invitrogen Corp.) or Expi293 cells (Life Technologies Corp.) by a method known to those skilled in the art, and purified by a method known to those skilled in the art using protein A.

TABLE 11
Engineered MRA antibodies

| | SEQ ID NO of | SEQ ID NO of |
|---|---|---|
| Antibody name | heavy chain | light chain |
| MRAH-G1T4/MRAVL007.12aa-k0 | 208 | 414 |
| MRAH-G1T4/MRAVL008.12aa-k0 | 208 | 415 |
| MRAH-G1T4/MRAVL009.12aa-k0 | 208 | 416 |
| MRAH-G1T4/MRAVL010.12aa-k0 | 208 | 417 |
| MRAH-G1T4/MRAVL011.12aa-k0 | 208 | 418 |
| MRAH-G1T4/MRAVL012.12aa-k0 | 208 | 419 |
| MRAH-G1T4/MRAVL013.12aa-k0 | 208 | 420 |
| MRAH-G1T4/MRAVL014.12aa-k0 | 208 | 421 |
| MRAH-G1T4/MRAVL015.12aa-k0 | 208 | 422 |
| MRAH-G1T4/MRAVL016.12aa-k0 | 208 | 423 |
| MRAH-G1T4/MRAVL017.12aa-k0 | 208 | 424 |
| MRAH-G1T4/MRAVL018.12aa-k0 | 208 | 425 |
| MRAH-G1T4/MRAVL039.12aa-k0 | 208 | 426 |
| MRAH-G1T4/MRAVL040.12aa-k0 | 208 | 427 |
| MRAH-G1T4/MRAVL041.12aa-k0 | 208 | 428 |
| MRAH-G1T4/MRAVL042.12aa-k0 | 208 | 429 |
| MRAH-G1T4/MRAVL043.12aa-k0 | 208 | 430 |
| MRAH-G1T4/MRAVL044.12aa-k0 | 208 | 431 |
| MRAH-G1T4/MRAVL045.12aa-k0 | 208 | 432 |
| MRAH-G1T4/MRAVL049.12aa-k0 | 208 | 433 |
| MRAH-G1T4/MRAVL050.12aa-k0 | 208 | 434 |
| MRAH-G1T4/MRAVL051.12aa-k0 | 208 | 435 |
| MRAH-G1T4/MRAVL052.12aa-k0 | 208 | 436 |
| MRAH-G1T4/MRAVL053.12aa-k0 | 208 | 437 |
| MRAH-G1T4/MRAVL054.12aa-k0 | 208 | 438 |
| MRAH-G1T4/MRAVL055.12aa-k0 | 208 | 439 |
| MRAH-G1T4/MRAVL056.12aa-k0 | 208 | 440 |
| MRAH-G1T4/MRAVL057.12aa-k0 | 208 | 441 |
| MRAH-G1T4/MRAVL058.12aa-k0 | 208 | 442 |
| MRAH-G1T4/MRAVL059.12aa-k0 | 208 | 443 |
| MRAH-G1T4/MRAVL060.12aa-k0 | 208 | 444 |
| MRAH-G1T4/MRAVL096.12aa-k0 | 208 | 445 |
| MRAH-G1T4/MRAVL097.12aa-k0 | 208 | 446 |
| MRAH-G1T4/MRAVL098.12aa-k0 | 208 | 447 |
| MRAH-G1T4/MRAVL099.12aa-k0 | 208 | 448 |
| MRAH-G1T4/MRAVL100.12aa-k0 | 208 | 449 |
| MRAH-G1T4/MRAVL101.12aa-k0 | 208 | 450 |
| MRAH-G1T4/MRAVL102.12aa-k0 | 208 | 451 |
| MRAH-G1T4/MRAVL103.12aa-k0 | 208 | 452 |
| MRAH-G1T4/MRAVL104.12aa-k0 | 208 | 453 |
| MRAH-G1T4/MRAVL105.12aa-k0 | 208 | 454 |
| MRAH-G1T4/MRAVL106.12aa-k0 | 208 | 455 |
| MRAH-G1T4/MRAVL107.12aa-k0 | 208 | 456 |
| MRAH-G1T4/MRAL-k0.108.12aa | 208 | 457 |
| MRAH-G1T4/MRAL-k0.109.12aa | 208 | 458 |
| MRAH-G1T4/MRAL-k0.110.12aa | 208 | 459 |
| MRAH-G1T4/MRAL-k0.111.12aa | 208 | 460 |
| MRAH-G1T4/MRAL-k0.112.12aa | 208 | 461 |
| MRAH-G1T4/MRAL-k0.113.12aa | 208 | 462 |
| MRAH-G1T4/MRAL-k0.115.12aa | 208 | 463 |
| MRAH-G1T4/MRAL-k0.116.12aa | 208 | 464 |
| MRAH-G1T4/MRAL-k0.117.12aa | 208 | 465 |
| MRAH-G1T4/MRAL-k0.118.12aa | 208 | 466 |
| MRAH-G1T4/MRAL-k0.119.12aa | 208 | 467 |
| MRAH-G1T4/MRAL-k0.120.12aa | 208 | 468 |
| MRAH-G1T4/MRAL-k0.121.12aa | 208 | 469 |
| MRAH-G1T4/MRAL-k0.122.12aa | 208 | 470 |
| MRAH-G1T4/MRAL-k0.123.12aa | 208 | 471 |
| MRAH-G1T4/MRAL-k0.124.12aa | 208 | 472 |
| MRAH-G1T4/MRAL-k0.125.12aa | 208 | 473 |
| MRAH-G1T4/MRAL-k0.126.12aa | 208 | 474 |
| MRAH-G1T4/MRAL-k0.127.12aa | 208 | 475 |
| MRAH-G1T4/MRAL-k0.128.12aa | 208 | 476 |
| MRAH-G1T4/MRAL-k0.129.12aa | 208 | 477 |
| MRAH-G1T4/MRAL-k0.130.12aa | 208 | 478 |

### 15-2. Protease Cleavage Evaluation of Anti-Human IL6R Neutralizing Antibody Harboring Protease Cleavage Sequence in its Antibody Light Chain Variable Region

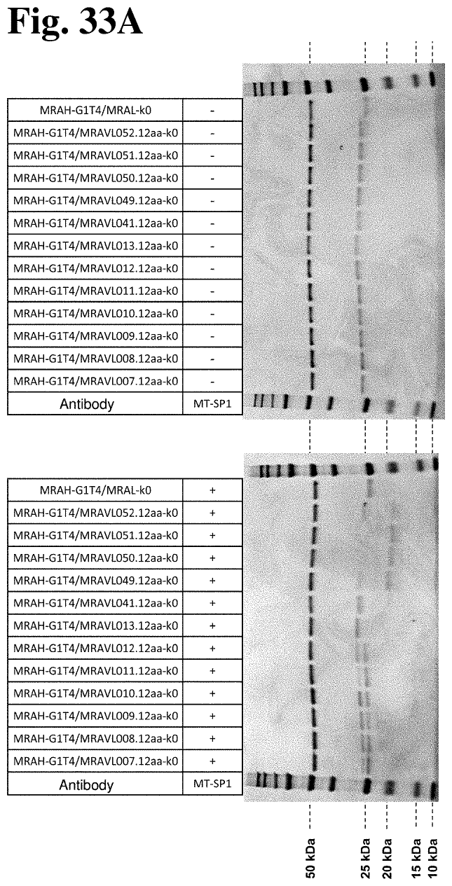

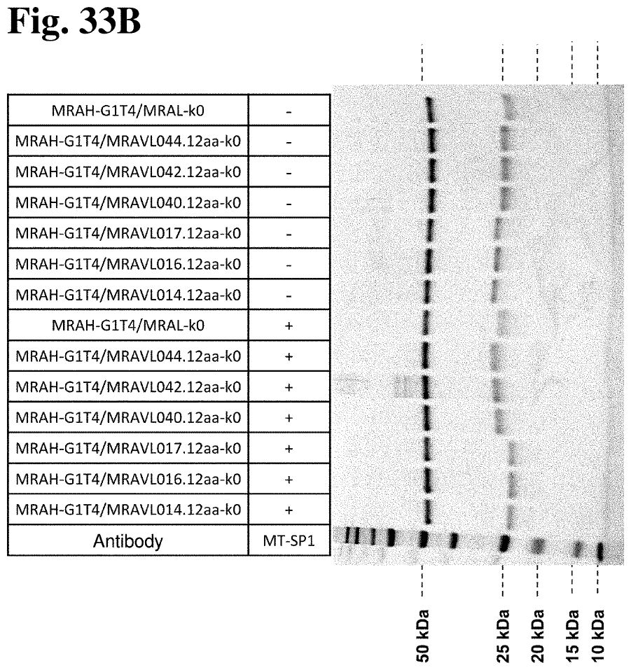

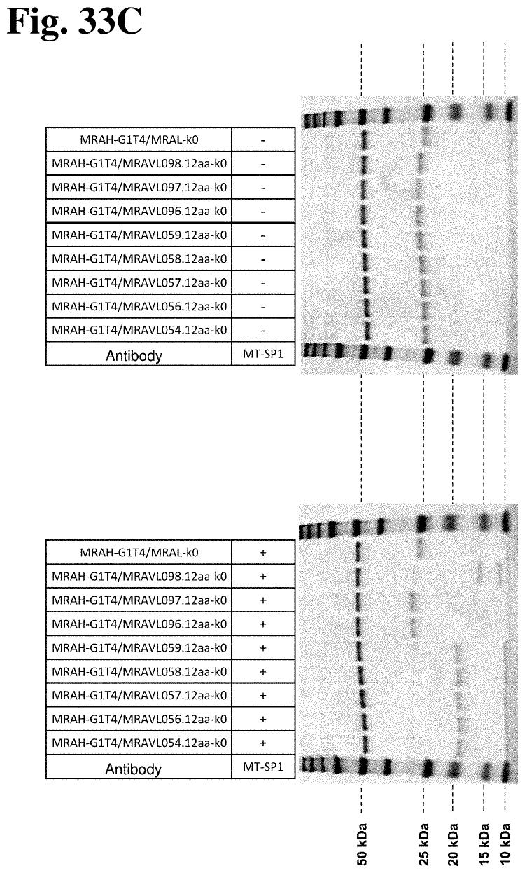

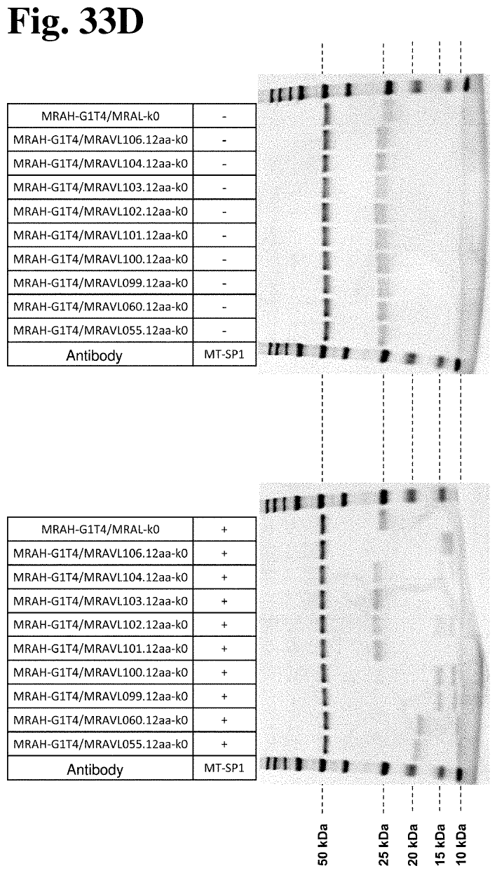

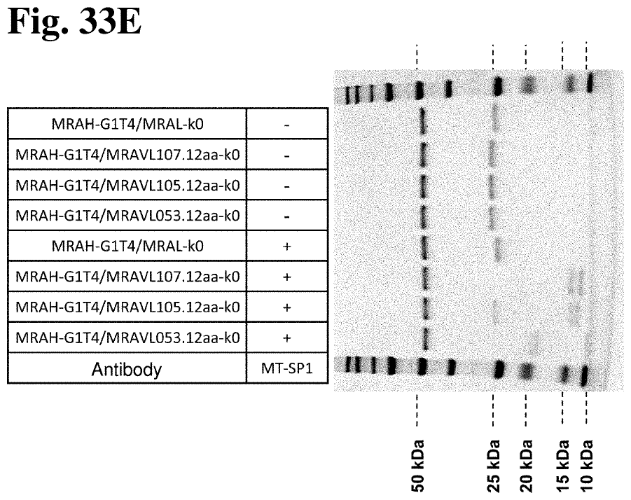

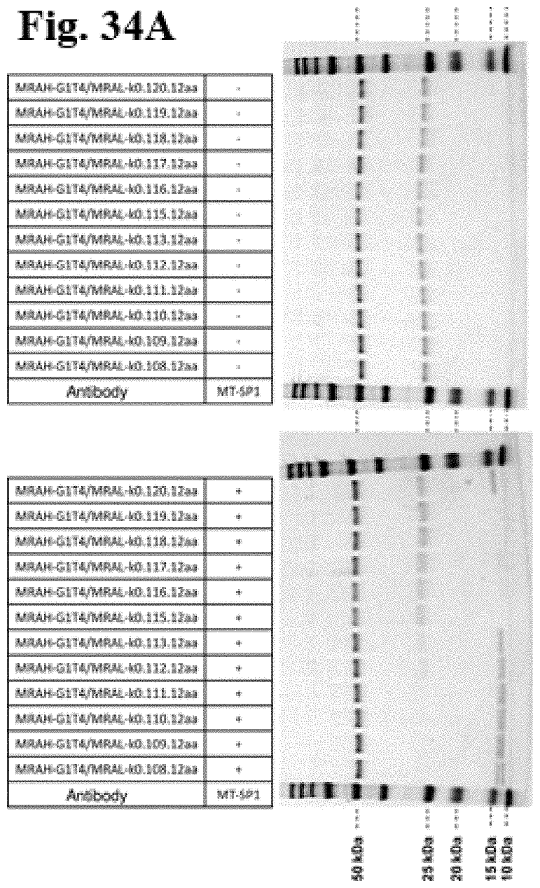

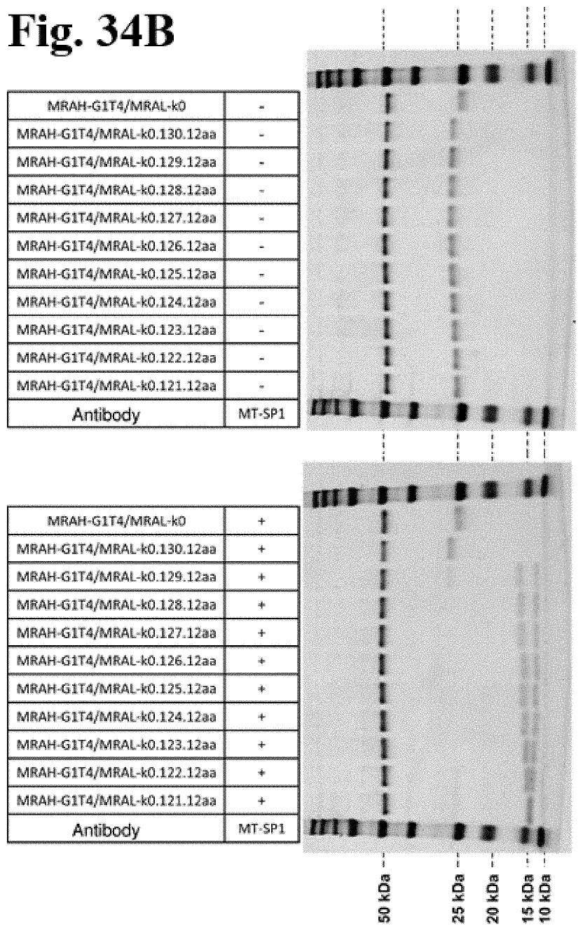

Whether the engineered MRA antibodies prepared in Example 15-1 would be cleaved by protease was verified. Recombinant Human Matriptase/ST14 Catalytic Domain (MT-SP1) (R&D Systems, Inc., 3946-SE-010) was used as the protease. 10 nM protease and 50 micro g/mL of each antibody were reacted in PBS under a condition of 37 degrees C. for 20 hours, followed by reducing SDS-PAGE. The results are shown in FIGS. 33A, 33B, 33C, 33D, 33E, 34A, and 34B. The protease-treated engineered MRA antibodies underwent cleavage at their light chains and generated a light chain band at a position with a smaller molecular weight than that of the light chains of protease-untreated engineered MRA antibodies (in the drawings, a band appearing around 25 kDa in the MT-SP1(−) lane).

### Example 16

16-1. Introduction of Protease Cleavage Sequence to Polypeptide with Incorporated VL Binding to IL-1R

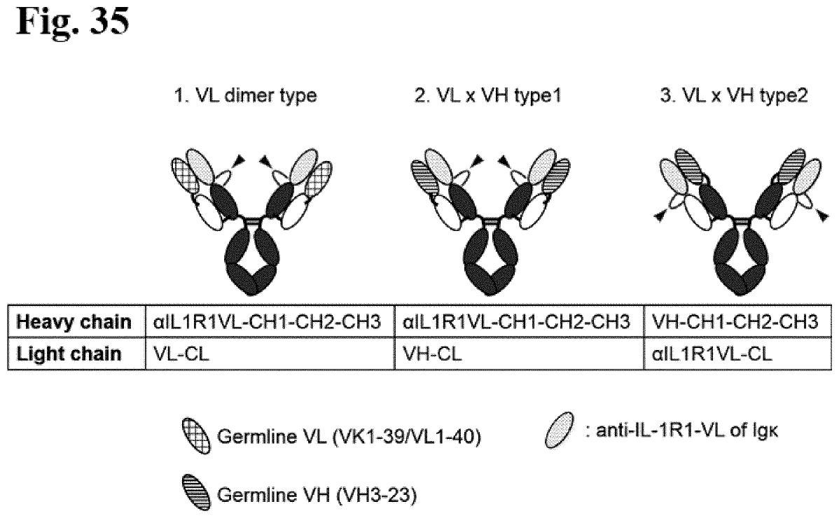

As shown in the previous Examples VHH could be used as antigen binding single-domain antibody in protease-activated polypeptide. VL single domain antibody, which binds to antigen alone, has been reported that VL single domain has further better physicochemical properties compared with VHH or VH single domain (J. Mol. Biol., 342 (2004), pp. 901-912, J. Mol. Biol. (2003) 325, 531-553). Thus we expected that VL single domain could be used for antigen binding domain in IgG-antibody-like molecule as shown in FIG. 35.

To construct protease-activated IgG antibody-like molecules, a protease cleavage sequence was inserted near the boundary between the anti-IL-1R1-VL (DOM4.122.23 (SEQ ID NO: 479) or DOM4.130.202 (SEQ ID NO: 480)), and either CH1 or Igkappa depending on the three IgG-antibody-like molecule formats shown in FIG. 35.

In the VL dimer type and VL×VH type1 format, three types of heavy chain constant regions shown in Table 13 (iSG1, kSG1, uPASG1) were designed such that peptide sequence i (SEQ ID NO: 508), k (SEQ ID NO: 509), or uPA (SEQ ID NO: 510) was inserted at a site near the boundary between VL DOM4.122.23 or DOM4.130.202, and CH1. i and k are reported sequences cleavable by MMP13, and uPA is cleavable by urokinase (uPA).

In the VL×VH type2 format, three types of light chain constant regions shown in Figure CK1B (iSK1, kSK1, uPASK1) were designed such that peptide sequence i (SEQ ID NO: 508), k (SEQ ID NO: 509), or uPA (SEQ ID NO: 510) was inserted at a site near the boundary between VL DOM4.122.23 or DOM4.130.202, and Igkappa.

Expression vectors encoding DOM4.122.23-iSG1 (SEQ ID NO: 492) DOM4.122.23-kSG1 (SEQ ID NO: 493), DOM4.122.23-uPASG1 (SEQ ID NO: 494), DOM4.130.202-iSG1 (SEQ ID NO: 495), DOM4.130.202-kSG1 (SEQ ID NO: 496), DOM4.130.202-uPASG1 (SEQ ID NO: 497), DOM4.122.23-iSK1 (SEQ ID NO: 502), DOM4.122.23-kSK1 (SEQ ID NO: 503), DOM4.122.23-uPASK1 (SEQ ID NO: 504), DOM4.130.202-iSK1 (SEQ ID NO: 505), DOM4.130.202-kSK1 (SEQ ID NO: 506), and DOM4.130.202-uPASK1 (SEQ ID NO: 507) were prepared by a method known to those skilled in the art.

Expression vector encoding VH3.23-SG1 (SEQ ID NO: 498) as heavy chain (variable region-constant region) having a human germline sequence was prepared by a method known to those skilled in the art.

Expression vectors encoding VK1.39-SK1 (SEQ ID NO: 499), VL1.40-lam1 (SEQ ID NO: 500), VH3.23-SK1 (SEQ ID NO: 501) as light chains (variable region-constant region) of various subclasses having a human germline sequence were prepared (VK1.39 (SEQ ID NO: 484), VL1.40 (SEQ ID NO: 485), VH3.23 (SEQ ID NO: 486), lam1 (SEQ ID NO: 488) by a method known to those skilled in the art).

16-2. Preparation of Polypeptide with Incorporated VL Binding and Cleavage Site to IL-1R

Protease-activated IgG antibody-like molecules shown in Table 12 below were expressed by transient expression using Expi 293 cells (Invitrogen Corp.) by a method known to those skilled in the art, and purified by a method known to those skilled in the art using protein A. Table 13 shows the name of each prepared antibody constant region, the insertion site of the amino acid sequence, and the inserted amino acid sequence. The insertion site is indicated by [insert].

TABLE 12
Protease-activated IgG antibody-like molecules

| IgG-antibody- | | | |
|---|---|---|---|
| like molecule | | SEQ ID NO of | SEQ ID NO of |
| format | IgG antibody-like molecule | heavy chain | light chain |
| VL dimer | DOM4.122.23-iSG1/VK1.39-SK1 | 492 | 499 |
| type | DOM4.122.23-kSG1/VK1.39-SK1 | 493 | |
| | DOM4.122.23-uPASG1/VK1.39-SK1 | 494 | |
| | DOM4.130.202-iSG1/VK1.39-SK1 | 495 | |
| | DOM4.130.202-kSG1/VK1.39-SK1 | 496 | |
| | DOM4.130.202-uPASG1/VK1.39-SK1 | 497 | |
| | DOM4.122.23-iSG1/VL1.40-lam1 | 492 | 500 |
| | DOM4.122.23-kSG1/VL1.40-lam1 | 493 | |
| | DOM4.122.23-uPASG1/VL1.40-lam1 | 494 | |
| | DOM4.130.202-iSG1/VL1.40-lam1 | 495 | |
| | DOM4.130.202-kSG1/VL1.40-lam1 | 496 | |
| | DOM4.130.202-uPASG1/VL1.40-lam1 | 497 | |
| VL × VH | DOM4.122.23-iSG1/VH3.23-SK1 | 492 | 501 |
| type1 | DOM4.122.23-kSG1/VH3.23-SK1 | 493 | |
| | DOM4.122.23-uPASG1/VH3.23-SK1 | 494 | |
| | DOM4.130.202-iSG1/VH3.23-SK1 | 495 | |
| | DOM4.130.202-kSG1/VH3.23-SK1 | 496 | |
| | DOM4.130.202-uPASG1/VH3.23-SK1 | 497 | |
| VL × VH | VH3.23-SG1/DOM4.122.23-iSK1 | 498 | 502 |
| type2 | VH3.23-SG1/DOM4.122.23-kSK1 | | 503 |
| | VH3.23-SG1/DOM4.122.23-uPASK1 | | 504 |
| | VH3.23-SG1/DOM4.130.202-iSK1 | | 505 |
| | VH3.23-SG1/DOM4.130.202-kSK1 | | 506 |
| | VH3.23-SG1/DOM4.130.202-uPASK1 | | 507 |

TABLE 13
The name of each prepared antibody
constant region, the insertion
site of the amino acid sequence
and the inserted amino acid sequence

| | | Inserted |
|---|---|---|
| Name of | | amino |
| Constant | Insertion | acid |
| region | site | sequence |
| iSG1 | A [insert] | GAGPQGLA |
| (SEQ ID | STKGPS | GQRGIVAG |
| NO: 481) | (SEQ ID | (SEQ ID |
| | NO: 623) | NO: 508) |
| kSG1 | A [insert] | GPPGPQG |
| (SEQ ID | STKGPS | LAGQRGI |
| NO: 482) | (SEQ ID | VGL |
| | NO: 623) | (SEQ ID |
| | | NO: 509) |
| UpaSG1 | A [insert] | TSTSGR |
| (SEQ ID | STKGPS | SANPRG |
| NO: 483) | (SEQ ID | (SEQ ID |
| | NO: 623) | NO: 510) |
| iSK1 | R [insert] | GAGPQGLA |
| (SEQ ID | TVAAPS | GQRGIVAG |
| NO: 489) | (SEQ ID | (SEQ ID |
| | NO: 624) | NO: 508) |
| KSK1 | R [insert] | GPPGPQGL |
| (SEQ ID | TVAAPS | AGQRGIVGL |
| NO: 490) | (SEQ ID | (SEQ ID |
| | NO: 624) | NO: 509) |
| uPASK1 | R [insert] | TSTSGR |
| (SEQ ID | TVAAPS | SANPRG |
| NO: 491) | (SEQ ID | (SEQ ID |
| | NO: 624) | NO: 510) |

### Example 17: Protease Cleavage Evaluation of IgG Antibody-Like Molecules with Anti-IL-1R1 Binding Domain

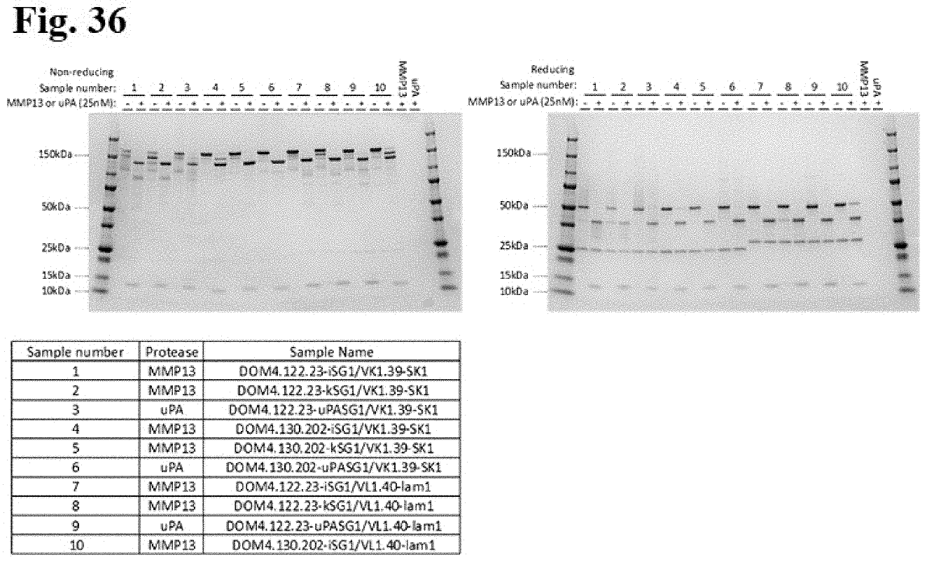

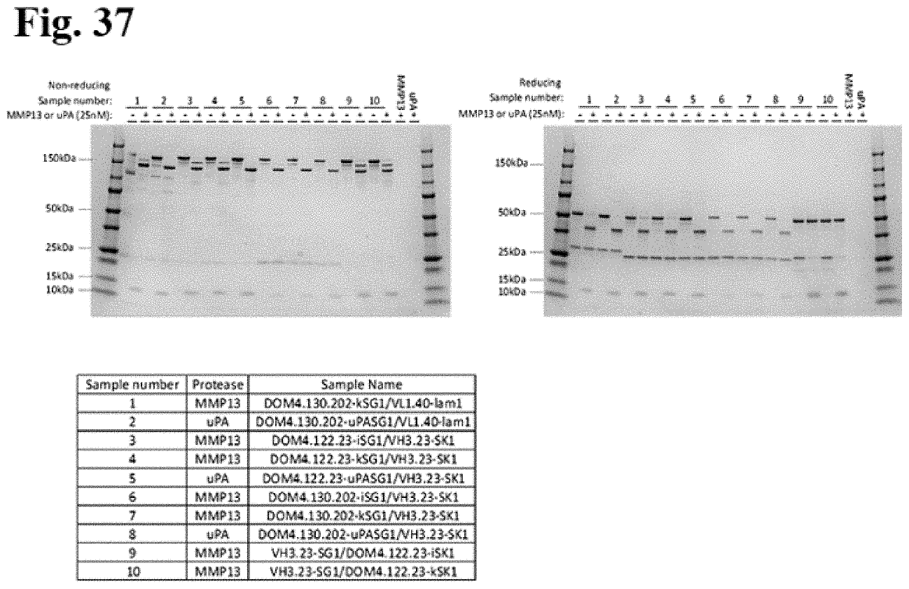

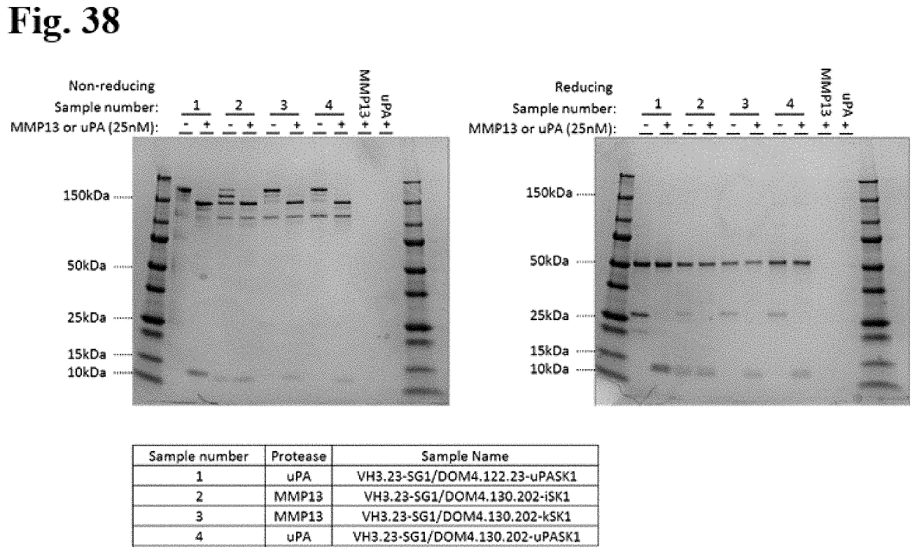

MMP13 (R&D systems, 511-MM-010) was activated with 1 micromolar of 4-aminophenylmercuric acetate (APMA, Sigma, A9563). Activated MMP13 or uPA (R&D systems, 1310-SE-010) were added to IgG antibody-like molecules with anti-IL-1R1 binding domain described in Example 16. The final concentration of IgG antibody-like molecules is 800 nM and the final concentration of proteases is 25 nM. Reaction mixtures were incubated at 37 degrees C. for 20 hours. The degree of the cleavage was evaluated by reducing and non-reducing SDS-PAGE. The protease cleavage of IgG antibody-like molecules was confirmed and correspond bands of anti-IL-1R1 domain antibody were observed between 15 kDa and 10 kDa (FIGS. 36, 37, and 38).

### Example 18: Cell Assay with IgG Antibody-Like Molecules with Anti-IL-1R1 Binding Domain

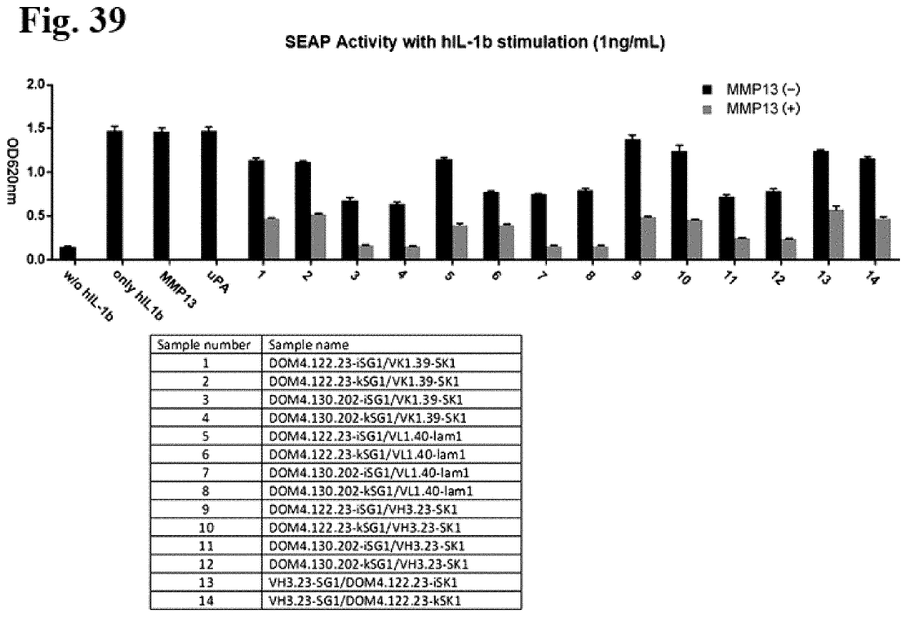

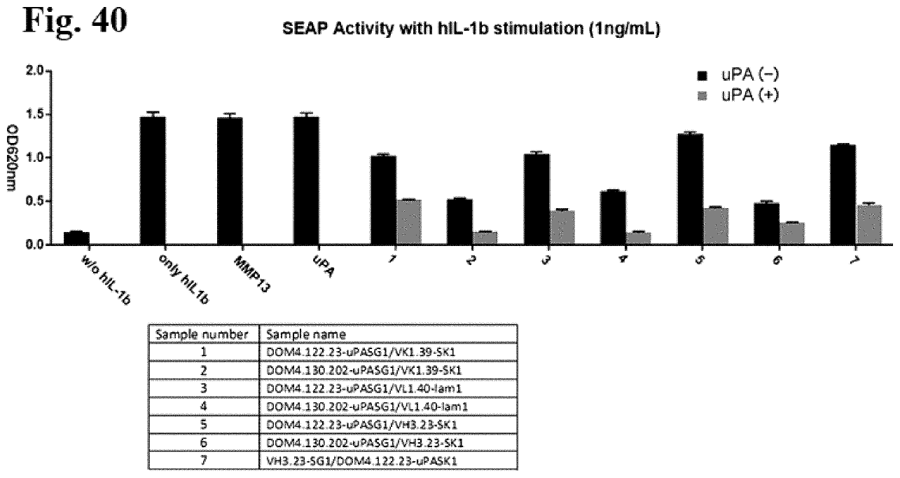

HEK-Blue™ IL-1beta cells were maintained in DMEM medium (Gibco) supplemented with 10% fetal bovine serum, 50 U/mL streptomycin, 50 micro g/mL penicillin, 100 micro g/mL Normocin™, 200 micro g/mL of Hygromycin B Gold and 100 micro g/mL of Zeocin™. During functional assay, the medium for cells was changed to assay medium (DMEM with 10% FBS, 50 U/mL streptomycin, 50 micro g/mL penicillin and 100 micro g/mL Normocin™) and seeded to 96-well plates with seeding density of 5E4 cells per well. Then, IgG antibody-like molecules reaction mixture, as described in Example 17, were diluted 100× with assay medium and incubated together with HEK-Blue™ IL-1beta cells for an hour at 37 degrees C. incubator supplied with 5% CO2. Human recombinant IL-1beta was then added to the wells to a final concentration of 1 ng/mL for cell stimulation overnight at 37 degrees C. incubator supplied with 5% CO2. Cell supernatant was mixed with QUANTI-Blue™ and incubated at 37 degrees C. for 30 minutes, after which the optical density at 620 nm was measured in a colorimetric plate reader. Neutralizing activity was enhanced after protease treatment with IgG antibody-like molecules with anti-IL-1R1 binding domain (FIGS. 39 and 40).

### Example 19: Preparation of Protease Cleavage Sequence to Polypeptide with Incorporated VL Binding to CD154

To construct protease-activated IgG antibody-like molecules, a protease cleavage sequence was inserted near the boundary between the anti-CD154 VL (DOM8.8 (SEQ ID NO: 533) and Igkappa. Expression vectors encoding heavy chain MORH-wtG4d (SEQ ID NO: 534), heavy chain MORH-G1d (SEQ ID NO: 535), MORH-IgG2dGK (SEQ ID NO: 536) and MDXH-mFa55 (SEQ ID NO: 537) were prepared by a method known to those skilled in the art. Heavy chain and protease cleavage sequence containing light chain DOM8.8-Pk0MT (SEQ ID NO: 538) were co-expressed by transient expression using Expi 293 cells (Invitrogen Corp.) by a method known to those skilled in the art, and purified by a method known to those skilled in the art using protein A.

TABLE 14

| | | | Inserted | |
|---|---|---|---|---|
| | | | protease | |
| | | | digestion | |
| | Heavy | Light | sequence | |
| | chain | chain | with | |
| | sequence | sequence | flexible | Insertion |
| Name | ID | ID | linker | position |
| MORH- | 534 | 538 | GSGLSGRS | KVEIK |
| wtG4d/ | | | DNHGSSG | [insert] |
| DOM8.8- | | | GSS | RTVAA |
| Pk0MT | | | (SEQ ID | (SEQ ID |
| | | | NO: 625) | NO: 626) |
| MORH- | 535 | 538 | GSGLSGRS | KVEIK |
| G1d/DOM8.8- | | | DNHGSSG | [insert] |
| Pk0MT | | | GSS | RTVAA |
| | | | (SEQ ID | (SEQ ID |
| | | | NO: 625) | NO: 626) |
| MORH- | 536 | 538 | GSGLSGRS | KVEIK |
| IgG2dGK/ | | | DNHGSSG | [insert] |
| DOM8.8- | | | GSS | RTVAA |
| Pk0MT | | | (SEQ ID | (SEQ ID |
| | | | NO: 625) | NO: 626) |
| MDXH- | 537 | 538 | GSGLSGR | KVEIK |
| mFa55/ | | | SDNHGSSG | [insert] |
| DOM8.8- | | | GSS | RTVAA |
| Pk0MT | | | (SEQ ID | (SEQ ID |
| | | | NO: 625) | NO: 626) |

### Example 20: Protease Cleavage Evaluation of IgG Antibody-Like Molecules Harboring Protease Cleavage Sequence

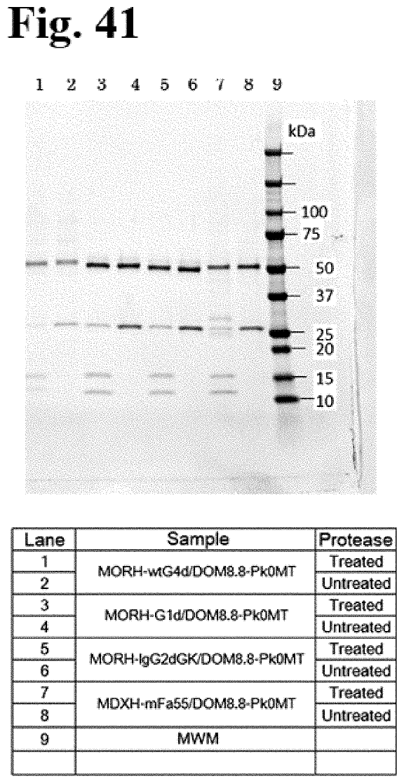

The IgG antibody-like molecule prepared in Example 19 was cleaved by protease in the same way as in Example 3, and the cleavage was evaluated by reducing SDS-PAGE. The protease concentration was set to 25 nM, and Octet RED (Pall ForteBio Corp.) was used in the assay. The results of evaluation by reducing SDS-PAGE are shown in FIG. 41 and protease cleavage was confirmed in each IgG-antibody like molecule.

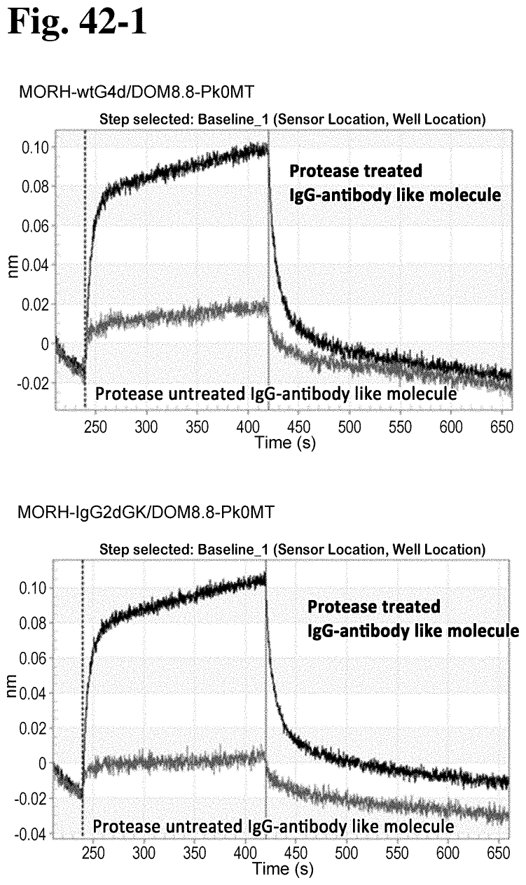

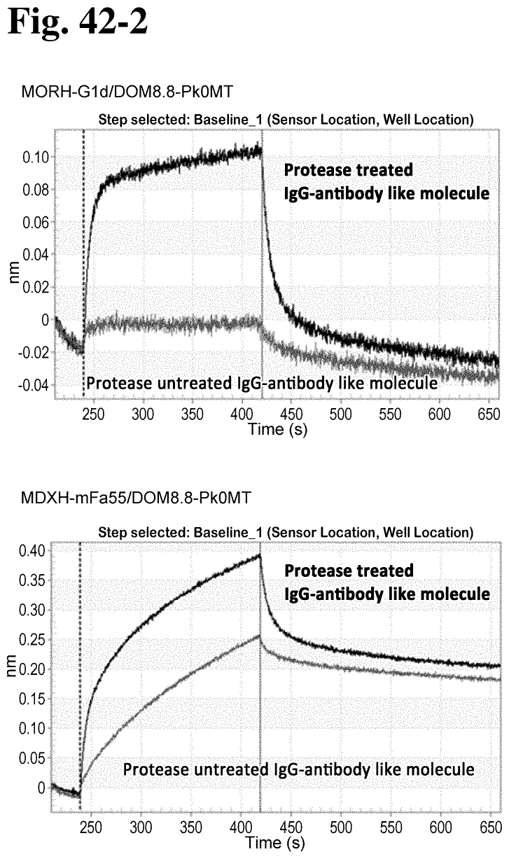

Next, the CD154 binding evaluation of VL released by protease treatment was conducted in the same way as in Example 3. N terminal Flag-tagged CD154 (SEQ ID NO: 539) was prepared by a method known to those skilled in the art. Then, Biotinylation of CD154 was conducted followed by the manufacture's protocol. Specifically, biotinylated CD154 was bound to a streptavidin sensor (Pall ForteBio Corp., 18-5021), and each cleaved IgG antibody-like molecule “Protease treated IgG-antibody like molecule” or “Protease untreated IgG-antibody like molecule” was allowed to act thereon, followed by binding evaluation at 27 degrees C. Sensorgrams showing continuous responses measured using Octet RED are shown in FIG. 42-1 and FIG. 42-2.

As a result, the IgG antibody-like molecules were confirmed to undergo protease cleavage at the protease cleavage sequence and retrieved antigen binding after protease cleavage.

### Example 21: Introduction of Amino Acid Alteration to Interface Site Between VH or VHH, and VL in Polypeptide to Attenuate Antigen Binding of Single Domain Antibody in the Absence of Proteases

As shown in the Example 4, even if VHH incorporated in a polypeptide does not lose its antigen binding activity immediately after association with particular VL, the antigen binding activity can be lost by introducing an association promoting mutation to an amino acid present at the interface between the VHH and the VL. In the case of VL single domain also could be introduced mutations to improve VH/VL association. As for enhancement of association between VH and VL several mutations have been reported in Nature Biotechnology volume 32, pages 191-198 (2014), Biophys. J. 75, 1473-1482 (1998), Protein Eng. Des. Sel. 23, 667-677 (2010), MAbs. 2017 February-March; 9(2): 182-212 and WO2013065708. Specifically the combinations of mutations were shown in the Table 15.

TABLE 15
Examples of mutations

| VH | CH1 | VL | CL |
|---|---|---|---|
| 39K, 62E | H172A, F174G | 1R, 38D, (36F) | L135Y, S176W |
| 39Y | — | 38R | — |
| | T192E | | N137K, S114A |
| | L143Q, S188V | | V133T, S176V |
| | F126C | | S121C |
| 39E | — | 38K | — |
| 39K | — | 38E | — |
| V37F, L45W | — | Y87A, F98M | — |

### Reference Example 1: Preparation of Biotinylated Plexin A1

Biotinylated plexin A1 (also referred to as biotin-labeled human plexin A1) was prepared by a method known to those skilled in the art. Specifically, a gene fragment encoding a specific sequence (AviTag sequence; SEQ ID NO: 36) to be biotinylated by biotin ligase and a gene fragment encoding a FLAG tag sequence (SEQ ID NO: 199; DYKDDDDK) were linked via a gene fragment encoding a linker constituted by glycine and serine to downstream of a gene fragment encoding the extracellular region of plexin A1. A gene fragment encoding a protein containing plexin A1 linked to the AviTag sequence and the FLAG tag sequence (SEQ ID NO: 200) was integrated to a vector for expression in animal cells. The constructed plasmid vector was transferred to FreeStyle 293 cells (Invitrogen Corp.) using 293Fectin (Invitrogen Corp.). In this operation, the cells were cotransfected with a gene for EBNA1 (SEQ ID NO: 57) expression and a gene for biotin ligase (BirA; SEQ ID NO: 58) expression, and biotin was further added thereto for the purpose of biotin-labeling plexin A1. The cells transfected according to the procedures mentioned above were cultured at 37 degrees C. under 8% CO₂ and caused to secrete the protein of interest (biotinylated plexin A1) into the culture supernatant. This cell culture solution was filtered through a 0.22 micro m bottle-top filter to obtain a culture supernatant.

A column was packed with Anti FLAG M2 agarose (Sigma-Aldrich Co. LLC, #A2220) to prepare a FLAG column. The FLAG column was equilibrated in advance with D-PBS(−). The culture supernatant was applied thereto to bind the biotinylated plexin A1 to the column. Subsequently, the biotinylated plexin A1 was eluted using FLAG peptide dissolved in D-PBS(−). Associates were removed from this eluate by gel filtration chromatography using HiLoad 26/600 Superdex 200 pg, 320 mL (GE Healthcare Japan Corp., 28-9893-36) to obtain purified biotinylated plexin A1.

The embodiments of the invention mentioned above are described in detail with reference to actual examples and illustrated examples with the aim of helping clear understanding. However, the description and illustration in the present specification should not be interpreted as limiting the scope of the present invention. The disclosure of all patent literatures and scientific literatures cited herein is explicitly incorporated herein by reference in its entirety.

### INDUSTRIAL APPLICABILITY

The polypeptide of the present invention comprising an antigen binding domain and a carrying moiety having a longer half-life in blood than that of the antigen binding domain and having an inhibiting domain that inhibits the binding activity of the antigen binding domain, and a pharmaceutical composition comprising the polypeptide can transport the antigen binding domain in blood while inhibited the antigen binding activity of the antigen binding domain. Also, use of the polypeptide of the present invention can allow the antigen binding domain to exert its antigen binding activity specifically at a disease site. Furthermore, since the antigen binding domain has a shorter half-life at the time of exerting its antigen binding activity than at the time of transport, the risk of acting systemically is decreased. Thus, the polypeptide and the pharmaceutical composition of the present invention are very useful in the treatment of a disease.

A single-domain antibody whose antigen binding activity is inhibited by associating with particular VL, VH or VHH can be screened for or produced as one example of the antigen binding domain to thereby efficiently produce the polypeptide of the present invention. Furthermore, a necessary antigen binding domain can be efficiently obtained when the polypeptide of the present invention is prepared by use of a library including the single-domain antibody whose antigen binding activity is inhibited by associating with particular VL, VH or VHH, as one example of the antigen binding domain that can be used in the polypeptide of the present invention.

### Sequence Listing

1. A polypeptide comprising an antigen binding domain that binds to IL-1R1 (IL-1R1 binding domain) and a carrying moiety, wherein the IL-R1 binding domain has a shorter half-life in blood than that of the carrying moiety that has an inhibiting domain that inhibits the IL-1R1 binding activity of the IL-1R1 binding domain.

2. The polypeptide according to claim 1, wherein the antigen binding domain is capable of being released from the polypeptide, and the antigen binding domain released from the polypeptide has higher antigen binding activity than that before the release.

3. The polypeptide according to claim 1 or 2, wherein the inhibiting domain of the carrying moiety associates with the antigen binding domain and thereby inhibits the antigen binding activity of the antigen binding domain.

4. The polypeptide according to claim 2 or 3, wherein the polypeptide comprises a cleavage site, wherein the cleavage site is cleaved so that the antigen binding domain becomes capable of being released from the polypeptide, or/and the association of the inhibiting domain of the carrying moiety with the antigen binding domain is canceled.

5. The polypeptide according to claim 4, wherein the cleavage site comprises a protease cleavage sequence.

6. The polypeptide according to any of claims 1 to 5, wherein the antigen binding domain comprises a single-domain antibody or IL-1R antagonist or is a single-domain antibody or IL-1R antagonist, wherein the inhibiting domain of the carrying moiety inhibits the antigen binding activity of the single-domain antibody or IL-1R antagonist.

7. The polypeptide according to any of claims 1 to 6, wherein the antigen binding domain comprises a single-domain antibody, and the inhibiting domain of the carrying moiety is VHH, antibody VH, or antibody VL, wherein the antigen binding activity of the single-domain antibody can be inhibited by the VHH, or the antibody VH, or the antibody VL.

8. The polypeptide according to any of claims 1 to 7, wherein the carrying moiety comprises an antibody constant region.

9. The polypeptide according to claim 8, wherein the polypeptide has a protease cleavage sequence, wherein the protease cleavage sequence is located near the boundary between the antigen binding domain and the antibody constant region.

10. A polypeptide comprising an IL-1R1 binding domain that has a shorter half-life in blood than that of a carrying moiety that has an inhibiting domain that inhibits the IL-1R1 binding activity of the IL-1R1 binding domain, wherein the IL-1R1 binding domain binds to an epitope within IL-1R1, and/or wherein the IL-1R1 domain competes for binding the epitope with a single-domain antibody VL selected from the group consisting of 1) and 2) below:
1) a single-domain antibody VL comprising the amino acid sequence of SEQ ID NO: 479, and
2) a single-domain antibody VL comprising the amino acid sequence of SEQ ID NO: 480.

11. A polypeptide comprising an antigen binding domain and a carrying moiety, wherein the antigen binding domain has a shorter half-life in blood than that of the carrying moiety that has an inhibiting domain that inhibits the antigen binding activity of the antigen binding domain, wherein the antigen is selected from the group consisting of IL-1alpha, IL-1beta and IL-1RAcP.

12. A pharmaceutical composition comprising the polypeptide of any of claims 1 to 11.

13. A pharmaceutical composition for treating a subject having an IL-1R1 mediated disease or disorder, comprising an effective amount of the polypeptide of any one of claims 1 to 11.

14. A polypeptide comprising an antigen binding domain VL and a carrying moiety, wherein the antigen binding domain VL has a shorter half-life in blood than that of the carrying moiety that has an inhibiting domain that inhibits the antigen binding activity of the antigen binding domain VL.

15. A polypeptide comprising an antigen binding domain VL and a carrying moiety, wherein the antigen binding domain VL has a shorter half-life in blood than that of the carrying moiety that has an inhibiting domain VL that inhibits the antigen binding activity of the antigen binding domain VL.
